(19) United States
(12) Patent Application Publication
(10) Pub. No.: US 2026/0232729 A1
(43) Pub. Date: Aug. 13, 2026

(54) METHODS FOR NON-VIRAL MANUFACTURING OF ENGINEERED IMMUNE CELLS

(71) Applicant: C3S2 GMBH, Muenchen (DE)

(72) Inventors: Mateusz Pawel POLTORAK, Grasbrunn (DE); Irina TREISE, Petershausen (DE); Sabine RADISCH, Bad Feilnbach (DE); Lisa DRESSLER, Rosenheim (DE); Katrin MANSKE, Munich (DE)

(73) Assignee: C3S2 GMBH, Muenchen (DE)

(21) Appl. No.: 19/152,681

(22) PCT Filed: Feb. 2, 2024
(86) PCT No.: PCT/EP2024/052653

§ 371 Date: Jul. 31, 2025

Related U.S. Application Data
(60) Provisional application No. 63/443,358, filed on Feb. 3, 2023.
(60) Provisional application No. 63/443,702, filed on Feb. 6, 2023.
(60) Provisional application No. 63/465,200, filed on May 9, 2023.

(51) Int. Cl. A61K 35/17; A61K 40/11; A61K 40/31; A61K 40/32; C07K 14/705; C12N 5/0783; C12N 9/12; C12N 9/22; C12N 15/113; C12N 15/87

(52) U.S. Cl. CPC A61K 35/17; A61K 40/11; A61K 40/31; A61K 40/32; C07K 14/7051; C12N 5/0636; C12N 9/1276; C12N 9/226; C12N 15/113; C12N 15/87; C12Y 207/07049; C12N 2310/20; C12N 2510/00

(57) ABSTRACT

Provided herein are methods for producing genetically engineered immune cells, such as T cells. In some aspects, the immune cells are genetically engineered by targeted integration of a transgene into a target site of a gene in the immune cells. In some embodiments, the genetically engineered immune cells are produced from a whole blood sample. In some aspects, the immune cells are genetically engineered following on-column stimulation of the immune cells. In some aspects, the immune cells are genetically engineered by non-viral gene delivery methods. Also provided herein are related cells, compositions, and uses.

### CROSS-REFERENCE TO RELATED APPLICATIONS

This application claims priority to U.S. Provisional Patent Application No. 63/443,358, filed Feb. 3, 2023, entitled “METHODS FOR NON-VIRAL MANUFACTURING OF ENGINEERED IMMUNE CELLS,” U.S. Provisional Patent Application No. 63/443,702, filed Feb. 6, 2023, entitled “METHODS FOR NON-VIRAL MANUFACTURING OF ENGINEERED IMMUNE CELLS,” and U.S. Provisional Patent Application No. 63/465,200, filed May 9, 2023, entitled, “METHODS FOR NON-VIRAL MANUFACTURING OF ENGINEERED IMMUNE CELLS,” which is herein incorporated by reference in its entirety for all purposes.

### INCORPORATION BY REFERENCE OF SEQUENCE LISTING

The present application is being filed along with a Sequence Listing in electronic format. The Sequence Listing is provided as a file entitled 735042027240SeqList.xml, created Feb. 1, 2024, which is 347,616 bytes in size. The information in the electronic format of the Sequence Listing is herein incorporated by reference in its entirety.

### FIELD

The present disclosure relates to methods for producing genetically engineered immune cells, such as T cells. In some aspects, the immune cells are genetically engineered by targeted integration of a transgene into a target site of a gene in the immune cells. In some embodiments, the genetically engineered immune cells are produced from a whole blood sample. In some aspects, the immune cells are genetically engineered following on-column stimulation of the immune cells. In some aspects, the immune cells are genetically engineered by non-viral gene delivery methods. Also provided herein are related cells, compositions, and uses.

### BACKGROUND

Various cell therapy methods are available for treating diseases and conditions. Among cell therapy methods are methods involving immune cells, such as T cells (e.g., CD4+ and CD8+ T cells), which may be genetically engineered with a recombinant receptor, such as a chimeric antigen receptor (CAR). Improved methods for producing engineered cells suitable for use in, for example, cell therapy, are needed. Provided are methods, cells, compositions, and uses that meet such needs.

### SUMMARY

Provided herein in some embodiments is a method for producing genetically engineered T cells, comprising: (a) adding a whole blood sample comprising a plurality of T cells to a stationary phase in an internal cavity of a chromatography column, the stationary phase comprising a selection agent that specifically binds to a selection marker expressed on the surface of the plurality of T cells, wherein specific binding of the selection agent to the selection marker effects the immobilization of the plurality of T cells on the stationary phase; (b) adding a T cell stimulatory reagent to the plurality of T cells immobilized on the stationary phase, wherein the T cell stimulatory reagent comprises (i) a primary agent that specifically binds to a member of a TCR/CD3 complex and (ii) a secondary agent that specifically binds to a T cell costimulatory molecule; (c) incubating the plurality of T cells immobilized on the stationary phase in the presence of the T cell stimulatory reagent under conditions to stimulate T cells of the plurality of T cells; (d) collecting T cells of the plurality of T cells from the chromatography column that are no longer immobilized after the incubating; and (e) introducing by non-viral gene delivery a nucleic acid molecule comprising a transgene encoding a recombinant protein under conditions for targeted integration of the transgene into a target site of a gene in one or more of the collected T cells; wherein the method produces genetically engineered T cells expressing the recombinant protein.

In some of any embodiments, the method comprises further incubating the collected T cells prior to the introducing of the nucleic acid molecule.

In some of any embodiments, the nucleic acid molecule is a DNA molecule. In some embodiments, the nucleic acid molecule is a modified DNA molecule. In some embodiments, the nucleic acid molecule is modified to enhance its stability.

In some of any embodiments, the nucleic acid molecule is a single-stranded DNA molecule or a double-stranded DNA molecule. In some of any embodiments, the nucleic acid molecule is a single-stranded DNA molecule. In some of any embodiments, the nucleic acid molecule is a double-stranded DNA molecule.

In some of any embodiments, the targeted integration is by Programmable Addition via Site-specific Targeting Elements (PASTE). In some of any embodiments, the PASTE comprises introducing one or more gene-editing agents for editing the gene in the one or more of the collected T cells.

In some of any embodiments, the targeted integration is by homology directed repair (HDR). In some of any embodiments, the HDR comprises introducing one or more gene-editing agents for inducing a genetic disruption in the gene in the one or more of the collected T cells.

In some of any embodiments, the introducing of the nucleic acid molecule and/or the one or more gene-editing agents is by electroporation. In some of any embodiments, the introducing of the nucleic acid molecule is by electroporation. In some of any embodiments, the introducing of the one or more gene-editing agents is by electroporation. In some of any embodiments, the introducing of the nucleic acid molecule and the one or more gene-editing agents is by electroporation.

In some of any embodiments, the conditions for targeted integration comprise cultivating the collected T cells under conditions to integrate the transgene into the target site.

Also provided herein in some embodiments is a method for producing genetically engineered T cells, comprising: (a) adding a whole blood sample comprising a plurality of T cells to a stationary phase in an internal cavity of a chromatography column, the stationary phase comprising a selection agent that specifically binds to a selection marker expressed on the surface of the plurality of T cells, wherein specific binding of the selection agent to the selection marker effects the immobilization of the plurality of T cells on the stationary phase; (b) adding a T cell stimulatory reagent to the plurality of T cells immobilized on the stationary phase, wherein the T cell stimulatory reagent comprises (i) a primary agent that specifically binds to a member of a TCR/CD3 complex and (ii) a secondary agent that specifically binds to a T cell costimulatory molecule; (c) incubating the plurality of T cells immobilized on the stationary phase in the presence of the T cell stimulatory reagent under conditions to stimulate T cells of the plurality of T cells; (d) collecting T cells of the plurality of T cells from the chromatography column that are no longer immobilized after the incubating; (e) further incubating the collected T cells; (f) after the further incubating, introducing into T cells of the collected T cells (i) a nucleic acid molecule comprising a transgene encoding a recombinant protein, wherein the nucleic acid molecule is a DNA molecule, and the introducing of the nucleic acid molecule is by non-viral gene delivery, and (ii) one or more gene-editing agents for inducing a genetic disruption in a gene in the T cells of the collected T cells, wherein the introducing of the nucleic acid molecule and/or the one or more gene-editing agents is by electroporation; and (g) cultivating the collected T cells under conditions to integrate by homology directed repair (HDR) the transgene into a target site of the gene in one or more of the collected T cells; wherein the method produces genetically engineered T cells expressing the recombinant protein.

In some of any embodiments, the introducing of the nucleic acid molecule is by electroporation. In some of any embodiments, the introducing of the one or more gene-editing agents is by electroporation. In some of any embodiments, the introducing of the nucleic acid molecule and the one or more gene-editing agents is by electroporation.

Also provided herein in some embodiments is a method for producing genetically engineered T cells, comprising: (a) adding a whole blood sample comprising a plurality of T cells to a stationary phase in an internal cavity of a chromatography column, the stationary phase comprising a selection agent that specifically binds to a selection marker expressed on the surface of the plurality of T cells, wherein specific binding of the selection agent to the selection marker effects the immobilization of the plurality of T cells on the stationary phase; (b) adding a T cell stimulatory reagent to the plurality of T cells immobilized on the stationary phase, wherein the T cell stimulatory reagent comprises (i) a primary agent that specifically binds to a member of a TCR/CD3 complex and (ii) a secondary agent that specifically binds to a T cell costimulatory molecule; (c) incubating the plurality of T cells immobilized on the stationary phase in the presence of the T cell stimulatory reagent under conditions to stimulate T cells of the plurality of T cells; (d) collecting T cells of the plurality of T cells from the chromatography column that are no longer immobilized after the incubating; (e) further incubating the collected T cells; (f) after the further incubating, introducing into T cells of the collected T cells (i) a nucleic acid molecule comprising a transgene encoding a recombinant protein, wherein the nucleic acid molecule is a DNA molecule, and the introducing of the nucleic acid molecule is by non-viral gene delivery, and (ii) one or more gene-editing agents for inducing a genetic disruption in a gene in the T cells of the collected T cells, wherein the introducing of the nucleic acid molecule and the one or more gene-editing agents is by electroporation; and (g) cultivating the collected T cells under conditions to integrate by homology directed repair (HDR) the transgene into a target site of the gene in one or more of the collected T cells; wherein the method produces genetically engineered T cells expressing the recombinant protein.

In some embodiments, the nucleic acid molecule is a modified DNA molecule. In some embodiments, the nucleic acid molecule is modified to enhance its stability.

In some of any embodiments, the nucleic acid molecule is a single-stranded DNA molecule or a double-stranded DNA molecule. In some of any embodiments, the nucleic acid molecule is a single-stranded DNA molecule. In some of any embodiments, the nucleic acid molecule is a double-stranded DNA molecule.

In some of any embodiments, the nucleic acid molecule is a double-stranded DNA molecule, a naked DNA molecule, and/or a closed-ended DNA molecule. In some of any embodiments, the nucleic acid molecule is a double-stranded DNA molecule. In some of any embodiments, the nucleic acid molecule is a naked DNA molecule. In some of any embodiments, the nucleic acid molecule is a closed-ended DNA molecule.

In some of any embodiments, the nucleic acid molecule is a naked DNA molecule and/or a closed-ended DNA molecule. In some of any embodiments, the nucleic acid molecule is a naked DNA molecule. In some of any embodiments, the nucleic acid molecule is a closed-ended DNA molecule. In some of any embodiments, the nucleic acid molecule is a naked closed-ended DNA molecule.

In some of any embodiments, the nucleic acid molecule is a naked, closed-ended, double-stranded DNA molecule. In some of any embodiments, the nucleic acid molecule is a closed-ended linear duplex (CELiD) DNA molecule, a minicircle DNA molecule, a minimalistic immunological-defined gene expression (MIDGE) DNA molecule, a ministring DNA molecule, a dumbbell-shaped linear duplex closed-ended DNA molecule, or a Doggybone™ DNA molecule. In some of any embodiments, the nucleic acid molecule is a closed-ended linear duplex (CELiD) DNA molecule. In some of any embodiments, the nucleic acid molecule is a minicircle DNA molecule. In some of any embodiments, the nucleic acid molecule is a minimalistic immunological-defined gene expression (MIDGE) DNA molecule. In some of any embodiments, the nucleic acid molecule is a ministring DNA molecule. In some of any embodiments, the nucleic acid molecule is a dumbbell-shaped linear duplex closed-ended DNA molecule. In some of any embodiments, the nucleic acid molecule is a Doggybone™ DNA molecule.

In some of any embodiments, the T cell stimulatory reagent is added in a cell medium. In some of any embodiments, the cell medium is a basal medium. In some of any embodiments, the cell medium is a serum free medium. In some of any embodiments, the cell medium comprises no cytokines or comprises recombinant IL-2, IL-7, and IL-15. In some of any embodiments, the cell medium comprises no cytokines. In some of any embodiments, the cell medium comprises recombinant IL-2, IL-7, and IL-15.

In some of any embodiments, the T cell stimulatory reagent comprises an oligomer of streptavidin or a streptavidin mutein molecule; the primary agent; and the secondary agent. In some of any embodiments, the primary agent is bound to a streptavidin or streptavidin mutein molecule of the oligomer. In some of any embodiments, the primary agent comprises a first streptavidin-binding partner that is bound to a streptavidin or streptavidin mutein molecule of the oligomer. In some of any embodiments, the secondary agent is bound to a streptavidin or streptavidin mutein molecule of the oligomer. In some of any embodiments, the secondary agent comprises a second streptavidin-binding partner that is bound to a streptavidin or streptavidin mutein molecule of the oligomer.

In some of any embodiments, the T cell stimulatory reagent comprises an oligomer of streptavidin or a streptavidin mutein molecule; the primary agent comprises a first streptavidin-binding partner that is bound to a streptavidin or streptavidin mutein molecule of the oligomer; and the secondary agent comprises a second streptavidin-binding partner that is bound to a streptavidin or streptavidin mutein molecule of the oligomer.

In some of any embodiments, the primary agent is an anti-CD3 agent. In some embodiments, the secondary agent is an anti-CD28 agent.

In some of any embodiments, the T cell stimulatory reagent is in soluble form.

Such a T cell stimulatory reagent is further described below and throughout the provided description.

In some of any embodiments, the T cell stimulatory reagent is added in an amount between or between about 0.1 μg and 20 μg, 0.4 μg and 8 μg, 0.8 μg and 4 μg, or 1 μg and 2 μg, each inclusive and each per 10⁶ T cells of the plurality of T cells immobilized or expected to be immobilized on the stationary phase. In some of any embodiments, the T cell stimulatory reagent is added in an amount between or between about 0.1 μg and 20 μg, inclusive, per 10⁶ T cells of the plurality of T cells immobilized or expected to be immobilized on the stationary phase. In some of any embodiments, the T cell stimulatory reagent is added in an amount between or between about 0.4 μg and 8 μg, inclusive, per 10⁶ T cells of the plurality of T cells immobilized or expected to be immobilized on the stationary phase. In some of any embodiments, the T cell stimulatory reagent is added in an amount between or between about 0.8 μg and 4 μg, inclusive, per 10⁶ T cells of the plurality of T cells immobilized or expected to be immobilized on the stationary phase. In some of any embodiments, the T cell stimulatory reagent is added in an amount between or between about 1 μg and 2 μg, inclusive, per 10⁶ T cells of the plurality of T cells immobilized or expected to be immobilized on the stationary phase.

In some of any embodiments, the T cell stimulatory reagent is added in an amount between or between about 0.1 μg and 20 μg, inclusive, per 10⁶ T cells of the plurality of T cells expected to be immobilized on the stationary phase. In some of any embodiments, the T cell stimulatory reagent is added in an amount between or between about 0.4 μg and 8 μg, inclusive, per 10⁶ T cells of the plurality of T cells expected to be immobilized on the stationary phase. In some of any embodiments, the T cell stimulatory reagent is added in an amount between or between about 0.8 μg and 4 μg, inclusive, per 10⁶ T cells of the plurality of T cells expected to be immobilized on the stationary phase. In some of any embodiments, the T cell stimulatory reagent is added in an amount between or between about 1 μg and 2 μg, inclusive, per 10⁶ T cells of the plurality of T cells expected to be immobilized on the stationary phase.

In some of any embodiments, the binding capacity of the stationary phase is between or between about 0.5 billion and 5 billion T cells expressing the selection marker, 0.5 billion and 3 billion T cells expressing the selection marker, or 1 billion and 2 billion T cells expressing the selection marker, each inclusive. In some of any embodiments, the binding capacity of the stationary phase is between or between about 0.5 billion and 5 billion T cells expressing the selection marker, inclusive. In some of any embodiments, the binding capacity of the stationary phase is between or between about 0.5 billion and 3 billion T cells expressing the selection marker, inclusive. In some of any embodiments, the binding capacity of the stationary phase is between or between about 1 billion and 2 billion T cells expressing the selection marker, inclusive.

In some of any embodiments, the T cell stimulatory reagent is added in an amount between or between about 0.1 mg and 20 mg, 0.4 mg and 8 mg, 0.8 mg and 4 mg, or 1 mg and 3 mg, each inclusive. In some of any embodiments, the T cell stimulatory reagent is added in an amount between or between about 0.1 mg and 20 mg, inclusive. In some of any embodiments, the T cell stimulatory reagent is added in an amount between or between about 0.4 mg and 8 mg, inclusive. In some of any embodiments, the T cell stimulatory reagent is added in an amount between or between about 0.8 mg and 4 mg, inclusive. In some of any embodiments, the T cell stimulatory reagent is added in an amount between or between about 1 mg and 3 mg, inclusive. In some of any embodiments, the T cell stimulatory reagent is added in an amount between or between about 1 mg and 2 mg, inclusive.

In some of any embodiments, the adding of the T cell stimulatory reagent is carried out within or within about 60 minutes, 30 minutes, or 15 minutes after the adding of the sample. In some of any embodiments, the adding of the T cell stimulatory reagent is carried out within or within about 60 minutes after the adding of the whole blood sample. In some of any embodiments, the adding of the T cell stimulatory reagent is carried out within or within about 30 minutes after the adding of the whole blood sample. In some of any embodiments, the adding of the T cell stimulatory reagent is carried out within or within about 15 minutes after the adding of the whole blood sample.

In some of any embodiments, the incubating is carried out in a cell medium. In some of any embodiments, the cell medium is a basal medium. In some of any embodiments, the cell medium is a serum free medium. In some of any embodiments, the cell medium comprises no cytokines or comprises recombinant IL-2, IL-7, and IL-15. In some of any embodiments, the cell medium comprises no cytokines. In some of any embodiments, the cell medium comprises recombinant IL-2, IL-7, and IL-15.

In some of any embodiments, the incubating is carried out at a temperature between or between about 35° C. and about 39° C. In some of any embodiments, the incubating is carried out at a temperature of or of about 37° C.

In some of any embodiments, the incubating is carried out for between or between about 0.5 hour and 8 hours, 2 hours and 6 hours, or 3 hours and 5 hours, each inclusive. In some of any embodiments, the incubating is carried out for between or between about 0.5 hour and 8 hours, inclusive. In some of any embodiments, the incubating is carried out for between or between about 2 hours and 6 hours, inclusive. In some of any embodiments, the incubating is carried out for between or between about 3 hours and 5 hours, inclusive. In some of any embodiments, the incubating is carried out for or for about 4 hours.

In some of any embodiments, the collecting comprises adding a wash buffer to the stationary phase to collect the T cells of the plurality of T cells.

In some of any embodiments, the wash buffer is a cell medium. In some of any embodiments, the cell medium is a basal medium. In some of any embodiments, the cell medium is a serum free medium. In some of any embodiments, the cell medium comprises no cytokines or comprises recombinant IL-2, IL-7, and IL-15. In some of any embodiments, the cell medium comprises no cytokines. In some of any embodiments, the cell medium comprises recombinant IL-2, IL-7, and IL-15.

In some of any embodiments, the wash buffer does not comprise a competition agent. In some of any embodiments, the competition agent is biotin.

In some of any embodiments, the collecting is carried out between or between about 0.5 hours and 8 hours, 2 hours and 6 hours, or 3 hours and 5 hours, each inclusive, after the adding of the T cell stimulatory reagent. In some of any embodiments, the collecting is carried out between or between about 0.5 hours and 8 hours, inclusive, after the adding of the T cell stimulatory reagent. In some of any embodiments, the collecting is carried out between or between about 2 hours and 6 hours, inclusive, after the adding of the T cell stimulatory reagent. In some of any embodiments, the collecting is carried out between or between about 3 hours and 5 hours, inclusive, after the adding of the T cell stimulatory reagent. In some of any embodiments, the collecting is carried out at or about 4 hours after the adding of the T cell stimulatory reagent.

In some of any embodiments, the further incubating is carried out in the presence of the T cell stimulatory reagent.

In some of any embodiments, the further incubating is carried out in a cell medium. In some of any embodiments, the cell medium is a basal medium. In some of any embodiments, the cell medium is a serum free medium. In some of any embodiments, the cell medium comprises no cytokines or comprises recombinant IL-2, IL-7, and IL-15. In some of any embodiments, the cell medium comprises no cytokines. In some of any embodiments, the cell medium comprises recombinant IL-2, IL-7, and IL-15.

In some of any embodiments, the further incubating is carried out at a temperature between or between about 35° C. and about 39° C. In some of any embodiments, the further incubating is carried out at a temperature of or of about 37° C.

In some of any embodiments, the further incubating is carried out for between or between about 10 hours and 30 hours, 16 hours and 24 hours, or 18 hours and 22 hours, each inclusive. In some of any embodiments, the further incubating is carried out for between or between about 10 hours and 30 hours, inclusive. In some of any embodiments, the further incubating is carried out for between or between about 16 hours and 24 hours, inclusive. In some of any embodiments, the further incubating is carried out for between or between about 18 hours and 22 hours, inclusive. In some of any embodiments, the further incubating is carried out for or for about 20 hours.

In some of any embodiments, the method comprises removing the T cell stimulatory reagent from the collected T cells prior to the introducing of the one or more gene-editing agents. In some of any embodiments, the method comprises removing the T cell stimulatory reagent from the collected T cells prior to the introducing of the nucleic acid molecule. In some of any embodiments, the removing is carried out after the further incubating. In some of any embodiments, the removing comprises washing the collected T cells.

In some of any embodiments, the T cell stimulatory reagent comprises an oligomer of streptavidin or a streptavidin mutein molecule; the primary agent comprises a first streptavidin-binding partner that is bound to a streptavidin or streptavidin mutein molecule of the oligomer; the secondary agent comprises a second streptavidin-binding partner that is bound to a streptavidin or streptavidin mutein molecule of the oligomer; and the method comprises disrupting the binding between the first and second streptavidin-binding partners and the streptavidin or streptavidin mutein molecules prior to the introducing of the one or more gene-editing agents. In some of any embodiments, the T cell stimulatory reagent comprises an oligomer of streptavidin or a streptavidin mutein molecule; the primary agent comprises a first streptavidin-binding partner that is bound to a streptavidin or streptavidin mutein molecule of the oligomer; the secondary agent comprises a second streptavidin-binding partner that is bound to a streptavidin or streptavidin mutein molecule of the oligomer; and the method comprises disrupting the binding between the first and second streptavidin-binding partners and the streptavidin or streptavidin mutein molecules prior to the introducing of the nucleic acid molecule. In some of any embodiments, the disrupting is carried out after the further incubating. In some of any embodiments, the disrupting is by adding a competition agent to the collected T cells that reverses the binding between the first and second streptavidin-binding partners and the streptavidin or streptavidin mutein molecules. In some of any embodiments, the competition agent is biotin.

In some of any embodiments, the introducing of the one or more gene-editing agents is carried out prior to or concurrently with the introducing of the nucleic acid molecule. In some of any embodiments, the introducing of the one or more gene-editing agents is carried out prior to the introducing of the nucleic acid molecule. In some of any embodiments, the introducing of the one or more gene-editing agents is carried out concurrently with the introducing of the nucleic acid molecule.

In some of any embodiments, the introducing of the one or more gene-editing agents is carried out between or between about 12 hours and 36 hours, 18 hours and 30 hours, or 22 hours and 26 hours, each inclusive, after the adding of the T cell stimulatory reagent. In some of any embodiments, the introducing of the one or more gene-editing agents is carried out between or between about 12 hours and 36 hours, inclusive, after the adding of the T cell stimulatory reagent. In some of any embodiments, the introducing of the one or more gene-editing agents is carried out between or between about 18 hours and 30 hours, inclusive, after the adding of the T cell stimulatory reagent. In some of any embodiments, the introducing of the one or more gene-editing agents is carried out between or between about 22 hours and 26 hours, inclusive, after the adding of the T cell stimulatory reagent. In some of any embodiments, the introducing of the one or more gene-editing agents is carried out at or about 24 hours after the adding of the T cell stimulatory reagent.

In some of any embodiments, the nucleic acid molecule is introduced in a cell medium comprising the nucleic acid molecule. In some of any embodiments, the cell medium is a basal medium. In some of any embodiments, the cell medium is a serum free medium. In some of any embodiments, the cell medium comprises no cytokines or comprises recombinant IL-2, IL-7, and IL-15. In some of any embodiments, the cell medium comprises no cytokines. In some of any embodiments, the cell medium comprises recombinant IL-2, IL-7, and IL-15.

In some of any embodiments, the introducing of the nucleic acid molecule is carried out between or between about 12 hours and 36 hours, 18 hours and 30 hours, or 22 hours and 26 hours, each inclusive, after the adding of the T cell stimulatory reagent. In some of any embodiments, the introducing of the nucleic acid molecule is carried out between or between about 12 hours and 36 hours, inclusive, after the adding of the T cell stimulatory reagent. In some of any embodiments, the introducing of the nucleic acid molecule is carried out between or between about 18 hours and 30 hours, inclusive, after the adding of the T cell stimulatory reagent. In some of any embodiments, the introducing of the nucleic acid molecule is carried out between or between about 22 hours and 26 hours, inclusive, after the adding of the T cell stimulatory reagent. In some of any embodiments, the introducing of the nucleic acid molecule is carried out at or about 24 hours after the adding of the T cell stimulatory reagent.

In some of any embodiments, the cultivating is carried out in the presence of the nucleic acid molecule.

In some of any embodiments, the cultivating is carried out in a cell medium. In some of any embodiments, the cell medium is a basal medium. In some of any embodiments, the cell medium is a serum free medium. In some of any embodiments, the cell medium comprises no cytokines or comprises recombinant IL-2, IL-7, and IL-15. In some of any embodiments, the cell medium comprises no cytokines. In some of any embodiments, the cell medium comprises recombinant IL-2, IL-7, and IL-15.

In some of any embodiments, the cultivating is carried out at a temperature between or between about 35° C. and about 39° C. In some of any embodiments, the cultivating is carried out at a temperature of or of about 37° C.

In some of any embodiments, the cultivating is carried out for between or between about 12 hours and 36 hours, 18 hours and 30 hours, or 22 hours and 26 hours, each inclusive. In some of any embodiments, the cultivating is carried out for between or between about 12 hours and 36 hours, inclusive. In some of any embodiments, the cultivating is carried out for between or between about 18 hours and 30 hours, inclusive. In some of any embodiments, the cultivating is carried out for between or between about 22 hours and 26 hours, inclusive. In some of any embodiments, the cultivating is carried out for or for about 24 hours.

In some of any embodiments, the method comprises harvesting the genetically engineered T cells expressing the recombinant protein.

In some of any embodiments, the harvesting is carried out between or between about 36 hours and 60 hours, 42 hours and 54 hours, or 46 hours and 50 hours, each inclusive, after the adding of the whole blood sample. In some of any embodiments, the harvesting is carried out between or between about 36 hours and 60 hours, inclusive, after the adding of the whole blood sample. In some of any embodiments, the harvesting is carried out between or between about 42 hours and 54 hours, inclusive, after the adding of the whole blood sample. In some of any embodiments, the harvesting is carried out between or between about 46 hours and 50 hours, inclusive, after the adding of the whole blood sample. In some of any embodiments, the harvesting is carried out at or about 48 hours after the adding of the whole blood sample.

In some of any embodiments, the harvesting is carried out between or between about 36 hours and 60 hours, 42 hours and 54 hours, or 46 hours and 50 hours, each inclusive, after the adding of the T cell stimulatory reagent. In some of any embodiments, the harvesting is carried out between or between about 36 hours and 60 hours, inclusive, after the adding of the T cell stimulatory reagent. In some of any embodiments, the harvesting is carried out between or between about 42 hours and 54 hours, inclusive, after the adding of the T cell stimulatory reagent. In some of any embodiments, the harvesting is carried out between or between about 46 hours and 50 hours, inclusive, after the adding of the T cell stimulatory reagent. In some of any embodiments, the harvesting is carried out at or about 48 hours after the adding of the T cell stimulatory reagent.

In some of any embodiments, the harvesting is carried out between or between about 12 hours and 36 hours, 18 hours and 30 hours, or 22 hours and 26 hours, each inclusive, after the introducing of the one or more gene-editing agents. In some of any embodiments, the harvesting is carried out between or between about 12 hours and 36 hours, inclusive, after the introducing of the one or more gene-editing agents. In some of any embodiments, the harvesting is carried out between or between about 18 hours and 30 hours, inclusive, after the introducing of the one or more gene-editing agents. In some of any embodiments, the harvesting is carried out between or between about 22 hours and 26 hours, inclusive, after the introducing of the one or more gene-editing agents. In some of any embodiments, the harvesting is carried out at or about 24 hours after the introducing of the one or more gene-editing agents.

In some of any embodiments, the harvesting is carried out between or between about 12 hours and 36 hours, 18 hours and 30 hours, or 22 hours and 26 hours, each inclusive, after the introducing of the nucleic acid molecule. In some of any embodiments, the harvesting is carried out between or between about 12 hours and 36 hours, inclusive, after the introducing of the nucleic acid molecule. In some of any embodiments, the harvesting is carried out between or between about 18 hours and 30 hours, inclusive, after the introducing of the nucleic acid molecule. In some of any embodiments, the harvesting is carried out between or between about 22 hours and 26 hours, inclusive, after the introducing of the nucleic acid molecule. In some of any embodiments, the harvesting is carried out at or about 24 hours after the introducing of the nucleic acid molecule.

In some of any embodiments, the method comprises formulating the harvested genetically engineered T cells for cryopreservation or administration to a subject. In some of any embodiments, the method comprises formulating the harvested genetically engineered T cells for cryopreservation. In some of any embodiments, the method comprises formulating the harvested genetically engineered T cells for administration to a subject.

In some of any embodiments, the harvested genetically engineered T cells are formulated in the presence of a cryoprotectant or a pharmaceutically acceptable excipient. In some of any embodiments, the harvested genetically engineered T cells are formulated in the presence of a cryoprotectant. In some of any embodiments, the harvested genetically engineered T cells are formulated in the presence of a pharmaceutically acceptable excipient.

In some of any embodiments, the plurality of T cells are primary T cells from a human subject.

In some of any embodiments, the selection marker is selected from the group consisting of CD3, CD4, CD8, CD45RA, CD27, CD28, and CCR7. In some of any embodiments, the selection marker is CD3, CD4, or CD8. In some of any embodiments, the selection marker is CD3. In some of any embodiments, the selection marker is CD4. In some of any embodiments, the selection marker is CD8.

In some of any embodiments, the selection agent comprises an antibody or antibody fragment that specifically binds to the selection marker. In some of any embodiments, the antibody or antibody fragment of the selection agent is a monovalent antibody fragment. In some of any embodiments, the antibody or antibody fragment of the selection agent is a Fab fragment.

In some of any embodiments, the T cell stimulatory reagent comprises an oligomer of streptavidin or a streptavidin mutein molecule; the primary agent comprises a first streptavidin-binding partner that is bound to a streptavidin or streptavidin mutein molecule of the oligomer; and the secondary agent comprises a second streptavidin-binding partner that is bound to a streptavidin or streptavidin mutein molecule of the oligomer.

In some of any embodiments, the T cell stimulatory reagent consists or consists essentially of the oligomer, primary agent, and secondary agent.

In some of any embodiments, the oligomer comprises between or between about 500 and 5000 tetramers, 1000 and 4000 tetramers, or 2000 and 3000 tetramers, each inclusive, of the streptavidin or streptavidin mutein molecule. In some of any embodiments, the oligomer comprises between or between about 500 and 5000 tetramers, inclusive, of the streptavidin or streptavidin mutein molecule. In some of any embodiments, the oligomer comprises between or between about 1000 and 4000 tetramers, inclusive, of the streptavidin or streptavidin mutein molecule. In some of any embodiments, the oligomer comprises between or between about 2000 and 3000 tetramers, inclusive, of the streptavidin or streptavidin mutein molecule. In some of any embodiments, the oligomer comprises at or about 2400 tetramers of the streptavidin or streptavidin mutein molecule.

In some of any embodiments, the oligomer is of the streptavidin mutein molecule.

In some of any embodiments, the streptavidin mutein molecule comprises the amino acid sequence IGAR (SEQ ID NO: 133) or VTAR (SEQ ID NO: 134) at sequence positions corresponding to positions 44 to 47 of the sequence of amino acids set forth in SEQ ID NO: 1. In some of any embodiments, the streptavidin mutein molecule comprises the amino acid sequence IGAR (SEQ ID NO: 133) at sequence positions corresponding to positions 44 to 47 of the sequence of amino acids set forth in SEQ ID NO: 1. In some of any embodiments, the streptavidin mutein molecule comprises the amino acid sequence VTAR (SEQ ID NO: 134) at sequence positions corresponding to positions 44 to 47 of the sequence of amino acids set forth in SEQ ID NO: 1.

In some of any embodiments, the streptavidin mutein molecule begins N-terminally in the region of amino acid positions 10 to 16 of SEQ ID NO: 1 and terminates C-terminally in the region of amino acid positions 133 to 142 of SEQ ID NO: 1.

In some of any embodiments, the streptavidin mutein molecule comprises the amino acid sequence set forth in any one of SEQ ID NO: 3-6, 27, 28, 104, 105, and 136. In some of any embodiments, the streptavidin mutein molecule comprises the amino acid sequence set forth in SEQ ID NO: 6.

In some of any embodiments, the first streptavidin-binding partner is at the C-terminus of the primary agent; and/or the second streptavidin-binding partner is at the C-terminus of the secondary agent. In some of any embodiments, the first streptavidin-binding partner is at the C-terminus of the primary agent. In some of any embodiments, the second streptavidin-binding partner is at the C-terminus of the secondary agent. In some of any embodiments, the first streptavidin-binding partner is at the C-terminus of the primary agent; and the second streptavidin-binding partner is at the C-terminus of the secondary agent.

In some of any embodiments, the first and/or second streptavidin-binding partner is a streptavidin-binding peptide. In some of any embodiments, the first streptavidin-binding partner is a streptavidin-binding peptide. In some of any embodiments, the second streptavidin-binding partner is a streptavidin-binding peptide. In some of any embodiments, the first and second streptavidin-binding partner is a streptavidin-binding peptide.

In some of any embodiments, the streptavidin-binding peptide of the first and/or second streptavidin-binding partner comprises the amino acid sequence set forth in any one of SEQ ID NO: 7, 8, and 15-19. In some of any embodiments, the streptavidin-binding peptide of the first streptavidin-binding partner comprises the amino acid sequence set forth in any one of SEQ ID NO: 7, 8, and 15-19. In some of any embodiments, the streptavidin-binding peptide of the second streptavidin-binding partner comprises the amino acid sequence set forth in any one of SEQ ID NO: 7, 8, and 15-19. In some of any embodiments, the streptavidin-binding peptide of the first and second streptavidin-binding partner comprises the amino acid sequence set forth in any one of SEQ ID NO: 7, 8, and 15-19.

In some of any embodiments, the streptavidin-binding peptide of the first and/or second streptavidin-binding partner comprises the amino acid sequence set forth in SEQ ID NO: 16. In some of any embodiments, the streptavidin-binding peptide of the first streptavidin-binding partner comprises the amino acid sequence set forth in SEQ ID NO: 16. In some of any embodiments, the streptavidin-binding peptide of the second streptavidin-binding partner comprises the amino acid sequence set forth in SEQ ID NO: 16. In some of any embodiments, the streptavidin-binding peptide of the first and second streptavidin-binding partner comprises the amino acid sequence set forth in SEQ ID NO: 16.

In some of any embodiments, the member of the TCR/CD3 complex is CD3.

In some of any embodiments, the T cell costimulatory molecule is CD28, CD90 (Thy-1), CD95 (Apo-/Fas), CD137 (4-1BB), CD154 (CD40L), ICOS, LAT, CD27, OX40, or HVEM. In some of any embodiments, the T cell costimulatory molecule is CD28.

In some of any embodiments, the primary agent comprises an antibody or antibody fragment that specifically binds to the member of the TCR/CD3 complex; and/or the secondary agent comprises an antibody or antibody fragment that specifically binds to the T cell costimulatory agent. In some of any embodiments, the primary agent comprises an antibody or antibody fragment that specifically binds to the member of the TCR/CD3 complex. In some of any embodiments, the secondary agent comprises an antibody or antibody fragment that specifically binds to the T cell costimulatory agent. In some of any embodiments, the primary agent comprises an antibody or antibody fragment that specifically binds to the member of the TCR/CD3 complex; and the secondary agent comprises an antibody or antibody fragment that specifically binds to the T cell costimulatory agent.

In some of any embodiments, the antibody or antibody fragment of the primary agent comprises a heavy chain, and the first streptavidin-binding partner is fused to the C-terminus of the heavy chain of the primary agent; and/or the antibody or antibody fragment of the secondary agent comprises a heavy chain, and the second streptavidin-binding partner is fused to the C-terminus of the heavy chain of the secondary agent. In some of any embodiments, the antibody or antibody fragment of the primary agent comprises a heavy chain, and the first streptavidin-binding partner is fused to the C-terminus of the heavy chain of the primary agent. In some of any embodiments, the antibody or antibody fragment of the secondary agent comprises a heavy chain, and the second streptavidin-binding partner is fused to the C-terminus of the heavy chain of the secondary agent. In some of any embodiments, the antibody or antibody fragment of the primary agent comprises a heavy chain, and the first streptavidin-binding partner is fused to the C-terminus of the heavy chain of the primary agent; and the antibody or antibody fragment of the secondary agent comprises a heavy chain, and the second streptavidin-binding partner is fused to the C-terminus of the heavy chain of the secondary agent.

In some of any embodiments, the antibody or antibody fragment of the primary and/or secondary agent is a monovalent antibody fragment. In some of any embodiments, the antibody or antibody fragment of the primary agent is a monovalent antibody fragment. In some of any embodiments, the antibody or antibody fragment of the secondary agent is a monovalent antibody fragment. In some of any embodiments, the antibody or antibody fragment of the primary and secondary agent is a monovalent antibody fragment.

In some of any embodiments, the antibody or antibody fragment of the primary and/or secondary agent is a Fab fragment. In some of any embodiments, the antibody or antibody fragment of the primary agent is a Fab fragment. In some of any embodiments, the antibody or antibody fragment of the secondary agent is a Fab fragment. In some of any embodiments, the antibody or antibody fragment of the primary and secondary agent is a Fab fragment.

In some of any embodiments, the primary agent comprises an anti-CD3 antibody or antibody fragment, and the secondary agent comprises an anti-CD28 antibody or antibody fragment. In some of any embodiments, the primary agent comprises an anti-CD3 Fab fragment, and the secondary agent comprises an anti-CD28 Fab fragment.

In some of any embodiments, the gene is the T cell receptor alpha constant (TRAC) gene. In some of any embodiments, the target site is within the sequence set forth in SEQ ID NO: 250.

In some of any embodiments, the nucleic acid molecule comprises a 5′ homology arm and a 3′ homology arm comprising sequences homologous to nucleic acid sequences surrounding the target site, the nucleic acid molecule comprising the structure [5′ homology arm]-[transgene]-[3′ homology arm].

In some of any embodiments, the 5′ homology arm and the 3′ homology arm comprise sequences homologous to sequences of the TRAC gene surrounding the target site.

In some of any embodiments, the 5′ homology arm comprises a sequence comprising at least or at least about 150, 200, 250, 300, 350, 400, 450, 500, 550, or 600 contiguous nucleotides of a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 248. In some of any embodiments, the 5′ homology arm comprises at least or at least about 150, 200, 250, 300, 350, 400, 450, 500, 550, or 600 contiguous nucleotides of the sequence set forth In SEQ ID NO: 248. In some of any embodiments, the 5′ homology arm comprises the sequence set forth in SEQ ID NO: 248.

In some of any embodiments, the 3′ homology arm comprises a sequence comprising at least or at least about 150, 200, 250, 300, 350, 400, 450, 500, 550, or 600 contiguous nucleotides of a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 249. In some of any embodiments, the 3′ homology arm comprises at least or at least about 150, 200, 250, 300, 350, 400, 450, 500, 550, or 600 contiguous nucleotides of the sequence set forth in SEQ ID NO: 249. In some of any embodiments, the 3′ homology arm comprises the sequence set forth in SEQ ID NO: 249.

In some of any embodiments, the 5′ homology arm comprises the sequence set forth in SEQ ID NO: 248, and the 3′ homology arm comprises the sequence set forth in SEQ ID NO: 249.

In some of any embodiments, transcription of the integrated transgene is under the control of a promoter comprised by the nucleic acid molecule. In some of any embodiments, the promoter is a human elongation factor 1 alpha (EF1α) promoter. In some of any embodiments, the promoter comprises the sequence set forth in SEQ ID NO: 247.

In some of any embodiments, the recombinant protein is a recombinant receptor. In some of any embodiments, the recombinant receptor is a T cell receptor or a chimeric antigen receptor. In some of any embodiments, the recombinant receptor is a T cell receptor. In some of any embodiments, the recombinant receptor is a chimeric antigen receptor.

In some of any embodiments, the one or more gene-editing agents comprise (i) a gene-editing nuclease or nuclease combination or (ii) a nucleic acid molecule comprising one or more sequences encoding the gene-editing nuclease or nuclease combination. In some of any embodiments, the one or more gene-editing agents comprise a gene-editing nuclease or nuclease combination.

In some of any embodiments, the gene-editing nuclease or nuclease combination specifically recognizes a nucleic acid sequence near or comprising the target site. In some of any embodiments, the gene-editing nuclease or nuclease combination specifically recognizes a nucleic acid sequence comprising the target site.

In some of any embodiments, the nucleic acid sequence comprising the target site comprises the sequence set forth in SEQ ID NO: 250.

In some of any embodiments, the gene-editing nuclease or nuclease combination is a zinc finger nuclease, a transcription activator-like effector nuclease, or a CRISPR-Cas combination. In some of any embodiments, the gene-editing nuclease or nuclease combination is a CRISPR-Cas combination.

In some of any embodiments, the CRISPR-Cas combination comprises a CRISPR-Cas nickase, reverse transcriptase, and serine integrase.

In some of any embodiments, the CRISPR-Cas combination comprises a guide RNA comprising a targeting sequence that is complementary to the nucleic acid sequence comprising the target site. In some of any embodiments, the CRISPR-Cas combination is a ribonucleoprotein complex comprising the guide RNA and a Cas protein. In some of any embodiments, the Cas protein is a S. pyogenes Cas protein.

In some of any embodiments, the CRISPR-Cas combination is a CRISPR-Cas9 combination or a CRISPR-Cas12 combination. In some of any embodiments, the CRISPR-Cas combination is a CRISPR-Cas9 combination. In some of any embodiments, the CRISPR-Cas combination is a CRISPR-Cas12 combination.

In some of any embodiments, the targeting sequence comprises the sequence set forth in any one of SEQ ID NO: 144-175. In some of any embodiments, the targeting sequence comprises the sequence set forth in SEQ ID NO: 148.

In some of any embodiments, the method is performed ex vivo.

Also provided herein in some embodiments is a genetically engineered T cell produced by any of the provided methods, wherein the genetically engineered T cell expresses the recombinant protein.

In some of any embodiments, the transgene is integrated into the target site of the gene in the genetically engineered T cell. In some of any embodiments, the gene is the T cell receptor alpha constant (TRAC) gene. In some of any embodiments, wherein the target site is within the sequence set forth in SEQ ID NO: 250.

In some of any embodiments, wherein the recombinant protein is a recombinant receptor. In some of any embodiments, the recombinant receptor is a T cell receptor or a chimeric antigen receptor. In some of any embodiments, the recombinant receptor is a T cell receptor. In some of any embodiments, the recombinant receptor is a chimeric antigen receptor.

Also provided herein in some embodiments is a population of T cells comprising a plurality of any of the provided genetically engineered T cells.

In some of any embodiments, the plurality of genetically engineered T cells are at least 10%, 15%, or 20% of the population of T cells. In some of any embodiments, the plurality of genetically engineered T cells are at least 10% of the population of T cells. In some of any embodiments, the plurality of genetically engineered T cells are at least 15% of the population of T cells. In some of any embodiments, the plurality of genetically engineered T cells are at least 20% of the population of T cells.

In some of any embodiments, the gene is disrupted in at least 85%, 90%, or 95% of the T cells of the population of T cells. In some of any embodiments, the gene is disrupted in at least 85% of the T cells of the population of T cells. In some of any embodiments, the gene is disrupted in at least 90% of the T cells of the population of T cells. In some of any embodiments, the gene is disrupted in at least 95% of the T cells of the population of T cells.

In some of any embodiments, the gene is the T cell receptor alpha constant (TRAC) gene.

Also provided herein in some embodiments is a pharmaceutical composition comprising any of the provided populations of T cells and a pharmaceutically acceptable excipient.

Also provided herein in some embodiments is a method of treatment, comprising administering to a subject having a disease or condition any of the provided pharmaceutical compositions.

In some of any embodiments, the recombinant protein is a recombinant receptor that targets an antigen expressed on a target cell associated with the disease or condition.

Also provided herein in some embodiments is a method of cytolytic killing of a target cell, comprising contacting a target cell with any of the provided populations.

Also provided herein in some embodiments is a method of cytolytic killing of a target cell, comprising contacting a target cell with any of the provided pharmaceutical compositions.

In some of any embodiments, the contacting is performed ex vivo.

In some of any embodiments, the contacting is performed in vivo. In some of any embodiments, the contacting is by administering the pharmaceutical composition to a subject having a disease or condition. In some of any embodiments, the target cell is associated with the disease or condition, and the recombinant protein is a recombinant receptor that targets an antigen expressed on the target cell.

In some of any embodiments, the recombinant receptor is a T cell receptor or a chimeric antigen receptor. In some of any embodiments, the recombinant receptor is a T cell receptor. In some of any embodiments, the recombinant receptor is a chimeric antigen receptor.

In some of any embodiments, the pharmaceutical composition is for use in treating a disease or disorder in a subject.

In some of any embodiments, the recombinant protein is a recombinant receptor that targets an antigen expressed on a cell associated with the disease or condition.

In some of any embodiments, the recombinant receptor is a T cell receptor or a chimeric antigen receptor. In some of any embodiments, the recombinant receptor is a T cell receptor. In some of any embodiments, the recombinant receptor is a chimeric antigen receptor.

Also provided herein in some embodiments is use of any of the provided pharmaceutical compositions for treating a disease or disorder in a subject.

Also provided herein in some embodiments is use of any of the provided pharmaceutical compositions for the manufacture of a medicament for treating a disease or disorder in a subject.

In some of any embodiments, the recombinant protein is a recombinant receptor that targets an antigen expressed on a cell associated with the disease or condition.

In some of any embodiments, the recombinant receptor is a T cell receptor or a chimeric antigen receptor. In some of any embodiments, the recombinant receptor is a T cell receptor. In some of any embodiments, the recombinant receptor is a chimeric antigen receptor.

### BRIEF DESCRIPTION OF THE DRAWINGS

FIG. 1A shows depletion, total nucleated cell count (TNC), and purity for positive fractions collected from multiple whole blood sample loadings onto an anti-CD3 affinity chromatography column.

FIG. 1B shows CD3 and CAR expression in cells following the on-column stimulation, elution, and non-viral engineering of T cells selected from a whole blood sample loaded onto an anti-CD3 affinity chromatography column.

FIG. 1C shows viability, knockout (KO) efficiency, and knock-in (KI) efficiency over time in cells following engineering.

FIG. 1D shows cell growth of total cells, KO cells, and KI cells over time following engineering.

### DETAILED DESCRIPTION

Provided herein in some embodiments are methods for producing genetically engineered immune cells, e.g., T cells. In some embodiments, the provided methods are any described herein, for instance in Section I. In some embodiments, the provided methods are performed ex vivo.

In some embodiments, the provided methods involve stimulating and engineering immune cells, e.g., T cells. In some embodiments, the provided methods involve selecting, stimulating, and engineering immune cells, e.g., T cells. In some embodiments, the stimulating is by on-column stimulation of the immune cells, e.g., T cells, wherein the immune cells, e.g., T cells, are immobilized on a stationary phase in an internal cavity of a chromatography column during at least a portion of incubation in the presence of a stimulatory reagent, e.g., T cell stimulatory reagent, that is added to the stationary phase. In some embodiments, the immune cells, e.g., T cells, are immobilized via specific binding of a selection agent contained by the stationary phase to a selection marker expressed on the surface of the immune cells, e.g., T cells. In some embodiments, the provided methods involve selecting the immune cells by adding a sample containing the immune cells, e.g., T cells, to the stationary phase prior to the stimulating, whereby the immune cells, e.g., T cells, become immobilized to the stationary phase via the selection agent for the on-column stimulation. In some embodiments, the sample is a whole blood sample.

In some embodiments, the immune cells, e.g., T cells, are collected from the chromatography column following the on-column stimulation. In some embodiments, the collected immune cells, e.g., T cells, are those that become no longer immobilized following the on-column stimulation. In some aspects, the on-column stimulation facilitates detachment of the immobilized immune cells, e.g., T cells, from the stationary phase.

In some embodiments, the collected immune cells, e.g., T cells, are engineered outside the chromatography column. In some embodiments, the collected immune cells, e.g., T cells, are engineered by targeted integration of a transgene encoding a recombinant protein. In some embodiments, the targeted integration involves inducing a genetic disruption in the immune cells, e.g., T cells. In some embodiments, the targeted integration is by homology directed repair (HDR). In some embodiments, the transgene is introduced by non-viral gene delivery. In some embodiments, the nucleic acid molecule is a DNA molecule. In some embodiments, the nucleic acid molecule is a naked DNA molecule.

In some embodiments, the nucleic acid molecule is a double-stranded DNA molecule. In some embodiments, the nucleic acid molecule is a single-stranded DNA molecule.

In some embodiments, the nucleic acid molecule is a modified DNA molecule. In some embodiments, the nucleic acid molecule is modified to enhance its stability.

In some embodiments, the nucleic acid molecule is a closed-ended DNA molecule. In some embodiments, the nucleic acid molecule is a naked closed-ended DNA molecule.

Certain available methods for producing engineered cells, such as those for use in cell therapies, e.g., recombinant receptor-expressing cells, may require considerable time to complete. In some aspects, the amount of time required for producing the engineered cells may impact the in vivo activity of the engineered cells following administration. Longer manufacturing times may result in reduced potency, persistence, or proliferative capacity of the engineered cells in vivo.

In some aspects, certain available methods may result in inefficient engineering of cells, for instance such that only a small proportion of cells subjected to the engineering method ultimately express a recombinant protein, e.g., recombinant receptor.

Particular available methods for engineering cells may also impact the in vivo activity of the engineered cells. For example, methods that result in random or semi-random integration of a transgene in the genome of the engineered cells, such as lentiviral transduction, may also impact the in vivo activity of the engineered cells. For instance, random or semi-random integration events may result in transcriptional activation or inactivation effects or the introduction of new splice variants. Improved methods for producing engineered cells are needed.

The provided embodiments offer various advantages. In some aspects, the provided methods reduce the amount of time required for producing engineered cells, such as to within 48 hours of initiating stimulation of immune cells prior to their engineering. In some aspects, the provided methods allow for more rapid manufacturing of engineered cells, for instance leading to improved engineered cell production turn-around times and ultimately reduced manufacturing costs. In some aspects, the provided methods allow sufficient time for transgene integration, but limit the amount of time in which engineered cells are stimulated or allowed to proliferate ex vivo.

In some aspects, the provided methods improve the efficiency with which cells are engineered. In some aspects, the provided methods involve engineering cells from a whole blood sample, rather than an apheresis or leukapheresis sample. In some aspects, the provided methods involving engineering cells from a whole blood sample result in higher recombinant protein expression than when engineering cells from an apheresis or leukapheresis sample. In some embodiments, recombinant protein expression is at least 1.1-, 1.2-, 1.3-, 1.4-, 1.5-, 1.6-, 1.7-, 1.8-, 1.9-, 2-, 2.5-, 3-, 3.5-, 4-, 4.5-, or 5-fold higher in cells produced from a whole blood sample than from an apheresis or leukapheresis sample. In some aspects, the use of a whole blood sample may reduce manipulation or processing of cells to be engineered. Reduced manipulation or processing may improve cell health, for instance such that transgene delivery, integration, and expression can be more readily effected during engineering, including engineering involving the electroporation of cells.

In some aspects, the provided methods employ methods for targeted integration of a transgene in the genome of the engineered cells. In some aspects, the provided methods avoid effects that may be associated with random or semi-random integration events.

In some aspects, the engineered cells produced from a whole blood sample by the provided methods have improved long-term effects on cytotoxic activity and increased proliferative capacity in vivo following administration, relative to engineered cells produced from an apheresis or leukapheresis sample. In some aspects, the engineered cells produced by the provided methods, as well as the provided genetically engineered cells, are those with improved potency, persistence, and/or proliferative capacity in vivo.

In some embodiments, the provided methods involve engineering one or more immune cells, e.g., T cells. In some embodiments, the engineering is by any of the methods described herein, for instance in Section I-C. In some embodiments, the provided methods involve targeted integration of a transgene into a target site of a gene in the one or more immune cells, e.g., T cells. In some embodiments, the provided methods involve introducing a nucleic acid molecule containing the transgene into the one or more immune cells, e.g., T cells. In some embodiments, the introducing of the nucleic acid molecule is under conditions for targeted integration of the transgene into the target site. In some embodiments, the introducing of the nucleic acid molecule is by non-viral gene delivery. In some embodiments, the nucleic acid molecule is a DNA molecule. In some embodiments, the nucleic acid molecule is a naked DNA molecule.

In some embodiments, the nucleic acid molecule is a double-stranded DNA molecule. In some embodiments, the nucleic acid molecule is a single-stranded DNA molecule.

In some embodiments, the nucleic acid molecule is a modified DNA molecule. In some embodiments, the nucleic acid molecule is modified to enhance its stability.

In some embodiments, the nucleic acid molecule is a closed-ended DNA molecule. In some embodiments, the nucleic acid molecule is a naked closed-ended DNA molecule.

In some embodiments, the introducing of the one or more gene-editing agents is by electroporation.

In some embodiments, the provided methods involve introducing one or more gene-editing agents for editing the gene in the one or more immune cells, e.g., T cells. In some embodiments, the introducing of the one or more gene-editing agents is by electroporation. In some embodiments, the introducing of the one or more gene-editing agents is carried out prior to the introducing of the nucleic acid molecule. In some embodiments, the introducing of the one or more gene-editing agents is carried out currently with the introducing of the nucleic acid molecule.

In some embodiments, the targeted integration is by homology directed repair (HDR). In some embodiments, the HDR involves introducing one or more gene-editing agents for inducing a genetic disruption in the gene in the one or more immune cells, e.g., T cells.

In some embodiments, the transgene encodes a recombinant protein. In some embodiments, the provided methods produce genetically engineered immune cells, e.g., T cells, expressing the recombinant protein. In some embodiments, the recombinant protein is a recombinant receptor. In some embodiments, the recombinant receptor is a T cell receptor (TCR). In some embodiments, the recombinant receptor is a chimeric antigen receptor (CAR).

In some embodiments, the provided methods involve stimulating a plurality of immune cells, e.g., T cells, containing the one or more immune cells, e.g., T cells. In some embodiments, the stimulating is by any of the methods described herein, for instance in Section I-B. In some embodiments, the stimulating is by on-column stimulation of the plurality of immune cells, e.g., T cells. In some embodiments, the provided methods involve incubating the plurality of immune cells, e.g., T cells, under conditions to stimulate immune cells, e.g., T cells, of the plurality of immune cells, e.g., T cells. In some embodiments, the incubating is by any of the methods described herein, for instance in Section I-B-2.

In some embodiments, the incubating is carried out in the presence of a stimulatory reagent. In some embodiments, the stimulatory reagent is any described herein, for instance in Section I-B-1. In some embodiments, the provided methods involve adding the stimulatory reagent to the plurality of immune cells, e.g., T cells.

In some embodiments, the stimulatory reagent contains a primary agent that specifically binds to a molecule to provide a primary activation signal to an immune cell, e.g., T cell. In some embodiments, the stimulatory reagent contains a secondary agent that specifically binds to a costimulatory molecule to provide a costimulatory signal to an immune cell, e.g., T cell. In some embodiments, the stimulatory reagent contains the primary agent and the secondary agent.

In some embodiments, the stimulatory reagent is a T cell stimulatory reagent. In some embodiments, the T cell stimulatory reagent contains a primary agent that specifically binds to a member of a TCR/CD3 complex. In some embodiments, the T cell stimulatory reagent contains a secondary agent that specifically binds to a T cell costimulatory molecule. In some embodiments, the T cell stimulatory reagent contains the primary agent and the secondary agent.

In some embodiments, the incubating occurs in an internal cavity of a chromatography column. In some embodiments, the stimulating is by on-column stimulation of the plurality of immune cells, e.g., T cells. In some embodiments, the plurality of immune cells, e.g., T cells, are immobilized on a stationary phase in the internal cavity of the chromatography column. In some embodiments, the stationary phase is any described herein, for instance in Section I-A-1.

In some embodiments, the immobilized plurality of immune cells, e.g., T cells, are incubated in the presence of the stimulatory reagent, e.g., T cell stimulatory reagent. In some embodiments, the stimulatory reagent, e.g., T cell stimulatory reagent, is added to the plurality of immune cells, e.g., T cells, immobilized on the stationary phase.

In some embodiments, the stationary phase contains a selection agent that specifically binds to a selection marker expressed on the surface of the plurality of immune cells, e.g., T cells. In some embodiments, specific binding of the selection agent to the selection marker effects the immobilization of the plurality of immune cells, e.g., T cells, on the stationary phase.

In some embodiments, the provided methods involve selecting the plurality of immune cells, e.g., T cells. In some embodiments, the selecting is by any of the methods described herein, for instance in Section I-A. In some embodiments, the provided methods involve adding a sample containing the plurality of immune cells, e.g., T cells, to the internal cavity of the chromatography column. In some embodiments, the sample is a whole blood sample. In some embodiments, the plurality of immune cells, e.g., T cells, become immobilized to the stationary phase. In some embodiments, specific binding of the selection agent to the selection marker effects the immobilization of the plurality of immune cells, e.g., T cells, on the stationary phase.

In some embodiments, the plurality of immune cells are a plurality of lymphocytes. In some embodiments, the plurality of immune cells are a plurality of T cells, B cells, or NK cells. In some embodiments, the plurality of immune cells are a plurality of T cells. In some embodiments, the plurality of T cells are CD4+ T cells. In some embodiments, the plurality of T cells are CD8+ T cells. In some embodiments, the plurality of T cells contain CD4+ T cells and CD8+ T cells.

In some embodiments, the plurality of immune cells, e.g., T cells, are primary cells, e.g., primary T cells, from a subject. In some embodiments, the subject is a human subject.

In some embodiments, the provided methods involve collecting immune cells, e.g., T cells, of the plurality of immune cells. In some embodiments, the collecting is by any of the methods described herein, for instance in Section I-B-3. In some embodiments, the collected immune cells, e.g., T cells, are collected from the chromatography column. In some embodiments, the collected immune cells, e.g., T cells, are immune cells, e.g., T cells, no longer immobilized on the stationary phase. In some embodiments, the collected immune cells, e.g., T cells, are immune cells, e.g., T cells, no longer immobilized on the stationary phase after the incubating. In some embodiments, the incubating results in immune cells, e.g., T cells, of the plurality of immune cells, e.g., T cells, becoming no longer immobilized on the stationary phase.

In some embodiments, the collected immune cells, e.g., T cells, contain the one or more immune cells, e.g., T cells, that are engineered. In some embodiments, the engineering is of one or more of the collected immune cells, e.g., T cells.

In some embodiments, the nucleic acid molecule is introduced into immune cells, e.g., T cells, of the collected immune cells, e.g., T cells. In some embodiments, the nucleic acid molecule is introduced into one or more of the collected immune cells, e.g., T cells.

In some embodiments, the one or more gene-editing agents are introduced into immune cells, e.g., T cells, of the collected immune cells, e.g., T cells. In some embodiments, the one or more gene-editing agents are introduced into one or more of the collected immune cells, e.g., T cells.

In some embodiments, the provided methods involve further incubating the collected immune cells, e.g., T cells. In some embodiments, the further incubating is by any of the methods described herein, for instance in Section I-B-4. In some embodiments, the further incubating is carried out prior to the engineering. In some embodiments, the further incubating is carried out prior to the introducing of the nucleic acid molecule. In some embodiments, the further incubating is carried out prior to the introducing of the one or more gene-editing agents.

In some embodiments, the further incubating is carried out in the presence of the stimulatory reagent. In some embodiments, the further incubating is not carried out in the internal cavity of the chromatography column. In some embodiments, the further incubating is carried out outside of the chromatography column.

In some embodiments, the conditions for targeted integration involve cultivating the one or more immune cells, e.g., T cells, under conditions to integrate the transgene into the target site. In some embodiments, the cultivating is by any of the methods described herein, for instance in Section I-C-5. In some embodiments, the cultivating is under conditions to integrate the transgene by HDR. In some embodiments, the cultivating is of the collected immune cells, e.g., T cells. In some embodiments, the cultivating is carried out in the presence of the nucleic acid molecule.

In some embodiments, the provided methods involve harvesting the genetically engineered immune cells, e.g., T cells, expressing the recombinant protein, for instance the recombinant receptor, e.g., TCR or CAR. In some embodiments, the harvesting is by any of the methods described herein, for instance in Section I-D.

In some embodiments, the provided methods involve formulating the harvested genetically engineered immune cells, e.g., T cells. In some embodiments, the formulating is by any of the methods described herein, for instance in Section I-E.

Also provided herein in some embodiments are genetically engineered immune cells produced by any of the provided methods. In some embodiments, the genetically engineered immune cell is a genetically engineered lymphocyte. In some embodiments, the genetically engineered immune cell is a genetically engineered T cell.

Also provided herein in some embodiments are pharmaceutical compositions containing any of the provided genetically engineered immune cells, e.g., T cells. In some embodiments, the provided pharmaceutical compositions are any described herein, for instance in Section II. In some embodiments, the pharmaceutical composition contains a pharmaceutically acceptable excipient. In some embodiments, the pharmaceutical composition is for use in treating a disease or condition in a subject.

Also provided herein in some embodiments are methods of treatment involving administering to a subject having a disease or condition any of the provided pharmaceutical compositions. In some embodiments, the provided methods are any described herein, for instance in Section II. Also provided herein in some embodiments are uses of the provided pharmaceutical compositions for treating a disease or condition in a subject. Also provided herein in some embodiments are uses of the provided pharmaceutical compositions for the manufacture of a medicament for treating a disease or condition in a subject. In some embodiments, the provided uses are any described herein, for instance in Section II.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHODS FOR PRODUCING ENGINEERED IMMUNE CELLS

Sections I-A to I-E describe exemplary steps of the provided methods. In some embodiments, the provided methods involve one or more of steps of selecting, stimulating, and engineering immune cells, e.g., T cells. In some embodiments, the provided methods involve stimulating and engineering immune cells, e.g., T cells. In some embodiments, the provided methods involve selecting, stimulating, and engineering immune cells, e.g., T cells.

In some embodiments, the immune cells, e.g., T cells, are stimulated on-column following selection of the immune cells, e.g., T cells, based on surface expression of a selection marker via column chromatography. In some embodiments, the immune cells, e.g., T cells, are collected from the chromatography column following the stimulating, after which the collected immune cells, e.g., T cells, are engineered by targeted integration of a transgene encoding a recombinant protein.

In some embodiments, the provided methods also involve one or more of steps of harvesting and formulating immune cells, e.g., T cells.

In some embodiments, any number of the steps of the provided methods are carried out in a closed system. In some embodiments, any number of the steps of the provided methods are automated.

### A. Selection

In some embodiments, the provided methods involve selecting immune cells, e.g., T cells. In some embodiments, the selecting is based on expression of a selection marker on the surface of the immune cells, e.g., T cells.

In some embodiments, the selecting is by column chromatography. In some embodiments, the selecting effects the immobilization of the immune cells, e.g., T cells, on a stationary phase in an internal cavity of a chromatography column. In some embodiments, specific binding of the selection agent to the selection marker effects the immobilization of the immune cells, e.g., T cells, on the stationary phase. In some embodiments, the stationary phase is any described herein, for instance in Section I-A-1.

In some embodiments, the selecting is carried out prior to a step of stimulating immune cells, e.g., T cells. In some embodiments, the immune cells, e.g., T cells, are immobilized on the stationary phase during the stimulating. In some embodiments, the selecting is carried out subsequent to a step of stimulating immune cells, e.g., T cells.

In some embodiments, the selecting is carried out prior to a step of engineering immune cells, e.g., T cells. In some embodiments, the selecting is carried out subsequent to a step of engineering immune cells, e.g., T cells.

In some embodiments, the selecting is performed using any of the methods described in WO2013/124474, WO2015/164675, WO2017/068425, WO2020/089343, WO2021/084050, US2015/0024411, US2017/0037369, US2019/0112576, and US2022/0002669.

In some embodiments, the selecting is carried out at a temperature that is above room temperature. In some embodiments, the selecting is carried out at a physiological temperature. In some embodiments, the selecting is carried out a temperature between or between about 30° C. and 39° C. In some embodiments, the selecting is carried out a temperature between or between about 35° C. and 39° C. In some embodiments, the selecting is carried out at or at about 37° C.

In some embodiments, the temperature is regulated by one or more heating elements configured to provide heat to the stationary phase. In some embodiments, the temperature is regulated using any of the methods or devices described in WO2020/089343, WO2021/084050, and US2022/0002669.

In some embodiments, the immune cells, e.g., T cells, are contained in a sample. In some embodiments, the provided methods involve adding the sample to the stationary phase. In some embodiments, the sample contains cell types in addition to the immune cells, e.g., in addition to the T cells. In some embodiments, the sample contains additional cells that do not express the selection marker, e.g., non-T cells.

In some embodiments, the sample is a biological sample. In some embodiments, the immune cells are primary cells, e.g., T cells, from a subject. In some embodiments, the subject is a human subject.

Exemplary samples include body fluids, such as blood, plasma, serum, cerebrospinal fluid, synovial fluid, urine, and sweat; tissue; and organ samples. Further exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, and other organs. In some embodiments, the sample is obtained directly from the subject. In some embodiments, the sample is a processed sample. In some embodiments, the sample is derived from any of the foregoing samples.

The sample can contain lymphocytes, including T cells, monocytes, granulocytes, B cells, and other nucleated white blood cells; red blood cells; and/or platelets. In some embodiments, the sample contains T cells.

In some embodiments, the sample is a whole blood sample, a buffy coat sample, a peripheral blood mononuclear cell (PBMC) sample, an unfractionated T cell sample, a lymphocyte sample, a white blood cell sample, an apheresis product, or a leukapheresis product. In some embodiments, the sample is a whole blood sample. In some embodiments, the sample is an apheresis product. In some embodiments, the sample is a leukapheresis product. In some embodiments, the whole blood sample from the subject is not processed before adding it to the stationary phase.

In some aspects, the sample is blood or a blood-derived sample. In particular embodiments, the sample is a whole blood sample. In aspects of the provided methods, manufacturing cells directly from a whole blood source sample can result in overall improved transduction efficiency compared to methods in which cells have first been processed, e.g., by apheresis or leukapheresis, from a blood sample from the subject.

In some embodiments, the immune cells, e.g., T cells, are obtained from the circulating blood of the subject by, e.g., apheresis or leukapheresis. In some embodiments, the sample is or is derived from an apheresis or leukapheresis product.

In some embodiments, the immune cells, e.g., T cells, obtained from the circulating blood of the subject are washed to, e.g., remove the plasma fraction and to place the immune cells, e.g., T cells, in an appropriate buffer or media for subsequent processing steps. In some embodiments, the immune cells, e.g., T cells, are washed with phosphate buffered saline (PBS). In some embodiments, the wash solution lacks calcium, magnesium, and/or many or all divalent cations. In some aspects, a washing step is accomplished using a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the immune cells, e.g., T cells, are resuspended in a variety of biocompatible buffers after washing, such as Ca²⁺/Mg²⁺ free PBS. In some embodiments, components of a blood sample are removed, and the immune cells, e.g., T cells, directly resuspended in culture media. In some embodiments, the sample is washed in order to remove one or more anti-coagulants, such as heparin, added during apheresis or leukapheresis.

### 1. Stationary Phases

In some embodiments, one or more steps of the provided methods involve the use of a stationary phase. In some embodiments, the stationary phase contains a selection agent. In some embodiments, the selection agent is any described herein, for instance in Section I-A-1-a.

In some embodiments, the stationary phase contains a chromatography matrix. In some embodiments, the chromatography matrix is suitable for cell separation using column chromatography. In some embodiments, the chromatography matrix is any described herein, for instance in Section I-A-1-b.

In some embodiments, the selection agent contains a binding partner. In some embodiments, the binding partner is for immobilization of the selection agent to the chromatography matrix.

In some embodiments, the stationary phase contains a selection reagent. In some embodiments, the selection reagent is any described herein, for instance in Section I-A-1-c. In some embodiments, the selection reagent contains a molecule or a plurality of molecules of streptavidin, avidin, a streptavidin analog or mutein, or an avidin analog or mutein.

In some embodiments, the selection agent is immobilized on the chromatography matrix. In some embodiments, the selection agent is immobilized directly on the chromatography matrix. In some embodiments, the binding partner of the selection agent is immobilized directly on the chromatography matrix.

In some embodiments, the selection reagent is immobilized on the chromatography matrix.

Methods for immobilizing the selection agent or selection reagent on the chromatography matrix can be identified and selected by one of ordinary skill in the art. In some instances, materials of the chromatography matrix, such as resins, can be activated in order to form covalent bonds with ligands containing amine, thiol, or hydroxyl groups. Such activated materials include epoxy-activated materials, such as epoxy-activated agarose, which is commercially available.

In some embodiments, the selection agent is immobilized indirectly on the chromatography matrix. In some embodiments, the selection agent is immobilized to the chromatography matrix via binding of the selection agent to the selection reagent immobilized on the chromatography matrix. In some embodiments, the binding partner of the selection agent is bound to the selection reagent. In some embodiments, the binding partner is bound to the molecule of the selection reagent that is streptavidin, avidin, a streptavidin analog or mutein, or an avidin analog or mutein.

In some aspects, the binding capacity of a stationary phase affects how much stationary phase is needed in order to select a certain number of immune cells, e.g., T cells, expressing the selection marker. The binding capacity can be used to determine or control the number of immobilized immune cells, e.g., T cells. In some aspects, the binding capacity of a stationary phase can be used to standardize the reagent amount, e.g., amount of stimulatory reagent, used in a single column.

In some embodiments, 1 mL of the stationary phase is capable of accommodating up to 0.1 billion±0.025 billion immune cells, e.g., T cells, expressing the selection marker. In some embodiments, the stationary phase is or is about 5 mL, 10 mL, 15 mL, 20 mL, 25 mL, 30 mL, 35 mL, or 40 mL. In some embodiments, the stationary phase is or is about 10 mL and is capable of accommodating up to 1 billion±0.25 billion immune cells, e.g., T cells, expressing the selection marker. In some embodiments, the stationary phase is or is about 20 mL and is capable of accommodating up to 2 billion±0.5 billion immune cells, e.g., T cells, expressing the selection marker. In some embodiments, the stationary phase is or is about 40 mL and is capable of accommodating between about 3 billion and about 5 billion immune cells, e.g., T cells, expressing the selection marker.

In some embodiments, the stationary phase has a binding capacity of between or between about 0.5 billion and 5 billion immune cells, e.g., T cells, expressing the selection marker. In some embodiments, the stationary phase has a binding capacity of between or between about 0.5 billion and 4 billion immune cells, e.g., T cells, expressing the selection marker. In some embodiments, the stationary phase has a binding capacity of between or between about 0.5 billion and 3 billion immune cells, e.g., T cells, expressing the selection marker. In some embodiments, the stationary phase has a binding capacity of between or between about 0.5 billion and 2 billion immune cells, e.g., T cells, expressing the selection marker. In some embodiments, the stationary phase has a binding capacity of between or between about 1 billion and 5 billion immune cells, e.g., T cells, expressing the selection marker. In some embodiments, the stationary phase has a binding capacity of between or between about 1 billion and 4 billion immune cells, e.g., T cells, expressing the selection marker. In some embodiments, the stationary phase has a binding capacity of between or between about 1 billion and 3 billion immune cells, e.g., T cells, expressing the selection marker. In some embodiments, the stationary phase has a binding capacity of between or between about 1 billion and 2 billion immune cells, e.g., T cells, expressing the selection marker, inclusive. In some embodiments, the stationary phase is 20 mL. In some embodiments, the stationary phase has a binding capacity of 2 billion±0.5 billion immune cells, e.g., T cells, expressing the selection marker.

In some embodiments, the binding capacity of the stationary phase is the maximum number of immune cells, e.g., T cells, expressing the selection marker bound to the stationary phase at given solvent and cell concentration conditions, when an excess of immune cells, e.g., T cells, expressing the selection marker are loaded onto the stationary phase. In some embodiments, the binding capacity is or is about 100 million±25 million immune cells, e.g., T cells, expressing the selection marker per mL of stationary phase.

In some embodiments, the static binding capacity is the maximum amount of immune cells, e.g., T cells, expressing the selection marker capable of being immobilized on the stationary phase, e.g., at certain solvent and cell concentration conditions. In some embodiments, the static binding capacity of the stationary phase ranges between about 75 million and about 125 million immune cells, e.g., T cells, expressing the selection marker per mL of stationary phase. In some embodiments, the static binding capacity of the stationary phase ranges between about 50 million and about 100 million immune cells, e.g., T cells, expressing the selection marker per mL of stationary phase. In some embodiments, the static binding capacity is or is about 100 million±25 million immune cells, e.g., T cells, expressing the selection marker per mL of stationary phase. In some embodiments, the static binding capacity of the stationary phase disclosed herein ranges between about 75 million and about 125 million immune cells, e.g., T cells, expressing the selection marker per mL of stationary phase. In some embodiments, the static binding capacity of the stationary phase is between about 10 million and about 20 million, between about 20 million and about 30 million, between about 30 million and about 40 million, between about 40 million and about 50 million, between about 50 million and about 60 million, between about 60 million and about 70 million, between about 70 million and about 80 million, between about 80 million and about 90 million, between about 90 million and about 100 million, between about 110 million and about 120 million, between about 120 million and about 130 million, between about 130 million and about 140 million, between about 140 million and about 150 million, between about 150 million and about 160 million, between about 160 million and about 170 million, between about 170 million and about 180 million, between about 180 million and about 190 million, or between about 190 million and about 200 million immune cells, e.g., T cells, expressing the selection marker per mL of stationary phase.

In some embodiments, the binding capacity of the stationary phase is the number of immune cells, e.g., T cells, expressing the selection marker that bind to the stationary phase under given flow conditions before a significant breakthrough of unbound immune cells, e.g., T cells, expressing the selection marker occurs. In one aspect, the binding capacity of the stationary phase is a dynamic binding capacity, e.g., the binding capacity under operating conditions in a packed chromatography column during sample application. In some embodiments, the dynamic binding capacity is determined by loading a sample containing a known concentration of the immune cells, e.g., T cells, expressing the selection marker and monitoring the flow-through, and the immune cells, e.g., T cells, expressing the selection marker will bind the stationary phase to a certain break point before unbound immune cells, e.g., T cells, expressing the selection marker will flow through the column. In some embodiments, the dynamic binding capacity is or is about 100 million+25 million immune cells, e.g., T cells, expressing the selection marker per mL of stationary phase. In some embodiments, the dynamic binding capacity of the stationary phase is between or is between about 75 million and about 125 million immune cells, e.g., T cells, expressing the selection marker per mL of stationary phase. In some embodiments, the dynamic binding capacity of the stationary phase ranges between about 50 million and about 100 million immune cells, e.g., T cells, expressing the selection marker per mL of stationary phase. In some embodiments, the dynamic binding capacity of the stationary phase is between about 10 million and about 20 million, between about 20 million and about 30 million, between about 30 million and about 40 million, between about 40 million and about 50 million, between about 50 million and about 60 million, between about 60 million and about 70 million, between about 70 million and about 80 million, between about 80 million and about 90 million, between about 90 million and about 100 million, between about 110 million and about 120 million, between about 120 million and about 130 million, between about 130 million and about 140 million, between about 140 million and about 150 million, between about 150 million and about 160 million, between about 160 million and about 170 million, between about 170 million and about 180 million, between about 180 million and about 190 million, or between about 190 million and about 200 million immune cells, e.g., T cells, expressing the selection marker per mL of stationary phase.

a. Selection Agents

In some embodiments, the selection marker is a lipid, a polysaccharide, or a nucleic acid. In some embodiments, the selection marker is a peptide or a protein, such as a receptor, e.g., a membrane receptor protein. In some embodiments, the selection marker is a peripheral membrane protein or an integral membrane protein. The selection marker can in some embodiments have one or more domains that span the membrane. As a few illustrative examples, a membrane protein with a transmembrane domain may be a G-protein coupled receptor, such as an odorant receptors, a rhodopsin receptor, a rhodopsin pheromone receptor, a peptide hormone receptor, a taste receptor, a GABA receptor, an opiate receptor, a serotonin receptor, a Ca2+ receptor, melanopsin, a neurotransmitter receptor, such as a ligand gated, a voltage gated or a mechanically gated receptor, including the acetylcholine, the nicotinic, the adrenergic, the norepinephrine, the catecholamines, the L-DOPA-, a dopamine and serotonin (biogenic amine, endorphin/enkephalin) neuropeptide receptor, a receptor kinase such as serine/threonine kinase, a tyrosine kinase, a porin/channel such as a chloride channel, a potassium channel, a sodium channel, an OMP protein, an ABC transporter (ATP-Binding Cassette-Transporter) such as amino acid transporter, the Na-glucose transporter, the Na/iodide transporter, an ion transporter such as Light Harvesting Complex, cytochrome c oxidase, ATPase Na/K, H/K, Ca, a cell adhesion receptor such as metalloprotease, an integrin, or a catherin.

In some embodiments, the selection marker is a molecule expressed by or defining a cell population, for instance a population or subpopulation of blood cells, e.g., lymphocytes (e.g., T cells, B cells, or NK cells), monocytes, or stem cells (e.g., CD34 positive peripheral stem cells or Nanog or Oct-4 expressing stem cells). In some embodiments, the selection marker is expressed on the surface of a target cell, e.g., a cell targeted for genetic engineering. In some embodiments, the selection marker is a molecule expressed on the surface of immune cells. In some embodiments, the selection marker is a molecule expressed on the surface of lymphocytes. In some embodiments, the selection marker is a molecule expressed on the surface of T cells, B cells, or NK cells. In some embodiments, the selection marker is a molecule expressed on the surface of T cells. Examples of T cells include cells such as CMV-specific CD8+ T cells, cytotoxic T cells, memory T cells, and regulatory T-cells (Treg). An illustrative example of Treg includes CD4 CD25 CD45RA Treg cells, and an illustrative example of memory T cells includes CD62L CD8+ specific central memory T cells. In some embodiments, the selection marker is CD25, CD28, CD62L, CCR7, CD27, CD127, CD3, CD4, CD8, CD57, CD45RA, or CD45RO. In some embodiments, the selection marker is CD3. In some embodiments, the selection marker is CD28. In some embodiments, the selection marker is CD4. In some embodiments, the selection marker is CD8.

In some embodiments, the selection agent contains an antibody, an antibody fragment, a proteinaceous molecule with antibody-like binding properties, a molecule containing Ig domains, a cytokine, a chemokine, an MHC molecules, an MHC-peptide complex, a receptor ligand, or a binding fragment of any of the foregoing, that specifically binds to the selection marker. In some embodiments, the selection agent contains an antibody. In some embodiments, the selection agent contains an antibody fragment. In some embodiments, the antibody fragment is selected from Fab fragments, Fv fragments, single-chain Fv fragments (scFv), divalent antibody fragments such as F(ab′)₂-fragments, diabodies, triabodies (Iliades, P., et al., FEBS Lett (1997) 409, 437-441), decabodies (Stone, E., et al., Journal of Immunological Methods (2007) 318, 88-94), and other domain antibodies (Holt, L. J., et al., Trends Biotechnol. (2003), 21, 11, 484-490).

In some embodiments, the selection agent binds to the selection marker in a monovalent manner. In some embodiments, the selection agent contains a monovalent antibody fragment, a proteinaceous binding molecule with antibody-like binding properties, an aptamer, or an MHC molecule. In some embodiments, the selection agent contains a monovalent antibody fragment. In some embodiments, the monovalent antibody fragment is a Fab fragment, Fv fragment, or single-chain Fv fragment (scFv). In some embodiments, the monovalent antibody fragment is a Fab fragment.

In some embodiments, the selection agent contains an antibody fragment that is a divalent antibody fragment. In some embodiments, the divalent antibody fragment is an F(ab′)2-fragment or a divalent single-chain Fv fragment.

In some embodiments, the selection agent contains a proteinaceous molecule with antibody-like binding properties. In some embodiments, the proteinaceous molecule with antibody-like binding properties is an aptamer, a mutein based on a polypeptide of the lipocalin family, a glubody, a protein based on the ankyrin scaffold, a protein based on the crystalline scaffold, an adnectin, or an avimer. Other exemplary proteinaceous molecules include an EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or a an immunoglobulin-like domain (for example, domain antibodies or camel heavy chain antibodies), a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, “Kappabodies” (cf. III. et al., Protein Eng (1997) 10, 949-57, a so called “minibody” (Martin et al., EMBO J (1994) 13, 5303-5309), a diabody (cf. Holliger et al., PNAS USA (1993)90, 6444-6448), a so called “Janusis” (cf. Traunecker et al., EMBO J (1991) 10, 3655-3659, or Traunecker et al., Int J Cancer (1992) Suppl 7, 51-52), a nanobody, a microbody, an affilin, an affibody, a knottin, ubiquitin, a zinc-finger protein, an autofluorescent protein, and a leucine-rich repeat protein. In some embodiments, the selection agent is a bivalent proteinaceous artificial binding molecule such as a dimeric lipocalin mutein that is also known as “duocalin”.

In some embodiments, the dissociation constant (K_{D}) of the binding between the selection agent and the selection marker is from about 10⁻² M to about 10⁻¹³ M, from about 10⁻³ M to about 10⁻¹² M, from about 10⁻⁴ M to about 10⁻¹¹ M, or from about 10⁻⁵M to about 10⁻¹⁰ M. In some embodiments, the dissociation constant (K_{D}) for the binding between the selection agent and the selection marker is from about 10⁻³ to about 10⁻⁷ M, e.g., is of low affinity. In some embodiments, the dissociation constant (K_{D}) for the binding between the selection agent and the selection marker is from about 10⁻⁷ to about 1×10⁻¹⁰ M, e.g., is of high affinity.

In some embodiments, when expressed in terms of the k_{off} rate (also called dissociation rate constant) for the binding between the selection agent and the selection marker, the k_{off} rate is about 0.5×10⁻⁴ sec⁻¹ or greater, about 1×10⁻⁴ sec⁻¹ or greater, about 2×10⁻⁴ sec⁻¹ or greater, about 3×10⁻⁴ sec⁻¹ or greater, about 4×10⁻⁴ sec⁻¹ of greater, about 5×10⁻⁴ sec⁻¹ or greater, about 1×10⁻³ sec⁻¹ or greater, about 1.5×10⁻³ sec⁻¹ or greater, about 2×10⁻³ sec⁻¹ or greater, about 3×10⁻³ sec⁻¹ or greater, about 4×10⁻³ sec⁻¹, about 5×10⁻³ sec-1 or greater, about 1×10⁻² sec or greater, or about 5×10⁻¹ sec⁻¹ or greater. It is within the level of one of ordinary skill in the art to empirically determine the k_{off} rate range suitable for a particular selection agent and selection marker interaction (see, e.g., U.S. Pat. No. 9,023,604). The K_{D}, k_{off}, and koₙ rate of the bond formed between the selection agent and the selection marker can be determined by any suitable means, for example by fluorescence titration, equilibrium dialysis, or surface plasmon resonance.

In some embodiments, the selection marker is a co-receptor. In some embodiments, the selection marker is a T cell co-receptor. In some embodiments, the selection marker is CD4. In some embodiments, the selection agent contains an anti-CD4 antibody, a divalent antibody fragment of an anti-CD4 antibody, a monovalent antibody fragment of an anti-CD4-antibody, or a proteinaceous CD4 binding molecule with antibody-like binding properties. In some embodiments, the anti-CD4 antibody, divalent antibody fragment of an anti-CD4 antibody, or monovalent antibody fragment of an anti-CD4 antibody (e.g., anti-CD4 Fab fragment) is derived from antibody 13B8.2 or a functionally active mutant of 13B8.2 that retains specific binding for CD4. Exemplary mutants of antibody 13B8.2 or m13B8.2 are described in U.S. Pat. Nos 7,482,000, U.S. Patent Appl. No. US2014/0295458, International Patent Application No. WO2013/124474, and Bes, C, et al. J Biol Chem 278, 14265-14273 (2003). The mutant Fab fragment termed “m13B8.2” carries the variable domain of the CD4 binding murine antibody 13B8.2 and a constant domain containing constant human CH1 domain of type gamma for the heavy chain and the constant human light chain domain of type kappa, as described in U.S. Pat. No. 7,482,000. In some embodiments, the anti-CD4 antibody, e.g., a mutant of antibody 13B8.2, contains the amino acid replacement H91A in the variable light chain, the amino acid replacement Y92A in the variable light chain, the amino acid replacement H35A in the variable heavy chain, and/or the amino acid replacement R53A in the variable heavy chain, each by Kabat numbering. In some embodiments, compared to variable domains of the 13B8.2 Fab fragment in m13B8.2, the His residue at position 91 of the light chain (position 93 in SEQ ID NO: 30) is mutated to Ala, and the Arg residue at position 53 of the heavy chain (position 55 in SEQ ID NO: 29) is mutated to Ala. In some embodiments, the selection agent contains an anti-CD4 Fab. In some embodiments, the anti-CD4 Fab contains a variable heavy chain having the sequence set forth in SEQ ID NO: 29 and a variable light chain having the sequence set forth in SEQ ID NO: 30. In some embodiments, the anti-CD4 Fab contains the CDRs of the variable heavy chain having the sequence set forth in SEQ ID NO: 29 and the CDRs of the variable light chain having the sequence set forth in SEQ ID NO: 30.

In some embodiments, the selection marker is CD8. In some embodiments, the selection agent contains an anti-CD8 antibody, a divalent antibody fragment of an anti-CD8 antibody, a monovalent antibody fragment of an anti-CD8 antibody, or a proteinaceous CD8 binding molecule with antibody-like binding properties. In some embodiments, the anti-CD8 antibody, divalent antibody fragment of an anti-CD8 antibody, or monovalent antibody fragment of an anti-CD8 antibody (e.g., anti-CD8 Fab fragment) is derived from antibody OKT8 (e.g., ATCC CRL-8014) or a functionally active mutant thereof that retains specific binding for CD8. In some embodiments, the selection agent contains an anti-CD8 Fab. In some embodiments, the anti-CD8 Fab contains a variable heavy chain having the sequence set forth in SEQ ID NO: 36 and a variable light chain having the sequence set forth in SEQ ID NO: 37. In some embodiments, the anti-CD8 Fab contains the CDRs of the variable heavy chain having the sequence set forth in SEQ ID NO: 36 and the CDRs of the variable light chain having the sequence set forth in SEQ ID NO: 37.

In some embodiments, the selection marker is a molecule containing an immunoreceptor tyrosine-based activation motif (ITAM). In some embodiments, the selection marker is a member of a T cell antigen receptor complex. In some embodiments, the selection marker is a member of a TCR/CD3 complex. In some embodiments, the selection marker is CD3. In some embodiments, the selection marker is a CD3 chain. In some embodiments, the selection marker is a CD3 zeta chain.

In some embodiments, the selection marker is CD3. In some embodiments, the selection agent contains an anti-CD3 antibody, a divalent antibody fragment of an anti-CD3 antibody, a monovalent antibody fragment of an anti-CD3 antibody, or a proteinaceous CD3 binding molecule with antibody-like binding properties. In some embodiments, the anti-CD3 antibody, divalent antibody fragment of an anti-CD3 antibody, or monovalent antibody fragment of an anti-CD3 antibody (e.g., anti-CD3 Fab fragment) is derived from antibody OKT3 (e.g., ATCC CRL-8001; see, e.g., Stemberger et al. pLoS One. 2012; 7(4): e35798) or a functionally active mutant thereof that retains specific binding for CD3. In some embodiments, the selection agent contains an anti-CD3 Fab. In some embodiments, the anti-CD3 Fab contains a variable heavy chain having the sequence set forth in SEQ ID NO: 31 and a variable light chain having the sequence set forth in SEQ ID NO: 32. In some embodiments, the anti-CD3 Fab contains the CDRs of the variable heavy chain having the sequence set forth in SEQ ID NO: 31 and the CDRs of the variable light chain having the sequence set forth in SEQ ID NO: 32.

In some embodiments, the selection marker is CD25. In some embodiments, the selection agent contains an anti-CD25 antibody, a divalent antibody fragment of an anti-CD25 antibody, a monovalent antibody fragment of an anti-CD25 antibody, or a proteinaceous CD25 binding molecule with antibody-like binding properties. In some embodiments, the selection agent contains an anti-CD25 Fab. In some embodiments, the anti-CD25 antibody, divalent antibody fragment of an anti-CD25 antibody, or monovalent antibody fragment of an anti-CD25 antibody (e.g., anti-CD25 Fab) is derived from antibody FRT5 (see, e.g., Stemberger et al. 2012. pLoS One. 2012; 7(4):e35798) or a functionally active mutant thereof that retains specific binding for CD25.

In some embodiments, the selection marker is CD62L. In some embodiments, the selection agent contains an anti-CD62L antibody, a divalent antibody fragment of an anti-CD62L antibody, a monovalent antibody fragment of an anti-CD62L antibody, or a proteinaceous CD62L binding molecule with antibody-like binding properties. In some embodiments, the selection agent contains an anti-CD62L Fab. In some embodiments, the anti-CD62L antibody, divalent antibody fragment of an anti-CD62L antibody, or monovalent antibody fragment of an anti-CD62L antibody (e.g., anti-CD62L Fab) is derived from antibody DREG56 (e.g., ATCC HB300; see, e.g., Stemberger et al. 2012, pLoS One. 2012; 7(4):e35798) or a functionally active mutant thereof that retains specific binding for CD62L.

In some embodiments, the selection marker is CD45RA. In some embodiments, the selection agent contains an anti-CD45RA antibody, a divalent antibody fragment of an anti-CD45RA antibody, a monovalent antibody fragment of an anti-CD45RA antibody, or a proteinaceous CD45RA binding molecule with antibody-like binding properties. In some embodiments, the selection agent contains an anti-CD45RA Fab. In some embodiments, the anti-CD45RA antibody, divalent antibody fragment of an anti-CD45RA antibody, or monovalent antibody fragment of an anti-CD45RA antibody (e.g., anti-CD45RA Fab fragment) is derived from antibody MEM56 (e.g., Millipore 05-1413; see, e.g., Stemberger et al. 2012, pLoS One. 2012; 7(4):e35798) or a functionally active mutant thereof that retains specific binding for CD45RA.

In some embodiments, the selection marker is a costimulatory molecule, an accessory molecule, a cytokine receptor, a chemokine receptor, an immune checkpoint molecule, or a member of the TNF family or TNF receptor family. In some embodiments, the selection marker is a costimulatory molecule. In some embodiments, the costimulatory molecule is CD28, CD90 (Thy-1), CD95 (Apo-/Fas), CD137 (4-1BB), CD154 (CD40L), ICOS, LAT, CD27, OX40, or HVEM.

In some embodiments, the selection marker is CD28. In some embodiments, the selection agent contains an anti-CD28 antibody, a divalent antibody fragment of an anti-CD28 antibody, a monovalent antibody fragment of an anti-CD28 antibody, or a proteinaceous CD28 binding molecule with antibody-like binding properties. In some embodiments, the anti-CD28 antibody, divalent antibody fragment of an anti-CD28 antibody, or monovalent antibody fragment of an anti-CD28 antibody (e.g., anti-CD28 Fab fragment) is derived from antibody CD28.3 (deposited as a synthetic single chain Fv construct under GenBank Accession No. AF451974.1; see also Vanhove et al, BLOOD, 15 Jul. 2003, Vol. 102, No. 2, pages 564-570), the variable heavy and light chains of which contain the amino acid sequences set forth in SEQ ID NO: 33 and 34, respectively. In some embodiments, the selection agent contains an anti-CD28 Fab. In some embodiments, the anti-CD28 Fab contains a variable heavy chain having the sequence set forth in SEQ ID NO: 33 and a variable light chain having the sequence set forth in SEQ ID NO: 34. In some embodiments, the anti-CD28 Fab contains the CDRs of the variable heavy chain having the sequence set forth in SEQ ID NO: 33 and the CDRs of the variable light chain having the sequence set forth in SEQ ID NO: 34.

In some embodiments, the selection marker is CD90. In some embodiments, the selection agent contains an anti-CD90 antibody, a divalent antibody fragment of an anti-CD90 antibody, a monovalent antibody fragment of an anti-CD90 antibody, or a proteinaceous CD90 binding molecule with antibody-like binding properties. In some embodiments, the selection agent contains an anti-CD90 Fab. In some embodiments, the anti-CD90 antibody, divalent antibody fragment of an anti-CD90 antibody, or monovalent antibody fragment of an anti-CD90 antibody (e.g., anti-CD90 Fab fragment) is derived from the anti-CD90 antibody G7 (Biolegend, cat. no. 105201).

In some embodiments, the selection marker is CD95. In some embodiments, the selection agent contains an anti-CD95 antibody, a divalent antibody fragment of an anti-CD95 antibody, a monovalent antibody fragment of an anti-CD95 antibody, or a proteinaceous CD95 binding molecule with antibody-like binding properties. In some embodiments, the selection agent contains an anti-CD95 Fab. In some embodiments, the anti-CD95 antibody, divalent antibody fragment of an anti-CD95 antibody, or monovalent antibody fragment of an anti-CD95 antibody (e.g., anti-CD95 Fab fragment) is derived from monoclonal mouse anti-human CD95 CH11 (Upstate Biotechnology, Lake Placid, NY), anti-CD95 mAb 7C11, or anti-APO-1, such as described in Paulsen et al. Cell Death & Differentiation 18.4 (2011): 619-631.

In some embodiments, the selection marker is CD137. In some embodiments, the selection agent contains an anti-CD137 antibody, a divalent antibody fragment of an anti-CD137 antibody, a monovalent antibody fragment of an anti-CD137 antibody, or a proteinaceous CD137 binding molecule with antibody-like binding properties. In some embodiments, the selection agent contains an anti-CD137 Fab. In some embodiments, the anti-CD137 antibody, divalent antibody fragment of an anti-CD137 antibody, or monovalent antibody fragment of an anti-CD137 antibody (e.g., anti-CD137 Fab fragment) is derived from LOB12, IgG2a or LOB12.3, IgG1 as described in Taraban et al. Eur J Immunol. 2002 December; 32(12):3617-27. See also, e.g., U.S. Pat. Nos. 6,569,997, 6,303,121, and Mittler et al. Immunol Res. 2004; 29(1-3):197-208.

In some embodiments, the selection marker is CD40. In some embodiments, the selection agent contains an anti-CD40 antibody, a divalent antibody fragment of an anti-CD40 antibody, a monovalent antibody fragment of an anti-CD40 antibody, or a proteinaceous CD40 binding molecule with antibody-like binding properties. In some embodiments, the selection agent contains an anti-CD40 Fab.

In some embodiments, the selection marker is CD40L. In some embodiments, the selection agent contains an anti-CD40L antibody, a divalent antibody fragment of an anti-CD40L antibody, a monovalent antibody fragment of an anti-CD40L antibody, or a proteinaceous CD40L binding molecule with antibody-like binding properties. In some embodiments, the selection agent contains an anti-CD40L Fab. In some embodiments, the anti-CD40L antibody, divalent antibody fragment of an anti-CD40L antibody, or monovalent antibody fragment of an anti-CD40L antibody (e.g., anti-CD40L Fab fragment) is derived from Hu5C8, as described in Blair et al. JEM vol. 191 no. 4 651-660. See also, e.g., WO1999061065, US20010026932, U.S. Pat. No. 7,547,438, and WO2001056603.

In some embodiments, the selection marker is ICOS. In some embodiments, the selection agent contains an anti-ICOS antibody, a divalent antibody fragment of an anti-ICOS antibody, a monovalent antibody fragment of an anti-ICOS antibody, or a proteinaceous ICOS binding molecule with antibody-like binding properties. In some embodiments, the selection agent contains an anti-ICO Fab. In some embodiments, the anti-ICOS antibody, divalent antibody fragment of an anti-ICOS antibody, or monovalent antibody fragment of an anti-ICOS antibody (e.g., anti-ICOS Fab fragment) is derived from any of the antibodies described in US20080279851 and Deng et al. Hybrid Hybridomics. 2004 June; 23(3):176-82.

In some embodiments, the selection marker is Linker for Activation of T cells (LAT). In some embodiments, the selection agent contains an anti-LAT antibody, a divalent antibody fragment of an anti-LAT antibody, a monovalent antibody fragment of an anti-LAT antibody, or a proteinaceous LAT binding molecule with antibody-like binding properties. In some embodiments, the selection agent contains an anti-LAT Fab.

In some embodiments, the selection marker is CD27. In some embodiments, the selection agent contains an anti-CD27 antibody, a divalent antibody fragment of an anti-CD27 antibody, a monovalent antibody fragment of an anti-CD27 antibody, or a proteinaceous CD27 binding molecule with antibody-like binding properties. In some embodiments, the selection agent contains an anti-CD27 Fab. In some embodiments, the anti-CD27 antibody, divalent antibody fragment of an anti-CD27 antibody, or monovalent antibody fragment of an anti-CD27 antibody (e.g., anti-CD27 Fab fragment) is derived from any of the antibodies described in WO2008051424.

In some embodiments, the selection marker is OX40. In some embodiments, the selection agent contains an anti-OX40 antibody, a divalent antibody fragment of an anti-OX40 antibody, a monovalent antibody fragment of an anti-OX40 antibody, or a proteinaceous OX40 binding molecule with antibody-like binding properties. In some embodiments, the selection agent contains an anti-OX40 Fab. In some embodiments, the anti-OX40 antibody, divalent antibody fragment of an anti-OX40 antibody, or monovalent antibody fragment of an anti-OX40 antibody (e.g., anti-OX40 Fab fragment) is derived from any of the antibodies described in WO2013038191 and Melero et al. Clin Cancer Res. 2013 Mar. 1; 19(5):1044-53.

In some embodiments, the selection marker is HVEM. In some embodiments, the selection agent contains an anti-HVEM antibody, a divalent antibody fragment of an anti-HVEM antibody, a monovalent antibody fragment of an anti-HVEM antibody, or a proteinaceous HVEM binding molecule with antibody-like binding properties. In some embodiments, the selection agent contains an anti-HVEM Fab. In some embodiments, the anti-HVEM antibody, divalent antibody fragment of an anti-HVEM antibody, or monovalent antibody fragment of an anti-HVEM antibody (e.g., anti-HVEM Fab fragment) is derived from any of the antibodies described in WO2006054961, WO2007001459, and Park et al. Cancer Immunol Immunother. 2012 February; 61(2):203-14.

In some embodiments, the selection agent further contains a binding partner. In some embodiments, the selection agent contains between 1 and 5, 1 and 4, 1 and 3, or 1 and 2 binding partners, each inclusive. In some embodiments, the selection agent contains exactly one binding partner. In some embodiments, the selection agent contains exactly two binding partners. In some embodiments, the selection agent contains exactly three binding partners. In some embodiments, the selection agent contains exactly four binding partners. In some embodiments, the selection agent contains exactly five binding partners.

In some embodiments, each binding partner of a selection agent containing multiple binding partners is individually selected from among the binding partners described herein, for instance any described in this section. In some embodiments, each binding partner of a selection agent containing multiple binding partners is the same and is any one of the binding partners described herein, for instance any described in this section.

Im some embodiments, the binding partner is hydrocarbon-based (including polymeric) and contains nitrogen-, phosphorus-, sulphur-, carben-, halogen- or pseudohalogen groups. In some embodiments, the binding partner is an alcohol, an organic acid, an inorganic acid, an amine, a phosphine, a thiol, a disulfide, an alkane, an amino acid, a peptide, an oligopeptide, a polypeptide, a protein, a nucleic acid, a lipid, a saccharide, an oligosaccharide, or a polysaccharide. As further examples, in some embodiments, the binding partner is a cation, an anion, a polycation, a polyanion, a polycation, an electrolyte, a polyelectrolyte, a carbon nanotube, or carbon nanofoam. As yet further examples, in some embodiments, the binding partner is a crown ether, an immunoglobulin or a fragment thereof, or a proteinaceous binding molecule with antibody-like functions.

In some embodiments, the binding partner includes a moiety known to one of ordinary skill in the art as an affinity tag. In some embodiments, the selection reagent includes a corresponding binding partner, for example an antibody or an antibody fragment known to bind to the affinity tag. As a few illustrative examples of known affinity tags, in some embodiments, the affinity tag includes dinitrophenol or digoxigenin, oligohistidine, polyhistidine, an immunoglobulin domain, glutathione-S-transferase (GST), chitin binding protein (CBP) or thioredoxin, calmodulin binding peptide (CBP), FLAG′-peptide, the HA-tag (SEQ ID NO: 20), the VSV-G-tag (SEQ ID NO: 21), the HSV-tag (SEQ ID NO: 22), the T7 epitope (SEQ ID NO: 23), maltose binding protein (MBP), the HSV epitope (SEQ ID NO: 24) of herpes simplex virus glycoprotein D, the “myc” epitope of the transcription factor c-myc (SEQ ID NO: 25), or the V5-tag (SEQ ID NO: 26). In some embodiments, the complex formed between the binding site of the selection reagent and the affinity tag, for instance between the corresponding binding partner of the selectio reagent, e.g., an antibody or antibody fragment, and the affinity tag, can be disrupted competitively by contacting the complex with a free binding partner, e.g., an unbound affinity tag.

In some embodiments, the affinity tag includes an oligonucleotide tag. In some some embodiments, the oligonucleotide tag hybridizes to an oligonucleotide linked to or included in the selection reagent with a complementary sequence.

In some embodiments, the binding partner is a lectin, protein A, protein G, a metal, a metal ion, nitrilo triacetic acid derivatives (NT A), RGD-motifs, a dextrane, polyethyleneimine (PEI), a redox polymer, a glycoprotein, an aptamer, a dye, amylose, maltose, cellulose, chitin, glutathione, calmodulin, gelatine, polymyxin, heparin, NAD, NADP, lysine, arginine, benzamidine, poly U, or oligo-dT. Lectins such as Concavalin A are known to bind to polysaccharides and glycosylated proteins. An illustrative example of a dye is a triazine dye, such as Cibacron blue F3G-A (CB) or Red HE-3B, which specifically binds NADH-dependent enzymes. Green A is known to bind to Co A proteins, human serum albumin, and dehydrogenases. The dyes 7-aminoactinomycin D and 4′,6-diamidino-2-phenylindole are known to bind to DNA. Cations of metals such as Ni, Cd, Zn, Co, or Cu can also be used to bind affinity tags, such as an oligohistidine-containing sequence, including the hexahistidine or the MAT tag (SEQ ID NO: 35), and N-methacryloyl-(L)-cysteine methyl ester.

In some embodiments, the binding between the binding partner and the binding site of the selection reagent occurs in the presence of a divalent, a trivalent, or a tetravalent cation. In some embodiments, the selection reagent includes a divalent, a trivalent, or a tetravalent cation, for instance held, e.g., complexed, by means of a suitable chelator. In some embodiments, the binding partner includes a moiety that complexes with a divalent, a trivalent, or a tetravalent cation. Examples of metal chelators include ethylenediamine, ethylene-diaminetetraacetic acid (EDTA), ethylene glycol tetraacetic acid (EGTA), diethylenetri-aminepentaacetic acid (DTPA), N,N-bis(carboxymethyl)glycine (also called nitrilotriacetic acid, NTA), 1,2-bis(o-aminophenoxy)ethane-N,N,N′,N′-tetraacetic acid (BAPTA), 2,3-dimer-capto-1-propanol (dimercaprol), porphine, and heme. As an example, EDTA can form a complex with most monovalent, divalent, trivalent, and tetravalent metal ions, such as silver (Ag⁺), calcium (Ca²⁺), manganese (Mn²⁺), copper (Cu²⁺), iron (Fe²⁺), cobalt (Co⁺), and zirconium (Zr⁴⁺), while BAPTA is specific for Ca²⁺. As an illustrative example, one of ordinary skill in the art can use methods involving the formation of a complex between an oligohistidine tag and copper (Cu²⁺), nickel (Ni²⁺), cobalt (Co²⁺), or zinc (Zn²⁺) ions, which are presented by means of the chelator nitrilotriacetic acid (NTA).

In some embodiments, the binding partner includes a calmodulin-binding peptide, and the selection reagent includes multimeric calmodulin, for instance as described in U.S. Pat. No. 5,985,658. In some embodiments, the binding partner includes a FLAG peptide, and the selection reagent includes an antibody that binds to the FLAG peptide. For instance, in some embodiments, the selection reagent includes the monoclonal antibody 4E11 that binds to the FLAG peptide, for instance as described in U.S. Pat. No. 4,851,341. In some embodiments, the binding partner includes an oligohistidine tag, and the selection reagent includes an antibody or a transition metal ion that binds the oligohistidine tag. In some embodiments, calmodulin, antibodies such as 4E11, chelated metal ions, and free chelators may be multimerized by methods involving, for example, biotinylation and complexation with streptavidin, avidin, or oligomers thereof, or by the introduction of carboxyl residues into a polysaccharide, e.g., dextran, for instance as described in Noguchi et al. (1992), Bioconjugate Chemistry 3: 132-137, in a first step, and linking calmodulin, antibodies, chelated metal ions, or free chelators via primary amino groups to the carboxyl groups in the polysaccharide, e.g. dextran, using carbodiimide chemistry in a second step. In some embodiments, the binding between the binding partner and the binding site of the selection reagent can be disrupted by metal ion chelation. The metal chelation may be accomplished by, for example, addition of EGTA or EDTA.

In some embodiments, the binding partner binds to a biotin-binding molecule. In some embodiments, the binding partner binds to the biotin-binding site of the molecule.

In some embodiments, the binding partner is a streptavidin or avidin binding partner. In some embodiments, the binding partner is a streptavidin-binding partner. In some embodiments, the streptavidin-binding partner is also an avidin-binding partner.

In some embodiments, the binding partner binds to a molecule that is streptavidin, avidin, a streptavidin analog or mutein, or an avidin analog or mutein. In some embodiments, the molecule is any of the streptavidin, avidin, streptavidin analog or mutein, and avidin analog or mutein molecules described herein, for instance in Section I-A-1-c. In some embodiments, the selection reagent contains the molecule. In some embodiments, the binding partner binds to a biotin-binding site of the molecule. In some embodiments, the binding partner binds to the natural biotin-binding site of the molecule (see, e.g., Qureshi et al. (2001), Journal of Biological Chemistry 276(49): 46422-46428; and Livnah et al. (1993), Proc Natl Acad Sci 90: 5076-5080; which describe the interactions of biotin with streptavidin and avidin, respectively). In some embodiments, the binding partner allows for the functionalization of reagents containing streptavidin, avidin, a streptavidin analog or mutein, or an avidin analog or mutein.

Binding partners that bind to streptavidin, avidin, a streptavidin analog or mutein, or an avidin analog or mutein, including that bind to the biotin-binding sites of these molecules, can be identified and selected by one of ordinary skill in the art. In some embodiments, the binding partner binds to a molecule that is streptavidin.

In some embodiments, the binding partner contains biotin. In some embodiments, the binding partner is biotin. In some embodiments, the biotin is D-biotin. In some embodiments, the binding partner contains a biotin analog or derivate. In some embodiments, the binding partner is a biotin analog or derivate. In some embodiments, the biotin analog or derivative is a structural analog of biotin. In some embodiments, the biotin analog or derivative binds to the biotin-binding site of streptavidin, avidin, a streptavidin analog or mutein, or an avidin analog or mutein. In some embodiments, the biotin analog or derivative binds to the biotin-binding site of streptavidin. In some embodiments, the biotin analog or derivative is desthiobiotin, iminobiotin, guanidinobiotin, diaminobiotin, lipoic acid, HABA (hydroxyazobenzene-benzoic acid), dimethyl-HABA, biotin sulfone, caproylamidobiotin, or biocytin (or any of the biotin analogs and derivatives described in, e.g., International Published PCT Appl. No. WO2008140573).

In some embodiments, the binding partner contains a streptavidin-binding peptide. In some embodiments, the binding partner is a streptavidin-binding peptide. In some embodiments, the streptavidin-binding peptide binds to the biotin-binding site of streptavidin, avidin, a streptavidin analog or mutein, or an avidin analog or mutein. In some embodiments, the streptavidin-binding peptide binds to the biotin-binding site of streptavidin. In some embodiments, the streptavidin-binding peptide contains an amino acid sequence with the formula set forth in SEQ ID NO: 9, such as contains the amino acid sequence set forth in SEQ ID NO: 10. In some embodiments, the streptavidin-binding peptide contains an amino acid sequence with the formula set forth in SEQ ID NO: 11, such as set forth in SEQ ID NO: 12. In some embodiments, the streptavidin-binding peptide contains the amino acid sequence set forth in SEQ ID NO: 7, also called Strep-tag®. In some embodiments, the sequence of the streptavidin-binding peptide is set forth in SEQ ID NO: 7. In some embodiments, the streptavidin-binding peptide contains the amino acid sequence set forth in SEQ ID NO: 8, also called Strep-tag® II. In some embodiments, the sequence of the streptavidin-binding peptide is set forth in SEQ ID NO: 8.

In some embodiments, the streptavidin-binding peptide may be further modified. In some embodiments, the streptavidin-binding peptide contains the amino acid sequence set forth in SEQ ID NO: 8 that is conjugated to a nickel charged trisNTA, also called His-STREPPER or His/Strep-tag®II Adapter.

In some embodiments, the streptavidin-binding peptide contains a sequential arrangement of two streptavidin-binding modules. In some embodiments, the streptavidin-binding peptide contains a sequential arrangement of exactly two streptavidin-binding modules. In some embodiments, the streptavidin-binding modules are separated from one another by no more than 50 amino acids, for instance for no more than 45, 40, 35, 30, 25, 20, 15, 10, or 5 amino acids. In some embodiments, the streptavidin-binding modules are directly connected to one another. In some embodiments, one streptavidin-binding module has three to eight amino acids and contains at least the sequence His-Pro-Xaa (SEQ ID NO: 9), where Xaa is glutamine, asparagine, or methionine. In some embodiments, another streptavidin-binding module has the same or different sequence from the first streptavidin-binding module, such as set forth in SEQ ID NO: 11 (see, e.g., International Published PCT Appl. No. WO02/077018; and U.S. Pat. No. 7,981,632). In some embodiments, one of the streptavidin-binding modules contains the amino acid sequence set forth in SEQ ID NO: 7. In some embodiments, each of the streptavidin-binding modules contains the amino acid sequence set forth in SEQ ID NO: 7. In some embodiments, one of the streptavidin-binding modules contains the amino acid sequence set forth in SEQ ID NO: 8. In some embodiments, each of the streptavidin-binding modules contains the amino acid sequence set forth in SEQ ID NO: 8. In some embodiments, the streptavidin-binding peptide contains an amino acid sequence having the formula set forth in SEQ ID NO: 13 or SEQ ID NO: 14. In some embodiments, the streptavidin-binding peptide contains the amino acid sequence set forth in any of SEQ ID NO: 15-19. In some embodiments, the sequence of the streptavidin-binding peptide is set forth in any of SEQ ID NO: 15-19. In some embodiments, the streptavidin-binding peptide contains the amino acid sequence set forth in SEQ ID NO: 16, also called Twin-Strep-tag®. In some embodiments, the sequence of the streptavidin-binding peptide is set forth in SEQ ID NO: 16.

b. Chromatography Matrices

In some embodiments, the chromatography matrix is essentially innocuous, e.g., is not detrimental to the health or viability of cells added to the chromatography matrix.

In some embodiments, the chromatography matrix includes a non-magnetic material or non-magnetizable material. In some embodiments, the chromatography matrix is void of any magnetically attractable matter.

In some embodiments, the chromatography matrix includes a monolithic matrix. In some embodiments, the chromatography matrix includes a membrane matrix. In some embodiments, the chromatography matrix includes a particulate matrix. In some embodiments, the chromatography matrix includes a beaded matrix.

In some embodiments, the chromatography matrix includes derivatized silica or a crosslinked gel. In some embodiments, the crosslinked gel is based on a natural polymer, for instance a polysaccharide. In some embodiments, the polysaccharide is crosslinked. Examples of a polysaccharide matrix include an agarose gel (for example, Superflow™ agarose or a Sepharose® material such as Superflow™ Sepharose® that is commercially available in different bead and pore sizes) or a gel of crosslinked dextrans. Further examples include a particulate cross-linked agarose matrix to which dextran is covalently bonded, for instance that is commercially available (in various bead sizes and with various pore sizes) as Sephadex® or Superdex®, both available from GE Healthcare. Further examples include Sephacryl®, which is also available in different bead and pore sizes from GE Healthcare.

In some embodiments, the crosslinked gel is based on a synthetic polymer. In some embodiments, the synthetic polymer is a polymer that has polar monomer units and which is therefore itself polar. In some embodiments, the synthetic polymer is hydrophilic. Examples of synthetic polymers include polyacrylamides, a styrene-divinylbenzene gel, and a copolymer of an acrylate and a diol or of an acrylamide and a diol. An illustrative example is a polymethacrylate gel, commercially available as a Fractogel®. A further example is a copolymer of ethylene glycol and methacrylate, commercially available as a Toyopearl®. In some embodiments, the chromatography matrix includes natural and synthetic polymer components, such as a composite matrix or a composite or a co-polymer of a polysaccharide and agarose, e.g., a polyacrylamide/agarose composite, or of a polysaccharide and N,N′-methylenebisacrylamide. An illustrative example of a copolymer of a dextran and N,N′-methylenebisacrylamide is the Sephacryl® series of material. A derivatized silica may include silica particles that are coupled to a synthetic or to a natural polymer. Examples of such embodiments include polysaccharide grafted silica, polyvinylpyrrolidone grafted silica, polyethylene oxide grafted silica, poly(2-hydroxyethylaspartamide) silica, and poly(N-isopropylacrylamide) grafted silica.

In some embodiments, the chromatography matrix includes a particulate matrix. In some embodiments, the chromatography matrix includes a polymeric resin, metal oxide, metalloid oxide, or mixed oxide. In some embodiments, particulates of the particulate matrix have a mean particle size of between or between about 5 μm and 600 μm, 5 μm and 400 μm, 5 μm and 200 μm, 5 μm and 150 μm, 5 μm and 125 μm, 5 μm and 100 μm, 5 μm and 75 μm, 5 μm and 50 μm, 5 μm and 25 μm, 25 μm and 600 μm, 25 μm and 400 μm, 25 μm and 200 μm, 25 μm and 150 μm, 25 μm and 125 μm, 25 μm and 100 μm, 25 μm and 75 μm, 25 μm and 50 μm, 50 μm and 600 μm, 50 μm and 400 μm, 50 μm and 200 μm, 50 μm and 150 μm, 50 μm and 125 μm, 50 μm and 100 μm, 50 μm and 75 μm, 75 μm and 600 μm, 75 μm and 400 μm, 75 μm and 200 μm, 75 μm and 150 μm, 75 μm and 125 μm, 75 μm and 100 μm, 100 μm and 600 μm, 100 μm and 400 μm, 100 μm and 200 μm, 100 μm and 150 μm, 100 μm and 125 μm, 125 μm and 600 μm, 125 μm and 400 μm, 125 μm and 200 μm, 125 μm and 150 μm, 150 μm and 600 μm, 150 μm and 400 μm, 150 μm and 200 μm, 200 μm and 600 μm, 200 μm and 400 μm, or 400 μm and 600 μm, each inclusive. In some embodiments, the particulates of the particulate matrix are between or between about 50 μm and 150 μm in diameter, inclusive. In some embodiments, the particulates of the particulate matrix are between or between about 75 μm and 125 μm in diameter, inclusive. In some embodiments, the particulates of the particulate matrix are between or between about 90 μm and 110 μm in diameter, inclusive.

In some embodiments, the chromatography matrix includes a chromatography resin. In some embodiments, the chromatography matrix includes chromatography resin beads, such as those commercially available as CytoSorb® (CytoSorbents™). In some embodiments, the resin includes a polystyrene resin. In some embodiments, the chromatography resin beads are between or between about 5 μm and 600 μm, 5 μm and 400 μm, 5 μm and 200 μm, 5 μm and 150 μm, 5 μm and 125 μm, 5 μm and 100 μm, 5 μm and 75 μm, 5 μm and 50 μm, 5 μm and 25 μm, 25 μm and 600 μm, 25 μm and 400 μm, 25 μm and 200 μm, 25 μm and 150 μm, 25 μm and 125 μm, 25 μm and 100 μm, 25 μm and 75 μm, 25 μm and 50 μm, 50 μm and 600 μm, 50 μm and 400 μm, 50 μm and 200 μm, 50 μm and 150 μm, 50 μm and 125 μm, 50 μm and 100 μm, 50 μm and 75 μm, 75 μm and 600 μm, 75 μm and 400 μm, 75 μm and 200 μm, 75 μm and 150 μm, 75 μm and 125 μm, 75 μm and 100 μm, 100 μm and 600 μm, 100 μm and 400 μm, 100 μm and 200 μm, 100 μm and 150 μm, 100 μm and 125 μm, 125 μm and 600 μm, 125 μm and 400 μm, 125 μm and 200 μm, 125 μm and 150 μm, 150 μm and 600 μm, 150 μm and 400 μm, 150 μm and 200 μm, 200 μm and 600 μm, 200 μm and 400 μm, or 400 μm and 600 μm in diameter, each inclusive. In some embodiments, the chromatography resin beads are between or between about 50 μm and 150 μm in diameter, inclusive. In some embodiments, the chromatography resin beads are between or between about 75 μm and 125 μm in diameter, inclusive. In some embodiments, the chromatography resin beads are between or between about 90 μm and 110 μm in diameter, inclusive.

In some embodiments, the chromatography matrix contains magnetically attractable matter, such as one or more magnetically attractable particles or a ferrofluid. Magnetically attractable particles may contain diamagnetic, ferromagnetic, paramagnetic, or superparamagnetic material. Superparamagnetic material responds to a magnetic field with an induced magnetic field without a resulting permanent magnetization. Magnetic particles based on iron oxide are commercially available as, for example, Dynabeads® from Dynal Biotech, magnetic MicroBeads from Miltenyi Biotec, and magnetic porous glass beads from CPG Inc., as well as from various other sources, such as Roche Applied Science, BIOCLON, BioSource International Inc., micromod, AMBION, Merck, Bangs Laboratories, Polysciences, or Novagen Inc. Magnetic nanoparticles based on superparamagnetic Co and FeCo, as well as ferromagnetic Co nanocrystals, have been described by, for example, Hutten, A. et al. (J. Biotech. (2004), 112, 47-63).

c. Selection Reagent

In some embodiments, the selection reagent contains a molecule to which the binding partner of the selection agent can bind.

In some cases, the selection reagent contains at least two chelating groups K that may be capable of binding to a transition metal ion. In some embodiments, the selection reagent may be capable of binding to an oligohistidine affinity tag, a glutathione-S-transferase, calmodulin or an analog thereof, calmodulin binding peptide (CBP), a FLAG-peptide, an HA-tag, maltose binding protein (MBP), an HSV epitope, a myc epitope, or a biotinylated carrier protein.

In some embodiments, the molecule is avidin, e.g., wild-type avidin. In some embodiments, the molecule is an avidin analog. In some embodiments, an avidin analog is a variant of wild-type avidin having one or more modified functional groups, but that contains a biotin-binding site. In some embodiments, the molecule is an avidin mutein. In some embodiments, an avidin mutein is a polypeptide distinguished from the sequence of wild-type avidin by one or more amino acid substitutions, deletions, or additions, but that contains a biotin-binding site. In some embodiments, the avidin analog is neutravidin, a deglycosylated avidin with modified arginines that can exhibit a more neutral pi and is available as an alternative to wild-type avidin. In some embodiments, the avidin analog is any of those commercially available as ExtrAvidin®, available through Sigma Aldrich, NeutrAvidin, available from Thermo Scientific or Invitrogen, and CaptAvidin™, available from Molecular Probes. In some embodiments, the avidin analog or mutein is any as described in International Published PCT Appl. No. WO2008/140573.

In some embodiments, the molecule is streptavidin, e.g., wild-type streptavidin. In some embodiments, streptavidin has the amino acid sequence disclosed by Argarana et al., Nucleic Acids Res. 14 (1986) 1871-1882 and set forth in SEQ ID NO: 1, or has an amino acid sequence that is a sequence present in homologs thereof from other Streptomyces species. In some embodiments, streptavidin has the amino acid sequence set forth in SEQ ID NO: 1.

In some embodiments, the molecule is a streptavidin analog. In some embodiments, a streptavidin analog is a variant of wild-type streptavidin having one or more modified functional groups, but that contains a biotin-binding site. In some embodiments, the molecule is a streptavidin mutein. In some embodiments, a streptavidin mutein is a polypeptide distinguished from the sequence of wild-type streptavidin by one or more amino acid substitutions, deletions, or additions, but that contains a biotin-binding site.

In some embodiments, the streptavidin mutein binds to a streptavidin-binding peptide, for instance any as described herein. In some embodiments, the streptavidin mutein binds to any of the streptavidin-binding peptides set forth in SEQ ID NO: 7, 8, and 15-19. In some embodiments, the binding affinity of the streptavidin-binding peptide to the streptavidin mutein is greater than 1×10⁻¹³ M, 1×10⁻¹² M, or 1×10⁻¹¹ M and less than 1×10⁻⁴ M, 5×10⁻⁴ M, 1×10⁻⁵ M, 5×10⁻⁵ M, 1×10⁻⁶ M, 5×10⁻⁶ M, or 1×10⁻⁷ M. In some embodiments, the streptavidin mutein binds to biotin, e.g., D-biotin. In some embodiments, the streptavidin mutein binds to a biotin analog or derivative, e.g., any as described herein. In some embodiments, the streptavidin mutein binds to biotin or to the biotin analog or derivative with greater affinity than to the streptavidin-binding peptide. In some embodiments, binding of the streptavidin-binding peptide to the streptavidin mutein, e.g., to the biotin-binding site of the streptavidin mutein, can be disrupted by the presence of biotin or the biotin analog or derivative. In some embodiments, the binding of the streptavidin mutein to the streptavidin-binding peptide of any of SEQ ID NO: 7, 8, and 15-19 is disrupted by the presence of biotin, e.g., D-biotin.

In some embodiments, the streptavidin mutein contains only a part of wild-type streptavidin. In some embodiments, the streptavidin mutein is a minimal streptavidin (in some instances referred to as a recombinant core streptavidin) wherein wild-type streptavidin is shortened at the N- and/or C-terminus. In some embodiments, the streptavidin mutein is any of the recombinant core streptavidins described in Sano et al. (1995), Journal of Biological Chemistry 270(47): 28204-28209. In some embodiments, the streptavidin mutein begins N-terminally in the region of amino acid positions 10 to 16 of SEQ ID NO: 1 and terminates C-terminally in the region of amino acid positions 133 to 142 of SEQ ID NO: 1. Reference to the position of residues in streptavidin or streptavidin muteins is with reference to the numbering of residues in SEQ ID NO: 1. In some embodiments, the sequence of the streptavidin mutein is set forth in any of SEQ ID NO: 2, 103, and 135. In some embodiments, the streptavidin mutein is an amino acid sequence from position Ala13 to Ser139 of SEQ ID NO: 1. In some embodiments, the sequence of the streptavidin mutein is set forth in SEQ ID NO: 135. In some embodiments, the streptavidin mutein contains an N-terminal methionine and an amino acid sequence from position Glu14 to Ser139 of SEQ ID NO: 1. In some embodiments, the sequence of the streptavidin mutein is set forth in SEQ ID NO: 2.

In some embodiments, the streptavidin mutein contains one or more amino acid substitutions compared to wild-type streptavidin, such as compared to the wild-type streptavidin sequence set forth in SEQ ID NO: 1. In some embodiments, the streptavidin mutein contains one or more amino acid substitutions compared to a streptavidin mutein that is a minimal streptavidin. In some embodiments, the streptavidin contains one or more amino acid substitutions compared to a streptavidin mutein, e.g., a minimal streptavidin, that begins N-terminally in the region of amino acid positions 10 to 16 of SEQ ID NO: 1 and terminates C-terminally in the region of amino acid positions 133 to 142 of SEQ ID NO: 1. In some embodiments, the streptavidin contains one or more amino acid substitutions compared to the streptavidin mutein set forth in any of SEQ ID NO: 2, 103, and 135.

In some embodiments, the streptavidin mutein binds to biotin and contains at least 1, 2, 3, 4, 5, 6, 7, 8, 9, 10, 11, 12, 13, 14, 15, 16, 17, 18, 19, or 20 amino acid differences compared to the sequence of amino acids set forth in SEQ ID NO: 1, 2, 103, or 135. In some embodiments, the streptavidin mutein binds to biotin and contains an amino acid sequence that exhibits at least 85%, 86%, 87%, 88%, 89%, 90%, 91%, 92%, 93%, 94%, 95%, 96%, 97%, 98%, 99% or more sequence identity to the sequence of amino acids set forth in SEQ ID NO: 1, 2, 103, or 135. In some embodiments, the amino acid substitutions are conservative or non-conservative mutations. In some embodiments, the streptavidin mutein is any as described in U.S. Pat. Nos. 5,168,049; 5,506,121; 6,022,951; 6,156,493; 6,165,750; 6,103,493; 6,368,813; and Internation Published PCT Appl. Nos. WO2014/076277, WO2008/140573, WO 86/02077, WO 98/40396, and WO 96/24606. In some embodiments, the streptavidin mutein is any as described in DE 19641876 A1; Howarth et al. (2006) Nat. Methods, 3:267-73; Zhang et al. (2015) Biochem. Biophys. Res. Commun., 463:1059-63; Fairhead et al. (2013) J. Mol. Biol., 426:199-214; Wu et al. (2005) J. Biol. Chem., 280:23225-31; Lim et al. (2010) Biochemistry, 50:8682-91); and Qureshi et al. (2001), Journal of Biological Chemistry 276(49): 46422-46428.

In some embodiments, the streptavidin mutein is any as described in U.S. Pat. No. 6,103,493. In some embodiments, the streptavidin mutein contains at least one mutation within the region corresponding to amino acid positions 44 to 53 of wild-type streptavidin, such as set forth in SEQ ID NO: 1. In some embodiments, “corresponding to” references amino acid positions with reference to the amino acid sequence of wild-type streptavidin, such as set forth in SEQ ID NO: 1. One of ordinary skill in the art would be able to identify these residues with methods involving, e.g., the alignment of sequences. In some embodiments, the streptavidin mutein contains a mutation at one or more of residues 44, 45, 46, and 47 of wild-type streptavidin. In some embodiments, the streptavidin mutein contains a replacement of Glu at position 44 with a hydrophobic aliphatic amino acid, e.g., Val, Ala, Ile, or Leu. In some embodiments, the streptavidin mutein contains any amino acid at position 45. In some embodiments, the streptavidin mutein contains an aliphatic amino acid, such as a hydrophobic aliphatic amino acid, at position 46. In some embodiments, the streptavidin mutein contains a replacement of Val at position 47 with a basic amino acid, e.g., Arg or Lys, such as Arg. In some embodiments, Ala is at position 46, Arg is at position 47, and Val or Ile is at position 44. In some embodiments, the streptavidin mutein contains residues Val⁴⁴-Thr⁴⁵-Ala⁴⁶-Arg⁴⁷ (SEQ ID NO: 134) at sequence positions corresponding to positions 44 to 47 of the sequence of amino acids set forth in SEQ ID NO: 1, such as set forth in exemplary streptavidin muteins containing the sequence of amino acids set forth in SEQ ID NO: 3, 4, or 104. In some embodiments, the streptavidin mutein contains residues Ile⁴⁴-Gly⁴⁵-Ala⁴⁶-Arg⁴⁷ (SEQ ID NO: 133) at sequence positions corresponding to positions 44 to 47 of the sequence of amino acids set forth in SEQ ID NO: 1, such as set forth in exemplary streptavidin muteins containing the sequence of amino acids set forth in SEQ ID NO: 5, 6, or 104. In some embodiments, the streptavidin mutein contains the amino acid sequence set forth in any of SEQ ID NO: 3-6, 104, and 105. In some embodiments, the streptavidin mutein is commercially available under the trademark Strep-Tactin® ml. In some embodiments, the streptavidin mutein is commercially available under the trademark Strep-Tactin® m2. In some embodiments, the streptavidin mutein contains the amino acid sequence set forth in SEQ ID NO: 4. In some embodiments, the streptavidin mutein contains the amino acid sequence set forth in SEQ ID NO: 6.

In some embodiment, the streptavidin mutein is any as described in International Published PCT Appl. No. WO 2014/076277. In some embodiments, the streptavidin mutein contains at least two cysteine residues in the region corresponding to amino acid positions 44 to 53 of the amino acid sequence set forth in SEQ ID NO: 1. In some embodiments, the cysteine residues are present at positions 45 and 52 to create a disulfide bridge connecting these amino acids. In some embodiments, amino acid 44 is glycine or alanine; amino acid 46 is alanine or glycine; and amino acid 47 is arginine. In some embodiments, the streptavidin mutein contains at least one mutation in the region corresponding to amino acids residues 115 to 121 of the amino acid sequence set forth in SEQ ID NO: 1. In some embodiments, the streptavidin mutein contains at least one mutation at amino acid position 117, 120, or 121 and/or a deletion of amino acids 118 and 119 and substitution of at least amino acid position 121.

In some embodiments, the streptavidin mutein contains a mutation at a position corresponding to position 117, which mutation can be to a large hydrophobic residue like Trp, Tyr, or Phe; to a charged residue like Glu, Asp, or Arg; to a hydrophilic residue like Asn or Gln; to the hydrophobic residues Leu, Met, or Ala; or the polar residues Thr, Ser, or His. In some embodiments, the mutation at position 117 is combined with a mutation at a position corresponding to position 120, which mutation can be to a small residue like Ser, Ala, or Gly, and a mutation at a position corresponding to position 121, which mutation can be to a hydrophobic residue, such as a bulky hydrophobic residue like Trp, Tyr, or Phe. In some embodiments, the mutation at position 117 is combined with a mutation at a position corresponding to position 120 of wild-type streptavidin set forth in SEQ ID NO: 1, which mutation can be a hydrophobic residue such as Leu, Ile, Met, or Val; or Tyr or Phe, and a mutation at a position corresponding to position 121 of SEQ ID NO: 1, which mutation can be to a small residue like Gly, Ala, or Ser, or with Gln, or with a hydrophobic residue like Leu, Val, Ile, Trp, Tyr, Phe, or Met. In some embodiments, the streptavidin mutein contains the residues Glu117, Gly120, and Tyr121 with reference to positions of the sequence of amino acids set forth in SEQ ID NO: 1. In some embodiments, the streptavidin mutein also contains residues Val⁴⁴-Thr⁴⁵-Ala⁴⁶-Arg⁴⁷ or residues Ile⁴⁴-Gly⁴⁵-Ala⁴⁶-Arg⁴⁷ at sequence positions corresponding to positions 44 to 47 of the sequence of amino acids set forth in SEQ ID NO: 1. In some embodiments, the streptavidin mutein contains the residues Val44, Thr45, Ala46, Arg47, Glu117, Gly120, and Tyr121. In some embodiments, the mutein streptavidin contains the sequence of amino acids set forth in any of SEQ ID NO: 27, 28, and 136, or a sequence of amino acids that exhibits at least 85%, 86%, 87%, 88%, 89%, 90%, 91%, 92%, 93%, 94%, 95%, 96%, 97%, 98%, 99% or more sequence identity to the sequence of amino acids set forth in any of SEQ ID NO: 27, 28, and 136, contains the residues Val44, Thr45, Ala46, Arg47, Glu117, Gly120 and Tyr121, and binds to biotin. In some embodiments, the streptavidin mutein contains the sequence of amino acids set forth in SEQ ID NO: 27. In some embodiments, the streptavidin mutein contains the sequence of amino acids set forth in SEQ ID NO: 28. In some embodiments, the streptavidin mutein contains the sequence of amino acids set forth in SEQ ID NO: 136.

In some embodiments, the streptavidin mutein contains the sequence of amino acids set forth in any of SEQ ID NO: 3-6, 27, 28, 104, 105, and 136, and the binding partner contains a streptavidin-binding peptide, wherein the sequence of the streptavidin-binding peptide is set forth in any of SEQ ID NO: 7, 8, and 15-19. In some embodiments, the streptavidin mutein contains the sequence of amino acids set forth in SEQ ID NO: 6, and the binding partner contains a streptavidin-binding peptide, wherein the sequence of the streptavidin-binding peptide is set forth in any of SEQ ID NO: 7, 8, and 15-19. In some embodiments, the streptavidin mutein contains the sequence of amino acids set forth in any of SEQ ID NO: 3-6, 27, 28, 104, 105, and 136, and the binding partner contains a streptavidin-binding peptide, wherein the sequence of the streptavidin-binding peptide is set forth in SEQ ID NO: 16. In some embodiments, the streptavidin mutein contains the sequence of amino acids set forth in SEQ ID NO: 6, and the binding partner contains a streptavidin-binding peptide, wherein the sequence of the streptavidin-binding peptide is set forth in SEQ ID NO: 16.

### B. Stimulation

In some embodiments, the provided methods involve stimulating immune cells, e.g., T cells. In some embodiments, the provided methods involve incubating the immune cells, e.g., T cells, under conditions to stimulate immune cells, e.g., T cells. In some embodiments, the incubating is carried out in the presence of a stimulatory reagent. In some embodiments, the provided methods involve adding the stimulatory reagent to the immune cells, e.g., T cells.

In some embodiments, the stimulating is carried out prior to a step of engineering immune cells, e.g., T cells. In some embodiments, the stimulating is carried out subsequent to a step of engineering immune cells, e.g., T cells.

### 1. Stimulatory Reagents

In some embodiments, the stimulatory reagent is any described in U.S. Pat. No. 11,274,278 and US2021/0032297, for instance any of the soluble multimerization reagents or oligomeric particle reagents described therein that are suitable for stimulating immune cells, e.g., T cells, including any that can be added to a stationary phase in an internal cavity of a chromatography column for stimulation of immune cells, e.g., T cells, immobilized on the stationary phase. In some embodiments, the T cell stimulatory reagent is a an oligomeric particle reagent composed of an oligomer of streptavidin or a streptavidin mutein molecules in which is attached one or more binding agents for stimulating T cells. In some embodiments, a primary agent (e.g., anti-CD3 agent) and a secondary agent (e.g., anti-CD28 agent) for stimulating the T cells are attached to the oligomer of streptavidin or streptavidin mutein molecules. In some embodiments, the T cell stimulatory reagent is in soluble form.

In some embodiments, the stimulatory reagent contains one or more binding agents. In some embodiments, the one or more binding agents are any described herein, for instance in Section I-B-1-b. In some embodiments, the one or more binding agents are selected from any of the selection agents described herein, for instance in Section I-A-1-a. In some embodiments, each of the one or more binding agents specifically binds to a molecule expressed on the surface of the immune cells, e.g., T cells. In some embodiments, the stimulatory reagent contains multiple binding agents that specifically bind to different molecules expressed on the surface of the immune cells, e.g., T cells. In some embodiments, the one or more binding agents include a primary agent and a secondary agent.

In some embodiments, the stimulatory reagent contains a primary agent that specifically binds to a molecule to provide a primary activation signal to an immune cell, e.g., T cell. In some embodiments, the stimulatory reagent contains a secondary agent that specifically binds to a costimulatory molecule to provide a costimulatory signal to an immune cell, e.g., T cell. In some embodiments, the stimulatory reagent contains the primary agent and the secondary agent.

In some embodiments, the stimulatory reagent is a T cell stimulatory reagent. In some embodiments, the T cell stimulatory reagent contains a primary agent that specifically binds to a member of a TCR/CD3 complex. In some embodiments, the T cell stimulatory reagent contains a secondary agent that specifically binds to a T cell costimulatory molecule. In some embodiments, the T cell stimulatory reagent contains the primary agent and the secondary agent.

In some embodiments, the one or more binding agents, e.g., primary and secondary agents, each contain a binding partner. The binding partners can be the same or different across the one or more binding agents, e.g., primary and secondary agents. In some embodiments, the stimulatory reagent contains a protein reagent having a binding site for the binding partner of each of the one or more binding agents, e.g., primary and secondary agents. In some embodiments, the protein reagent is any described herein, for instance in Section I-B-1-a. In some embodiments, the binding partner of each of the one or more binding agents, e.g., primary and secondary agents, is bound to the protein reagent.

In some embodiments, the protein reagent contains a plurality of binding sites for the binding partner of each of the one or more binding agents, e.g., primary and secondary agents. Thus, in some embodiments, the protein reagent allows for the multimerization of the one or more binding agents, e.g., primary and secondary agents, thereon, in some aspects for causing an avidity effect for the binding to molecules targeted by the one or more binding agents, e.g., primary and secondary agents. The plurality of binding sites can be the same or different across the protein reagent.

In some embodiments, the stimulatory reagent contains a weight ratio of protein reagent to each of the one or more binding agents, e.g., primary and secondary agents, that is between about 10:1 and 2:1, 9:1 and 2:1, 8:1 and 2:1, 7:1 and 2:1, 6:1 and 2:1, 5:1 and 2:1, 4:1 and 2:1, 3:1 and 2:1, 10:1 and 3:1, 9:1 and 3:1, 8:1 and 3:1, 7:1 and 3:1, 6:1 and 3:1, 5:1 and 3:1, 4:1 and 3:1, 10:1 and 4:1, 9:1 and 4:1, 8:1 and 4:1, 7:1 and 4:1, 6:1 and 4:1, 5:1 and 4:1, 10:1 and 5:1, 9:1 and 5:1, 8:1 and 5:1, 7:1 and 5:1, 6:1 and 5:1, 10:1 and 6:1, 9:1 and 6:1, 8:1 and 6:1, 7:1 and 6:1, 10:1 and 7:1, 9:1 and 7:1, 8:1 and 7:1, 10:1 and 8:1, 9:1 and 8:1, or 10:1 and 9:1, each inclusive. In some embodiments, the stimulatory reagent contains a weight ratio of protein reagent to each of the one or more binding agents, e.g., primary and secondary agents, that is between about 10:1 and 2:1, inclusive. In some embodiments, the stimulatory reagent contains a weight ratio of protein reagent to each of the one or more binding agents, e.g., primary and secondary agents, that is between about 8:1 and 2:1, inclusive. In some embodiments, the stimulatory reagent contains a weight ratio of protein reagent to each of the one or more binding agents, e.g., primary and secondary agents, that is between about 8:1 and 4:1, inclusive. In some embodiments, the weight ratio to protein reagent is different across the one or more binding agents, e.g., primary and secondary agents. In some embodiments, the weight ratio to protein reagent is the same across the one or more binding agents, e.g., primary and secondary agents. In some embodiments, the stimulatory reagent contains a weight ratio of protein reagent to each of the one or more binding agents, e.g., primary and secondary agents, that is about 6:1.

In some embodiments, the stimulatory reagent is prepared by mixing the protein reagent and each of the one or more binding agents, e.g., primary and secondary agents, at a weight ratio to each other of between about 10:1 and 2:1, 9:1 and 2:1, 8:1 and 2:1, 7:1 and 2:1, 6:1 and 2:1, 5:1 and 2:1, 4:1 and 2:1, 3:1 and 2:1, 10:1 and 3:1, 9:1 and 3:1, 8:1 and 3:1, 7:1 and 3:1, 6:1 and 3:1, 5:1 and 3:1, 4:1 and 3:1, 10:1 and 4:1, 9:1 and 4:1, 8:1 and 4:1, 7:1 and 4:1, 6:1 and 4:1, 5:1 and 4:1, 10:1 and 5:1, 9:1 and 5:1, 8:1 and 5:1, 7:1 and 5:1, 6:1 and 5:1, 10:1 and 6:1, 9:1 and 6:1, 8:1 and 6:1, 7:1 and 6:1, 10:1 and 7:1, 9:1 and 7:1, 8:1 and 7:1, 10:1 and 8:1, 9:1 and 8:1, or 10:1 and 9:1, each inclusive. In some embodiments, the stimulatory reagent is prepared by mixing the protein reagent and each of the one or more binding agents, e.g., primary and secondary agents, at a weight ratio to each other of between about 10:1 and 2:1, inclusive. In some embodiments, the stimulatory reagent is prepared by mixing the protein reagent and each of the one or more binding agents, e.g., primary and secondary agents, at a weight ratio to each other of between about 8:1 and 2:1, inclusive. In some embodiments, the stimulatory reagent is prepared by mixing the protein reagent and each of the one or more binding agents, e.g., primary and secondary agents, at a weight ratio to each other of between about 8:1 and 4:1, inclusive. In some embodiments, the weight ratio to protein reagent is different across the one or more binding agents, e.g., primary and secondary agents. In some embodiments, the weight ratio to protein reagent is the same across the one or more binding agents, e.g., primary and secondary agents. In some embodiments, the stimulatory reagent is prepared by mixing the protein reagent and each of the one or more binding agents, e.g., primary and secondary agents, at a weight ratio to each other of about 6:1. In some embodiments, the mixing is performed at room temperature.

In some embodiments, the stimulatory reagent contains the one or more binding agents, e.g., primary and secondary agents, at a weight ratio to each other of 4:1 to 1:1. In some embodiments, the stimulatory reagent contains the one or more binding agents, e.g., primary and secondary agents, at a weight ratio to each other of 3:1 to 1:1. In some embodiments, the stimulatory reagent contains the one or more binding agents, e.g., primary and secondary agents, at a weight ratio to each other of 2:1 to 1:1. In some embodiments, the stimulatory reagent contains the one or more binding agents, e.g., primary and secondary agents, at a weight ratio to each other of about 1:1, e.g., equal parts by weight of the one or more binding agents, e.g., primary and secondary agents.

In some embodiments, the stimulatory reagent is prepared by mixing the the one or more binding agents, e.g., primary and secondary agents, at a weight ratio to each other of 4:1 to 1:1. In some embodiments, the stimulatory reagent is prepared by mixing the one or more binding agents, e.g., primary and secondary agents, at a weight ratio to each other of 3:1 to 1:1. In some embodiments, the stimulatory reagent is prepared by mixing the one or more binding agents, e.g., primary and secondary agents, at a weight ratio to each other of 2:1 to 1:1. In some embodiments, the stimulatory reagent is prepared by mixing the one or more binding agents, e.g., primary and secondary agents, at a weight ratio to each other of about 1:1, e.g., equal parts by weight of the one or more binding agents, e.g., primary and secondary agents.

In some embodiments, the binding partner is bound to a biotin-binding site of the molecule or molecules of the protein reagent to which it is bound. In some embodiments, the biotin-binding site is the natural biotin-binding site of the molecule or molecules (see, e.g., Qureshi et al. (2001), Journal of Biological Chemistry 276(49): 46422-46428; and Livnah et al. (1993), Proc Natl Acad Sci 90: 5076-5080; which describe the interactions of biotin with streptavidin and avidin, respectively).

In some embodiments, the complex formed between the binding partner of each of the one or more binding agents, e.g., primary and secondary agents, and the protein reagent can be of any desired strength and affinity. In some embodiments, the complex is reversible. In some embodiments, the binding partner is reversibly bound to the molecule or molecules of the protein reagent to which it is bound. Exemplary binding partners and molecules for reversible binding are described herein as well as in, e.g., U.S. Pat. Nos. 5,168,049; 5,506,121; 6,103,493; 7,776,562; 7,981,632; 8,298,782; 8,735,540; and 9,023,604; and International Published PCT Appl. Nos. WO2013/124474 and WO2014/076277.

In some embodiments, the binding affinity of the binding partner to the molecule or molecules of the protein reagent to which it is bound is reduced compared to the binding affinity of biotin to streptavidin, which has a dissociation constant (K_{d}) on the order of ~10⁻⁴ mol/L. Binding affinity can be determined by any suitable method. In some embodiments, the binding affinity of the binding partner to the molecule or molecules of the protein reagent to which it is bound is greater than 1×10⁻¹³ M, 1×10⁻¹² M, or 1×10⁻¹¹ M and less than 1×10⁻⁴ M, 5×10⁻⁴ M, 1×10⁻⁵ M, 5×10⁻⁵ M, 1×10⁻⁶ M, 5×10⁻⁶ M, or 1×10⁻⁷ M.

In some embodiments, the binding partner contains biotin, e.g., D-biotin, and the molecule or molecules of the protein reagent to which it is bound are analogs or muteins of streptavidin or avidin that have reduced affinity for biotin, compared to streptavidin or avidin.

In some embodiments, the binding partner contains a biotin analog or derivative, e.g., any as described herein, having reduced affinity for streptavidin or avidin compared to biotin, and the molecule or molecules of the protein reagent to which it is bound are streptavidin or avidin. In some embodiments, the binding partner contains a biotin analog or derivative, e.g., any as described herein, and the molecule or molecules of the protein reagent to which it is bound are analogs or muteins of streptavidin or avidin that have reduced affinity for the biotin analog or derivative, compared to biotin.

In some embodiments, the binding partner contains a streptavidin-binding peptide, e.g., any as described herein, having reduced affinity for streptavidin or avidin compared to biotin, and the molecule or molecules of the protein reagent to which it is bound are streptavidin or avidin. In some embodiments, the binding partner contains a streptavidin-binding peptide, e.g., any as described herein, and the molecule or molecules of the protein reagent to which it is bound are analogs or muteins of streptavidin or avidin that have reduced affinity for the streptavidin-binding peptide, compared to biotin. In some embodiments, the binding partner contains a streptavidin-binding peptide, e.g., any as described herein, and the molecule or molecules of the protein reagent to which it is bound are muteins of streptavidin that have reduced affinity for the streptavidin-binding peptide, compared to biotin.

In some embodiments, the binding of the binding partner to the molecule or molecules of the protein reagent is disrupted by the presence of biotin, e.g., D-biotin. In some embodiments, the binding of the binding partner to the molecule or molecules of the protein reagent is disrupted by the presence of a biotin analog or derivative, e.g., any as described herein. For example, binding of the streptavidin-binding peptides known as Strep-tag®, Strep-tag® II, and Twin-Strep-tag® to streptavidin muteins known as StrepTactin® ml or m2 or StrepTactin XT® are disrupted by the presence of biotin, e.g., D-biotin, iminobiotin, lipoic acid, desthiobiotin, diaminobiotin, HABA, and dimethyl-HABA (see, e.g., U.S. Pat. Nos. 5,506,121 and 6,103,493, and International Published PCT Appl. No. WO2014/076277). Other combinations of molecules and binding partners whose binding can be disrupted by the presence of biotin or a biotin analog or derivative can be identified and selected by one of ordinary skill in the art.

In some embodiments, the stimulatory reagent is not immobilized on a solid support. In some embodiments, the stimulatory reagent is in soluble form. In some embodiments, the stimulatory reagent is soluble in a cell medium, e.g., any described herein.

In some embodiments, the stimulatory reagent contains a weight ratio of protein reagent:primary agent (e.g., anti-CD3 binding agent):secondary agent (e.g., anti-CD28 binding agent) that is between about 10:1:1 and 2:1:1, inclusive. In some embodiments, the stimulatory reagent contains a weight ratio of protein reagent:primary agent:secondary agent that is between about 8:1:1 and 2:1:1, inclusive. In some embodiments, the stimulatory reagent contains a weight ratio of protein reagent:primary agent:secondary agent that is between about 8:1:1 and 4:1:1, inclusive. In some embodiments, the stimulatory reagent contains a weight ratio of protein reagent:primary agent:secondary agent that is about 6:1:1. In some embodiments, 4 μg of the stimulatory reagent contains about 3 μg of protein reagent, 0.5 μg of anti-CD3 binding agent, and 0.5 μg of anti-CD28 binding agent.

a. Protein Reagents

In some embodiments, the protein reagent contains a molecule to which a binding partner of the one or more binding agents, e.g., primary and secondary agents, can bind. In some embodiments, the protein reagent contains a plurality of molecules to which the binding partner can bind. In some embodiments, the binding partner is bound to one of the plurality of molecules. In some embodiments, the binding partner is bound to two of the plurality of molecules.

In some embodiments, the molecule is any described herein that can bind to a binding partner of the one or more binding agents. In some embodiments, the molecule is any of the streptavidin, avidin, streptavidin analog or mutein, and avidin analog or mutein molecules described herein. In some embodiments, the molecule is streptavidin. In some embodiments, the molecule is any of the streptavidin mutein molecules described herein.

In some embodiments, each molecule of the protein reagent is individually selected from among any of the streptavidin, avidin, streptavidin analog or mutein, and avidin analog or mutein molecules described herein. In some embodiments, the protein reagent contains a mixture of any of the streptavidin, avidin, streptavidin analog or mutein, and avidin analog or mutein molecules described herein.

In some embodiments, each molecule of the protein reagent is individually selected from among any of the streptavidin and streptavidin analog or mutein molecules described herein. In some embodiments, the protein reagent contains a mixture of any of the streptavidin and streptavidin analog or mutein molecules described herein.

In some embodiments, each molecule of the protein reagent is the same and is any one of the streptavidin, avidin, streptavidin analog or mutein, and avidin analog or mutein molecules described herein. In some embodiments, each molecule of the protein reagent is the same and is streptavidin. In some embodiments, each molecule of the protein reagent is the same and is any one of the streptavidin mutein molecules described herein.

In some cases, the protein reagent contains at least two chelating groups K that may be capable of binding to a transition metal ion. In some embodiments, the protein reagent may be capable of binding to an oligohistidine affinity tag, a glutathione-S-transferase, calmodulin or an analog thereof, calmodulin binding peptide (CBP), a FLAG-peptide, an HA-tag, maltose binding protein (MBP), an HSV epitope, a myc epitope, or a biotinylated carrier protein.

In some embodiments, the molecule is avidin, e.g., wild-type avidin. In some embodiments, the molecule is an avidin analog. In some embodiments, an avidin analog is a variant of wild-type avidin having one or more modified functional groups, but that contains a biotin-binding site. In some embodiments, the molecule is an avidin mutein. In some embodiments, an avidin mutein is a polypeptide distinguished from the sequence of wild-type avidin by one or more amino acid substitutions, deletions, or additions, but that contains a biotin-binding site. In some embodiments, the avidin analog is neutravidin, a deglycosylated avidin with modified arginines that can exhibit a more neutral pi and is available as an alternative to wild-type avidin. In some embodiments, the avidin analog is any of those commercially available as ExtrAvidin®, available through Sigma Aldrich, NeutrAvidin, available from Thermo Scientific or Invitrogen, and CaptAvidin™, available from Molecular Probes. In some embodiments, the avidin analog or mutein is any as described in International Published PCT Appl. No. WO2008/140573.

In some embodiments, the molecule is streptavidin, e.g., wild-type streptavidin. In some embodiments, streptavidin has the amino acid sequence disclosed by Argarana et al., Nucleic Acids Res. 14 (1986) 1871-1882 and set forth in SEQ ID NO: 1, or has an amino acid sequence that is a sequence present in homologs thereof from other Streptomyces species. In some embodiments, streptavidin has the amino acid sequence set forth in SEQ ID NO: 1.

In some embodiments, the molecule is a streptavidin analog. In some embodiments, a streptavidin analog is a variant of wild-type streptavidin having one or more modified functional groups, but that contains a biotin-binding site. In some embodiments, the molecule is a streptavidin mutein. In some embodiments, a streptavidin mutein is a polypeptide distinguished from the sequence of wild-type streptavidin by one or more amino acid substitutions, deletions, or additions, but that contains a biotin-binding site.

In some embodiments, the streptavidin mutein binds to a streptavidin-binding peptide, for instance any as described herein. In some embodiments, the streptavidin mutein binds to any of the streptavidin-binding peptides set forth in SEQ ID NO: 7, 8, and 15-19. In some embodiments, the binding affinity of the streptavidin-binding peptide to the streptavidin mutein is greater than 1×10⁻¹³ M, 1×10⁻¹² M, or 1×10⁻¹¹ M and less than 1×10⁻⁴ M, 5×10⁻⁴ M, 1×10⁻⁵ M, 5×10⁻⁵ M, 1×10⁻⁶ M, 5×10⁻⁶ M, or 1×10⁻⁷ M. In some embodiments, the streptavidin mutein binds to biotin, e.g., D-biotin. In some embodiments, the streptavidin mutein binds to a biotin analog or derivative, e.g., any as described herein. In some embodiments, the streptavidin mutein binds to biotin or to the biotin analog or derivative with greater affinity than to the streptavidin-binding peptide. In some embodiments, binding of the streptavidin-binding peptide to the streptavidin mutein, e.g., to the biotin-binding site of the streptavidin mutein, can be disrupted by the presence of biotin or the biotin analog or derivative. In some embodiments, the binding of the streptavidin mutein to the streptavidin-binding peptide of any of SEQ ID NO: 7, 8, and 15-19 is disrupted by the presence of biotin, e.g., D-biotin.

In some embodiments, the streptavidin mutein contains only a part of wild-type streptavidin. In some embodiments, the streptavidin mutein is a minimal streptavidin (in some instances referred to as a recombinant core streptavidin) wherein wild-type streptavidin is shortened at the N- and/or C-terminus. In some embodiments, the streptavidin mutein is any of the recombinant core streptavidins described in Sano et al. (1995), Journal of Biological Chemistry 270(47): 28204-28209. In some embodiments, the streptavidin mutein begins N-terminally in the region of amino acid positions 10 to 16 of SEQ ID NO: 1 and terminates C-terminally in the region of amino acid positions 133 to 142 of SEQ ID NO: 1. Reference to the position of residues in streptavidin or streptavidin muteins is with reference to the numbering of residues in SEQ ID NO: 1. In some embodiments, the sequence of the streptavidin mutein is set forth in any of SEQ ID NO: 2, 103, and 135. In some embodiments, the streptavidin mutein is an amino acid sequence from position Ala13 to Ser139 of SEQ ID NO: 1. In some embodiments, the sequence of the streptavidin mutein is set forth in SEQ ID NO: 135. In some embodiments, the streptavidin mutein contains an N-terminal methionine and an amino acid sequence from position Glu14 to Ser139 of SEQ ID NO: 1. In some embodiments, the sequence of the streptavidin mutein is set forth in SEQ ID NO: 2.

In some embodiments, the streptavidin mutein contains one or more amino acid substitutions compared to wild-type streptavidin, such as compared to the wild-type streptavidin sequence set forth in SEQ ID NO: 1. In some embodiments, the streptavidin mutein contains one or more amino acid substitutions compared to a streptavidin mutein that is a minimal streptavidin. In some embodiments, the streptavidin contains one or more amino acid substitutions compared to a streptavidin mutein, e.g., a minimal streptavidin, that begins N-terminally in the region of amino acid positions 10 to 16 of SEQ ID NO: 1 and terminates C-terminally in the region of amino acid positions 133 to 142 of SEQ ID NO: 1. In some embodiments, the streptavidin contains one or more amino acid substitutions compared to the streptavidin mutein set forth in any of SEQ ID NO: 2, 103, and 135.

In some embodiments, the streptavidin mutein binds to biotin and contains at least 1, 2, 3, 4, 5, 6, 7, 8, 9, 10, 11, 12, 13, 14, 15, 16, 17, 18, 19, or 20 amino acid differences compared to the sequence of amino acids set forth in SEQ ID NO: 1, 2, 103, or 135. In some embodiments, the streptavidin mutein binds to biotin and contains an amino acid sequence that exhibits at least 85%, 86%, 87%, 88%, 89%, 90%, 91%, 92%, 93%, 94%, 95%, 96%, 97%, 98%, 99% or more sequence identity to the sequence of amino acids set forth in SEQ ID NO: 1, 2, 103, or 135. In some embodiments, the amino acid substitutions are conservative or non-conservative mutations. In some embodiments, the streptavidin mutein is any as described in U.S. Pat. Nos. 5,168,049; 5,506,121; 6,022,951; 6,156,493; 6,165,750; 6,103,493; 6,368,813; and Internation Published PCT Appl. Nos. WO2014/076277, WO2008/140573, WO 86/02077, WO 98/40396, and WO 96/24606. In some embodiments, the streptavidin mutein is any as described in DE 19641876 A1; Howarth et al. (2006) Nat. Methods, 3:267-73; Zhang et al. (2015) Biochem. Biophys. Res. Commun., 463:1059-63; Fairhead et al. (2013) J. Mol. Biol., 426:199-214; Wu et al. (2005) J. Biol. Chem., 280:23225-31; Lim et al. (2010) Biochemistry, 50:8682-91); and Qureshi et al. (2001), Journal of Biological Chemistry 276(49): 46422-46428.

In some embodiments, the streptavidin mutein is any as described in U.S. Pat. No. 6,103,493. In some embodiments, the streptavidin mutein contains at least one mutation within the region corresponding to amino acid positions 44 to 53 of wild-type streptavidin, such as set forth in SEQ ID NO: 1. In some embodiments, “corresponding to” references amino acid positions with reference to the amino acid sequence of wild-type streptavidin, such as set forth in SEQ ID NO: 1. One of ordinary skill in the art would be able to identify these residues with methods involving, e.g., the alignment of sequences. In some embodiments, the streptavidin mutein contains a mutation at one or more of residues 44, 45, 46, and 47 of wild-type streptavidin. In some embodiments, the streptavidin mutein contains a replacement of Glu at position 44 with a hydrophobic aliphatic amino acid, e.g., Val, Ala, Ile, or Leu. In some embodiments, the streptavidin mutein contains any amino acid at position 45. In some embodiments, the streptavidin mutein contains an aliphatic amino acid, such as a hydrophobic aliphatic amino acid, at position 46. In some embodiments, the streptavidin mutein contains a replacement of Val at position 47 with a basic amino acid, e.g., Arg or Lys, such as Arg. In some embodiments, Ala is at position 46, Arg is at position 47, and Val or Ile is at position 44. In some embodiments, the streptavidin mutein contains residues Val⁴⁴-Thr⁴⁵-Ala⁴⁶-Arg⁴⁷ (SEQ ID NO: 134) at sequence positions corresponding to positions 44 to 47 of the sequence of amino acids set forth in SEQ ID NO: 1, such as set forth in exemplary streptavidin muteins containing the sequence of amino acids set forth in SEQ ID NO: 3, 4, or 104. In some embodiments, the streptavidin mutein contains residues Ile⁴⁴-Gly⁴⁵-Ala⁴⁶-Arg⁴⁷ (SEQ ID NO: 133) at sequence positions corresponding to positions 44 to 47 of the sequence of amino acids set forth in SEQ ID NO: 1, such as set forth in exemplary streptavidin muteins containing the sequence of amino acids set forth in SEQ ID NO: 5, 6, or 104. In some embodiments, the streptavidin mutein contains the amino acid sequence set forth in any of SEQ ID NO: 3-6, 104, and 105. In some embodiments, the streptavidin mutein is commercially available under the trademark Strep-Tactin® ml. In some embodiments, the streptavidin mutein is commercially available under the trademark Strep-Tactin® m2. In some embodiments, the streptavidin mutein contains the amino acid sequence set forth in SEQ ID NO: 4. In some embodiments, the streptavidin mutein contains the amino acid sequence set forth in SEQ ID NO: 6.

In some embodiment, the streptavidin mutein is any as described in International Published PCT Appl. No. WO 2014/076277. In some embodiments, the streptavidin mutein contains at least two cysteine residues in the region corresponding to amino acid positions 44 to 53 of the amino acid sequence set forth in SEQ ID NO: 1. In some embodiments, the cysteine residues are present at positions 45 and 52 to create a disulfide bridge connecting these amino acids. In some embodiments, amino acid 44 is glycine or alanine; amino acid 46 is alanine or glycine; and amino acid 47 is arginine. In some embodiments, the streptavidin mutein contains at least one mutation in the region corresponding to amino acids residues 115 to 121 of the amino acid sequence set forth in SEQ ID NO: 1. In some embodiments, the streptavidin mutein contains at least one mutation at amino acid position 117, 120, or 121 and/or a deletion of amino acids 118 and 119 and substitution of at least amino acid position 121.

In some embodiments, the streptavidin mutein contains a mutation at a position corresponding to position 117, which mutation can be to a large hydrophobic residue like Trp, Tyr, or Phe; to a charged residue like Glu, Asp, or Arg; to a hydrophilic residue like Asn or Gln; to the hydrophobic residues Leu, Met, or Ala; or the polar residues Thr, Ser, or His. In some embodiments, the mutation at position 117 is combined with a mutation at a position corresponding to position 120, which mutation can be to a small residue like Ser, Ala, or Gly, and a mutation at a position corresponding to position 121, which mutation can be to a hydrophobic residue, such as a bulky hydrophobic residue like Trp, Tyr, or Phe. In some embodiments, the mutation at position 117 is combined with a mutation at a position corresponding to position 120 of wild-type streptavidin set forth in SEQ ID NO: 1, which mutation can be a hydrophobic residue such as Leu, Ile, Met, or Val; or Tyr or Phe, and a mutation at a position corresponding to position 121 of SEQ ID NO: 1, which mutation can be to a small residue like Gly, Ala, or Ser, or with Gln, or with a hydrophobic residue like Leu, Val, Ile, Trp, Tyr, Phe, or Met. In some embodiments, the streptavidin mutein contains the residues Glu117, Gly120, and Tyr121 with reference to positions of the sequence of amino acids set forth in SEQ ID NO: 1. In some embodiments, the streptavidin mutein also contains residues Val⁴⁴-Thr⁴⁵-Ala⁴⁶-Arg⁴⁷ or residues Ile⁴⁴-Gly⁴⁵-Ala⁴⁶-Arg⁴⁷ at sequence positions corresponding to positions 44 to 47 of the sequence of amino acids set forth in SEQ ID NO: 1. In some embodiments, the streptavidin mutein contains the residues Val44, Thr45, Ala46, Arg47, Glu117, Gly120, and Tyr121. In some embodiments, the mutein streptavidin contains the sequence of amino acids set forth in any of SEQ ID NO: 27, 28, and 136, or a sequence of amino acids that exhibits at least 85%, 86%, 87%, 88%, 89%, 90%, 91%, 92%, 93%, 94%, 95%, 96%, 97%, 98%, 99% or more sequence identity to the sequence of amino acids set forth in any of SEQ ID NO: 27, 28, and 136, contains the residues Val44, Thr45, Ala46, Arg47, Glu117, Gly120 and Tyr121, and binds to biotin. In some embodiments, the streptavidin mutein contains the sequence of amino acids set forth in SEQ ID NO: 27. In some embodiments, the streptavidin mutein contains the sequence of amino acids set forth in SEQ ID NO: 28. In some embodiments, the streptavidin mutein contains the sequence of amino acids set forth in SEQ ID NO: 136.

In some embodiments, the streptavidin mutein contains the sequence of amino acids set forth in any of SEQ ID NO: 3-6, 27, 28, 104, 105, and 136, and the binding partner contains a streptavidin-binding peptide, wherein the sequence of the streptavidin-binding peptide is set forth in any of SEQ ID NO: 7, 8, and 15-19. In some embodiments, the streptavidin mutein contains the sequence of amino acids set forth in SEQ ID NO: 6, and the binding partner contains a streptavidin-binding peptide, wherein the sequence of the streptavidin-binding peptide is set forth in any of SEQ ID NO: 7, 8, and 15-19. In some embodiments, the streptavidin mutein contains the sequence of amino acids set forth in any of SEQ ID NO: 3-6, 27, 28, 104, 105, and 136, and the binding partner contains a streptavidin-binding peptide, wherein the sequence of the streptavidin-binding peptide is set forth in SEQ ID NO: 16. In some embodiments, the streptavidin mutein contains the sequence of amino acids set forth in SEQ ID NO: 6, and the binding partner contains a streptavidin-binding peptide, wherein the sequence of the streptavidin-binding peptide is set forth in SEQ ID NO: 16.

In some embodiments, the protein reagent contains a plurality of molecules, for instance a plurality of any of the described streptavidin, avidin, streptavidin analog or mutein, and avidin analog or mutein molecules. In some embodiments, the plurality of molecules is a mixture of molecules each independently selected from any of the streptavidin, avidin, streptavidin analog or mutein, and avidin analog or mutein molecules described herein. In some embodiments, the plurality of molecules is a mixture of any of the streptavidin and streptavidin mutein molecules described herein.

In some embodiments, each of the plurality of molecules is the same and is any one of the streptavidin, avidin, streptavidin analog or mutein, and avidin analog or mutein molecules described herein. In some embodiments, each of the plurality of molecules is the same and is streptavidin. In some embodiments, each of the plurality of molecules is the same and is any one of the streptavidin mutein molecules described herein.

In some embodiments, the plurality of molecules contains between 100 and 50,000, between 500 and 10,000, between 1,000 and 20,000, between 500 and 5,000, between 300 and 7,500, between 1,500 and 7,500, between 500 and 3,500, between 1,000 and 5,000, between 1,500 and 2,500, between 1,500 and 2,500, between 2,000 and 3,000, between 2,500 and 3,500, between 2,000 and 4,000, or between 2,000 and 5,000 tetramers, each inclusive, of the molecule or mixture of molecules. In some embodiments, the plurality of molecules contains between or between about 500 and 7500 tetramers, inclusive, of the molecule or mixture of molecules. In some embodiments, the plurality of molecules contains between or between about 500 and 5000 tetramers, 1000 and 4000 tetramers, or 2000 and 3000 tetramers, each inclusive, of the molecule or mixture of molecules. In some embodiments, the plurality of molecules contains between or between about 500 and 5000 tetramers, inclusive, of the molecule or mixture of molecules. In some embodiments, the plurality of molecules contains between or between about 1000 and 4000 tetramers, inclusive, of the molecule or mixture of molecules. In some embodiments, the plurality of molecules contains between or between about 2000 and 3000 tetramers, inclusive, of the molecule or mixture of molecules. In some embodiments, the plurality of molecules contains about 2500 tetramers of the molecule or mixture of molecules. In any of the foregoing embodiments, the number of tetramers is the number of tetramers of the molecule. In any of the foregoing embodiments, the number of tetramers is the number of tetramers of the mixture of molecules.

In some embodiments, the protein reagent has a radius of between 5 nm and 150 nm, between 25 nm and 150 nm, between 50 nm and 150 nm, between 75 nm and 125 nm, between 80 nm and 140 nm, between 85 nm and 135 nm, between 80 nm and 120 nm, between 80 nm and 115 nm, or between 90 nm and 110 nm, inclusive. In some embodiments, the protein reagent has a radius of between 50 nm and 150 nm, inclusive. In some embodiments, the protein reagent has a radius of between 75 nm and 125 nm, inclusive. In some embodiments, the protein reagent has a radius of between 80 nm and 120 nm, inclusive. In some embodiments, the protein reagent has a radius of between 90 nm and 110 nm, inclusive.

In some embodiments, the radius is the hydrodynamic radius, radius of gyration, Stokes radius, Stokes-Einstein radius, or the effective hydrated radius in solution. In some embodiments, the radius is the hydrodynamic radius. In some embodiments, the radius is the Stokes radius.

In some embodiments, the protein reagent is an oligomer of the plurality of molecules. In some embodiments, the oligomer is generated by linking individual molecules of the protein reagent. In some embodiments, the oligomer is generated by linking monomers, dimers, trimers, or tetramers of the molecule. In some embodiments, the molecules are directly linked to one another. In some embodiments, the molecules are indirectly linked to one another. Oligomers can be generated using any suitable method, such as any described in U.S. Pat. No. 7,776,562 and Published U.S. Patent Appl. No. 2021/0032297. In some embodiments, molecules of the plurality of molecules are crosslinked by a polysaccharide or a bifunctional linker.

In some cases, molecules of the plurality of molecules are crosslinked by a polysaccharide. In some embodiments, the oligomer is prepared by the introduction of carboxyl residues into a polysaccharide, e.g., dextran, for instance as described in Noguchi et al, Bioconjugate Chemistry (1992) 3, 132-137 in a first step. In some embodiments, the molecules of the protein reagent, e.g., the streptavidin, avidin, streptavidin analog or mutein, or avidin analog or mutein molecules, may then be linked via primary amino groups of internal lysine residues and/or the free N-terminus to the carboxyl groups in the dextran backbone using carbodiimide chemistry in a second step.

In some embodiments, molecules of the plurality of molecules are crosslinked by a bifunctional linker. Suitable bifunctional linkers can be identified and selected by one of ordinary skill in the art. In some embodiments, the linker is a heterobifunctional linker. In some embodiments, molecules of the plurality of molecules, e.g., the streptavidin, avidin, streptavidin analog or mutein, or avidin analog or mutein molecules, such as the streptavidin mutein molecules, are crosslinked by an amine-to-thiol crosslinker. Exemplary crosslinking reagents include sulfosuccinimidyl 4-(N-maleimidomethyl)cyclohexane-1-carboxylate (sulfo SMCC) or Succinimidyl-6-[(β-maleimidopropionamido)hexanoate (SMPH), and their use in generating oligomers is described in, e.g., US2021/0032297.

b. Binding Agents

In some embodiments, the one or more binding agents are suitable for the stimulation of immune cells, e.g., T cells. In some embodiments, the one or more binding agents are immobilized on the protein reagent of the stimulatory reagent. In some embodiments, the one or more binding agents are individually selected from among any of the binding agents described herein. In some embodiments, the one or more binding agents include 2, 3, 4, 5, 6, 7, 8, 9, or 10 different binding agents, which can target the same or different molecules. For instance, in some embodiments, the one or more binding agents include a primary agent and a secondary agent that target different molecules from one another.

In some embodiments, one of the one or more binding agents is a primary agent that binds to a molecule expressed on the surface of immune cells, e.g., T cells, and thereby provides a primary activation signal to the immune cells, e.g., T cells. In some embodiments, the molecule is a member of a TCR/CD3 complex. In some embodiments, the molecule is CD3.

In some embodiments, one of the one or more binding agents is a secondary agent that binds to a second molecule expressed on the surface of the immune cells, e.g., T cells. In some embodiments, the second molecule is a costimulatory molecule. In some embodiments, the secondary agent binds and thereby provides a costimulatory signal to the immune cells, e.g., T cells. In some embodiments, the costimulatory molecule is CD28, CD90 (Thy-1), CD95 (Apo-/Fas), CD137 (4-1BB), CD154 (CD40L), ICOS, LAT, CD27, OX40, or HVEM. In some embodiments, the costimulatory molecule is CD28.

In some embodiments, the binding agent binds to a molecule expressed on the surface of an immune cell, e.g., T cell. A wide variety of, for example, antibodies or antibody fragments that target cell surface molecules are available and suitable for use as part of the binding agents herein and can be identified and selected by one of ordinary skill in the art for use accordingly.

In some embodiments, the binding agent is monovalent. In some embodiments, the binding agent contains two or more binding sites for binding to the molecule expressed on the surface of the immune cell (also referred to herein as the cell surface molecule). In some embodiments, the binding agent is divalent.

In some embodiments, the dissociation constant (K_{D}) of the binding between the binding agent and the cell surface molecule is from about 10⁻² M to about 10⁻¹³ M, from about 10⁻³ M to about 10⁻¹² M, from about 10⁻⁴ M to about 10⁻¹¹M, or from about 10⁻⁵M to about 10⁻¹⁰ M. In some embodiments, the dissociation constant (K_{D}) for the binding between the binding agent and the cell surface molecule is from about 10⁻³ to about 10⁻⁷M, e.g., is of low affinity. In some embodiments, the dissociation constant (K_{D}) for the binding between the binding agent and the cell surface molecule is from about 10⁻⁷ to about 1×10⁻¹° M, e.g., is of high affinity.

In some embodiments, the dissociation of the binding between the binding agent and the cell surface molecule occurs sufficiently fast to, for example, allow the immune cell, e.g., T cell, to be only transiently associated with the binding agent after disruption of the reversible bond between the protein reagent and the binding agent. In some embodiments, when expressed in terms of the k_{off} rate (also called dissociation rate constant) for the binding between the binding agent and the cell surface molecule, the k_{off} rate is about 0.5×10⁻⁴ sec⁻¹ or greater, about 1×10⁻⁴ sec⁻¹ or greater, about 2×10⁻⁴ sec⁻¹ or greater, about 3×10⁻⁴ sec⁻¹ or greater, about 4×10⁻⁴ sec⁻¹ of greater, about 5×10⁻⁴ sec⁻¹ or greater, about 1×10⁻³ sec⁻¹ or greater, about 1.5×10⁻³ sec⁻¹ or greater, about 2×10⁻³ sec⁻¹ or greater, about 3×10⁻³ sec⁻¹ or greater, about 4×10⁻³ sec 1, about 5×10⁻³ sec⁻¹ or greater, about 1×10⁻² sec or greater, or about 5×10⁻¹ sec⁻¹ or greater. It is within the level of one of ordinary skill in the art to empirically determine the k_{off} rate range suitable for a particular binding agent and cell surface molecule interaction (see, e.g., U.S. Pat. No. 9,023,604). For example, a binding agent with a higher k_{off} rate of, for example, greater than 4.0×10⁻⁴ sec⁻¹ may be used so that after the disruption of the binding to the protein reagent, most of the binding agent can be removed or dissociated from the immune cell, e.g., T cell, within one hour. In other cases, a binding agent with a lower k_{off} rate of, for example, 1.0×10⁻⁴ sec⁻¹, may be used so that after the disruption of the binding to the protein reagent, most of the binding agent may be removed or dissociated from the immune cell, e.g., T cell, within about 3 and a half hours.

The K_{D}, k_{off}, and kₒₙ rate of the bond formed between the binding agent and the cell surface molecule can be determined by any suitable means, for example by fluorescence titration, equilibrium dialysis, or surface plasmon resonance.

In some embodiments, the receptor is a lipid, a polysaccharide, or a nucleic acid. In some embodiments, the cell surface molecule is a peptide or a protein, such as a receptor, e.g., a membrane receptor protein. In some embodiments, the cell surface molecule is a peripheral membrane protein or an integral membrane protein. The cell surface molecule can in some embodiments have one or more domains that span the membrane. As a few illustrative examples, a membrane protein with a transmembrane domain may be a G-protein coupled receptor, such as an odorant receptors, a rhodopsin receptor, a rhodopsin pheromone receptor, a peptide hormone receptor, a taste receptor, a GABA receptor, an opiate receptor, a serotonin receptor, a Ca2+ receptor, melanopsin, a neurotransmitter receptor, such as a ligand gated, a voltage gated or a mechanically gated receptor, including the acetylcholine, the nicotinic, the adrenergic, the norepinephrine, the catecholamines, the L-DOPA-, a dopamine and serotonin (biogenic amine, endorphin/enkephalin) neuropeptide receptor, a receptor kinase such as serine/threonine kinase, a tyrosine kinase, a porin/channel such as a chloride channel, a potassium channel, a sodium channel, an OMP protein, an ABC transporter (ATP-Binding Cassette-Transporter) such as amino acid transporter, the Na-glucose transporter, the Na/iodide transporter, an ion transporter such as Light Harvesting Complex, cytochrome c oxidase, ATPase Na/K, H/K, Ca, a cell adhesion receptor such as metalloprotease, an integrin, or a catherin.

In some embodiments, the cell surface molecule is a molecule expressed by or defining a cell population, for instance a population or subpopulation of blood cells, e.g., lymphocytes (e.g., T cells, B cells, or NK cells), monocytes, or stem cells (e.g., CD34 positive peripheral stem cells or Nanog or Oct-4 expressing stem cells). In some embodiments, the cell surface molecule is expressed on the surface of a target cell, e.g., a cell targeted for genetic engineering. In some embodiments, the cell surface molecule is a molecule expressed on the surface of immune cells. In some embodiments, the cell surface molecule is a molecule expressed on the surface of lymphocytes. In some embodiments, the cell surface molecule is a molecule expressed on the surface of T cells, B cells, or NK cells. In some embodiments, the cell surface molecule is a molecule expressed on the surface of T cells. Examples of T cells include cells such as CMV-specific CD8+ T cells, cytotoxic T cells, memory T cells, and regulatory T-cells (Treg). An illustrative example of Treg includes CD4 CD25 CD45RA Treg cells, and an illustrative example of memory T cells includes CD62L CD8+ specific central memory T cells.

In some embodiments, the binding agent contains an antibody, an antibody fragment, a proteinaceous molecule with antibody-like binding properties, a molecule containing Ig domains, a cytokine, a chemokine, an MHC molecules, an MHC-peptide complex, a receptor ligand, or a binding fragment of any of the foregoing, that specifically binds to the cell surface molecule. In some embodiments, the binding agent contains an antibody. In some embodiments, the binding agent contains an antibody fragment. In some embodiments, the antibody fragment is selected from Fab fragments, Fv fragments, single-chain Fv fragments (scFv), divalent antibody fragments such as F(ab′)₂-fragments, diabodies, triabodies (Iliades, P., et al., FEBS Lett (1997) 409, 437-441), decabodies (Stone, E., et al., Journal of Immunological Methods (2007) 318, 88-94), and other domain antibodies (Holt, L. J., et al., Trends Biotechnol. (2003), 21, 11, 484-490).

In some embodiments, the binding agent binds to the cell surface molecule in a monovalent manner. In some embodiments, the binding agent contains a monovalent antibody fragment, a proteinaceous binding molecule with antibody-like binding properties, an aptamer, or an MHC molecule. In some embodiments, the binding agent contains a monovalent antibody fragment. In some embodiments, the monovalent antibody fragment is a Fab fragment, Fv fragment, or single-chain Fv fragment (scFv). In some embodiments, the monovalent antibody fragment is a Fab fragment.

In some embodiments, the binding agent contains an antibody fragment that is a divalent antibody fragment. In some embodiments, the divalent antibody fragment is an F(ab′)2-fragment or a divalent single-chain Fv fragment.

In some embodiments, the binding agent contains a proteinaceous molecule with antibody-like binding properties. In some embodiments, the proteinaceous molecule with antibody-like binding properties is an aptamer, a mutein based on a polypeptide of the lipocalin family, a glubody, a protein based on the ankyrin scaffold, a protein based on the crystalline scaffold, an adnectin, or an avimer. Other exemplary proteinaceous molecules include an EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or a an immunoglobulin-like domain (for example, domain antibodies or camel heavy chain antibodies), a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, “Kappabodies” (cf. Ill. et al., Protein Eng (1997) 10, 949-57, a so called “minibody” (Martin et al., EMBO J (1994) 13, 5303-5309), a diabody (cf. Holliger et al., PNAS USA (1993)90, 6444-6448), a so called “Janusis” (cf. Traunecker et al., EMBO J (1991) 10, 3655-3659, or Traunecker et al., Int J Cancer (1992) Suppl 7, 51-52), a nanobody, a microbody, an affilin, an affibody, a knottin, ubiquitin, a zinc-finger protein, an autofluorescent protein, and a leucine-rich repeat protein. In some embodiments, the binding agent is a bivalent proteinaceous artificial binding molecule such as a dimeric lipocalin mutein that is also known as “duocalin”.

In some embodiments, the cell surface molecule is a molecule containing an immunoreceptor tyrosine-based activation motif (ITAM). In some embodiments, the cell surface molecule is a member of a T cell antigen receptor complex. In some embodiments, the cell surface molecule is a member of a TCR/CD3 complex. In some embodiments, the cell surface molecule is CD3. In some embodiments, the cell surface molecule is a CD3 chain. In some embodiments, the cell surface molecule is a CD3 zeta chain.

In some embodiments, the cell surface molecule is CD3. In some embodiments, the binding agent, e.g., primary agent, contains an anti-CD3 antibody, a divalent antibody fragment of an anti-CD3 antibody, a monovalent antibody fragment of an anti-CD3 antibody, or a proteinaceous CD3 binding molecule with antibody-like binding properties. In some embodiments, the anti-CD3 antibody, divalent antibody fragment of an anti-CD3 antibody, or monovalent antibody fragment of an anti-CD3 antibody (e.g., anti-CD3 Fab fragment) is derived from antibody OKT3 (e.g., ATCC CRL-8001; see, e.g., Stemberger et al. pLoS One. 2012; 7(4): e35798) or a functionally active mutant thereof that retains specific binding for CD3. In some embodiments, the binding agent, e.g., primary agent, contains an anti-CD3 Fab. In some embodiments, the anti-CD3 Fab contains a variable heavy chain having the sequence set forth in SEQ ID NO: 31 and a variable light chain having the sequence set forth in SEQ ID NO: 32. In some embodiments, the anti-CD3 Fab contains the CDRs of the variable heavy chain having the sequence set forth in SEQ ID NO: 31 and the CDRs of the variable light chain having the sequence set forth in SEQ ID NO: 32.

In some embodiments, the cell surface molecule is a costimulatory molecule, an accessory molecule, a cytokine receptor, a chemokine receptor, an immune checkpoint molecule, or a member of the TNF family or TNF receptor family. In some embodiments, the cell surface molecule is a costimulatory molecule. In some embodiments, the costimulatory molecule is CD28, CD90 (Thy-1), CD95 (Apo-/Fas), CD137 (4-1BB), CD154 (CD40L), ICOS, LAT, CD27, OX40, or HVEM.

In some embodiments, the cell surface molecule is CD28. In some embodiments, the binding agent, e.g., secondary agent, contains an anti-CD28 antibody, a divalent antibody fragment of an anti-CD28 antibody, a monovalent antibody fragment of an anti-CD28 antibody, or a proteinaceous CD28 binding molecule with antibody-like binding properties. In some embodiments, the anti-CD28 antibody, divalent antibody fragment of an anti-CD28 antibody, or monovalent antibody fragment of an anti-CD28 antibody (e.g., anti-CD28 Fab fragment) is derived from antibody CD28.3 (deposited as a synthetic single chain Fv construct under GenBank Accession No. AF451974.1; see also Vanhove et al, BLOOD, 15 Jul. 2003, Vol. 102, No. 2, pages 564-570), the variable heavy and light chains of which contain the amino acid sequences set forth in SEQ ID NO: 33 and 34, respectively. In some embodiments, the binding agent, e.g., secondary agent, contains an anti-CD28 Fab. In some embodiments, the anti-CD28 Fab contains a variable heavy chain having the sequence set forth in SEQ ID NO: 33 and a variable light chain having the sequence set forth in SEQ ID NO: 34. In some embodiments, the anti-CD28 Fab contains the CDRs of the variable heavy chain having the sequence set forth in SEQ ID NO: 33 and the CDRs of the variable light chain having the sequence set forth in SEQ ID NO: 34.

In some embodiments, the cell surface molecule is CD90. In some embodiments, the binding agent, e.g., secondary agent, contains an anti-CD90 antibody, a divalent antibody fragment of an anti-CD90 antibody, a monovalent antibody fragment of an anti-CD90 antibody, or a proteinaceous CD90 binding molecule with antibody-like binding properties. In some embodiments, the binding agent, e.g., secondary agent, contains an anti-CD90 Fab. In some embodiments, the anti-CD90 antibody, divalent antibody fragment of an anti-CD90 antibody, or monovalent antibody fragment of an anti-CD90 antibody (e.g., anti-CD90 Fab fragment) is derived from the anti-CD90 antibody G7 (Biolegend, cat. no. 105201).

In some embodiments, the cell surface molecule is CD95. In some embodiments, the binding agent, e.g., secondary agent, contains an anti-CD95 antibody, a divalent antibody fragment of an anti-CD95 antibody, a monovalent antibody fragment of an anti-CD95 antibody, or a proteinaceous CD95 binding molecule with antibody-like binding properties. In some embodiments, the binding agent, e.g., secondary agent, contains an anti-CD95 Fab. In some embodiments, the anti-CD95 antibody, divalent antibody fragment of an anti-CD95 antibody, or monovalent antibody fragment of an anti-CD95 antibody (e.g., anti-CD95 Fab fragment) is derived from monoclonal mouse anti-human CD95 CH11 (Upstate Biotechnology, Lake Placid, NY), anti-CD95 mAb 7C11, or anti-APO-1, such as described in Paulsen et al. Cell Death & Differentiation 18.4 (2011): 619-631.

In some embodiments, the cell surface molecule is CD137. In some embodiments, the binding agent, e.g., secondary agent, contains an anti-CD137 antibody, a divalent antibody fragment of an anti-CD137 antibody, a monovalent antibody fragment of an anti-CD137 antibody, or a proteinaceous CD137 binding molecule with antibody-like binding properties. In some embodiments, the binding agent, e.g., secondary agent, contains an anti-CD137 Fab. In some embodiments, the anti-CD137 antibody, divalent antibody fragment of an anti-CD137 antibody, or monovalent antibody fragment of an anti-CD137 antibody (e.g., anti-CD137 Fab fragment) is derived from LOB12, IgG2a or LOB12.3, IgG1 as described in Taraban et al. Eur J Immunol. 2002 December; 32(12):3617-27. See also, e.g., U.S. Pat. Nos. 6,569,997, 6,303,121, and Mittler et al. Immunol Res. 2004; 29(1-3):197-208.

In some embodiments, the cell surface molecule is CD40. In some embodiments, the binding agent, e.g., secondary agent, contains an anti-CD40 antibody, a divalent antibody fragment of an anti-CD40 antibody, a monovalent antibody fragment of an anti-CD40 antibody, or a proteinaceous CD40 binding molecule with antibody-like binding properties. In some embodiments, the binding agent, e.g., secondary agent, contains an anti-CD40 Fab.

In some embodiments, the cell surface molecule is CD40L. In some embodiments, the binding agent, e.g., secondary agent, contains an anti-CD40L antibody, a divalent antibody fragment of an anti-CD40L antibody, a monovalent antibody fragment of an anti-CD40L antibody, or a proteinaceous CD40L binding molecule with antibody-like binding properties. In some embodiments, the binding agent, e.g., secondary agent, contains an anti-CD40L Fab. In some embodiments, the anti-CD40L antibody, divalent antibody fragment of an anti-CD40L antibody, or monovalent antibody fragment of an anti-CD40L antibody (e.g., anti-CD40L Fab fragment) is derived from Hu5C8, as described in Blair et al. JEM vol. 191 no. 4 651-660. See also, e.g., WO1999061065, US20010026932, U.S. Pat. No. 7,547,438, and WO2001056603.

In some embodiments, the cell surface molecule is ICOS. In some embodiments, the binding agent, e.g., secondary agent, contains an anti-ICOS antibody, a divalent antibody fragment of an anti-ICOS antibody, a monovalent antibody fragment of an anti-ICOS antibody, or a proteinaceous ICOS binding molecule with antibody-like binding properties. In some embodiments, the binding agent, e.g., secondary agent, contains an anti-ICO Fab. In some embodiments, the anti-ICOS antibody, divalent antibody fragment of an anti-ICOS antibody, or monovalent antibody fragment of an anti-ICOS antibody (e.g., anti-ICOS Fab fragment) is derived from any of the antibodies described in US20080279851 and Deng et al. Hybrid Hybridomics. 2004 June; 23(3):176-82.

In some embodiments, the cell surface molecule is Linker for Activation of T cells (LAT). In some embodiments, the binding agent, e.g., secondary agent, contains an anti-LAT antibody, a divalent antibody fragment of an anti-LAT antibody, a monovalent antibody fragment of an anti-LAT antibody, or a proteinaceous LAT binding molecule with antibody-like binding properties. In some embodiments, the binding agent, e.g., secondary agent, contains an anti-LAT Fab.

In some embodiments, the cell surface molecule is CD27. In some embodiments, the binding agent, e.g., secondary agent, contains an anti-CD27 antibody, a divalent antibody fragment of an anti-CD27 antibody, a monovalent antibody fragment of an anti-CD27 antibody, or a proteinaceous CD27 binding molecule with antibody-like binding properties. In some embodiments, the binding agent, e.g., secondary agent, contains an anti-CD27 Fab. In some embodiments, the anti-CD27 antibody, divalent antibody fragment of an anti-CD27 antibody, or monovalent antibody fragment of an anti-CD27 antibody (e.g., anti-CD27 Fab fragment) is derived from any of the antibodies described in WO2008051424.

In some embodiments, the cell surface molecule is OX40. In some embodiments, the binding agent, e.g., secondary agent, contains an anti-OX40 antibody, a divalent antibody fragment of an anti-OX40 antibody, a monovalent antibody fragment of an anti-OX40 antibody, or a proteinaceous OX40 binding molecule with antibody-like binding properties. In some embodiments, the binding agent, e.g., secondary agent, contains an anti-OX40 Fab. In some embodiments, the anti-OX40 antibody, divalent antibody fragment of an anti-OX40 antibody, or monovalent antibody fragment of an anti-OX40 antibody (e.g., anti-OX40 Fab fragment) is derived from any of the antibodies described in WO2013038191 and Melero et al. Clin Cancer Res. 2013 Mar. 1; 19(5):1044-53.

In some embodiments, the cell surface molecule is HVEM. In some embodiments, the binding agent, e.g., secondary agent, contains an anti-HVEM antibody, a divalent antibody fragment of an anti-HVEM antibody, a monovalent antibody fragment of an anti-HVEM antibody, or a proteinaceous HVEM binding molecule with antibody-like binding properties. In some embodiments, the binding agent, e.g., secondary agent, contains an anti-HVEM Fab. In some embodiments, the anti-HVEM antibody, divalent antibody fragment of an anti-HVEM antibody, or monovalent antibody fragment of an anti-HVEM antibody (e.g., anti-HVEM Fab fragment) is derived from any of the antibodies described in WO2006054961, WO2007001459, and Park et al. Cancer Immunol Immunother. 2012 February; 61(2):203-14.

In some embodiments, the binding agent further contains a binding partner. In some embodiments, the binding agent contains between 1 and 5, 1 and 4, 1 and 3, or 1 and 2 binding partners, each inclusive. In some embodiments, the binding agent contains exactly one binding partner. In some embodiments, the binding agent contains exactly two binding partners. In some embodiments, the binding agent contains exactly three binding partners. In some embodiments, the binding agent contains exactly four binding partners. In some embodiments, the binding agent contains exactly five binding partners.

Exemplary binding partners are described in this section. In some embodiments, each binding partner of a binding agent containing multiple binding partners is individually selected from among the described binding partners. In some embodiments, each binding partner of a binding agent containing multiple binding partners is the same and is any one of the binding partners described herein.

Im some embodiments, the binding partner is hydrocarbon-based (including polymeric) and contains nitrogen-, phosphorus-, sulphur-, carben-, halogen- or pseudohalogen groups. In some embodiments, the binding partner is an alcohol, an organic acid, an inorganic acid, an amine, a phosphine, a thiol, a disulfide, an alkane, an amino acid, a peptide, an oligopeptide, a polypeptide, a protein, a nucleic acid, a lipid, a saccharide, an oligosaccharide, or a polysaccharide. As further examples, in some embodiments, the binding partner is a cation, an anion, a polycation, a polyanion, a polycation, an electrolyte, a polyelectrolyte, a carbon nanotube, or carbon nanofoam. As yet further examples, in some embodiments, the binding partner is a crown ether, an immunoglobulin or a fragment thereof, or a proteinaceous binding molecule with antibody-like functions.

In some embodiments, the binding partner includes a moiety known to one of ordinary skill in the art as an affinity tag. In some embodiments, the protein reagent includes a corresponding binding partner, for example an antibody or an antibody fragment known to bind to the affinity tag. As a few illustrative examples of known affinity tags, in some embodiments, the affinity tag includes dinitrophenol or digoxigenin, oligohistidine, polyhistidine, an immunoglobulin domain, glutathione-S-transferase (GST), chitin binding protein (CBP) or thioredoxin, calmodulin binding peptide (CBP), FLAG′-peptide, the HA-tag (SEQ ID NO: 20), the VSV-G-tag (SEQ ID NO: 21), the HSV-tag (SEQ ID NO: 22), the T7 epitope (SEQ ID NO: 23), maltose binding protein (MBP), the HSV epitope (SEQ ID NO: 24) of herpes simplex virus glycoprotein D, the “myc” epitope of the transcription factor c-myc (SEQ ID NO: 25), or the V5-tag (SEQ ID NO: 26). In some embodiments, the complex formed between the binding site of the protein reagent and the affinity tag, for instance between the corresponding binding partner of the selectio reagent, e.g., an antibody or antibody fragment, and the affinity tag, can be disrupted competitively by contacting the complex with a free binding partner, e.g., an unbound affinity tag.

In some embodiments, the affinity tag includes an oligonucleotide tag. In some some embodiments, the oligonucleotide tag hybridizes to an oligonucleotide linked to or included in the protein reagent with a complementary sequence.

In some embodiments, the binding partner is a lectin, protein A, protein G, a metal, a metal ion, nitrilo triacetic acid derivatives (NT A), RGD-motifs, a dextrane, polyethyleneimine (PEI), a redox polymer, a glycoprotein, an aptamer, a dye, amylose, maltose, cellulose, chitin, glutathione, calmodulin, gelatine, polymyxin, heparin, NAD, NADP, lysine, arginine, benzamidine, poly U, or oligo-dT. Lectins such as Concavalin A are known to bind to polysaccharides and glycosylated proteins. An illustrative example of a dye is a triazine dye, such as Cibacron blue F3G-A (CB) or Red HE-3B, which specifically binds NADH-dependent enzymes. Green A is known to bind to Co A proteins, human serum albumin, and dehydrogenases. The dyes 7-aminoactinomycin D and 4′,6-diamidino-2-phenylindole are known to bind to DNA. Cations of metals such as Ni, Cd, Zn, Co, or Cu can also be used to bind affinity tags, such as an oligohistidine-containing sequence, including the hexahistidine or the MAT tag (SEQ ID NO: 35), and N-methacryloyl-(L)-cysteine methyl ester.

In some embodiments, the binding between the binding partner and the binding site of the protein reagent occurs in the presence of a divalent, a trivalent, or a tetravalent cation. In some embodiments, the protein reagent includes a divalent, a trivalent, or a tetravalent cation, for instance held, e.g., complexed, by means of a suitable chelator. In some embodiments, the binding partner includes a moiety that complexes with a divalent, a trivalent, or a tetravalent cation. Examples of metal chelators include ethylenediamine, ethylene-diaminetetraacetic acid (EDTA), ethylene glycol tetraacetic acid (EGTA), diethylenetri-aminepentaacetic acid (DTPA), N,N-bis(carboxymethyl)glycine (also called nitrilotriacetic acid, NTA), 1,2-bis(o-aminophenoxy)ethane-N,N,N′,N′-tetraacetic acid (BAPTA), 2,3-dimer-capto-1-propanol (dimercaprol), porphine, and heme. As an example, EDTA can form a complex with most monovalent, divalent, trivalent, and tetravalent metal ions, such as silver (Ag⁺), calcium (Ca²⁺), manganese (Mn²⁺), copper (Cu²⁺), iron (Fe²⁺), cobalt (Co⁺), and zirconium (Zr⁴⁺), while BAPTA is specific for Ca²⁺. As an illustrative example, one of ordinary skill in the art can use methods involving the formation of a complex between an oligohistidine tag and copper (Cu²⁺), nickel (Ni²⁺), cobalt (Co²⁺), or zinc (Zn²⁺) ions, which are presented by means of the chelator nitrilotriacetic acid (NTA).

In some embodiments, the binding partner includes a calmodulin-binding peptide, and the protein reagent includes multimeric calmodulin, for instance as described in U.S. Pat. No. 5,985,658. In some embodiments, the binding partner includes a FLAG peptide, and the protein reagent includes an antibody that binds to the FLAG peptide. For instance, in some embodiments, the protein reagent includes the monoclonal antibody 4E11 that binds to the FLAG peptide, for instance as described in U.S. Pat. No. 4,851,341. In some embodiments, the binding partner includes an oligohistidine tag, and the protein reagent includes an antibody or a transition metal ion that binds the oligohistidine tag. In some embodiments, calmodulin, antibodies such as 4E11, chelated metal ions, and free chelators may be multimerized by methods involving, for example, biotinylation and complexation with streptavidin, avidin, or oligomers thereof, or by the introduction of carboxyl residues into a polysaccharide, e.g., dextran, for instance as described in Noguchi et al. (1992), Bioconjugate Chemistry 3: 132-137, in a first step, and linking calmodulin, antibodies, chelated metal ions, or free chelators via primary amino groups to the carboxyl groups in the polysaccharide, e.g. dextran, using carbodiimide chemistry in a second step. In some embodiments, the binding between the binding partner and the binding site of the protein reagent can be disrupted by metal ion chelation. The metal chelation may be accomplished by, for example, addition of EGTA or EDTA.

In some embodiments, the binding partner binds to a biotin-binding molecule. In some embodiments, the binding partner binds to the biotin-binding site of the molecule.

In some embodiments, the binding partner is a streptavidin or avidin binding partner. In some embodiments, the binding partner is a streptavidin-binding partner. In some embodiments, the streptavidin-binding partner is also an avidin-binding partner.

In some embodiments, the binding partner binds to a molecule that is streptavidin, avidin, a streptavidin analog or mutein, or an avidin analog or mutein. In some embodiments, the molecule is any of the streptavidin, avidin, streptavidin analog or mutein, and avidin analog or mutein molecules described in Section I-B-1-a. In some embodiments, the protein reagent contains the molecule. In some embodiments, the binding partner binds to a biotin-binding site of the molecule. In some embodiments, the binding partner binds to the natural biotin-binding site of the molecule (see, e.g., Qureshi et al. (2001), Journal of Biological Chemistry 276(49): 46422-46428; and Livnah et al. (1993), Proc Natl Acad Sci 90: 5076-5080; which describe the interactions of biotin with streptavidin and avidin, respectively). In some embodiments, the binding partner allows for the functionalization of reagents containing streptavidin, avidin, a streptavidin analog or mutein, or an avidin analog or mutein.

Binding partners that bind to streptavidin, avidin, a streptavidin analog or mutein, or an avidin analog or mutein, including that bind to the biotin-binding sites of these molecules, can be identified and selected by one of ordinary skill in the art. In some embodiments, the binding partner binds to a molecule that is streptavidin.

In some embodiments, the binding partner contains biotin. In some embodiments, the binding partner is biotin. In some embodiments, the biotin is D-biotin. In some embodiments, the binding partner contains a biotin analog or derivate. In some embodiments, the binding partner is a biotin analog or derivate. In some embodiments, the biotin analog or derivative is a structural analog of biotin. In some embodiments, the biotin analog or derivative binds to the biotin-binding site of streptavidin, avidin, a streptavidin analog or mutein, or an avidin analog or mutein. In some embodiments, the biotin analog or derivative binds to the biotin-binding site of streptavidin. In some embodiments, the biotin analog or derivative is desthiobiotin, iminobiotin, guanidinobiotin, diaminobiotin, lipoic acid, HABA (hydroxyazobenzene-benzoic acid), dimethyl-HABA, biotin sulfone, caproylamidobiotin, or biocytin (or any of the biotin analogs and derivatives described in, e.g., International Published PCT Appl. No. WO2008140573).

In some embodiments, the binding partner contains a streptavidin-binding peptide. In some embodiments, the binding partner is a streptavidin-binding peptide. In some embodiments, the streptavidin-binding peptide binds to the biotin-binding site of streptavidin, avidin, a streptavidin analog or mutein, or an avidin analog or mutein. In some embodiments, the streptavidin-binding peptide binds to the biotin-binding site of streptavidin. In some embodiments, the streptavidin-binding peptide contains an amino acid sequence with the formula set forth in SEQ ID NO: 9, such as contains the amino acid sequence set forth in SEQ ID NO: 10. In some embodiments, the streptavidin-binding peptide contains an amino acid sequence with the formula set forth in SEQ ID NO: 11, such as set forth in SEQ ID NO: 12. In some embodiments, the streptavidin-binding peptide contains the amino acid sequence set forth in SEQ ID NO: 7, also called Strep-tag®. In some embodiments, the sequence of the streptavidin-binding peptide is set forth in SEQ ID NO: 7. In some embodiments, the streptavidin-binding peptide contains the amino acid sequence set forth in SEQ ID NO: 8, also called Strep-tag® II. In some embodiments, the sequence of the streptavidin-binding peptide is set forth in SEQ ID NO: 8.

In some embodiments, the streptavidin-binding peptide may be further modified. In some embodiments, the streptavidin-binding peptide contains the amino acid sequence set forth in SEQ ID NO: 8 that is conjugated to a nickel charged trisNTA, also called His-STREPPER or His/Strep-tag®II Adapter.

In some embodiments, the streptavidin-binding peptide contains a sequential arrangement of two streptavidin-binding modules. In some embodiments, the streptavidin-binding peptide contains a sequential arrangement of exactly two streptavidin-binding modules. In some embodiments, the streptavidin-binding modules are separated from one another by no more than 50 amino acids, for instance for no more than 45, 40, 35, 30, 25, 20, 15, 10, or 5 amino acids. In some embodiments, the streptavidin-binding modules are directly connected to one another. In some embodiments, one streptavidin-binding module has three to eight amino acids and contains at least the sequence His-Pro-Xaa (SEQ ID NO: 9), where Xaa is glutamine, asparagine, or methionine. In some embodiments, another streptavidin-binding module has the same or different sequence from the first streptavidin-binding module, such as set forth in SEQ ID NO: 11 (see, e.g., International Published PCT Appl. No. WO02/077018; and U.S. Pat. No. 7,981,632). In some embodiments, one of the streptavidin-binding modules contains the amino acid sequence set forth in SEQ ID NO: 7. In some embodiments, each of the streptavidin-binding modules contains the amino acid sequence set forth in SEQ ID NO: 7. In some embodiments, one of the streptavidin-binding modules contains the amino acid sequence set forth in SEQ ID NO: 8. In some embodiments, each of the streptavidin-binding modules contains the amino acid sequence set forth in SEQ ID NO: 8. In some embodiments, the streptavidin-binding peptide contains an amino acid sequence having the formula set forth in SEQ ID NO: 13 or SEQ ID NO: 14. In some embodiments, the streptavidin-binding peptide contains the amino acid sequence set forth in any of SEQ ID NO: 15-19. In some embodiments, the sequence of the streptavidin-binding peptide is set forth in any of SEQ ID NO: 15-19. In some embodiments, the streptavidin-binding peptide contains the amino acid sequence set forth in SEQ ID NO: 16, also called Twin-Strep-tag®. In some embodiments, the sequence of the streptavidin-binding peptide is set forth in SEQ ID NO: 16.

### 2. Incubation

In some aspects, the incubating is carried out in accordance with techniques such as those described in U.S. Pat. No. 6,040,177; Klebanoff et al. (2012) J Immunother. 35(9):651-660; Terakura et al. (2012) Blood 1:72-82; and Wang et al. (2012) J Immunother. 35(9):689-701. In some embodiments, the incubating is carried out using any of the methods described in WO2021/084050, U.S. Pat. No. 11,274,278, US2019/0112576, US2021/0032297, and US2022/0002669.

In some embodiments, the provided methods involve the on-column stimulation of immune cells, e.g., T cells. In some embodiments, “on-column” refers to one or more immune cells, e.g., T cells, being immobilized on a stationary phase contained in an internal cavity of a chromatography column during at least a portion of the incubating. For instance, in some embodiments, the stationary phase contains a selection agent that specifically binds to a selection marker expressed on the surface of the immune cells, e.g., T cells. In some embodiments, the specific binding of the selection agent to the selection marker effects the immobilization of the immune cells, e.g., T cells, on the stationary phase.

In some embodiments, the immune cells, e.g., T cells, are not immobilized or become no longer immobilized on the stationary phase during a portion of the incubating. In some embodiments, a portion of the incubating is performed while the immune cells, e.g., T cells, are present in the internal cavity, though not necessarily immobilized on the stationary phase. In some embodiments, stimulation can also be continued following elution of the immune cells, e.g., T cells, from the chromatography column, for instance by further incubating the immune cells, e.g., T cells, such as in the presence of the stimulatory reagent following elution.

In some embodiments, the incubating is initiated within or within about 120 minutes after adding the sample to the internal cavity. In some embodiments, the incubating is initiated within or within about 90 minutes after adding the sample to the internal cavity. In some embodiments, the incubating is initiated within or within about 60 minutes after adding the sample to the internal cavity. In some embodiments, the incubating is initiated within or within about 45 minutes after adding the sample to the internal cavity. In some embodiments, the incubating is initiated within or within about 30 minutes after adding the sample to the internal cavity. In some embodiments, the incubating is initiated within or within about 20 minutes after adding the sample to the internal cavity. In some embodiments, the incubating is initiated within or within about 15 minutes after adding the sample to the internal cavity. In some embodiments, the incubating is initiated within or within about 10 minutes after adding the sample to the internal cavity.

In some embodiments, immune cells, e.g., T cells, of the sample are allowed sufficient time to penetrate the stationary phase prior to the initiation of incubation, for instance prior to the addition of the stimulatory reagent to the stationary phase. In some embodiments, immune cells, e.g., T cells, of the sample are allowed sufficient time to become immobilized on the stationary phase, for instance via binding to the selection agent of the stationary phase, prior to the initiation of incubation. In some embodiments, the incubating is initiated at least 5, 10, or 15 minutes following the addition of the sample. In some embodiments, the stationary phase is washed at least one time following the addition of the sample and prior to the initiation of incubation.

In some embodiments, the incubating is initiated by the adding of the stimulatory reagent to the immune cells, e.g., T cells.

In some embodiments, the incubating is in the presence of between or between about 0.1 μg and 20 μg of the stimulatory reagent per 10⁶ cells of the immune cells, of the immune cells immobilized on the stationary phase, or of estimated cell counts of any of the foregoing. In some embodiments, the incubating is in the presence of between or between about 0.1 μg and 16 μg of the stimulatory reagent per 10⁶ cells of the immune cells, of the immune cells immobilized on the stationary phase, or of estimated cell counts of any of the foregoing. In some embodiments, the incubating is in the presence of between or between about 0.1 μg and 12 μg of the stimulatory reagent per 10⁶ cells of the immune cells, of the immune cells immobilized on the stationary phase, or of estimated cell counts of any of the foregoing. In some embodiments, the incubating is in the presence of between or between about 0.1 μg and 8 μg of the stimulatory reagent per 10⁶ cells of the immune cells, of the immune cells immobilized on the stationary phase, or of estimated cell counts of any of the foregoing. In some embodiments, the incubating is in the presence of between or between about 0.1 μg and 6 μg of the stimulatory reagent per 10⁶ cells of the immune cells, of the immune cells immobilized on the stationary phase, or of estimated cell counts of any of the foregoing. In some embodiments, the incubating is in the presence of between or between about 0.5 μg and 20 μg of the stimulatory reagent per 10⁶ cells of the immune cells, of the immune cells immobilized on the stationary phase, or of estimated cell counts of any of the foregoing. In some embodiments, the incubating is in the presence of between or between about 0.5 μg and 16 μg of the stimulatory reagent per 10⁶ cells of the immune cells, of the immune cells immobilized on the stationary phase, or of estimated cell counts of any of the foregoing. In some embodiments, the incubating is in the presence of between or between about 0.5 μg and 12 μg of the stimulatory reagent per 10⁶ cells of the immune cells, of the immune cells immobilized on the stationary phase, or of estimated cell counts of any of the foregoing. In some embodiments, the incubating is in the presence of between or between about 0.5 μg and 8 μg of the stimulatory reagent per 10⁶ cells of the immune cells, of the immune cells immobilized on the stationary phase, or of estimated cell counts of any of the foregoing. In some embodiments, the incubating is in the presence of between or between about 0.5 μg and 6 μg of the stimulatory reagent per 10⁶ cells of the immune cells, of the immune cells immobilized on the stationary phase, or of estimated cell counts of any of the foregoing. In some embodiments, the incubating is in the presence of between or between about 1 μg and 20 μg of the stimulatory reagent per 10⁶ cells of the immune cells, of the immune cells immobilized on the stationary phase, or of estimated cell counts of any of the foregoing. In some embodiments, the incubating is in the presence of between or between about 1 μg and 16 μg of the stimulatory reagent per 10⁶ cells of the immune cells, of the immune cells immobilized on the stationary phase, or of estimated cell counts of any of the foregoing. In some embodiments, the incubating is in the presence of between or between about 1 μg and 12 μg of the stimulatory reagent per 10⁶ cells of the immune cells, of the immune cells immobilized on the stationary phase, or of estimated cell counts of any of the foregoing. In some embodiments, the incubating is in the presence of between or between about 1 μg and 8 μg of the stimulatory reagent per 10⁶ cells of the immune cells, of the immune cells immobilized on the stationary phase, or of estimated cell counts of any of the foregoing. In some embodiments, the incubating is in the presence of between or between about 1 μg and 6 μg of the stimulatory reagent per 10⁶ cells of the immune cells, of the immune cells immobilized on the stationary phase, or of estimated cell counts of any of the foregoing. In some embodiments, the incubating is in the presence of between or between about 2 μg and 20 μg of the stimulatory reagent per 10⁶ cells of the immune cells, of the immune cells immobilized on the stationary phase, or of estimated cell counts of any of the foregoing. In some embodiments, the incubating is in the presence of between or between about 2 μg and 16 μg of the stimulatory reagent per 10⁶ cells of the immune cells, of the immune cells immobilized on the stationary phase, or of estimated cell counts of any of the foregoing. In some embodiments, the incubating is in the presence of between or between about 2 μg and 12 μg of the stimulatory reagent per 10⁶ cells of the immune cells, of the immune cells immobilized on the stationary phase, or of estimated cell counts of any of the foregoing. In some embodiments, the incubating is in the presence of between or between about 2 μg and 8 μg of the stimulatory reagent per 10⁶ cells of the immune cells, of the immune cells immobilized on the stationary phase, or of estimated cell counts of any of the foregoing. In some embodiments, the incubating is in the presence of between or between about 2 μg and 6 μg of the stimulatory reagent per 10⁶ cells of the immune cells, of the immune cells immobilized on the stationary phase, or of estimated cell counts of any of the foregoing. In some embodiments, the incubating is in the presence of between or between about 3 μg and 5 μg of the stimulatory reagent per 10⁶ cells of the immune cells, of the immune cells immobilized on the stationary phase, or of estimated cell counts of any of the foregoing. n some embodiments, the incubating is in the presence of between or between about 3.5 μg and 4.5 μg of the stimulatory reagent per 10⁶ cells of the immune cells, of the immune cells immobilized on the stationary phase, or of estimated cell counts of any of the foregoing. In some embodiments, the incubating is in the presence of about 4 μg of the stimulatory reagent per 10⁶ cells of the immune cells, of the immune cells immobilized on the stationary phase, or of estimated cell counts of any of the foregoing. In any of the foregoing embodiments, the amount of the stimulatory reagent is per 10⁶ cells of the immune cells. In any of the foregoing embodiments, the amount of the stimulatory reagent is per 10⁶ cells of the estimated count of the immune cells immobilized on the stationary phase. In any of the foregoing embodiments, the amount of the stimulatory reagent is per 10⁶ cells of the binding capacity of the stationary phase. In some embodiments, the amount of stimulatory reagent that is present in any of the described compositions containing the stimulatory reagent, e.g., those contacted to the immune cells or added to the internal cavity, is any of those described in the foregoing embodiments.

In some embodiments, the stimulatory reagent is added in an amount between or between about 0.1 μg and 20 μg, inclusive, per 10⁶ immune cells, e.g., T cells, of the immune cells, e.g., T cells immobilized or expected to be immobilized on the stationary phase. In some embodiments, the stimulatory reagent is added in an amount between or between about 0.4 μg and 8 μg, inclusive, per 10⁶ immune cells, e.g., T cells, of the immune cells, e.g., T cells immobilized or expected to be immobilized on the stationary phase. In some embodiments, the stimulatory reagent is added in an amount between or between about 0.8 μg and 4 μg, inclusive, per 10⁶ immune cells, e.g., T cells, of the immune cells, e.g., T cells immobilized or expected to be immobilized on the stationary phase. In some embodiments, the stimulatory reagent is added in an amount between or between about 1 μg and 2 μg, inclusive, per 10⁶ immune cells, e.g., T cells, of the immune cells, e.g., T cells immobilized or expected to be immobilized on the stationary phase.

In some embodiments, the stimulatory reagent is added in an amount between or between about 0.1 mg and 20 mg, 0.1 mg and 15 mg, 0.1 mg and 10 mg, 0.1 mg and 9 mg, 0.1 mg and 8 mg, 0.1 mg and 7 mg, 0.1 mg and 6 mg, 0.1 mg and 5 mg, 0.1 mg and 4 mg, 0.1 mg and 3 mg, 0.1 mg and 2 mg, 0.1 mg and 1 mg, 0.4 mg and 20 mg, 0.4 mg and 15 mg, 0.4 mg and 10 mg, 0.4 mg and 9 mg, 0.4 mg and 8 mg, 0.4 mg and 7 mg, 0.4 mg and 6 mg, 0.4 mg and 5 mg, 0.4 mg and 4 mg, 0.4 mg and 3 mg, 0.4 mg and 2 mg, 0.4 mg and 1 mg, 0.8 mg and 20 mg, 0.8 mg and 15 mg, 0.8 mg and 10 mg, 0.8 mg and 9 mg, 0.8 mg and 8 mg, 0.8 mg and 7 mg, 0.8 mg and 6 mg, 0.8 mg and 5 mg, 0.8 mg and 4 mg, 0.8 mg and 3 mg, 0.8 mg and 2 mg, 0.8 mg and 1 mg, 1 mg and 20 mg, 1 mg and 15 mg, 1 mg and 10 mg, 1 mg and 9 mg, 1 mg and 8 mg, 1 mg and 7 mg, 1 mg and 6 mg, 1 mg and 5 mg, 1 mg and 4 mg, 1 mg and 3 mg, 1 mg and 2 mg, 2 mg and 20 mg, 2 mg and 15 mg, 2 mg and 10 mg, 2 mg and 9 mg, 2 mg and 8 mg, 2 mg and 7 mg, 2 mg and 6 mg, 2 mg and 5 mg, 2 mg and 4 mg, 2 mg and 3 mg, 3 mg and 20 mg, 3 mg and 15 mg, 3 mg and 10 mg, 3 mg and 9 mg, 3 mg and 8 mg, 3 mg and 7 mg, 3 mg and 6 mg, 3 mg and 5 mg, 3 mg and 4 mg, 4 mg and 20 mg, 4 mg and 15 mg, 4 mg and 10 mg, 4 mg and 9 mg, 4 mg and 8 mg, 4 mg and 7 mg, 4 mg and 6 mg, 4 mg and 5 mg, 5 mg and 20 mg, 5 mg and 15 mg, 5 mg and 10 mg, 5 mg and 9 mg, 5 mg and 8 mg, 5 mg and 7 mg, 5 mg and 6 mg, 6 mg and 20 mg, 6 mg and 15 mg, 6 mg and 10 mg, 6 mg and 9 mg, 6 mg and 8 mg, 6 mg and 7 mg, 7 mg and 20 mg, 7 mg and 15 mg, 7 mg and 10 mg, 7 mg and 9 mg, 7 mg and 8 mg, 8 mg and 20 mg, 8 mg and 15 mg, 8 mg and 10 mg, 8 mg and 9 mg, 9 mg and 20 mg, 9 mg and 15 mg, 9 mg and 10 mg, 10 mg and 20 mg, 10 mg and 15 mg, or 15 mg and 20 mg, each inclusive. In some embodiments, the stimulatory reagent is added in an amount between or between about 0.1 mg and 20 mg, inclusive. In some embodiments, the stimulatory reagent is added in an amount between or between about 0.4 mg and 8 mg, inclusive. In some embodiments, the stimulatory reagent is added in an amount between or between about 0.8 mg and 4 mg, inclusive. In some embodiments, the stimulatory reagent is added in an amount between or between about 1 mg and 3 mg, inclusive.

In some embodiments, the incubating can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to stimulate the immune cells, e.g., T cells.

In some embodiments, the incubating is carried out in a cell medium. In some embodiments, the stimulatory reagent is added to the stationary phase in a cell medium.

In some embodiments, the cell medium is a serum free medium. In some embodiments, the serum free medium is a defined or well-defined cell culture medium. In certain embodiments, the serum free medium is a controlled culture medium that has been processed, e.g., filtered, to remove inhibitors and/or growth factors. In some embodiments, the serum free medium contains proteins. In some embodiments, the serum-free medium contains serum albumin, hydrolysates, growth factors, hormones, carrier proteins, and/or attachment factors. In some embodiments, the serum free medium comprises glutamine.

In some embodiments, the cell medium is a basal medium. In some embodiments, the basal medium is without any recombinant cytokines. In some embodiments, the basal medium is serum-free. In some embodiments, the basal medium is free of serum derived from human. In some embodiments, the basal medium contains a mixture of inorganic salts, sugars, amino acids, and, optionally, vitamins, organic acids and/or buffers or other well known cell culture nutrients. In addition to nutrients, the basal medium can also help maintain pH and osmolality. A wide variety of commercially available basal media are well known to those skilled in the art, and include Dulbeccos' Modified Eagles Medium (DMEM), Roswell Park Memorial Institute Medium (RPMI), Iscove modified Dulbeccos' medium and Hams medium. In some embodiments, the basal medium is Iscove's Modified Dulbecco's Medium, RPMI-1640, or a-MEM.

In some embodiments, the basal medium is a balanced salt solution (e.g., PBS, DPBS, HBSS, EBSS). In some embodiments, the basal medium is selected from Dulbecco's Modified Eagle's Medium (DMEM), Minimal Essential Medium (MEM), Basal Medium Eagle (BME), F-10, F-12, RPMI 1640, Glasgow's Minimal Essential Medium (GMEM), alpha Minimal Essential Medium (alpha MEM), Iscove's Modified Dulbecco's Medium, and M199. In some embodiments, the basal medium is a complex medium (e.g., RPMI-1640, IMDM). In some embodiments, the basal medium is OpTmizer™ CTS™ T-Cell Expansion Basal Medium (ThermoFisher).

In certain embodiments, the basal media is supplemented with additional additives. In some embodiments, the basal media is not supplemented with any additional additives. Additives to cell culture media include nutrients, sugars, e.g., glucose, amino acids, vitamins, or additives such as ATP and NADH.

In some embodiments, the cell medium contains one or more cytokines. In certain embodiments, the one or more cytokines are recombinant cytokines. In particular embodiments, the one or more cytokines are human recombinant cytokines. In certain embodiments, the one or more cytokines bind to receptors that are expressed by T cells. In particular embodiments, the one or more cytokines include a member of the 4-alpha-helix bundle family of cytokines. In some embodiments, members of the 4-alpha-helix bundle family of cytokines include interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines include IL-15. In particular embodiments, the one or more cytokines include IL-7. In particular embodiments, the one or more cytokines include IL-2. In particular embodiments, the one or more cytokines are selected from IL-2, IL-15, and IL-7. In particular embodiments, the cell medium contains recombinant IL-2, IL-15, and IL-7.

In certain embodiments, the amount or concentration of the one or more cytokines are measured and/or quantified with International Units (IU). International units may be used to quantify vitamins, hormones, cytokines, vaccines, blood products, and similar biologically active substances. In some embodiments, IU are or include units of measure of the potency of biological preparations by comparison to an international reference standard of a specific weight and strength, e.g., WHO 1st International Standard for Human IL-2, 86/504. International Units are the only recognized and standardized method to report biological activity units that are published and are derived from an international collaborative research effort. In particular embodiments, the IU for population, sample, or source of a cytokine may be obtained through product comparison testing with an analogous WHO standard product. For example, in some embodiments, the IU/mg of a population, sample, or source of human recombinant IL-2, IL-7, or IL-15 is compared to the WHO standard IL-2 product (NIBSC code: 86/500), the WHO standard IL-17 product (NIBSC code: 90/530), and the WHO standard IL-15 product (NIBSC code: 95/554), respectively.

In particular embodiments, the ED50 of recombinant human IL-2 or IL-15 is equivalent to the concentration required for the half-maximal stimulation of cell proliferation (XTT cleavage) with CTLL-2 cells. In certain embodiments, the ED50 of recombinant human IL-7 is equivalent to the concentration required for the half-maximal stimulation for proliferation of PHA-activated human peripheral blood lymphocytes. Details relating to assays and calculations of IU for IL-2 are discussed in Wadhwa et al., Journal of Immunological Methods (2013), 379 (1-2): 1-7; and Gearing and Thorpe, Journal of Immunological Methods (1988), 114 (1-2): 3-9; details relating to assays and calculations of IU for IL-15 are discussed in Soman et al. Journal of Immunological Methods (2009) 348 (1-2): 83-94.

In some embodiments, the cell medium contains IL-2, e.g., human recombinant IL-2, at a concentration between 1 IU/mL and 500 IU/mL, between 10 IU/mL and 250 IU/mL, between 50 IU/mL and 200 IU/mL, between 50 IU/mL and 150 IU/mL, between 75 IU/mL and 125 IU/mL, between 100 IU/mL and 200 IU/mL, or between 10 IU/mL and 100 IU/mL. In particular embodiments, the cell medium contains recombinant IL-2 at a concentration at or at about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, 150 IU/mL, 160 IU/mL, 170 IU/mL, 180 IU/mL, 190 IU/mL, or 100 IU/mL. In some embodiments, the cell medium contains about 100 IU/mL of recombinant IL-2, e.g., human recombinant IL-2.

In some embodiments, the cell medium contains recombinant IL-7, e.g., human recombinant IL-7, at a concentration between 100 IU/mL and 2,000 IU/mL, between 500 IU/mL and 1,000 IU/mL, between 100 IU/mL and 500 IU/mL, between 500 IU/mL and 750 IU/mL, between 750 IU/mL and 1,000 IU/mL, or between 550 IU/mL and 650 IU/mL. In particular embodiments, the cell medium contains IL-7 at a concentration at or at about 50 IU/mL, 100 IU/mL, 150 IU/mL, 200 IU/mL, 250 IU/mL, 300 IU/mL, 350 IU/mL, 400 IU/mL, 450 IU/mL, 500 IU/mL, 550 IU/mL, 600 IU/mL, 650 IU/mL, 700 IU/mL, 750 IU/mL, 800 IU/mL, 750 IU/mL, 750 IU/mL, 750 IU/mL, or 1,000 IU/mL. In particular embodiments, the cell medium contains about 600 IU/mL of IL-7, e.g., human recombinant IL-7.

In some embodiments, the cell medium contains recombinant IL-15, e.g., human recombinant IL-15, at a concentration between 1 IU/mL and 500 IU/mL, between 10 IU/mL and 250 IU/mL, between 50 IU/mL and 200 IU/mL, between 50 IU/mL and 150 IU/mL, between 75 IU/mL and 125 IU/mL, between 100 IU/mL and 200 IU/mL, or between 10 IU/mL and 100 IU/mL. In particular embodiments, the cell medium contains recombinant IL-15 at a concentration at or at about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, 150 IU/mL, 160 IU/mL, 170 IU/mL, 180 IU/mL, 190 IU/mL, or 200 IU/mL. In some embodiments, the cell medium contains about 100 IU/mL of recombinant IL-15, e.g., human recombinant IL-15.

In some embodiments, the cell medium contains no cytokines.

In some embodiments, following the initiation of the incubating, the immune cells, e.g., T cells, are incubated in the internal cavity of the chromatography. In some embodiments, the incubating is performed for, for about, or for less than one day. In some embodiments, the incubating is performed for, for about, or for less than, 24, 23, 22, 21, 20, 19, 18, 17, 16, 15, 14, 13, 12, 11, 10, 9, 8, 7, 6, 5, 4, 3, or 2 hours. In some embodiments, the incubating is performed for between or between about 2 to 24, 3 to 24, 4 to 24, 5, to 24, 6 to 24, 7 to 24, 8 to 24, 9 to 24, 10 to 24, 11 to 24, 12 to 24, 13 to 24, 14 to 24, 15 to 24, 16 to 24, 17 to 24, 18 to 24, 19 to 24, 20 to 24, 21 to 24, 22 to 24, 23 to 24, 2 to 23, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, or 2 to 3 hours. In some embodiments, the incubating is performed for, for about, or for less than, 24 hours. In some embodiments, the incubating is performed for, for about, or for less than, 12 hours. In some embodiments, the incubating is performed for, for about, or for less than, 5 hours. In some embodiments, the incubating is performed for, for about, or for less than, 4 hours. In some embodiments, the incubating is performed for, for about, or for less than, 2 hours.

In some embodiments, the incubating is carried out for between or between about 1 hour and 8 hours, inclusive. In some embodiments, the incubating is carried out for between or between about 2 hours and 6 hours, inclusive. In some embodiments, the incubating is carried out for between or between about 3 hours and 5 hours, inclusive. In some embodiments, the incubating is carried out for or for about 4 hours. In some embodiments, the incubating is carried out for or for about 4.5 hours.

In some embodiments, the incubating is carried out at a temperature that is above room temperature. In some embodiments, the incubating is carried out at a physiological temperature. In some embodiments, the incubating is carried out a temperature between or between about 35° C. and 39° C. In some embodiments, the incubating is carried out at or at about 37° C.

In some embodiments, the temperature is regulated by one or more heating elements configured to provide heat to the stationary phase. In some embodiments, the oxygen and carbon dioxide content of the stationary phase is controlled using gas exchange. In some embodiments, the temperature or gas exchange is regulated using any of the methods or devices described in WO2020/089343, WO2021/084050, and US2022/0002669.

In some embodiments, the incubating facilitates downregulation of the selection marker used for immune cell selection, e.g., T cell selection, in some instances resulting in spontaneous detachment or release of the immune cell, e.g., T cell, from the stationary phase. The release or detachment of the immune cells, e.g., T cells, can occur without any additional steps or reagents. In some aspects, the immune cells, e.g., T cells, can be collected using wash buffer that does not contain a competition agent to, e.g., facilitate detachment of the immune cells, e.g., T cells, from the stationary phase.

### 3. Collection

In some embodiments, the provided methods involve collecting immune cells, e.g., T cells. In some embodiments, the immune cells, e.g., T cells, are collected from the chromatography column. In some embodiments, the collecting includes eluting the immune cells, e.g., T cells, from the chromatography column.

In some embodiments, the collecting is carried out at, at about, or at least 2, 3, 4, 5, 6, 7, 8, 9, 10, 11, 12, 13, 14, 15, 16, 17, 18, 19, 20, 21, 22, 23, or 24 hours after the adding of the stimulatory reagent. In some embodiments, the collecting is carried out within or within about 2, 3, 4, 5, 6, 7, 8, 9, 10, 11, 12, 13, 14, 15, 16, 17, 18, 19, 20, 21, 22, 23, or 24 hours after the adding of the stimulatory reagent. In some embodiments, the collecting is carried out about 2 to 24, 3 to 24, 4 to 24, 5, to 24, 6 to 24, 7 to 24, 8 to 24, 9 to 24, 10 to 24, 11 to 24, 12 to 24, 13 to 24, 14 to 24, 15 to 24, 16 to 24, 17 to 24, 18 to 24, 19 to 24, 20 to 24, 21 to 24, 22 to 24, 23 to 24, 2 to 23, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, or 2 to 3 hours after the adding of the stimulatory reagent. In some embodiments, the collecting is carried out about 2 to 24 hours after the adding of the stimulatory reagent. In some embodiments, the collecting is carried out about 2 to 12 hours after the adding of the stimulatory reagent. In some embodiments, the collecting is carried out about 1 to 8 hours after the adding of the stimulatory reagent. In some embodiments, the collecting is carried out about 2 to 6 hours after the adding of the stimulatory reagent. In some embodiments, the collecting is carried out about 3 to 5 hours after the adding of the stimulatory reagent. In some embodiments, the collecting is carried out about 4.5 hours after the adding of the stimulatory reagent.

In some embodiments, the collecting is carried out at, at about, or at least 2, 3, 4, 5, 6, 7, 8, 9, 10, 11, 12, 13, 14, 15, 16, 17, 18, 19, 20, 21, 22, 23, or 24 hours after initiation of the incubation. In some embodiments, the collecting is carried out within or within about 2, 3, 4, 5, 6, 7, 8, 9, 10, 11, 12, 13, 14, 15, 16, 17, 18, 19, 20, 21, 22, 23, or 24 hours after initiation of the incubating. In some embodiments, the collecting is carried out about 2 to 24, 3 to 24, 4 to 24, 5, to 24, 6 to 24, 7 to 24, 8 to 24, 9 to 24, 10 to 24, 11 to 24, 12 to 24, 13 to 24, 14 to 24, 15 to 24, 16 to 24, 17 to 24, 18 to 24, 19 to 24, 20 to 24, 21 to 24, 22 to 24, 23 to 24, 2 to 23, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, or 2 to 3 hours after initiation of the incubating. In some embodiments, the collecting is carried out about 2 to 24 hours after initiation of the incubating. In some embodiments, the collecting is carried out about 2 to 12 hours after initiation of the incubating. In some embodiments, the collecting is carried out about 1 to 8 hours after initiation of the incubating. In some embodiments, the collecting is carried out about 2 to 6 hours after initiation of the incubating. In some embodiments, the collecting is carried out about 3 to 5 hours after initiation of the incubating. In some embodiments, the collecting is carried out about 4.5 hours after initiation of the incubating.

In some embodiments, the collecting involves adding a wash buffer to the stationary phase to collect the immune cells, e.g., T cells. In some embodiments, the wash buffer is a cell medium. In some embodiments, the cell medium is any described in Section I-B-2.

In some embodiments, the collecting can be performed without the addition of a competition agent to elute the immune cells, e.g., T cells, from the stationary phase. In some embodiments, the wash buffer does not contain a competition agent to elute the immune cells, e.g., T cells, from the stationary phase. In some embodiments, the wash buffer contains a competition agent to elute the immune cells, e.g., T cells, from the stationary phase.

In some embodiments, the competition agent facilitates detachment of the immune cells, e.g., T cells, from the stationary phase. In some embodiments, the competition agent disrupts the immobilization of the immune cells, e.g., T cells, on the stationary phase. In some embodiments, the competition agent disrupts the immobilization of the selection agent on the chromatography matrix of the stationary phase. For instance, in some embodiments, the stationary phase contains a molecule that is any of the streptavidin, avidin, streptavidin analog or mutein, or avidin analog or mutein molecules described herein, and the binding partner of the selection agent is any of the binding partners, e.g., streptavidin or avidin binding partners, such as streptavidin-binding peptides, described herein that reversibly binds to the molecule, for instance with reduced binding affinity compared to that of streptavidin to biotin, or such that the binding is disrupted in the presence of biotin. In some embodiments, the competition agent has higher binding affinity for the molecule than does the binding partner of the selection agent. In some embodiments, the competition agent disrupts the binding of the binding partner of the selection agent to the molecule. In some embodiments, the competition agent is biotin, e.g., D-biotin. In some embodiments, the competition agent is a biotin analog or derivative, e.g., any as described herein.

In some embodiments, the chromatography column and collection containers are connected in a closed system. In some embodiments, the closed system is sterile. In some embodiments, the selection, stimulation, and/or elution steps are performed by an automated system with minimal or no manual, such as human, operation or interference. 4. Further Incubation

In some embodiments, the provided methods involve further incubating the immune cells, e.g., T cells. In some embodiments, the further incubating is carried out in the presence of the stimulatory reagent. In some embodiments, the further incubating is carried out after the immune cells, e.g., T cells, are collected from the chromatography column. Thus, in some aspects, the stimulating of the immune cells, e.g., T cells, is continued following collection of the immune cells, e.g., T cells, from the chromatography column. In some embodiments, the further incubating is carried out prior to engineering the immune cells, e.g., T cells.

In some embodiments, the further incubating is carried out in the presence of the stimulatory reagent. In some embodiments, the stimulatory reagent is present at a concentration or amount that is any described in Section I-B-2. In some embodiments, the stimulatory reagent added for the incubating is not removed prior to the further incubating. In some embodiments, the further incubating is in the presence of the same medium that was present during the incubating. In some embodiments, the further incubating is carried out in the cell medium present in the chromatography column that is eluted by the collecting, including any stimulatory reagent present therein.

In some embodiments, the further incubating can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to stimulate the immune cells.

In some embodiments, the further incubating is carried out in a cell medium. In some embodiments, the cell medium is any described in Section I-B-2.

In some embodiments, the further incubating is carried out at a temperature that is above room temperature. In some embodiments, the further incubating is carried out at a physiological temperature. In some embodiments, the further incubating is carried out a temperature between or between about 35° C. and 39° C. In some embodiments, the further incubating is carried out at or at about 37° C.

In some embodiments, the further incubating is carried out for between or between about 2 hours and 30 hours, 2 hours and 26 hours, 2 hours and 22 hours, 2 hours and 18 hours, 2 hours and 14 hours, 2 hours and 10 hours, 2 hours and 6 hours, 2 hours and 4 hours, 4 hours and 30 hours, 4 hours and 26 hours, 4 hours and 22 hours, 4 hours and 18 hours, 4 hours and 14 hours, 4 hours and 10 hours, 4 hours and 6 hours, 6 hours and 30 hours, 6 hours and 26 hours, 6 hours and 22 hours, 6 hours and 18 hours, 6 hours and 14 hours, 6 hours and 10 hours, 10 hours and 30 hours, 10 hours and 26 hours, 10 hours and 22 hours, 10 hours and 18 hours, 10 hours and 14 hours, 14 hours and 30 hours, 14 hours and 26 hours, 14 hours and 22 hours, 14 hours and 18 hours, 18 hours and 30 hours, 18 hours and 26 hours, 18 hours and 22 hours, 22 hours and 30 hours, 22 hours and 26 hours, or 26 hours and 30 hours, each inclusive. In some embodiments, the further incubating is carried out for between or between about 10 hours and 30 hours, inclusive. In some embodiments, the further incubating is carried out for between or between about 16 hours and 24 hours, inclusive. In some embodiments, the further incubating is carried out for between or between about 18 hours and 22 hours, inclusive. In some embodiments, the further incubating is carried out for at or about 20 hours.

In some embodiments, the further incubating occurs in an incubator. In some embodiments, the immune cells, e.g., T cells, are transferred into a container for the further incubating. In some embodiments, the container is a vial. In particular embodiments, the container is a bag. In some embodiments, the immune cells, e.g., T cells, are transferred into the container under closed or sterile conditions. In some embodiments, the container, e.g., the vial or bag, is then placed into an incubator for all or a portion of the further incubating. In particular embodiments, the incubator is set at, at about, or at least 16° C., 24° C., or 35° C. In some embodiments, the incubator is set at 37° C., at about at 37° C., or at 37° C.±2° C., ±1° C., ±0.5° C., or 0.1° C.

In certain embodiments, the further incubating is performed under static conditions, such as conditions that do not involve centrifugation, shaking, rotating, rocking, or perfusion of media. In some embodiments, the further incubating is performed under gentle mixing conditions, e.g., involving rocking.

In some embodiments, the provided methods involve removing the stimulatory reagent from the immune cells, e.g., T cells. In some embodiments, the removing is subsequent to the further incubating. In some embodiments, the removing is carried out prior to the engineering. In some embodiments, the removing is carried out prior to the introducing of the nucleic acid molecule. In some embodiments, the removing is carried out prior to the introducing of the one or more gene-editing agents. In some embodiments, the removing terminates the stimulation of the immune cells, e.g., T cells. In some embodiments, the removing involves washing the immune cells, e.g., T cells, for instance using any of the cell media described herein, either with or without the presence of a competition agent.

In some embodiments, the provided methods involve disrupting the binding between the one or more binding agents and the protein reagent of the stimulatory reagent. In someembodiments, the disrupting is carried out subsequent to the further incubating. In some embodiments, the disrupting is carried out prior to the engineering. In some embodiments, the disrupting is carried out prior to the introducing of the nucleic acid molecule. In some embodiments, the disrupting is carried out prior to the introducing of the one or more gene-editing agents. In some embodiments, the disrupting terminates the stimulation of the immune cells, e.g., T cells. In some embodiments, the disrupting is by adding a competition agent to reverse the binding between the one or more binding agents and the protein reagent of the stimulatory reagent. In some embodiments, the competition agent is biotin or a biotin analog, e.g., any as described herein, and the one or more binding agents and the protein reagent are any described herein for which binding can be disrupted by biotin or the biotin analog.

### C. Engineering

In some embodiments, the provided methods involve engineering immune cells, e.g., T cells. In some embodiments, the provided methods involve targeted integration of a transgene into a target site of a gene in the immune cells, e.g., T cells. In some embodiments, the provided methods involve introducing a nucleic acid molecule containing the transgene into the immune cells, e.g., T cells. In some embodiments, the introducing of the nucleic acid molecule is under conditions for targeted integration of the transgene into the target site. In some embodiments, the introducing of the nucleic acid molecule is by non-viral gene delivery.

In some embodiments, the provided methods do not involve inducing a genetic disruption. In some embodiments, the targeted integration is by methods that do not induce a genetic disruption. In some embodiments, the targeted integration is by Programmable Addition via Site-specific Targeting Elements (PASTE), such as described in, e.g., WO2022/159892 and US20220154224. In some embodiments, the PASTE involves introducing one or more gene-editing agents for editing the gene in the immune cells.

In some embodiments, the provided methods involve inducing a targeted genetic disruption. In some embodiments, the provided methods involve homology-dependent repair (HDR) using a nucleic acid molecule containing the transgene, thereby targeting integration of the transgene at the target site.

In some cases, the provided methods involve introducing one or more targeted genetic disruptions, e.g., DNA breaks, at the target site by gene editing techniques, combined with targeted integration of the transgene by HDR. In some aspects, the one or more targeted genetic disruptions are carried out by introduction of one or more gene-editing agents capable of introducing the genetic disruptions. In some embodiments, the HDR step entails a disruption or a break, e.g., a double-stranded break, in the DNA at the target site. In some embodiments, the DNA break is induced by employing gene editing methods, e.g., targeted nucleases. In some embodiments, the methods generate an engineered immune cell, e.g., T cell, that is knocked-out for expression of the gene containing the target site. In some aspects, after carrying out the methods, the engineered immune cell, e.g. T cell, contains the transgene operably linked to an endogenous transcriptional regulatory element of the gene.

In some aspects, the provided methods involve introducing the one or more gene-editing agents and introducing into the immune cells, e.g., T cells, a nucleic acid molecule containing a transgene and one or more homology arms. In some aspects, the transgene contains a sequence of nucleotides encoding a recombinant protein. In some embodiments, the nucleic acid sequence is targeted for integration within the target site via homology directed repair (HDR).

In some aspects, the provided methods involve introducing a nucleic acid molecule comprising the transgene into an immune cell, e.g., T cell, having a genetic disruption within the gene having the target site, wherein the genetic disruption has been induced by one or more gene-editing agents capable of inducing a genetic disruption within the gene, and wherein the nucleic acid sequence is targeted for integration within the gene via HDR.

In some aspects, the provided methods involve generating a targeted DNA break using gene editing methods and/or targeted nucleases, followed by HDR based on one or more nucleic acid molecules that contain homology sequences that are homologous to sequences within or near the gene linked to the transgene, and in some cases nucleic acid sequences encoding other molecules, to specifically target and integrate the transgene at or near the DNA break. Thus, in some aspects, the provided methods involve a step of inducing a targeted genetic disruption and introducing the nucleic acid molecule containing the transgene into the immune cell, such as a T cell (e.g., by HDR).

In some embodiments, the targeted integration of the transgene by HDR occurs at one or more target sites in the gene. In some aspects, the targeted integration occurs within the open reading frame sequence of the gene. In some aspects, targeted integration of the transgene results in a knock-out of the gene, e.g., such that the expression of the gene is eliminated.

In some aspects, the transgene has been integrated into the gene, e.g., by homology-directed repair (HDR), within an exon of an open reading frame or a partial sequence thereof of the gene, such that the transgene is in-frame with the sequence of the exon. In some aspects, all or a portion of the gene, such as the portion upstream of the integrated transgene, in the modified locus and the recombinant protein are expressed, in some cases separated by a multicistronic element.

In some aspects, the provided methods allow the recombinant protein to be expressed under the control of an endogenous transcriptional regulatory element of the gene, e.g., an endogenous promotor of the gene. In some aspects, the provided methods allow the transgene to be operably linked to the endogenous regulatory or control elements, e.g., cis regulatory elements, such as the promoter, or the 5′ and/or 3′ untranslated regions (UTRs) of the gene. Thus, in some aspects, the provided methods allow the recombinant protein, e.g., CAR, to be expressed, and/or the expression is conditionally, temporally, and/or quantitatively regulated similarly to the gene.

In some embodiments, a nucleic acid molecule is introduced into the immune cell, e.g., T cell, prior to, simultaneously with, or subsequent to introduction of the one or more gene-editing agents. In the presence of one or more targeted genetic disruptions, e.g., DNA breaks, the nucleic acid molecule can be used as a DNA repair template, to effectively integrate the transgene, at or near the site of the targeted genetic disruption by HDR, based on homology between the endogenous gene sequence surrounding the genetic disruption and the one or more homology arms, such as the 5′ and/or 3′ homology arms included in the nucleic acid molecule.

In some aspects, the nucleic acid molecule and the one or more gene-editing agents are introduced simultaneously. In some embodiments, the introducing of the one or more gene-editing agents is carried out concurrently with the introducing of the nucleic acid molecule. In some aspects, the two introducing steps can be performed sequentially. the introducing of the one or more gene-editing agents is carried out prior to the introducing of the nucleic acid molecule.

In some embodiments, the gene editing and HDR steps are performed simultaneously and/or in one experimental reaction. In some embodiments, the gene editing and HDR steps are performed consecutively or sequentially, in one or consecutive experimental reactions. In some embodiments, the gene editing and HDR steps are performed in separate experimental reactions, simultaneously or at different times.

Any method for introducing the one or more gene-editing agents can be employed as described, depending on the particular agents used. In some aspects, the agent is an RNA-guided nuclease such as a clustered regularly interspersed short palindromic nucleic acid (CRISPR)-Cas system, such as CRISPR-Cas9 or CRISPR-Cas12 system, specific for the gene. In some embodiments, an agent containing a Cas, e.g., Cas9 or Cas12, and a guide RNA (gRNA) containing a targeting sequence, which targets a region of the gene, is introduced into the immune cell. In some embodiments, the agent is or comprises a ribonucleoprotein (RNP) complex of Cas, e.g., Cas9 or Cas12, and gRNA containing the gene-targeted targeting sequence (Cas/gRNA RNP). In some embodiment, the introduction includes contacting the agent with the immune cells in vitro. In some embodiments, the introduction further can include effecting delivery of the agent and/or the nucleic acid molecule, such as a template for HDR, into the immune cells. In various embodiments, the provided methods utilize direct delivery of ribonucleoprotein (RNP) complexes of Cas, e.g., Cas9 or Cas12, and gRNA to immune cells, for example by electroporation. In some cases, electroporation of the immune cells to be modified includes cold-shocking the cells, e.g., at 320 C, following electroporation of the immune cells and prior to plating.

In some embodiments, the step of introducing the nucleic acid molecule and the step of introducing the one or more gene-editing agents (e.g., Cas/gRNA RNP) can occur simultaneously or sequentially in any order. In some of any embodiments, the nucleic acid molecule is introduced into the immune cells, e.g., T cells, after introducing the one or more gene-editing agents (e.g., Cas/gRNA RNP).

Any method for introducing the nucleic acid molecule can be employed as described, depending on the particular methods used for delivery of the nucleic acid molecule to immune cells. In some of any embodiments, non-viral gene delivery methods are employed.

In some embodiments, the nucleic acid molecule is introduced in one or more collected T cells by non-viral gene delivery. In some embodiments, the nucleic acid molecule comprises a transgene encoding a recombinant protein. In some embodiments, the introducing is under conditions for targeted integration of the transgene into a target site of a gene in one or more collected T cells.

In some embodiments, the provided methods comprise introducing by non-viral gene delivery a nucleic acid molecule comprising a transgene encoding a recombinant protein under conditions for targeted integration of the transgene into a target site of a gene in one or more collected T cells.

In some embodiments, the nucleic acid molecule is a DNA molecule. In some embodiments, the nucleic acid molecule is a naked DNA molecule. In some embodiments, the nucleic acid molecule, e.g., naked DNA molecule, is a non-viral, capsid-free DNA molecule.

In some embodiments, the nucleic acid molecule is a double-stranded DNA molecule. In some embodiments, the nucleic acid molecule is a single-stranded DNA molecule.

In some embodiments, the nucleic acid molecule, e.g., naked DNA molecule, is a modified DNA molecule. In some embodiments, the nucleic acid molecule, e.g., naked DNA molecule, is modified to enhance its stability.

In some embodiments, the nucleic acid molecule, e.g., naked DNA molecule, is a non-viral, capsid-free DNA molecule with covalently-closed ends (also referred to herein as a “closed-ended DNA” or a “ceDNA” molecule). In some embodiments, the nucleic acid molecule, e.g., naked DNA molecule, is a ceDNA molecule. In some embodiments, the nucleic acid molecule is a naked ceDNA molecule.

In some embodiments, the nucleic acid molecule, e.g., naked DNA molecule, is a capsid free and linear duplex DNA molecule.

In some embodiments, the nucleic acid molecule, e.g., naked DNA molecule, comprises at least one sequence homologous to the nucleic acid sequences surrounding the target site (also herein referred to herein as a “homology arm”). In some embodiments, the nucleic acid molecule, e.g., naked DNA molecule, comprises a 5′ homology arm and a 3′ homology arm comprising sequences homologous to nucleic acid sequences surrounding the target site. In some embodiments, the nucleic acid molecule, e.g., naked DNA molecule, comprises the structure [5′ homology arm]-[transgene]-[3′ homology arm].

In some embodiments, the nucleic acid molecule, e.g., naked DNA molecule, has a nick or a gap. In some embodiments, the nucleic acid molecule, e.g., naked DNA molecule, comprises at least one inverted tandem repeats (also herein referred to as an “ITR”). In some embodiments, the nucleic acid molecule, e.g., naked DNA molecule, comprises at least one homology arm. In some embodiments, the nucleic acid molecule, e.g., naked DNA molecule, comprises at least one ITR, at least one homology arm, and the transgene. In some embodiments, the structure of the nucleic acid molecule, e.g., naked DNA molecule, is [5′ ITR]-[homology arm (5′)]-[transgene]-[homology arm (3′)]-[ITR 3′].

In some embodiments, the ITR is an ITR derived from an AAV serotype, an ITR derived from an ITR of goose virus, an ITR derived from a B19 virus ITR, or a wild-type ITR from a parvovirus. In some embodiments, the AAV serotype is selected from the group consisting of AAV1, AAV2, AAV3, AAV4, AAV5, AAV6, AAV7, AAV8, AAV9, AAV 10, AAV11, and AAV 12. In some embodiments, the ITR is a mutant ITR.

In some embodiments, the nucleic acid molecule, e.g., the naked DNA molecule, comprises an additional ITR which differs from the first ITR. In some embodiments, the nucleic acid molecule, e.g., naked DNA molecule, comprises two mutant ITRs in both 5′ and 3′ ends of the transgene. In some embodiments, the two mutant ITRs are symmetric mutants.

In some embodiments, the nucleic acid molecule, e.g., naked DNA molecule, is selected from the group consisting of a closed-ended linear duplex (CELiD) DNA molecule, a minicircle DNA molecule, a minimalistic immunological-defined gene expression (MIDGE) DNA molecule, a ministring DNA molecule, a dumbbell-shaped linear duplex closed-ended DNA molecule, or a Doggybone™ DNA molecule. In some embodiments, the nucleic acid molecule, e.g., naked DNA molecule, is a CELiD DNA molecule. In some embodiments, the nucleic acid molecule, e.g., naked DNA molecule, is a MIDGE DNA molecule. In some embodiments, the nucleic acid molecule, e.g., naked DNA molecule, is a ministring DNA. In some embodiments, the nucleic acid molecule, e.g., naked DNA molecule, is a dumbbell-shaped linear duplex closed-ended DNA molecule. In some embodiments, the nucleic acid molecule, e.g., naked DNA molecule, is a Doggybone™ DNA molecule.

In some embodiments, the nucleic acid molecule, e.g., naked DNA molecule, is a Doggybone™ DNA molecule. In some embodiments, the Doggybone™ DNA (dbDNA™) molecule is a proprietary synthetic closed linear double-stranded DNA molecule. In some embodiments, the closed linear DNA molecule is double-stranded DNA that is covalently closed at each end. The double stranded section of the closed linear DNA molecule can be complementary. When denatured, closed linear DNA may form a single stranded circle. The DNA may be closed at each end by any suitable structure, including a cruciform, a hairpin, or a hairpin loop, depending on preference. The end of the closed linear DNA may be composed of a non-complementary sequence. In some embodiments, the non-complementary sequence forces the DNA into a single stranded configuration at the cruciform, hairpin, or hairpin loop.

In some embodiments, the nucleic acid molecule, e.g., naked DNA molecule, is a closed-ended linear duplex (CELiD) DNA molecule. In some embodiments, the CELiD DNA molecule is a linear duplex molecule. In some embodiments, the CELiD DNA molecule is double-stranded DNA that is covalently closed at each end. In some embodiments, the CELiD DNA molecule may comprise heterologous DNA flanked by ITRs. In some embodiments, the heterologous DNA may encode a protein. In some embodiments, the CELiD DNA molecule is a linear, duplex DNA molecule comprising heterologous DNA flanked by inverted terminal repeats (ITRs), at least one of which comprises an AAV Rep protein binding site and an AAV trs site, wherein the linear, duplex DNA molecule has covalently closed ends. In some embodiments, because the ends are covalently closed, the CELiD DNA molecule is exonuclease resistant. In some embodiments, each single, linear strand of heterologous DNA in the duplex molecule is between two, full-length AAV ITRs. In some embodiments, because each linear strand of a CELiD DNA molecule pairs with a complementary strand, the final, duplex CELiD molecule comprises a total of four ITRs.

In some embodiments, the nucleic acid molecule, e.g., naked DNA molecule, is a minicircle DNA molecule. In some embodiments, the minicircle DNA molecule is a circular DNA molecule. In some embodiments, the circular DNA molecule contains at least one gene encoding a recombinant protein. In some embodiments, the minicircle DNA molecule is circular DNA having an attR site and genetically engineered gene expression cassette, and said gene expression cassette comprises a promoter, base sequence encoding immunoglobulin κ chain signal peptide, base sequence encoding Flag tag, the gene encoding a recombinant protein, base sequence encoding His6 tag, stop codon, and polyA tailing signal linked sequentially.

In some embodiments, the nucleic acid molecule, e.g., naked DNA molecule, is a minimalistic immunological-defined gene expression (MIDGE) DNA molecule. In some embodiments, the MIDGE DNA molecule is a circular DNA molecule. In some embodiments, the MIDGE DNA molecule is a circular DNA molecule that is double-stranded. In some embodiments, the MIDGE DNA molecule comprises an expression cassette containing a promoter, a gene of interest, and an RNA-stabilizing sequence, e.g., a poly A sequence. The complementary sense and antisense strands encoding the transgene can be connected at both the 5′ and 3′ ends of the double-stranded MIDGE DNA molecule by a single-stranded hairpin DNA having non-complementary sequence loop structures, so that the MIDGE DNA molecule has a “dumbbell” shape. The MIDGE DNA molecule can be resistant to enzymatic digestion and relatively stable in cells and serum.

In some embodiments, the nucleic acid molecule, e.g., naked DNA molecule, is a ministring DNA molecule. In some embodiments, the ministring DNA molecule is an enhanced linear covalently closed (LCC) minivector. In some embodiments, the ministring DNA molecule is double-stranded. In some embodiments, the ministring DNA molecule comprises LCC ends, minimal transgene expression cassette elements, and DNA targeting sequences (DTS) at both ends.

In some embodiments, the nucleic acid molecule, e.g., naked DNA molecule, is a dumbbell-shaped linear duplex closed-ended DNA molecule. In some embodiments, the dumbbell-shaped linear duplex closed-ended DNA molecule is a linear, duplex molecule. In some embodiments, the dumbbell-shaped linear duplex closed-ended DNA molecule is covalently closed at each end. In some embodiments, the dumbbell-shaped linear duplex closed-ended DNA molecule comprises two hairpin structures of ITRs in the 5′ and 3′ ends of an expression cassette.

In some embodiments, the provided methods comprise introducing the nucleic acid molecule under conditions for targeted integration of the transgene into a target site of a gene in one or more collected T cells. Such methods can be referred to as “DNA knock-in systems.” In some embodiments, the provided methods allow transgenes to be inserted at a defined target site. In some embodiments, the provided methods allow for gene editing techniques using large transgenes (<5 kb) to be inserted at defined target sites in a genome of a host cell. In some embodiments, homology arms disclosed herein can be, for example, 50 base pairs to two thousand base pairs. In some embodiments, targeted insertion of the transgene to the target site is with excellent efficiency (higher on-target) and excellent specificity (lower off-target).

In some embodiments, the nucleic acid molecule, e.g., naked DNA molecule, has covalently closed ends. In some embodiments, the nucleic acid molecule, e.g., naked DNA molecule, is resistant to exonuclease digestion (e.g., exonuclease I or exonuclease III), e.g., for over an hour at 37° C.

In some embodiments, following electroporation and entry into a T cell, the nucleic acid molecule, e.g., naked DNA molecule, is translocated to the nucleus where expression of the transgene can occur. In some embodiments, the nucleic acid molecule, e.g., naked DNA molecule, is translocated to the nucleus where expression of the transgene located between the two ITRs can occur. In some embodiments, the nucleic acid molecule, e.g., naked DNA molecule, is present in sufficient amounts to transfect the T cell or a plurality of T cells and to provide sufficient levels of gene transfer and expression without undue adverse effects.

In some embodiments, the nucleic acid molecule, e.g., naked DNA molecule, is introduced by electroporation. In some embodiments, electroporation, also referred to as electro gene transfer, gene electro injection, gene electro transfer, or electrically mediated gene therapy, causes temporary destabilization of the cell membrane. In some embodiments, DNA molecules in the surrounding media of the destabilized membrane are able to penetrate into the cytoplasm and nucleoplasm of the T cell.

In some embodiments, the nucleic acid molecule comprises about 500 to 1000 base pairs of homology on either side of the transgene and/or the target site. In some embodiments, the nucleic acid molecule comprises about 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the nucleic acid molecule comprises at least 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the nucleic acid molecule comprises no more than 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the nucleic acid molecule comprises one or more mutations, e.g., silent mutations, that prevent Cas, e.g., Cas9 or Cas12, from recognizing and cleaving the nucleic acid molecule. The nucleic acid molecule may comprise, e.g., at least 1, 2, 3, 4, 5, 10, 20, or 30 silent mutations relative to the corresponding sequence in the genome of the cell to be altered. In some embodiments, the nucleic acid molecule comprises at most 2, 3, 4, 5, 10, 20, 30, or 50 silent mutations relative to the corresponding sequence in the genome of the cell to be altered. In some embodiments, the cDNA comprises one or more mutations, e.g., silent mutations that prevent Cas, e.g., Cas9 or Cas12, from recognizing and cleaving the nucleic acid molecule. The nucleic acid molecule may comprise, e.g., at least 1, 2, 3, 4, 5, 10, 20, or 30 silent mutations relative to the corresponding sequence in the genome of the cell to be altered. In some embodiments, the nucleic acid molecule comprises at most 2, 3, 4, 5, 10, 20, 30, or 50 silent mutations relative to the corresponding sequence in the genome of the cell to be altered.

In some embodiments, the nucleic acid molecule comprises about 150 to 1000 nucleotides of homology on either side of the transgene and/or the target site. In some embodiments, the nucleic acid molecule comprises about 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the nucleic acid molecule comprises at least 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the nucleic acid molecule comprises at most 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene.

In some embodiments, the nucleic acid molecule is introduced into the immune cells after introduction with the one or more gene-editing agents, such as Cas/gRNA RNP, e.g., that has been introduced via electroporation. In some embodiments, the nucleic acid molecule is introduced immediately after the introduction of the one or more gene-editing agents capable of inducing a genetic disruption. In some embodiments, the nucleic acid molecule is introduced into the immune cells within at or about 30 seconds, within at or about 1 minute, within at or about 2 minutes, within at or about 3 minutes, within at or about 4 minutes, within at or about 5 minutes, within at or about 6 minutes, within at or about 6 minutes, within at or about 8 minutes, within at or about 9 minutes, within at or about 10 minutes, within at or about 15 minutes, within at or about 20 minutes, within at or about 30 minutes, within at or about 40 minutes, within at or about 50 minutes, within at or about 60 minutes, within at or about 90 minutes, within at or about 2 hours, within at or about 3 hours or within at or about 4 hours after the introduction of one or more gene-editing agents capable of inducing a genetic disruption. In some embodiments, the nucleic acid molecule is introduced into immune cells at time between at or about 15 minutes and at or about 4 hours after introducing the one or more gene-editing agents, such as between at or about 15 minutes and at or about 3 hours, between at or about 15 minutes and at or about 2 hours, between at or about 15 minutes and at or about 1 hour, between at or about 15 minutes and at or about 30 minutes, between at or about 30 minutes and at or about 4 hours, between at or about 30 minutes and at or about 3 hours, between at or about 30 minutes and at or about 2 hours, between at or about 30 minutes and at or about 1 hour, between at or about 1 hour and at or about 4 hours, between at or about 1 hour and at or about 3 hours, between at or about 1 hour and at or about 2 hours, between at or about 2 hours and at or about 4 hours, between at or about 2 hours and at or about 3 hours or between at or about 3 hours and at or about 4 hours. In some embodiments, the nucleic acid molecule is introduced into immune cells at or about 2 hours after the introduction of the one or more gene-editing agents, such as Cas/gRNA RNP, e.g. that has been introduced via electroporation.

In some embodiments, the introducing of the one or more gene-editing agents is carried out concurrently with the introducing of the nucleic acid molecule. In some embodiments, the one or more gene-editing agents and the nucleic acid molecule are introduced by electroporation simultaneously. In some embodiments, the one or more gene-editing agents and the nucleic acid molecule are introduced in a cell medium comprising the one or more gene-editing agents and the nucleic acid molecule together. In somem embodiments, the cell medium is present during the electroporation.

In some embodiments, the introducing of the one or more gene-editing agents is carried out after the adding of the stimulatory reagent. In some embodiments, the introducing of the one or more gene-editing agents is carried out between or between about 6 hours and 36 hours, 6 hours and 30 hours, 6 hours and 24 hours, 6 hours and 18 hours, 6 hours and 12 hours, 12 hours and 36 hours, 12 hours and 30 hours, 12 hours and 24 hours, 12 hours and 18 hours, 18 hours and 36 hours, 18 hours and 30 hours, 18 hours and 24 hours, 24 hours and 36 hours, 24 hours and 30 hours, or 30 hours and 36 hours, each inclusive after the adding of the stimulatory reagent. In some embodiments, the introducing of the one or more gene-editing agents is carried out between or between about 12 hours and 36 hours, inclusive, after the adding of the stimulatory reagent. In some embodiments, the introducing of the one or more gene-editing agents is carried out between or between about 18 hours and 30 hours, inclusive, after the adding of the stimulatory reagent. In some embodiments, the introducing of the one or more gene-editing agents is carried out between or between about 22 hours and 26 hours, inclusive, after the adding of the stimulatory reagent. In some embodiments, the introducing of the one or more gene-editing agents is carried out at or about 24 hours after the adding of the stimulatory reagent.

In some embodiments, the introducing of the nucleic acid molecule is carried out after the adding of the stimulatory reagent. In some embodiments, the introducing of the nucleic acid molecule is carried out between or between about 6 hours and 36 hours, 6 hours and 30 hours, 6 hours and 24 hours, 6 hours and 18 hours, 6 hours and 12 hours, 12 hours and 36 hours, 12 hours and 30 hours, 12 hours and 24 hours, 12 hours and 18 hours, 18 hours and 36 hours, 18 hours and 30 hours, 18 hours and 24 hours, 24 hours and 36 hours, 24 hours and 30 hours, or 30 hours and 36 hours, each inclusive after the adding of the stimulatory reagent. In some embodiments, the introducing of the nucleic acid molecule is carried out between or between about 12 hours and 36 hours, inclusive, after the adding of the stimulatory reagent. In some embodiments, the introducing of the nucleic acid molecule is carried out between or between about 18 hours and 30 hours, inclusive, after the adding of the stimulatory reagent. In some embodiments, the introducing of the nucleic acid molecule is carried out between or between about 22 hours and 26 hours, inclusive, after the adding of the stimulatory reagent. In some embodiments, the introducing of the nucleic acid molecule is carried out at or about 24 hours after the adding of the stimulatory reagent.

In some embodiments, the nucleic acid molecule is introduced in a cell medium containing the nucleic acid molecule. In some embodiments, the cell medium is any described herein, for instance in Section I-B-2.

In some embodiments, the introducing during any portion of the process or all of the process can be at a temperature of 30° C.±2° C. to 39° C.±2° C., such as at least or about at least 30° C.±2° C., 32° C.±2° C., 34° C.±2° C. or 37° C.±2° C. In some embodiments, at least a portion of the introducing is at 30° C.±2° C. and at least a portion of the introducing is at 37° C. 20° C.

### 1. Target Site

Exemplary target sites for integration are described in WO2021/26018, US20220315921, US20220315932, US20220265718, US20220251575, US20220315928, US20220282285, and US20220315946. In some embodiments, the target site is at a T cell stimulation-associated locus, such as any described in WO2021/260186.

In some embodiments, the gene containing the target site is the T cell receptor alpha constant region (TRAC) gene (IMGT nomenclature). In some embodiments, the endogenous TCR Ca is encoded by the TRAC gene. Exemplary human TCR Ca polypeptide sequences are set forth in SEQ ID NO: 137 and 138 (see UniProtKB Accession No. P01848 or Genbank Accession No. CAA26636.1; mRNA sequence set forth in SEQ ID NO: 139, GenBank: X02592.1). In humans, an exemplary genomic locus of TRAC comprises an open reading frame that contains 4 exons and 3 introns. An exemplary mRNA transcript of TRAC can span the sequence corresponding to coordinates Chromosome 14: 22, 547, 506-22,552,154, on the forward strand, with reference to human genome version GRCh38 (UCSC Genome Browser on Human December 2013 (GRCh38/hg38) Assembly). Table 1 sets forth the coordinates of the exons and introns of the open reading frames and the untranslated regions of the transcript of an exemplary human TRAC locus. In some embodiments, the target site is in exon 1 of the TRAC gene. In some embodiments, the target site in the TRAC gene is set forth in SEQ ID NO: 250.

TABLE 1
Coordinates of exons and introns of exemplary human
TRAC locus (GRCh38, Chromosome 14, forward strand).

| | Start (GrCh38) | End (GrCh38) | Length |
|---|---|---|---|
| 5′ UTR and Exon 1 | 22,547,506 | 22,547,778 | 273 |
| Intron 1-2 | 22,547,779 | 22,549,637 | 1,859 |
| Exon 2 | 22,549,638 | 22,549,682 | 45 |
| Intron 2-3 | 22,549,683 | 22,550,556 | 874 |
| Exon 3 | 22,550,557 | 22,550,664 | 108 |
| Intron 3-4 | 22,550,665 | 22,551,604 | 940 |
| Exon 4 and 3′ UTR | 22,551,605 | 22,552,154 | 550 |

In some embodiments, the gene containing the target site is the T cell receptor beta constant region (TRBC) gene. In some embodiments, the endogenous TCR CP is encoded by TRBC1 or TRBC2 genes (IMGT nomenclature). Exemplary human TCR CP polypeptide sequences are set forth in SEQ ID NO: 140-142 (see UniProtKB Accession No. P01850, AOA5B9 or AOAOG2JNG9; mRNA sequence set forth in SEQ ID NO: 143; GenBank: X00437.1).

In humans, an exemplary genomic locus of TRBC1 comprises an open reading frame that contains 4 exons and 3 introns. An exemplary mRNA transcript of TRBC1 can span the sequence corresponding to coordinates Chromosome 7: 142, 791, 694-142, 793, 368, on the forward strand, with reference to human genome version GRCh38 (UCSC Genome Browser on Human December 2013 (GRCh38/hg38) Assembly). Table 2 sets forth the coordinates of the exons and introns of the open reading frames and the untranslated regions of the transcript of an exemplary human TRBC1 locus.

TABLE 2
Coordinates of exons and introns of exemplary human
TRBC1 locus (GRCh38, Chromosome 7, forward strand).

| | Start (GrCh38) | End (GrCh38) | Length |
|---|---|---|---|
| 5′ UTR and Exon 1 | 142,791,694 | 142,792,080 | 387 |
| Intron 1-2 | 142,792,081 | 142,792,521 | 441 |
| Exon 2 | 142,792,522 | 142,792,539 | 18 |
| Intron 2-3 | 142,792,540 | 142,792,691 | 152 |
| Exon 3 | 142,792,692 | 142,792,798 | 107 |
| Intron 3-4 | 142,792,799 | 142,793,120 | 322 |
| Exon 4 and 3′ UTR | 142,793,121 | 142,793,368 | 248 |

In humans, an exemplary genomic locus of TRBC2 comprises an open reading frame that contains 4 exons and 3 introns. An exemplary mRNA transcript of TRBC2 can span the sequence corresponding to coordinates Chromosome 7: 142, 801, 041-142, 802, 748, on the forward strand, with reference to human genome version GRCh38 (UCSC Genome Browser on Human December 2013 (GRCh38/hg38) Assembly). Table 3 sets forth the coordinates of the exons and introns of the open reading frames and the untranslated regions of the transcript of an exemplary human TRBC2 locus.

TABLE 3
Coordinates of exons and introns of exemplary human
TRBC2 locus (GRCh38, Chromosome 7, forward strand).

| | Start (GrCh38) | End (GrCh38) | Length |
|---|---|---|---|
| 5′ UTR and Exon 1 | 142,801,041 | 142,801,427 | 387 |
| Intron 1-2 | 142,801,428 | 142,801,943 | 516 |
| Exon 2 | 142,801,944 | 142,801,961 | 18 |
| Intron 2-3 | 142,801,962 | 142,802,104 | 143 |
| Exon 3 | 142,802,105 | 142,802,211 | 107 |
| Intron 3-4 | 142,802,212 | 142,802,502 | 291 |
| Exon 4 and 3′ UTR | 142,802,503 | 142,802,748 | 246 |

In some of any embodiments, the genetic disruption is targeted at, near, or within an open reading frame of the gene. In some embodiments, the genetic disruption is targeted at, near, or within the gene or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the gene.

In some aspects, the target site is within an exon of the open reading frame of the gene. In some aspects, the target site is within an intron of the open reading frame of the gene. In some aspects, the target site is within a regulatory or control element, e.g., a promoter, 5′ untranslated region (UTR) or 3′ UTR, of the gene. In some embodiments, the target site is within the gene or any exon or intron of the gene contained therein. In some aspects, the target site is at or near the junction or border between an exon and an intron, or an exon and a regulatory or control element, e.g., a promoter, 5′ untranslated region (UTR) or 3′ UTR, of the gene. In some aspects, the target site is within an intron of the open reading frame of the gene.

In some embodiments, the target site is selected such that after integration of the transgene, the cell is knocked out for, reduced and/or eliminated expression from the gene.

In some embodiments, a genetic disruption, e.g., DNA break, is targeted within an exon of the gene or open reading frame thereof. In certain embodiments, the genetic disruption is within the first exon, second exon, third exon, or fourth exon of the gene or open reading frame thereof. In some of any embodiments, the genetic disruption is within the first exon of the gene or open reading frame thereof. In some embodiments, the genetic disruption is within 500 base pairs (bp) downstream from the 5′ end of the first exon in the gene or open reading frame thereof. In some of any embodiments, the genetic disruption is between the 5′ nucleotide of exon 1 and upstream of the 3′ nucleotide of exon 1. In certain embodiments, the genetic disruption is within 400 bp, 350 bp, 300 bp, 250 bp, 200 bp, 150 bp, 100 bp, or 50 bp downstream from the 5′ end of the first exon in the gene or open reading frame thereof. In some of any embodiments, the genetic disruption is between 1 bp and 400 bp, between 50 and 300 bp, between 100 bp and 200 bp, or between 100 bp and 150 bp downstream from the 5′ end of the first exon in the gene or open reading frame thereof, each inclusive. In certain embodiments, the genetic disruption is between 100 bp and 150 bp downstream from the 5′ end of the first exon in the gene or open reading frame thereof, inclusive.

In some aspects, the target site is within an exon, such as exons corresponding to early coding regions. In some embodiments, the target site is within or in close proximity to exons corresponding to early coding region, e.g., exon 1, 2, 3, 4 or 5 of the open reading frame of the gene, or including sequence immediately following a transcription start site, within exon 1, 2, 3, 4 or 5, or within less than 500, 450, 400, 350, 300, 250, 200, 150, 100 or 50 bp of exon 1, 2, 3, 4 or 5. In some aspects, the target site is within a regulatory or control element, e.g., a promoter, of the gene.

### 2. Genetic Disruption

In some embodiments, one or more targeted genetic disruptions are induced in the gene. In some embodiments, the targeted genetic disruption is induced in or near an exon of the gene. In some embodiments, the targeted genetic disruption is induced in or near an intron of the gene. In some embodiments, the targeted genetic disruption is induced in or near a promoter of the gene.

In some embodiments, genetic disruption results in a DNA break, such as a double-strand break (DSB) or a cleavage, or a nick, such as a single-strand break (SSB), at one or more target site in the gene. In some embodiments, at the site of the genetic disruption, e.g., DNA break or nick, action of cellular DNA repair mechanisms can result in knock-out, insertion, missense or frameshift mutation, such as a biallelic frameshift mutation, deletion of all or part of the gene; or, in the presence of a repair template, e.g., the nucleic acid molecule, can alter the DNA sequence based on the repair template, such as integration or insertion of the transgene contained in the the nucleic acid molecule. In some embodiments, the genetic disruption can be targeted to one or more exons of a gene or portion thereof. In some embodiments, the genetic disruption can be targeted near a desired site of targeted integration of exogenous sequences, e.g., the transgene. In some embodiments, the modified gene after integration of the transgene comprises a deletion, an insertion, a frameshift mutation or a nonsense mutation in the open reading frame of the gene. In some aspects, the endogenous gene product of the gene is not produced, or is truncated, or is non-functional in the immune cell. In some aspects, the endogenous gene product of the gene is produced in full length or is functional in the immune cell.

In some embodiments, the genetic disruption is carried by introducing one or more gene-editing agents capable of inducing a genetic disruption. In some embodiments, such gene-editing agents comprise a DNA binding protein or DNA-binding nucleic acid that specifically binds to or hybridizes to the gene. In some embodiments, the gene-editing agent comprises various components, such as a fusion protein comprising a DNA-targeting protein and a nuclease or an RNA-guided nuclease. In some embodiments, the gene-editing agents can target one or more target sites or target locations. In some aspects, a pair of single stranded breaks (e.g., nicks) on each side of the target site can be generated.

In some embodiments, the genetic disruption occurs at a target site (also known as “target position,” “target DNA sequence” or “target location”). In some embodiments, the target site includes a site on a target DNA (e.g., genomic DNA) that is modified by the one or more gene-editing agents capable of inducing a genetic disruption, e.g., a Cas, e.g., Cas9 or Cas12, molecule complexed with a gRNA that specifies the target site. For example, the target site can include locations in the DNA at the gene, where cleavage or DNA breaks occur. In some aspects, integration of nucleic acid sequences by HDR can occur at or near the target site or target sequence. In some embodiments, a target site can be a site between two nucleotides, e.g., adjacent nucleotides, on the DNA into which one or more nucleotides is added. In some embodiments, the target site is within a target sequence (e.g., the sequence to which the gRNA binds). In some embodiments, a target site is upstream or downstream of a target sequence.

Methods for generating a genetic disruption, including those described herein, can involve the use of one or more gene-editing agents capable of inducing a genetic disruption, such as engineered systems to induce a genetic disruption, a cleavage and/or a double strand break (DSB) or a nick (e.g., a single strand break (SSB)) at a target site or target position in the endogenous or genomic DNA such that repair of the break by an error born process such as non-homologous end joining (NHEJ) or repair by HDR using repair template can result in the insertion of a transgene at or near the target site or position. In some aspects, the one or more gene-editing agents can be used in combination with the nucleic acid molecule for homology directed repair (HDR) mediated targeted integration of the transgene.

In some embodiments, the one or more gene-editing agents capable of inducing a genetic disruption comprises a DNA binding protein or DNA-binding nucleic acid that specifically binds to or hybridizes to a particular site or position in the genome, e.g., a target site or target position. In some aspects, the targeted genetic disruption, e.g., DNA break or cleavage, at the gene is achieved using a protein or a nucleic acid is coupled to or complexed with a gene editing nuclease, such as in a chimeric or fusion protein. In some embodiments, the one or more gene-editing agents capable of inducing a genetic disruption comprises an RNA-guided nuclease, or a fusion protein comprising a DNA-targeting protein and a nuclease.

In some embodiments, the gene-editing agent comprises various components, such as an RNA-guided nuclease, or a fusion protein comprising a DNA-targeting protein and a nuclease. In some embodiments, the targeted genetic disruption is carried out using a DNA-targeting molecule that includes a DNA-binding protein such as one or more zinc finger protein (ZFP) or transcription activator-like effectors (TALEs), fused to a nuclease, such as an endonuclease. In some embodiments, the targeted genetic disruption is carried out using RNA-guided nucleases such as a clustered regularly interspaced short palindromic nucleic acid (CRISPR)-associated nuclease (Cas) system (including Cas and/or Cfpl). In some embodiments, the targeted genetic disruption is carried using gene-editing agents capable of inducing a genetic disruption, such as sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to the at least one target site(s), sequence of a gene or a portion thereof. Exemplary ZFNs, TALEs, and TALENs are described in, e.g., Lloyd et al., Frontiers in Immunology, 4(221): 1-7 (2013).

Zinc finger proteins (ZFPs), transcription activator-like effectors (TALEs), and CRISPR system binding domains can be “engineered” to bind to a predetermined nucleotide sequence, for example via engineering (altering one or more amino acids) of the recognition helix region of a naturally occurring ZFP or TALE protein. Engineered DNA binding proteins (ZFPs or TALEs) are proteins that are non-naturally occurring. Rational criteria for design include application of substitution rules and computerized algorithms for processing information in a database storing information of existing ZFP and/or TALE designs and binding data. See, e.g., U.S. Pat. Nos. 6,140,081; 6,453,242; and 6,534,261; see also WO 98/53058; WO 98/53059; WO 98/53060; WO 02/016536 and WO 03/016496 and U.S. Pub. No. 20110301073.

In some embodiments, the one or more gene-editing agents specifically target the at least one target site at or near the gene. In some embodiments, the gene-editing agent comprises a ZFN, TALEN or a CRISPR/Cas combination that specifically binds to, recognizes, or hybridizes to the target site. In some embodiments, the CRISPR/Cas system includes an engineered crRNA/tracr RNA (“single guide RNA”) to guide specific cleavage. In some embodiments, the gene-editing agent comprises nucleases based on the Argonaute system (e.g., from T. thermophilus, known as ‘TtAgo’ (Swarts et al., (2014) Nature 507(7491): 258-261). Targeted cleavage using any of the nuclease systems described herein can be exploited to insert the sequences of a transgene into a specific target location, using either HDR or NHEJ-mediated processes.

In some embodiments, a “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3, and 6) on a zinc finger recognition helix. Thus, for example, the ZFP or ZFP-containing molecule is non-naturally occurring, e.g., is engineered to bind to a target site of choice.

In some cases, the DNA-targeting molecule is or comprises a zinc-finger DNA binding domain fused to a DNA cleavage domain to form a zinc-finger nuclease (ZFN). For example, fusion proteins comprise the cleavage domain (or cleavage half-domain) from at least one Type IIS restriction enzyme and one or more zinc finger binding domains, which may or may not be engineered. In some cases, the cleavage domain is from the Type IIS restriction endonuclease FokI, which generally catalyzes double-stranded cleavage of DNA, at 9 nucleotides from its recognition site on one strand and 13 nucleotides from its recognition site on the other. See, e.g., U.S. Pat. Nos. 5,356,802; 5,436,150 and 5,487,994; Li et al. (1992) Proc. Natl. Acad. Sci. USA 89:4275-4279; Li et al. (1993) Proc. Natl. Acad. Sci. USA 90:2764-2768; Kim et al. (1994) Proc. Natl. Acad. Sci. USA 91:883-887; Kim et al. (1994) J. Biol. Chem. 269: 978-982. Some gene-specific engineered zinc fingers are available commercially. For example, a platform called CompoZr, for zinc-finger construction is available that provides specifically targeted zinc fingers for thousands of targets. See, e.g., Gaj et al., Trends in Biotechnology, 2013, 31(7), 397-405. In some cases, commercially available zinc fingers are used or are custom designed. In some embodiments, the one or more target sites can be targeted for genetic disruption by engineered ZFNs.

Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from different bacterial species. The new modular proteins have the advantage of displaying more sequence variability than TAL repeats. In some embodiments, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In some embodiments, critical amino acids 12 and 13 can be mutated towards other amino acid residues in order to modulate their specificity towards nucleotides A, T, C and G and in particular to enhance this specificity.

In some embodiments, a “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205.

In some embodiments, a “TALE-nuclease” (TALEN) is a fusion protein comprising a nucleic acid binding domain typically derived from a Transcription Activator Like Effector (TALE) and a nuclease catalytic domain that cleaves a nucleic acid target sequence. The catalytic domain comprises a nuclease domain or a domain having endonuclease activity, like for instance I-TevI, ColE7, NucA and Fok-I. In a particular embodiment, the TALE domain can be fused to a meganuclease like for instance I-CreI and I-OnuI or functional variant thereof. In some embodiments, the TALEN is a monomeric TALEN. A monomeric TALEN is a TALEN that does not require dimerization for specific recognition and cleavage, such as the fusions of engineered TAL repeats with the catalytic domain of I-TevI described in WO2012138927. TALENs have been described and used for gene targeting and gene modifications (see, e.g., Boch et al. (2009) Science 326(5959): 1509-12; Moscou and Bogdanove (2009) Science 326(5959): 1501; Christian et al. (2010) Genetics 186(2): 757-61; Li et al. (2011) Nucleic Acids Res 39(1): 359-72). In some embodiments, one or more sites in the gene can be targeted for genetic disruption by engineered TALENs.

In some embodiments, a “TtAgo” is a prokaryotic Argonaute protein thought to be involved in gene silencing. TtAgo is derived from the bacteria Thermus thermophilus. See, e.g. Swarts et al., (2014) Nature 507(7491): 258-261, G. Sheng et al., (2013) Proc. Natl. Acad. Sci. U.S.A. 111, 652). A “TtAgo system” is all the components required including e.g. guide DNAs for cleavage by a TtAgo enzyme.

In some embodiments, an engineered zinc finger protein, TALE protein or CRISPR/Cas system is not found in nature and whose production results primarily from an empirical process such as phage display, interaction trap or hybrid selection. See e.g., U.S. Pat. Nos. 5,789,538; 5,925,523; 6,007,988; 6,013,453; 6,200,759; WO 95/19431; WO 96/06166; WO 98/53057; WO 98/54311; WO 00/27878; WO 01/60970; WO 01/88197 and WO 02/099084.

Zinc finger and TALE DNA-binding domains can be engineered to bind to a predetermined nucleotide sequence, for example via engineering (altering one or more amino acids) of the recognition helix region of a naturally occurring zinc finger protein or by engineering of the amino acids involved in DNA binding (the repeat variable diresidue or RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, e.g., U.S. Pat. Nos. 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983; 20130196373; 20140120622; 20150056705; 20150335708; 20160030477 and 20160024474, the disclosures of which are incorporated by reference in their entireties.

In some embodiments, the targeted genetic disruption, e.g., DNA break, of the gene is carried out by delivering or introducing one or more gene-editing agents capable of inducing a genetic disruption, e.g., Cas, e.g., Cas9 or Cas12, and/or gRNA components, to a cell, using any of a number of known delivery method or vehicle for introduction or transfer to cells. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, nucleic acid sequence encoding one or more components of one or more gene-editing agents capable of inducing a genetic disruption, e.g., DNA break, is introduced into the cells, e.g., by any methods for introducing nucleic acids into a cell described herein or known. In some embodiments, a vector encoding components of one or more gene-editing agents capable of inducing a genetic disruption such as a CRISPR guide RNA and/or a Cas, e.g., Cas9 or Cas12, enzyme can be delivered into the cell.

In some embodiments, the one or more gene-editing agents capable of inducing a genetic disruption, e.g., one or more gene-editing agents that is a Cas/gRNA, is introduced into the cell as a ribonucleoprotein (RNP) complex. RNP complexes include a sequence of ribonucleotides, such as an RNA or a gRNA molecule, and a protein, such as a Cas, e.g., Cas9 or Cas12, protein or variant thereof. For example, the Cas, e.g., Cas9 or Cas12, protein is delivered as RNP complex that comprises a Cas, e.g., Cas9 or Cas12, protein and a gRNA molecule targeting the target sequence, e.g., using electroporation or other physical delivery method. In some embodiments, the RNP is delivered into the cell via electroporation or other physical means, e.g., particle gun, Calcium Phosphate transfection, cell compression or squeezing. In some embodiments, the RNP can cross the plasma membrane of a cell without the need for additional delivery agents (e.g., small molecule agents, lipids, etc.). In some embodiments, delivery of the one or more gene-editing agents capable of inducing genetic disruption, e.g., CRISPR/Cas, as an RNP offers an advantage that the targeted disruption occurs transiently, e.g., in cells to which the RNP is introduced, without propagation of the agent to cell progenies. For example, delivery by RNP minimizes the agent from being inherited to its progenies, thereby reducing the chance of off-target genetic disruption in the progenies. In such cases, the genetic disruption and the integration of transgene can be inherited by the progeny cells, but without the agent itself, which may further introduce off-target genetic disruptions, being passed on to the progeny cells.

In some embodiments, the RNP complexes include a gRNA that has been modified to include a 3′ poly-A tail and a 5′ Anti-Reverse Cap Analog (ARCA) cap.

Agents and components capable of inducing a genetic disruption, e.g., a Cas, e.g., Cas9 or Cas12, molecule and gRNA molecule, can be introduced into target cells in a variety of forms using a variety of delivery methods and formulations, as set forth in Table 4, or methods described in, e.g., WO 2015/161276; WO2017/193107, WO2017/093969, US 2015/0056705, US 2016/0272999, US 2017/0211075; or US 2017/0016027. As described further herein, the delivery methods and formulations can be used to deliver template polynucleotides and/or other agents to the cell (such as those required for engineering the cells) in prior or subsequent steps of the methods described herein. When a Cas, e.g., Cas9 or Cas12, or gRNA component is encoded as DNA for delivery, the DNA may typically but not necessarily include a control region, e.g., comprising a promoter, to effect expression. Useful promoters for Cas, e.g., Cas9 or Cas12, molecule sequences include, e.g., CMV, EF-1α, EFS, MSCV, PGK, or CAG promoters. Useful promoters for gRNAs include, e.g., H1, EF-1α, tRNA or U6 promoters. Promoters with similar or dissimilar strengths can be selected to tune the expression ofcomponents. Sequences encoding a Cas, e.g., Cas9 or Cas12, molecule may comprise a nuclear localization signal (NLS), e.g., an SV40 NLS. In some embodiments a promoter for a Cas, e.g., Cas9 or Cas12, molecule or a gRNA molecule may be, independently, inducible, tissue specific, or cell specific. In some embodiments, a gene-editing agent capable of inducing a genetic disruption is introduced RNP complexes.

TABLE 4
Exemplary Delivery Methods

| Elements | | |
|---|---|---|
| Cas | gRNA | |
| Molecule(s) | molecule(s) | Comments |
| DNA | DNA | In this embodiment, a Cas, e.g., Cas9 or Cas12, molecule |
| | | and a gRNA are transcribed from DNA. In this |
| | | embodiment, they are encoded on separate molecules. |
| DNA | | In this embodiment, a Cas, e.g., Cas9 or Cas12, molecule |
| | | and a gRNA are transcribed from DNA, here from a |
| | | single molecule. |
| DNA | RNA | In this embodiment, a Cas, e.g., Cas9 or Cas12, molecule |
| | | is transcribed from DNA, and a gRNA is provided as in |
| | | vitro transcribed or synthesized RNA |
| mRNA | RNA | In this embodiment, a Cas, e.g., Cas9 or Cas12, molecule |
| | | is translated from in vitro transcribed mRNA, and a |
| | | gRNA is provided as in vitro transcribed or synthesized |
| | | RNA. |
| mRNA | DNA | In this embodiment, a Cas, e.g., Cas9 or Cas12, molecule |
| | | is translated from in vitro transcribed mRNA, and a |
| | | gRNA is transcribed from DNA. |
| Protein | DNA | In this embodiment, a Cas, e.g., Cas9 or Cas12, molecule |
| | | is provided as a protein, and a gRNA is transcribed from |
| | | DNA. |
| Protein | RNA | In this embodiment, a Cas, e.g., Cas9 or Cas12, molecule |
| | | is provided as a protein, and a gRNA is provided as |
| | | transcribed or synthesized RNA. |

In some aspects, a CRISPR enzyme (e.g., Cas, e.g., Cas9 or Cas12, nuclease) in combination with (and optionally complexed with) a guide sequence is delivered to the cell. For example, one or more elements of a CRISPR system is derived from a type I, type II, or type III CRISPR system. For example, one or more elements of a CRISPR system are derived from a particular organism comprising an endogenous CRISPR system, such as Streptococcus pyogenes, Staphylococcus aureus or Neisseria meningitides.

In some embodiments, a Cas9 nuclease (e.g., that encoded by mRNA from Staphylococcus aureus or from Streptococcus pyogenes, e.g. pCW-Cas9, Addgene #50661, Wang et al. (2014) Science, 3:343-80-4; or nuclease or nickase lentiviral vectors available from Applied Biological Materials (ABM; Canada) as Cat. No. K002, K003, K005 or K006) and a guide RNA specific to the target locus are introduced into cells.

In some embodiments, delivery via electroporation comprises mixing the cells with the Cas- and/or gRNA-encoding DNA or RNP complex in a cartridge, chamber or cuvette and applying one or more electrical impulses of defined duration and amplitude. In some embodiments, delivery via electroporation is performed using a system in which cells are mixed with the Cas- and/or gRNA-encoding DNA in a vessel connected to a device (e.g., a pump) which feeds the mixture into a cartridge, chamber or cuvette wherein one or more electrical impulses of defined duration and amplitude are applied, after which the cells are delivered to a second vessel.

a. CRISPR/Cas

In some embodiments, the targeted genetic disruption, e.g., DNA break, at the gene is carried out using clustered regularly interspaced short palindromic repeats (CRISPR) and CRISPR-associated (Cas) proteins. See Sander and Joung (2014) Nature Biotechnology, 32(4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracr RNA or an active partial tracr RNA), a tracr-mate sequence (encompassing a “direct repeat” and a tracr RNA-processed partial direct repeat in the context of an endogenous CRISPR system), a guide sequence (also referred to as a “spacer” in the context of an endogenous CRISPR system), and/or other sequences and transcripts from a CRISPR locus.

In some aspects, the CRISPR/Cas nuclease or CRISPR/Cas nuclease system includes a non-coding guide RNA (gRNA), which sequence-specifically binds to DNA, and a Cas protein (e.g., Cas9 or Cas12), with nuclease functionality.

In some embodiments, the Cas is selected from the group consisting of Cas3, Cas9, Cas10, Cas12, Cas12a, and Cas13. In some embodiments, the Cas is Cas9 or a variant thereof. In some embodiments, the Cas is Cas9. In some embodiments, the Cas is an enhanced specificity Cas9 (eSpCas9). In some embodiments, the Cas is a high fidelity Cas9 (HiFi Cas9). In some embodiments, the Cas9 is from a bacteria selected from the group consisting of Streptococcus pyogenes, Staphylococcus aureus, Neisseria meningitides, Campylobacter jejuni, and Streptococcus thermophilis. In some embodiments, the Cas9 or a variant thereof is from Streptococcuspyogenes. In some embodiments, the Cas9 is from Streptococcuspyogenes. In some embodiments, the Cas is Cas12. In some embodiments, the Cas is Cas12a.

i. Guide RNA (gRNA)

In some embodiments, the one or more gene-editing agents capable of inducing a genetic disruption comprises at least one of: a guide RNA (gRNA) having a targeting sequence that is complementary with a target site at the gene or at least one nucleic acid encoding the gRNA.

In some aspects, a “gRNA molecule” is a nucleic acid that promotes the specific targeting or homing of a gRNA molecule/Cas molecule complex to a target nucleic acid, such as a locus on the genomic DNA of a cell. gRNA molecules can be unimolecular (having a single RNA molecule), sometimes referred to herein as “chimeric” gRNAs, or modular (comprising more than one, and typically two, separate RNA molecules). In general, a guide sequence, e.g., guide RNA, is any polynucleotide sequences comprising at least a sequence portion that has sufficient complementarity with a target polynucleotide sequence to hybridize with the target sequence at the target site and direct sequence-specific binding of the CRISPR complex to the target sequence. In some embodiments, in the context of formation of a CRISPR complex, “target sequence” is a sequence to which a guide sequence is designed to have complementarity, where hybridization between the target sequence and a domain, e.g., targeting sequence, of the guide RNA promotes the formation of a CRISPR complex. Full complementarity is not necessarily required, provided there is sufficient complementarity to cause hybridization and promote formation of a CRISPR complex. Generally, a guide sequence is selected to reduce the degree of secondary structure within the guide sequence. Secondary structure may be determined by any suitable polynucleotide folding algorithm.

In some embodiments, a guide RNA (gRNA) specific to a target locus of interest is used to RNA-guided nucleases, e.g., Cas, to induce a DNA break at the target site or target position. Methods for designing gRNAs and exemplary targeting sequences can include those described in, e.g., International PCT Pub. Nos. WO2015/161276, WO2017/193107 and WO2017/093969.

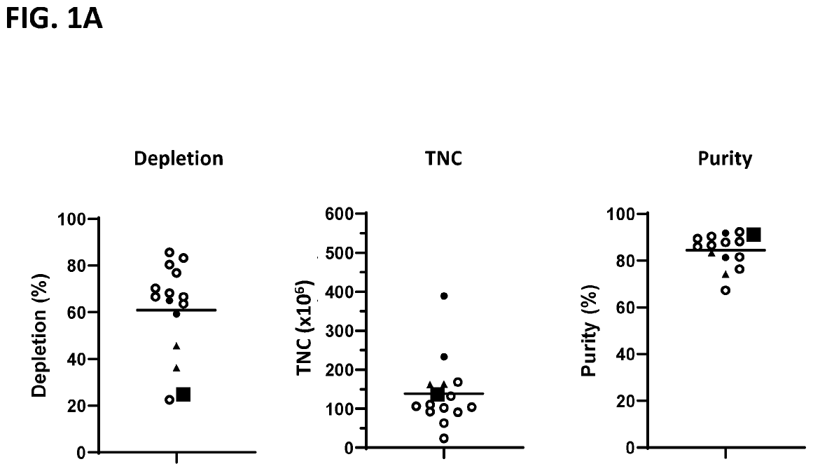

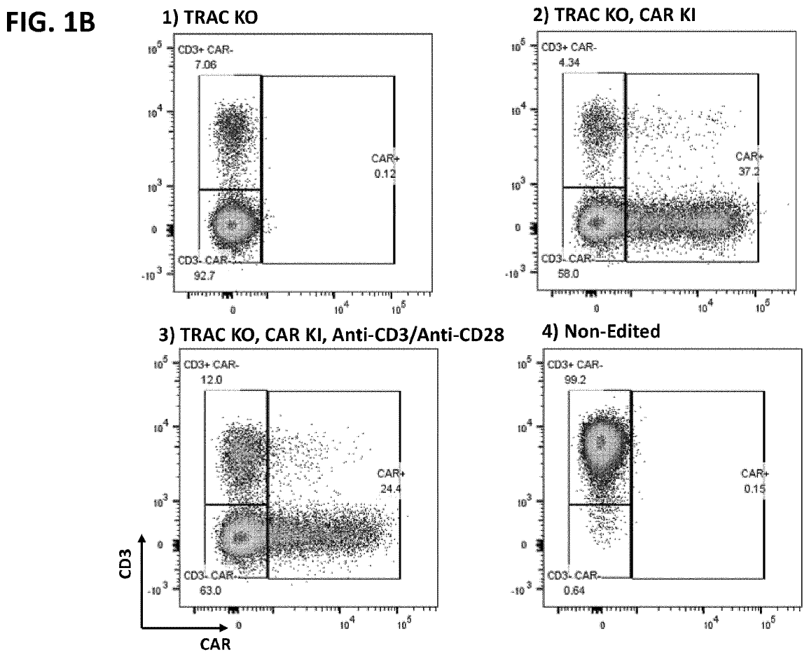

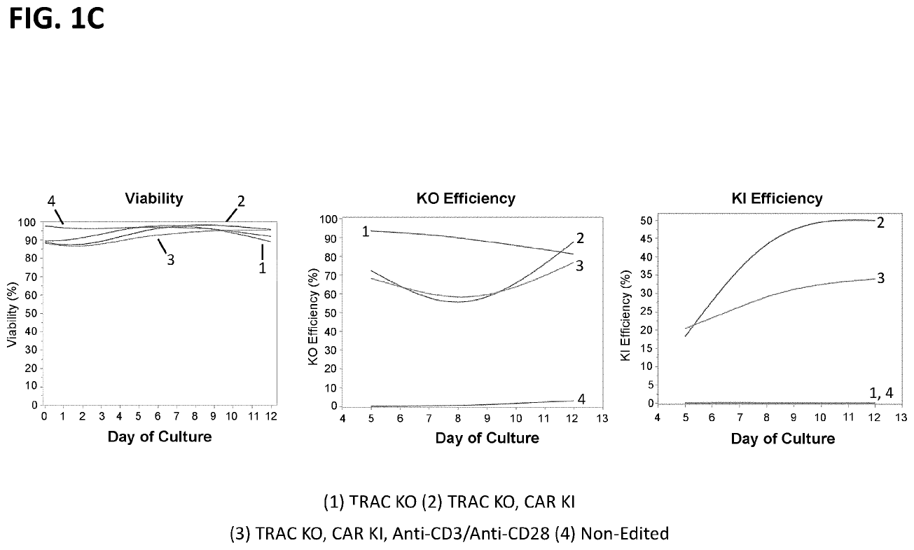

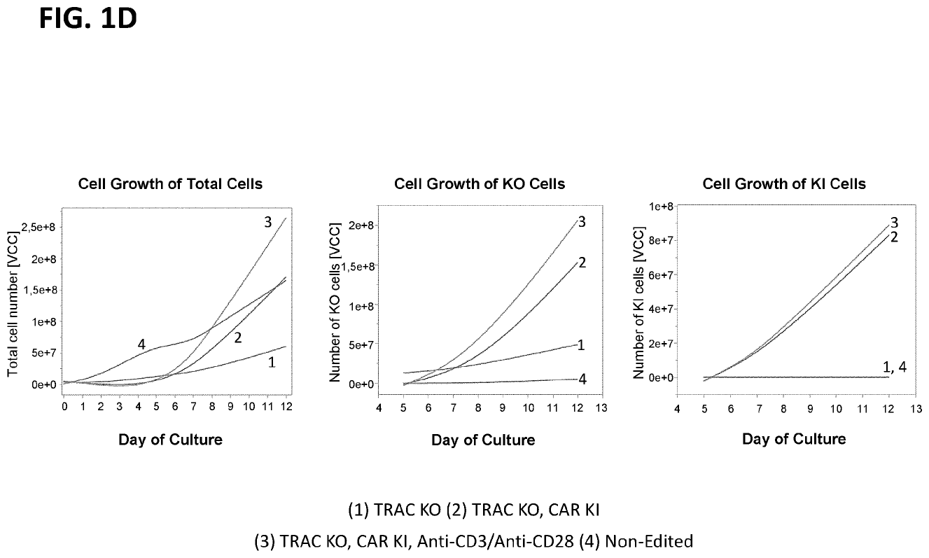

Several exemplary gRNA structures, with sequences indicated thereon, are described in WO2015/161276, e.g., in FIGS. 1A-1G therein. While not wishing to be bound by theory, with regard to the three dimensional form, or intra- or inter-strand interactions of an active form of a gRNA, regions of high complementarity are sometimes shown as duplexes in WO2015/161276, e.g., in FIGS. 1A-1G therein and other depictions provided herein.

In some cases, the gRNA is a unimolecular or chimeric gRNA comprising, from 5′ to 3′: a targeting sequence which is complementary to a target nucleic acid; a first complementarity domain; a linking domain; a second complementarity domain (which is complementary to the first complementarity domain); a proximal domain; and optionally, a tail domain.

In some cases, the gRNA is a modular gRNA comprising first and second strands. In these cases, the first strand preferably includes, from 5′ to 3′: a targeting sequence (which is complementary to a target nucleic acid) and a first complementarity domain. The second strand generally includes, from 5′ to 3′: optionally, a 5′ extension domain; a second complementarity domain; a proximal domain; and optionally, a tail domain.

In some embodiments, the targeting sequence comprises a nucleotide sequence that is complementary, e.g., at least 80, 85, 90, 95, 98 or 99% complementary, e.g., fully complementary, to the target sequence on the target nucleic acid. The strand of the target nucleic acid comprising the target sequence is referred to herein as the “complementary strand” of the target nucleic acid. Guidance on the selection of targeting sequences can be found, e.g., in Fu et al., Nat Biotechnol 2014 March; 32(3):279-284 and Sternberg et al., Nature 2014, 507:62-67. Examples of the placement of targeting sequences include those described in WO2015/161276, e.g., in FIGS. 1A-1G therein.

The targeting sequence is part of an RNA molecule and will therefore comprise the base uracil (U), while any DNA encoding the gRNA molecule will comprise the base thymine (T). While not wishing to be bound by theory, In some embodiments, it is believed that the complementarity of the targeting sequence with the target sequence contributes to specificity of the interaction of the gRNA molecule/Cas molecule complex with a target nucleic acid. It is understood that in a targeting sequence and target sequence pair, the uracil bases in the targeting sequence will pair with the adenine bases in the target sequence. In some embodiments, the targeting sequence itself comprises in the 5′ to 3′ direction, an optional secondary domain, and a core domain. In some embodiments, the core domain is fully complementary with the target sequence. In some embodiments, the targeting sequence is 5 to 50 nucleotides in length. The strand of the target nucleic acid with which the targeting sequence is complementary is referred to herein as the complementary strand. Some or all of the nucleotides of the domain can have a modification, e.g., to render it less susceptible to degradation, improve bio-compatibility, etc. By way of non-limiting example, the backbone of the target sequence can be modified with a phosphorothioate, or other modification(s). In some cases, a nucleotide of the targeting sequence can comprise a 2′ modificatio n, e.g., a 2-acetylation, e.g., a 2′ methylation, or other modification(s).

In various embodiments, the targeting sequence is 16-26 nucleotides in length (i.e. it is 16 nucleotides in length, or 17 nucleotides in length, or 18, 19, 20, 21, 22, 23, 24, 25 or 26 nucleotides in length.

In some embodiments, gRNA sequences that are or comprise a targeting sequence targeting the target site in a particular gene are designed or identified. A genome-wide gRNA database for CRISPR genome editing is publicly available, which contains exemplary single guide RNA (sgRNA) sequences targeting constitutive exons of genes in the human genome or mouse genome (see e.g., genescript.com/gRNA-database.html; see also, Sanjana et al. (2014) Nat. Methods, 11:783-4). In some aspects, the gRNA sequence is or comprises a sequence with minimal off-target binding to a non-target site or position.

In some embodiments, the gRNA can target a site at the gene near a desired site of targeted integration of a transgene. In some aspects, the gRNA can target a site based on the amount of sequences encoding the gene that is desired for regulation of expression of the transgene in a manner, time, or extent similar to the regulation of the gene. In some aspects, the gRNA can target a site based on the amount of sequences encoding the gene that is desired for expression in the cell expressing the transgene. In some aspects, the gRNA can target a site such that upon integration of the transgene, expression of the endogenous gene product encoded by the gene is retained. In some aspects, the endogenous gene product is not expressed (e.g., is knocked-out) following targeting by the gRNA and subsequent HDR. In some aspects, the gRNA can target a site within an exon of the open reading frame of the gene. In some aspects, the gRNA can target a site within an intron of the open reading frame of the gene. In some aspects, the gRNA can target a site within or downstream of a regulatory or control element, e.g., a promoter, of the gene. In some aspects, the target site at the gene that is targeted by the gRNA can be any target sites described herein. In some embodiments, the gRNA can target a site within or in close proximity to exons corresponding to early coding region, e.g., exon 1, 2, 3, 4 or 5 of the open reading frame of the gene, or including sequence immediately following a transcription start site, within exon 1, 2, 3, 4 or 5, or within less than 500, 450, 400, 350, 300, 250, 200, 150, 100 or 50 bp of exon 1, 2, 3, 4 or 5. In some embodiments, the gRNA can target a site at or near exon 2 of the gene or within less than 500, 450, 400, 350, 300, 250, 200, 150, 100 or 50 bp of exon 2.

Exemplary targeting sequences contained within the gRNA for targeting the genetic disruption of the human TRAC, TRBC1 or TRBC2 include those described in, e.g., WO2015/161276, WO2017/193107, WO2017/093969, WO 2019/195492, US2016/272999 and US2015/056705 or a targeting sequence that can bind to the target sequences described in the foregoing. Exemplary targeting sequences contained within the gRNA for targeting the genetic disruption of the human TRAC locus using S. pyogenes or S. aureus Cas, e.g., Cas9 or Cas12, can include any of those set forth in SEQ ID NO: 144-175. Exemplary targeting sequences contained within the gRNA for targeting the genetic disruption of the human TRBC1 or TRBC2 locus using S. pyogenes or S. aureus Cas, e.g., Cas9 or Cas12, can include any of those set forth in SEQ ID NO: 176-233.

In some embodiments, the gRNA for targeting TRAC, TRBC1 and/or TRBC2 include any that are described herein, or are described elsewhere e.g., in WO2015/161276, WO2017/193107, WO2017/093969, WO 2019/195492, US2016/272999 and US2015/056705 or a targeting sequence that can bind to the target sequences described in the foregoing. In some embodiments, the gRNA for targeting the TRAC gene locus can be obtained by in vitro transcription of the sequence AGCGCTCTCGTACAGAGTTGGCATTATAATACGACTCACTATAGGGGAGAATCA AAATCGGTGAATGTTTTAGAGCTAGAAATAGCAAGTTAAAATAAGGCTAGTCCG TTATCAACTTGAAAAAGTGGCACCGAGTCGGTGCTTTTTTT (set forth in SEQ ID NO: 234; bold and underlined portion is complementary to the target site in the TRAC locus), or chemically synthesized, where the gRNA had the sequence 5′-GAG AAU CAA AAU CGG UGA AUG UUU UAG AGC UAG AAA UAG CAA GUU AAA AUA AGG CUA GUC CGU UAU CAA CUU GAA AAA GUG GCA CCG AGU CGG UGC UUU U-3′ (set forth in SEQ ID NO: 235; see Osborn et al., Mol Ther. 24(3):570-581 (2016)). Other exemplary gRNA sequences to generate a genetic disruption of the endogenous genes encoding TCR domains or regions, e.g., TRAC, TRBC1 and/or TRBC2 are described, e.g., in WO2015/161276, WO2017/193107, WO2017/093969, WO 2019/195492, US2016/272999 and US2015/056705.

Exemplary methods for gene editing of the endogenous TCR loci include those described in, e.g. U.S. Publication Nos. US2011/0158957, US2014/0301990, US2015/0098954, US2016/0208243; US2016/272999 and US2015/056705; International PCT Publication Nos. WO2014/191128, WO2015/136001, WO2015/161276, WO2016/069283, WO2016/016341, WO2017/193107, and WO2017/093969; and Osborn et al. (2016) Mol. Ther. 24(3):570-581. Any of the known methods can be used to generate a genetic disruption of the endogenous genes encoding TCR domains or regions can be used in the provided methods.

In some embodiments, targeting sequences include those for introducing a genetic disruption at the TRAC, TRBC1 and/or TRBC2 loci using S. pyogenes Cas, e.g., Cas9 or Cas12, or using N. meningitidis Cas, e.g., Cas9 or Cas12. In some embodiments, targeting sequences include those for introducing a genetic disruption at the TRAC, TRBC1 and/or TRBC2 loci using S. pyogenes Cas, e.g., Cas9 or Cas12. Any of the targeting sequences can be used with a S. pyogenes Cas, e.g., Cas9 or Cas12, molecule that generates a double stranded break (Cas, e.g., Cas9 or Cas12, nuclease) or a single-stranded break (Cas, e.g., Cas9 or Cas12, nickase).

In some embodiments, dual targeting is used to create two nicks on opposite DNA strands by using S. pyogenes Cas, e.g., Cas9 or Cas12, nickases with two targeting sequences that are complementary to opposite DNA strands, e.g., a gRNA comprising any minus strand targeting sequence may be paired with any gRNA comprising a plus strand targeting sequence. In some embodiments, the two gRNAs are oriented on the DNA such that PAMs face outward and the distance between the 5′ ends of the gRNAs is 0-50 bp. In some embodiments, two gRNAs are used to target two Cas, e.g., Cas9 or Cas12, nucleases or two Cas, e.g., Cas9 or Cas12, nickases, for example, using a pair of Cas, e.g., Cas9 or Cas12, molecule/gRNA molecule complex guided by two different gRNA molecules to cleave the target sequence with two single stranded breaks on opposing strands of the target sequence. In some embodiments, the two Cas, e.g., Cas9 or Cas12, nickases can include a molecule having HNH activity, e.g., a Cas, e.g., Cas9 or Cas12, molecule having the RuvC activity inactivated, e.g., a Cas, e.g., Cas9 or Cas12, molecule having a mutation at D10, e.g., the D10A mutation, a molecule having RuvC activity, e.g., a Cas, e.g., Cas9 or Cas12, molecule having the HNH activity inactivated, e.g., a Cas, e.g., Cas9 or Cas12, molecule having a mutation at H840, e.g., a H840A, or a molecule having RuvC activity, e.g., a Cas, e.g., Cas9 or Cas12, molecule having the HNH activity inactivated, e.g., a Cas, e.g., Cas9 or Cas12, molecule having a mutation at N863, e.g., N863A. In some embodiments, each of the two gRNAs are complexed with a D10A Cas, e.g., Cas9 or Cas12, nickase.

Methods for selection and validation of target sequences as well as off-target analyses are described, e.g., in Mali et al., 2013 Science 339 (6121): 823-826; Hsu et al. Nat Biotechnol, 31(9): 827-32; Fu et al., Nat Biotechnol 2014 March; 32(3):279-284; Heigwer et al., 2014 Nat Methods 11(2):122-3; Bae et al., Bioinformatics. 2014 May 15; 30(10):1473-5; Xiao A et al., Bioinformatics. 2014 Apr. 15; 30(8):1180-1182.

In some embodiments, a software tool can be used to optimize the choice of gRNA within a user's target sequence, e.g., to minimize total off-target activity across the genome. Off target activity may be other than cleavage. For example, for each possible gRNA choice using S. pyogenes Cas, e.g., Cas9 or Cas12, software tools can identify all potential off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA can then be ranked according to its total predicted off-target cleavage; the top-ranked gRNAs represent those that are likely to have the greatest on-target and the least off-target cleavage. Other functions, e.g., automated reagent design for gRNA vector construction, primer design for the on-target Surveyor assay, and primer design for high-throughput detection and quantification of off-target cleavage via next-generation sequencing, can also be included in the tool. Candidate gRNA molecules can be evaluated by art-known methods or as described herein.

In some embodiments, gRNAs for use with S. pyogenes, S. aureus, and N. meningitidis Cas, e.g., Cas9 or Cas12, are identified using a DNA sequence searching algorithm, e.g., using a custom gRNA design software based on the public tool cas-offinder (Bae et al. Bioinformatics. 2014; 30(10): 1473-1475). The custom gRNA design software scores guides after calculating their genome-wide off-target propensity. Typically matches ranging from perfect matches to 7 mismatches are considered for guides ranging in length from 17 to 24. In some aspects, once the off-target sites are computationally determined, an aggregate score is calculated for each guide and summarized in a tabular output using a web-interface. In addition to identifying potential gRNA sites adjacent to PAM sequences, the software also can identify all PAM adjacent sequences that differ by 1, 2, 3 or more nucleotides from the selected gRNA sites. In some embodiments, genomic DNA sequences for each gene are obtained from the UCSC Genome browser and sequences can be screened for repeat elements using the publicly available RepeatMasker program. RepeatMasker searches input DNA sequences for repeated elements and regions of low complexity. The output is a detailed annotation of the repeats present in a given query sequence.

Following identification, gRNAs can be ranked into tiers based on one or more of their distance to the target site, their orthogonality and presence of a 5′ G (based on identification of close matches in the human genome containing a relevant PAM, e.g., in the case of S. pyogenes, a NGG PAM, in the case of S. aureus, NNGRR (e.g., a NNGRRT or NNGRRV) PAM, and in the case of N. meningtidis, a NNNNGATT or NNNNGCTT PAM). Orthogonality refers to the number of sequences in the human genome that contain a minimum number of mismatches to the target sequence. A “high level of orthogonality” or “good orthogonality” may, for example, refer to 20-mer targeting sequences that have no identical sequences in the human genome besides the intended target, nor any sequences that contain one or two mismatches in the target sequence. Targeting sequences with good orthogonality are selected to minimize off-target DNA cleavage. It is to be understood that this is a non-limiting example and that a variety of strategies could be utilized to identify gRNAs for use with S. pyogenes, S. aureus and N. meningitidis or other Cas, e.g., Cas9 or Cas12, enzymes.

In some embodiments, gRNAs for use with the S. pyogenes Cas, e.g., Cas9 or Cas12, can be identified using the publicly available web-based ZiFiT server (Fu et al., Nat Biotechnol 2014 March; 32(3):279-284, for the original references see Sander et al., 2007, NAR 35:W599-605; Sander et al., 2010, NAR 38: W462-8). In addition to identifying potential gRNA sites adjacent to PAM sequences, the software also identifies all PAM adjacent sequences that differ by 1, 2, 3 or more nucleotides from the selected gRNA sites. In some aspects, genomic DNA sequences for each gene can be obtained from the UCSC Genome browser and sequences can be screened for repeat elements using the publicly available Repeat-Masker program. RepeatMasker searches input DNA sequences for repeated elements and regions of low complexity. The output is a detailed annotation of the repeats present in a given query sequence.

ii. Cas9

Cas9 molecules of a variety of species can be used in the methods and compositions described herein. While the S. pyogenes, S. aureus, N. meningitidis, and S. thermophilus Cas9 molecules are the subject of much of the disclosure herein, Cas9 molecules of, derived from, or based on the Cas9 proteins of other species listed herein can be used as well. In other words, while the much of the description herein uses S. pyogenes, S. aureus, N. meningitidis, and S. thermophilus Cas9 molecules, Cas9 molecules from the other species can replace them. Such species include: Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteo bacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae. Examples of Cas9 molecules can include those described in, e.g., WO2015/161276, WO2017/193107, WO2017/093969, US2016/272999 and US2015/056705.

A Cas9 molecule, or Cas9 polypeptide, as that term is used herein, refers to a molecule or polypeptide that can interact with a gRNA molecule and, in concert with the gRNA molecule, homes or localizes to a site which comprises a target sequence and PAM sequence. Cas9 molecule and Cas9 polypeptide, as those terms are used herein, refer to naturally occurring Cas9 molecules and to engineered, altered, or modified Cas9 molecules or Cas9 polypeptides that differ, e.g., by at least one amino acid residue, from a reference sequence, e.g., the most similar naturally occurring Cas9 molecule.

Crystal structures have been determined for two different naturally occurring bacterial Cas9 molecules (Jinek et al., Science, 343(6176):1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014 Sep. 25; 513(7519):569-73).

Exemplary Cas9 molecules, their structure and variants include those described in, e.g., WO2015/161276, e.g., in FIGS. 2A-2G and 8A-8B therein, and WO2017/193107, WO2017/093969, US2016/272999 and US2015/056705.

Nucleic acids encoding the Cas9 molecules or Cas9 polypeptides, e.g., an eaCas9 molecule or eaCas9 polypeptide, can be used in connection with any of the embodiments provided herein.

Exemplary nucleic acids encoding Cas9 molecules or Cas9 polypeptides are described in Cong et al., Science 2013, 399(6121):819-823; Wang et al., Cell 2013, 153(4):910-918; Mali et al., Science 2013, 399(6121):823-826; Jinek et al., Science 2012, 337(6096):816-821, and WO2015/161276, e.g., in FIG. 8 therein.

In some embodiments, a nucleic acid encoding a Cas9 molecule or Cas9 polypeptide can be a synthetic nucleic acid sequence. For example, the synthetic nucleic acid molecule can be chemically modified. In some embodiments, the Cas9 mRNA has one or more (e.g., all of the following properties: it is capped, polyadenylated, substituted with 5-methylcytidine and/or pseudouridine. In addition, or alternatively, the synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. In addition, or alternatively, a nucleic acid encoding a Cas9 molecule or Cas9 polypeptide may comprise a nuclear localization sequence (NLS). Nuclear localization sequences are known.

In some embodiments, the Cas9 molecule comprises by a sequence that is or comprises any of SEQ ID NO: 236-244 or a sequence that exhibits at least 85%, 86%, 87%, 88%, 89%, 90%, 91%, 92%, 92%, 94%, 95%, 96%, 97%, 98%, 99% or more sequence identity to any of SEQ ID NO: 236-244. Exemplary Cas9 molecule includes a Cas9 molecule of S. Pyogenes, S. aureus or N. meningitidis. In some embodiments, a Cas9 molecule or Cas9 polypeptide comprises regions 1-5, together with sufficient additional Cas9 molecule sequence to provide a biologically active molecule, e.g., a Cas9 molecule having at least one activity described herein. In some embodiments, each of regions 1-6, independently, have, 50%, 60%, 70%, or 80% homology with the corresponding residues of a Cas9 molecule or Cas9 polypeptide described herein, e.g., set forth in SEQ ID NO: 236-244 or a sequence disclosed in WO2015/161276, e.g., from FIGS. 2A-2G or from FIGS. 7A-7B therein.

If any of the foregoing Cas9 sequences are fused with a peptide or polypeptide at the C-terminus, it is understood that the stop codon will be removed.

Various types of Cas molecules or Cas polypeptides can be used to practice the inventions disclosed herein. In some embodiments, Cas molecules of Type II Cas systems are used. In other embodiments, Cas molecules of other Cas systems are used. For example, Type I or Type III Cas molecules may be used. Exemplary Cas molecules (and Cas systems) are described, e.g., in Haft et al., pLoS Computational Biology 2005, 1(6): e60 and Makarova et al., Nature Review Microbiology 2011, 9:467-477, the contents of both references are incorporated herein by reference in their entirety. Exemplary Cas molecules (and Cas systems) include those described in, e.g., WO2015/161276, WO2017/193107, WO2017/093969, US2016/272999 and US2015/056705.

iii. Cpf1

In some embodiments, the guide RNA or gRNA promotes the specific association targeting of an RNA-guided nuclease such as a Cas9 or a Cpf1 to a target sequence such as a genomic or episomal sequence in a cell. In general, gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, in some embodiments by duplexing). gRNAs and their component parts are described throughout the literature, in some embodiments in Briner et al. Molecular Cell (2014) 56(2), 333-339, which is incorporated by reference.

Guide RNAs, whether unimolecular or modular, generally include a targeting sequence that is fully or partially complementary to a target, and are typically 10⁻³⁰ nucleotides in length, and in certain embodiments are 16-24 nucleotides in length (in some embodiments, 16, 17, 18, 19, 20, 21, 22, 23 or 24 nucleotides in length). In some aspects, the targeting sequences are at or near the 5′ terminus of the gRNA in the case of a Cas9 gRNA, and at or near the 3′ terminus in the case of a Cpf1 gRNA. While the foregoing description has focused on gRNAs for use with Cas9, it should be appreciated that other RNA-guided nucleases have been (or may in the future be) discovered or invented which utilize gRNAs that differ in some ways from those described to this point. In some embodiments, Cpf1 (“CRISPR from Prevotella and Franciscella 1”) is a recently discovered RNA-guided nuclease that does not require a tracrRNA to function. (Zetsche et al., 2015, Cell 163, 759-771, incorporated by reference herein). A gRNA for use in a Cpf1 genome editing system generally includes a targeting sequence and a complementarity domain (alternately referred to as a “handle”). It should also be noted that, in gRNAs for use with Cpf1, the targeting sequence is usually present at or near the 3′ end, rather than the 5′ end as described above in connection with Cas9 gRNAs (the handle is at or near the 5′ end of a Cpf1 gRNA).

Although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting sequences, and skilled artisans will appreciate that a given targeting sequence can be incorporated in any suitable gRNA, including a unimolecular or chimeric gRNA, or a gRNA that includes one or more chemical modifications and/or sequential modifications (substitutions, additional nucleotides, truncations, etc.). Thus, in some aspects in this disclosure, gRNAs may be described solely in terms of their targeting sequences.

More generally, some aspects of the present disclosure relate to systems, methods and compositions that can be implemented using multiple RNA-guided nucleases. Unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

While Cas9 and Cpf1 share similarities in structure and function, it should be appreciated that certain Cpf1 activities are mediated by structural domains that are not analogous to any Cas9 domains. In some embodiments, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

Nucleic acids encoding RNA-guided nucleases, e.g., Cas9, Cpf1 or functional fragments thereof, are provided herein. Exemplary nucleic acids encoding RNA-guided nucleases include those described in, for example, Cong et al., Science 2013, 399(6121):819-823; Wang et al., Cell 2013, 153(4):910-918; Mali et al., Science 2013, 399(6121):823-826; Jinek et al., Science 2012, 337(6096):816-821.

Any of the Cas9 molecules, gRNA molecules, Cas9 molecule/gRNA molecule complexes, can be evaluated by art-known methods or as described herein. For example, exemplary methods for evaluating the endonuclease activity of Cas9 molecule are described, e.g., in Jinek et al., Science 2012, 337(6096):816-821, WO2015/161276, WO2017/193107, WO2017/093969, US2016/272999 and US2015/056705.

### 3. Homology-Directed Repair (HDR)

In some aspects, the provided methods involve targeted integration of the transgene at a target site. In some aspects, homology-directed repair (HDR) can mediate the site specific integration of the transgene at the target site. In some embodiments, the presence of a genetic disruption (e.g., a DNA break) and a nucleic acid molecule containing one or more homology arms (e.g., containing nucleic acid sequences homologous sequences surrounding the genetic disruption) can induce or direct HDR, with homologous sequences acting as a template for DNA repair. Based on homology between the endogenous gene sequence surrounding the genetic disruption and the 5′ and/or 3′ homology arms included in the nucleic acid molecule, cellular DNA repair machinery can use the nucleic acid molecule to repair the DNA break and resynthesize genetic information at the site of the genetic disruption, thereby effectively inserting or integrating the transgene in the nucleic acid molecule at or near the site of the genetic disruption. In some embodiments, the genetic disruption at the gene can be generated by any of the methods for generating a targeted genetic disruption described herein.

In some embodiments, the nucleic acid molecule is a polynucleotide containing a transgene, such as exogenous or heterologous nucleic acid sequences, encoding a recombinant protein, e.g., recombinant receptor or a portion thereof (e.g., one or more regions or domains of the recombinant receptor), and homology sequences (e.g., homology arms) that are homologous to sequences at or near the endogenous genomic site at the gene. In some aspects, the transgene in the nucleic acid molecule comprise sequence of nucleotides encoding a recombinant receptor or a portion thereof. In some aspects, upon targeted integration of the transgene, the gene in the engineered cell is modified such that the modified gene contains the transgene.

Alteration of nucleic acid sequences at the target site can occur by HDR with an exogenously provided nucleic acid molecule. For example, the nucleic acid molecule provides for alteration of the target sequence, such as insertion of the transgene contained within the nucleic acid molecule. In some embodiments, a plasmid or a vector can be used as a template for homologous recombination.

In some embodiments, “recombination” includes a process of exchange of genetic information between two polynucleotides. In some embodiments, “homologous recombination (HR)” includes a specialized form of such exchange that takes place, for example, during repair of double-strand breaks in cells via homology-directed repair mechanisms. This process requires nucleotide sequence homology, uses a template polynucleotide to template repair of a target DNA (e.g., the one that experienced the double-strand break, such as target site in the endogenous gene), and is variously known as “non-crossover gene conversion” or “short tract gene conversion,” because it leads to the transfer of genetic information from the template polynucleotide to the target. In some embodiments, such transfer can involve mismatch correction of heteroduplex DNA that forms between the broken target and the template polynucleotide, and/or “synthesis-dependent strand annealing,” in which the template polynucleotide is used to resynthesize genetic information that will become part of the target, and/or related processes. Such specialized HR often results in an alteration of the sequence of the target molecule such that part or all of the sequence of the template polynucleotide is incorporated into the target polynucleotide.

In some embodiments, a nucleic acid molecule, e.g., polynucleotide containing transgene, is integrated into the genome of a cell via homology-independent mechanisms. The methods comprise creating a double-stranded break (DSB) in the genome of a cell and cleaving the nucleic acid molecule using a nuclease, such that the nucleic acid molecule is integrated at the site of the DSB. In some embodiments, the nucleic acid molecule is integrated via non-homology dependent methods (e.g., NHEJ). Upon in vivo cleavage the nucleic acid molecules can be integrated in a targeted manner into the genome of a cell at the location of a DSB. The nucleic acid molecule can include one or more of the same target sites for one or more of the nucleases used to create the DSB. Thus, the nucleic acid molecule may be cleaved by one or more of the same nucleases used to cleave the endogenous gene into which integration is desired. In some embodiments, the nucleic acid molecule includes different nuclease target sites from the nucleases used to induce the DSB. As described herein, the genetic disruption of the target site or target position can be created by any know methods or any methods described herein, such as ZFNs, TALENs, CRISPR/Cas system, or TtAgo nucleases.

In some embodiments, DNA repair mechanisms can be induced by a nuclease after (1) a single double-strand break, (2) two single strand breaks, (3) two double stranded breaks with a break occurring on each side of the target site, (4) one double stranded break and two single strand breaks with the double strand break and two single strand breaks occurring on each side of the target site (5) four single stranded breaks with a pair of single stranded breaks occurring on each side of the target site, or (6) one single stranded break.

In canonical HDR, a double-stranded nucleic acid molecule is introduced, comprising homologous sequence to the target site that will either be directly incorporated into the target site or used as a template to insert the transgene or correct the sequence of the target site. After resection at the break, repair can progress by different pathways, e.g., by the double Holliday junction model (or double strand break repair, DSBR, pathway) or the synthesis-dependent strand annealing (SDSA) pathway.

In the double Holliday junction model, strand invasion by the two single stranded overhangs of the target site to the homologous sequences in the nucleic acid molecule occurs, resulting in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the target site, e.g., insertion of the transgene in the nucleic acid molecule. Crossover with the nucleic acid molecule may occur upon resolution of the junctions.

In the SDSA pathway, only one single stranded overhang invades the nucleic acid molecule and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

In alternative HDR, a single strand nucleic acid molecule is introduced. A nick, single strand break, or double strand break at the target site, for altering a desired target site, is mediated by a nuclease molecule, and resection at the break occurs to reveal single stranded overhangs. Incorporation of the sequence of the nucleic acid molecule to correct or alter the target site of the DNA typically occurs by the SDSA pathway, as described herein.

“Alternative HDR”, or alternative homology-directed repair, in some embodiments, refers to the process of repairing DNA damage using a homologous nucleic acid (e.g., an endogenous homologous sequence, e.g., a sister chromatid, or an exogenous nucleic acid, e.g., a nucleic acid molecule). Alternative HDR is distinct from canonical HDR in that the process utilizes different pathways from canonical HDR, and can be inhibited by the canonical HDR mediators, RAD51 and BRCA2. Also, alternative HDR uses a single-stranded or nicked homologous nucleic acid for repair of the break. “Canonical HDR”, or canonical homology-directed repair, in some embodiments, refers to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA In a normal cell, HDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2 and the homologous nucleic acid is typically double-stranded. Unless indicated otherwise, the term “HDR” in some embodiments encompasses canonical HDR and alternative HDR.

In some embodiments, double strand cleavage is effected by a nuclease, e.g., a Cas, e.g., Cas9 or Cas12, molecule having cleavage activity associated with an HNH-like domain and cleavage activity associated with a RuvC-like domain, e.g., an N-terminal RuvC-like domain, e.g., a wild type Cas, e.g., Cas9 or Cas12. Such embodiments require only a single gRNA.

In some embodiments, one single strand break, or nick, is effected by a nuclease molecule having nickase activity, e.g., a Cas, e.g., Cas9 or Cas12, nickase. A nicked DNA at the target site can be a substrate for alternative HDR.

In some embodiments, two single strand breaks, or nicks, are effected by a nuclease, e.g., Cas, e.g., Cas9 or Cas12, molecule, having nickase activity, e.g., cleavage activity associated with an HNH-like domain or cleavage activity associated with an N-terminal RuvC-like domain. Such embodiments usually require two gRNAs, one for placement of each single strand break. In some embodiments, the Cas, e.g., Cas9 or Cas12, molecule having nickase activity cleaves the strand to which the gRNA hybridizes, but not the strand that is complementary to the strand to which the gRNA hybridizes. In some embodiments, the Cas, e.g., Cas9 or Cas12, molecule having nickase activity does not cleave the strand to which the gRNA hybridizes, but rather cleaves the strand that is complementary to the strand to which the gRNA hybridizes. In some embodiments, the nickase has HNH activity, e.g., a Cas, e.g., Cas9 or Cas12, molecule having the RuvC activity inactivated, e.g., a Cas, e.g., Cas9 or Cas12, molecule having a mutation at D10, e.g., the D10A mutation. D10A inactivates RuvC; therefore, the Cas, e.g., Cas9 or Cas12, nickase has (only) HNH activity and will cut on the strand to which the gRNA hybridizes (e.g., the complementary strand, which does not have the NGG PAM on it). In some embodiments, a Cas, e.g., Cas9 or Cas12, molecule having an H840, e.g., an H840A, mutation can be used as a nickase. H840A inactivates HNH; therefore, the Cas, e.g., Cas9 or Cas12, nickase has (only) RuvC activity and cuts on the non-complementary strand (e.g., the strand that has the NGG PAM and whose sequence is identical to the gRNA). In some embodiments, the Cas, e.g., Cas9 or Cas12, molecule is an N-terminal RuvC-like domain nickase, e.g., the Cas, e.g., Cas9 or Cas12, molecule comprises a mutation at N863, e.g., N863A.

In some embodiments, in which a nickase and two gRNAs are used to position two single strand nicks, one nick is on the + strand and one nick is on the -strand of the target DNA. The PAMs are outwardly facing. The gRNAs can be selected such that the gRNAs are separated by, from about 0-50, 0-100, or 0-200 nucleotides. In some embodiments, there is no overlap between the target sequences that are complementary to the targeting sequences of the two gRNAs. In some embodiments, the gRNAs do not overlap and are separated by as much as 50, 100, or 200 nucleotides. In some embodiments, the use of two gRNAs can increase specificity, e.g., by decreasing off-target binding (Ran et al., Cell. 2013 Sep. 12; 154(6):1380-9).

In some embodiments, a single nick can be used to induce HDR, e.g., alternative HDR. It is contemplated herein that a single nick can be used to increase the ratio of HR to NHEJ at a given cleavage site, such as target site. In some embodiments, a single strand break is formed in the strand of the DNA at the target site to which the targeting sequence of said gRNA is complementary. In some embodiments, a single strand break is formed in the strand of the DNA at the target site other than the strand to which the targeting sequence of said gRNA is complementary.

In some embodiments, other DNA repair pathways such as single strand annealing (SSA), single-stranded break repair (SSBR), mismatch repair (MMR), base excision repair (BER), nucleotide excision repair (NER), intrastrand cross-link (ICL), translesion synthesis (TLS), error-free postreplication repair (PRR) can be employed by the cell to repair a double-stranded or single-stranded break created by the nucleases.

Targeted integration results in the transgene, e.g., sequences between the homology arms, being integrated into the genome. The transgene may be integrated anywhere at or near one of the at least one target site(s) or site in the genome. In some embodiments, the transgene is integrated at or near one of the at least one target site(s), for example, within 300, 250, 200, 150, 100, 50, 10, 5, 4, 3, 2, 1 or fewer base pairs upstream or downstream of the site of cleavage, such as within 100, 50, 10, 5, 4, 3, 2, 1 base pairs of either side of the target site, such as within 50, 10, 5, 4, 3, 2, 1 base pairs of either side of the target site. In some embodiments, the integrated sequence comprising the transgene does not include any vector sequences (e.g., viral vector sequences). In some embodiments, the integrated sequence includes a portion of the vector sequences (e.g., viral vector sequences).

The double strand break or single strand break (such as target site) in one of the strands should be sufficiently close to the target integration site, e.g., site for targeted integration, such that an alteration is produced in the desired region, such as insertion of transgene or correction of a mutation occurs. In some embodiments, the distance is not more than 10, 25, 50, 100, 200, 300, 350, 400 or 500 nucleotides. In some embodiments, it is believed that the break should be sufficiently close to the target integration site such that the break is within the region that is subject to exonuclease-mediated removal during end resection. In some embodiments, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 300, 350, 400 or 500 nucleotides of the region desired to be altered, e.g., site for targeted insertion. The break, e.g., a double strand or single strand break, can be positioned upstream or downstream of the region desired to be altered, e.g., site for targeted insertion. In some embodiments, a break is positioned within the region desired to be altered, e.g., within a region defined by at least two mutant nucleotides. In some embodiments, a break is positioned immediately adjacent to the region desired to be altered, e.g., immediately upstream or downstream of target integration site.

In some embodiments, a single strand break is accompanied by an additional single strand break, positioned by a second gRNA molecule. For example, the targeting domains are configured such that a cleavage event, e.g., the two single strand breaks, are positioned within 1, 2, 3, 4, 5, 10, 15, 20, 25, 30, 35, 40, 45, 50, 60, 70, 80, 90, 100, 150, 200, 300, 350, 400 or 500 nucleotides of a target integration site. In some embodiments, the first and second gRNA molecules are configured such, that when guiding a Cas, e.g., Cas9 or Cas12, nickase, a single strand break will be accompanied by an additional single strand break, positioned by a second gRNA, sufficiently close to one another to result in alteration of the desired region. In some embodiments, the first and second gRNA molecules are configured such that a single strand break positioned by said second gRNA is within 10, 20, 30, 40, or 50 nucleotides of the break positioned by said first gRNA molecule, e.g., when the Cas, e.g., Cas9 or Cas12, is a nickase. In some embodiments, the two gRNA molecules are configured to position cuts at the same position, or within a few nucleotides of one another, on different strands, e.g., essentially mimicking a double strand break.

In some embodiments, in which a gRNA (unimolecular, chimeric, or modular gRNA) and Cas, e.g., Cas9 or Cas12, nuclease induce a double strand break for the purpose of inducing HDR-mediated insertion of transgene or correction, the cleavage site, such as target site, is between 0-200 bp (e.g., 0-175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target integration site. In some embodiments, the cleavage site, such as target site such as target site, is between 0-100 bp (e.g., 0 to 75, 0 to 50, 0 to 25, 25 to 100, 25 to 75, 25 to 50, 50 to 100, 50 to 75 or 75 to 100 bp) away from the site for targeted integration.

In some embodiments, one can promote HDR by using nickases to generate a break with overhangs. In some embodiments, the single stranded nature of the overhangs can enhance the cell's likelihood of repairing the break by HDR as opposed to, e.g., NHEJ.

Specifically, in some embodiments, HDR is promoted by selecting a first gRNA that targets a first nickase to a first target site, and a second gRNA that targets a second nickase to a second target site which is on the opposite DNA strand from the first target site and offset from the first nick. In some embodiments, the targeting domain of a gRNA molecule is configured to position a cleavage event sufficiently far from a preselected nucleotide, e.g., the nucleotide of a coding region, such that the nucleotide is not altered. In some embodiments, the targeting domain of a gRNA molecule is configured to position an intronic cleavage event sufficiently far from an intron/exon border, or naturally occurring splice signal, to avoid alteration of the exonic sequence or unwanted splicing events. In some embodiments, the targeting domain of a gRNA molecule is configured to position in an early exon, to allow in-frame integration of the transgene at or near one of the at least one target site(s).

In some embodiments, a double strand break can be accompanied by an additional double strand break, positioned by a second gRNA molecule. In some embodiments, a double strand break can be accompanied by two additional single strand breaks, positioned by a second gRNA molecule and a third gRNA molecule. In some embodiments, two gRNAs, e.g., independently, unimolecular, chimeric, or modular gRNA, are configured to position a double-strand break on both sides of a target integration site, e.g., site for targeted integration.

### 4. Nucleic Acid Molecule

In some embodiments, the nucleic acid molecule contains a transgene. In some embodiments, the nucleic acid molecule contains one or more homology sequences (e.g., homology arms) linked to and/or flanking the transgene. In some embodiments, the nucleic acid molecule includes nucleic acid sequences, such as a transgene, between the homology arms, for insertion or integration into the genome of a cell. The transgene in the nucleic acid molecule may comprise one or more sequences encoding a functional polypeptide (for example, a recombinant receptor or a portion thereof), with or without a promoter or other regulatory elements.

In some embodiments, a nucleic acid molecule, e.g., a polynucleotide containing a transgene and homology sequences (e.g., homology arms) that are homologous to sequences at or near the endogenous genomic site for targeted integration, can be employed by molecules and machinery involved in cellular DNA repair processes, such as homologous recombination, as a repair template. In some aspects, a nucleic acid molecule having homology with sequences at or near one or more target sites in the endogenous DNA can be used to alter the structure of a target DNA, such as a target site at the gene, for targeted insertion of the transgene.

In some embodiments, the nucleic acid molecule alters the sequence of the target site, e.g., results in insertion or integration of the transgene between the homology arms, into the genome of the cell. In some aspects, targeted integration results in an in-frame integration of the coding portion of the transgene with one or more exons of the open reading frame of the gene, e.g., in-frame with the adjacent exon at the integration site. For example, in some cases, the in-frame integration results in a portion of the endogenous open reading frame and the recombinant protein to be expressed, in some cases separated by a multicistronic element, such as a 2A element. Thus, the modified gene can express a polypeptide encoded by the endogenous gene and the recombinant protein, which can be separated into 2 different polypeptides by virtue of the multicistronic element.

In some embodiments, the nucleic acid molecule includes sequences that correspond to or is homologous to a site on the target sequence that is cleaved, e.g., by one or more gene-editing agents capable of introducing a genetic disruption. In some embodiments, the nucleic acid molecule includes sequences that correspond to or is homologous to both, a first site on the target sequence that is cleaved in a first agent capable of introducing a genetic disruption, and a second site on the target sequence that is cleaved in a second agent capable of introducing a genetic disruption.

In some embodiments, a nucleic acid molecule comprises the following components: [5′ homology arm]-[transgene]-[3′ homology arm]. The homology arms provide for recombination into the chromosome, thus effectively inserting or integrating the transgene, e.g., that encodes a the recombinant receptor or portion thereof, into the genomic DNA at or near the cleavage site, such as target sites. In some embodiments, the homology arms flank the sequences at the target site of genetic disruption.

In some embodiments, the nucleic acid molecule contains at least one promoter that is operatively linked to control expression of the recombinant protein. In some examples, the nucleic acid molecule contains two, three, or more promoters operatively linked to control expression of the recombinant protein. In some embodiments, the nucleic acid molecule can contain regulatory sequences, such as transcription and translation initiation and termination codons, which are specific to the type of host (e.g., bacterium, fungus, plant, or animal) into which the nucleic acid molecule is to be introduced, as appropriate and taking into consideration whether the nucleic acid molecule is DNA- or RNA-based. In some embodiments, the nucleic acid molecule can contain regulatory/control elements, such as a promoter, an enhancer, an intron, a polyadenylation signal, a Kozak consensus sequence, internal ribosome entry sites (IRES), a 2A sequence, and splice acceptor or donor. In some embodiments, the nucleic acid molecule can contain a nonnative promoter operably linked to the transgene. In some embodiments, the promoter is selected from among an RNA pol I, pol II or pol III promoter. In some embodiments, the promoter is recognized by RNA polymerase II (e.g., a CMV, SV40 early region or adenovirus major late promoter). In another embodiment, the promoter is recognized by RNA polymerase III (e.g., a U6 or H1 promoter). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, the promoter is or comprises a constitutive promoter. Exemplary constitutive promoters include simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2):748-755). In some embodiments, the promoter is a tissue-specific promoter. In another embodiment, the promoter is a viral promoter. In another embodiment, the promoter is a non-viral promoter. In some embodiments, exemplary promoters can include, but are not limited to, human elongation factor 1 alpha (EF1α) promoter (SEQ ID NO: 247) or a modified form thereof or the MND promoter.

In another embodiment, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof. In some embodiments, the nucleic acid molecule does not include a regulatory element, e.g., promoter.

In some cases, the nucleic acid molecule contains a signal sequence that encodes a signal peptide. In some aspects, the signal sequence may encode a signal peptide derived from a native polypeptide. In other aspects, the signal sequence may encode a heterologous or non-native signal peptide, such as the exemplary signal peptide of the GMCSFR alpha chain set forth in SEQ ID NO: 40 and encoded by the nucleotide sequence set forth in SEQ ID NO: 41. In some cases, the nucleic acid molecule contains a signal sequence that encodes a signal peptide. Non-limiting exemplary signal peptides include, for example, the GMCSFR alpha chain signal peptide set forth in SEQ ID NO: 40 and encoded by the nucleotide sequence set forth in SEQ ID NO: 40, or the CD8 alpha signal peptide set forth in SEQ ID NO: 42.

In some embodiments, the nucleic acid molecule contains a nucleic acid sequence encoding one or more additional polypeptides, e.g., one or more marker(s) and/or one or more effector molecules. In some embodiments, the one or more marker(s) includes a transduction marker, a surrogate marker and/or a resistance marker or selection marker. Among additional nucleic acid sequences introduced, e.g., encoding for one or more additional polypeptide(s), include nucleic acid sequences that can improve the efficacy of therapy, such as by promoting viability and/or function of transferred cells; nucleic acid sequences to provide a genetic marker for selection and/or evaluation of the cells, such as to assess in vivo survival or localization; nucleic acid sequences to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also WO 1992008796 and WO 1994028143 describing the use of bifunctional selectable fusion genes derived from fusing a dominant positive selectable marker with a negative selectable marker, and U.S. Pat. No. 6,040,177.

In some embodiments, the marker is a transduction marker or a surrogate marker. A transduction marker or a surrogate marker can be used to detect cells that have been introduced with the nucleic acid molecule. In some embodiments, the transduction marker can indicate or confirm modification of a cell. In some embodiments, the surrogate marker is a protein that is made to be co-expressed on the cell surface with the recombinant protein, e.g. CAR. In particular embodiments, such a surrogate marker is a surface protein that has been modified to have little or no activity. In certain embodiments, the surrogate marker is encoded on the same polynucleotide that encodes the recombinant protein. In some embodiments, the transgene is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence. Extrinsic marker genes may in some cases be utilized in connection with engineered cell to permit detection or selection of cells and, in some cases, also to promote cell elimination and/or cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO: 43 or 44) or a prostate-specific membrane antigen (PSMA) or modified form thereof, such as a truncated PSMA (tPSMA). In some aspects, tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19. An exemplary polypeptide for a truncated EGFR (e.g. tEGFR) comprises the sequence of amino acids set forth in SEQ ID NO: 43 or 44 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 43 or 44.

In some embodiments, the marker is or comprises a detectable protein, such as a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, codon-optimized, stabilized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), 0-galactosidase, chloramphenicol acetyltransferase (CAT), 0-glucuronidase (GUS) or variants thereof. In some aspects, expression of the enzyme can be detected by addition of a substrate that can be detected upon the expression and functional activity of the enzyme.

In some embodiments, the marker is a resistance maker or selection marker. In some embodiments, the resistance maker or selection marker is or comprises a polypeptide that confers resistance to exogenous agents or drugs. In some embodiments, the resistance marker or selection marker is an antibiotic resistance gene. In some embodiments, the resistance marker or selection marker is an antibiotic resistance gene confers antibiotic resistance to a mammalian cell. In some embodiments, the resistance marker or selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

a. Transgene

In some embodiments, the nucleic acid molecule contains a transgene. In some embodiments, the transgene encodes a recombinant protein. In some embodiments, the recombinant protein is a recombinant receptor or a portion thereof, such as any recombinant receptor described herein, or one or more regions, domains, or chains of such recombinant receptor.

In some aspects, the transgene also contains non-coding, regulatory, or control sequences, e.g., sequences required for permitting, modulating and/or regulating expression of the encoded polypeptide or fragment thereof or sequences required to modify a polypeptide. In some embodiments, the transgene does not comprise an intron or lacks one or more introns as compared to a corresponding nucleic acid in the genome if the transgene is derived from a genomic sequence. In some embodiments, the transgene does not comprise an intron. In some of embodiments, all or a portion of the transgene is codon-optimized, e.g., for expression in human cells.

In some embodiments, the transgene also includes a signal sequence encoding a signal peptide, a regulatory or control elements, such as a promoter, and/or one or more multicistronic elements, e.g., a ribosome skip element or an internal ribosome entry site (IRES). In some embodiments, the signal sequence can be placed 5′ of the sequence of nucleotides encoding the recombinant protein.

In some of any embodiments, one or more regulatory/control elements, such as a promoter, an enhancer, an intron, a polyadenylation signal, a Kozak consensus sequence, internal ribosome entry sites (IRES), a 2A sequence, and splice acceptor or donor can be included in the vectors. In some embodiments, the promoter is selected from among an RNA pol I, pol II or pol III promoter. In some embodiments, the promoter is recognized by RNA polymerase II (such as a CMV, SV40 early region or adenovirus major late promoter). In another embodiment, the promoter is recognized by RNA polymerase III (such as a U6 or H1 promoter).

In certain embodiments, the promoter is a regulated promoter (such as inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1a promoter (EF1α), mouse phosphoglycerate kinase 1 promoter (PGK), and chicken 3-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2):748-755). In some embodiments, the promoter is a tissue-specific promoter. In another embodiment, the promoter is a viral promoter. In another embodiment, the promoter is a non-viral promoter. In some embodiments, exemplary promoters can include, but are not limited to, human elongation factor 1 alpha (EF1α) promoter or a modified form thereof (e.g., EF1α promoter with HTLV1 enhancer) or the MND promoter. In some embodiments, the promoter is an EF1α promoter (SEQ ID NO: 247) In some embodiments, the polynucleotide and/or vector does not include a regulatory element, e.g. promoter.

Any of the recombinant receptors and/or the additional polypeptide(s) described herein can be encoded by one or more polynucleotides containing one or more nucleic acid sequences encoding recombinant receptors, in any combinations, orientation or arrangements. For example, one, two, three or more polynucleotides can encode one, two, three or more different polypeptides, e.g., recombinant receptors or portions or components thereof, and/or one or more additional polypeptide(s), e.g., a marker and/or an effector molecule. In some embodiments, one polynucleotide contains a nucleic acid sequence encoding a recombinant receptor, e.g., CAR, or portion or components thereof, and a nucleic acid sequence encoding one or more additional polypeptide(s). In some embodiments, one vector or construct contains a nucleic acid sequence encoding a recombinant receptor, e.g., CAR, or portion or components thereof, and a separate vector or construct contains a nucleic acid sequence encoding one or more additional polypeptide(s). In some embodiments, the nucleic acid sequence encoding the recombinant receptor and the nucleic acid sequence encoding the one or more additional polypeptide(s) are operably linked to two different promoters. In some embodiments, the nucleic acid encoding the recombinant receptor is present upstream of the nucleic acid encoding the one or more additional polypeptide(s). In some embodiments, the nucleic acid encoding the recombinant receptor is present downstream of the nucleic acid encoding one or more additional polypeptide(s).

In certain cases, one polynucleotide contains nucleic acid sequences encode two or more different polypeptide chains, e.g., a recombinant receptor and one or more additional polypeptide(s), e.g., a marker and/or an effector molecule. In some embodiments, the nucleic acid sequences encoding two or more different polypeptide chains, e.g., a recombinant receptor and one or more additional polypeptide(s), are present in two separate polynucleotides. For example, two separate polynucleotides are provided, and each can be individually transferred or introduced into the cell for expression in the cell. In some embodiments, the nucleic acid sequences encoding the marker and the nucleic acid sequences encoding the recombinant receptor are present or inserted at different locations within the genome of the cell. In some embodiments, the nucleic acid sequences encoding the marker and the nucleic acid sequences encoding the recombinant receptor are operably linked to two different promoters.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, the nucleic acid sequences encoding the recombinant receptor and the nucleic acid sequences encoding the one or more additional polypeptide(s) are operably linked to the same promoter and are optionally separated by an internal ribosome entry site (IRES), or a nucleic acid encoding a self-cleaving peptide or a peptide that causes ribosome skipping, such as a 2A element. For example, an exemplary marker, and optionally a ribosome skipping sequence sequence, can be any as disclosed in PCT Pub. No. WO2014031687.

In some embodiments, transcription units can be engineered as a bicistronic unit containing an IRES, which allows coexpression of gene products (e.g. encoding the recombinant receptor and the additional polypeptide) by a message from a single promoter. Alternatively, in some cases, a single promoter may direct expression of an RNA that contains, in a single open reading frame (ORF), two or three genes (e.g. encoding the marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, e.g., de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 45), equine rhinitis A virus (E2A, e.g., SEQ ID NO: 46), Thosea asigna virus (T2A, e.g., SEQ ID NO: 47 or 48), and porcine teschovirus-1 (P2A, e.g., SEQ ID NO: 49 or 50) as described in U.S. Patent Pub. No. 20070116690.

i. Chimeric Antigen Receptors (CARs)

In some embodiments, the recombinant protein is a chimeric antigen receptor (CAR). In some embodiments, chimeric receptors, such as a chimeric antigen receptors, contain one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, WO2016/0046724, WO2016/014789, WO2016/090320, WO2016/094304, WO2017/025038, WO2017/173256, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, 8,479,118, and 9,765,342, and European patent application number EP2537416, and/or those described by Sadelain et al., Cancer Discov. 2013 April; 3(4): 388-398; Davila et al. (2013) pLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

Exemplary antigen receptors, e.g., CARs, also include any described in Marofi et al., Stem Cell Res Ther 12: 81 (2021); Townsend et al., J Exp Clin Cancer Res 37: 163 (2018); Ma et al., Int J Biol Sci 15(12): 2548-2560 (2019); Zhao and Cao, Front Immunol 10: 2250 (2019); Han et al., J Cancer 12(2): 326-334 (2021); Specht et al., Cancer Res 79: 4 Supplement, Abstract P2-09-13; Byers et al., Journal of Clinical Oncology 37, no. 15_suppl (2019); Panowski et al., Cancer Res 79 (13 Supplement) 2326 (2019); and Sauer et al., Blood 134 (Supplement 1): 1932 (2019); or can contain any of the antibodies or antigen-binding fragments described in U.S. Pat. Nos. 8,153,765; 8,603,477, 8,008,450; U.S. Pub. No. US20120189622 or US20100260748; and International PCT Publication Nos. WO2006099875, WO2009080829, WO2012092612, WO2014210064.

Further exemplary antigen receptors, e.g., CARs, such as anti-BCMA CARs, include the CARs of idecabtagene vicleucel, ABECMA©, BCMA02, JCARH125, JNJ-68284528 (LCAR-B38M; ciltacabtagene autoleucel; CARVYKTI™) (Janssen/Legend), P-BCMA-101 (Poseida), PBCAR269A (Poseida), P-BCMA-Allol (Poseida), Allo-715 (Pfizer/Allogene), CT053 (Carsgen), Descartes-08 (Cartesian), PHE885 (Novartis), ARI-002 (Hospital Clinic Barcelona, IDIBAPS), and CTX120 (CRISPR Therapeutics). In a particular embodiment, the CAR is the CAR of idecabtagene vicleucel cells. In a particular embodiment, the CAR is the CAR of ABECMA® cells (cells used in ABECMA® immunotherapy). In a particular embodiment, the CAR is the CAR of ciltacabtagene autoleucel cells. In a particular embodiment, the CAR is the CAR of CARVYKTI™ cells (cells used in CARVYKTI™ immunotherapy).

Exemplary antigen receptors, e.g., CARs, also include the CARs of FDA-approved products BREYANZI® (lisocabtagene maraleucel), TECARTUS™ (brexucabtagene autoleucel), KYMRIAH™ (tisagenlecleucel), and YESCARTA™ (axicabtagene ciloleucel), ABECMA® (idecabtagene vicleucel), and CARVYKTI™ (ciltacabtagene autoleucel). In some of any of the provided embodiments, the CAR is the CAR of BREYANZI® (lisocabtagene maraleucel), TECARTUS™ (brexucabtagene autoleucel), KYMRIAH™ (tisagenlecleucel), YESCARTA™ (axicabtagene ciloleucel), ABECMA® (idecabtagene vicleucel), or CARVYKTI™ (ciltacabtagene autoleucel). In some of any of the provided embodiments, the CAR is the CAR of BREYANZI® (lisocabtagene maraleucel, see Sehgal et al., 2020, Journal of Clinical Oncology 38:15_suppl, 8040; Teoh et al., 2019, Blood 134(Supplement_1):593; and Abramson et al., 2020, The Lancet 396(10254): 839-852). In some of any of the provided embodiments, the CAR is the CAR of TECARTUS™ (brexucabtagene autoleucel, see Mian and Hill, 2021, Expert Opin Biol Ther; 21(4):435-441; and Wang et al., 2021, Blood 138(Supplement 1):744). In some of any of the provided embodiments, the CAR is the CAR of KYMRIAH™ (tisagenlecleucel, see Bishop et al., 2022, N Engl J Med 386:629:639; Schuster et al., 2019, N Engl J Med 380:45-56; Halford et al., 2021, Ann Pharmacother 55(4):466-479; Mueller et al., 2021, Blood Adv. 5(23):4980-4991; and Fowler et al., 2022, Nature Medicine 28:325-332). In some of any of the provided embodiments, the CAR is the CAR of YESCARTA™ (axicabtagene ciloleucel, see Neelapu et al., 2017, N Engl J Med 377(26):2531-2544; Jacobson et al., 2021, The Lancet 23(1):P91-103; and Locke et al., 2022, N Engl J Med 386:640-654). In some of any of the provided embodiments, the CAR is the CAR of ABECMA® (idecabtagene vicleucel, see Raje et al., 2019, N Engl J Med 380:1726-1737; and Munshi et al., 2021, N Engl J Med 384:705-716). In some of any of the provided embodiments, the CAR is the CAR of CARVYKTI™ (ciltacabtagene autoleucel, see Berdeja et al., Lancet. 2021 Jul. 24; 398(10297):314-324; and Martin, Abstract #549 [Oral], presented at 2021 American Society of Hematology (ASH) Annual Meeting & Exposition)).

The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Ra), IL-13 receptor alpha 2 (IL-13Ra2), kinase insert domain receptor (kdr), kappa light chain, L1 cell adhesion molecule (L1-CAM), CE7 epitope of Li-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30. In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments, the antigen or antigen binding domain is CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723. Exemplary antibody or antibody fragments that bind to CD19 are also described in WO 2014/031687, US 2016/0152723, and WO 2016/033570.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 June 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 5, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 5
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26-- | H26--H35B | H30--H35B |
| (Kabat | | H32 . . . 34 | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of antibodies are described using various numbering schemes, although it is understood that an antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the antibodies included in the CARs are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In some embodiments, the antigen-binding domain in the CARs is or comprises an antibody fragment comprising a variable heavy chain (V_{H}) and a variable light chain (V_{L}) region. In particular embodiments, the antibodies are single-chain antibody fragments comprising a heavy chain variable (V_{H}) region and/or a light chain variable (V_{L}) region, such as scFvs.

In some embodiments, the scFv is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDRH1 and H2 set forth in SEQ ID NO: 51 and 52, respectively, and CDRH3 set forth in SEQ ID NO: 53 or 54 and CDRL1 set forth in SEQ ID NO: 55 and CDR L2 set forth in SEQ ID NO: 56 or 57 and CDR L3 set forth in SEQ ID NO: 58 or 59. In some embodiments, the FMC63 antibody comprises the heavy chain variable region (V_{H}) comprising the amino acid sequence of SEQ ID NO: 60 and the light chain variable region (V_{L}) comprising the amino acid sequence of SEQ ID NO: 61.

In some embodiments, the scFv comprises a variable light chain containing the CDRL1 sequence of SEQ ID NO: 55, a CDRL2 sequence of SEQ ID NO: 56, and a CDRL3 sequence of SEQ ID NO: 58 and/or a variable heavy chain containing a CDRH1 sequence of SEQ ID NO: 51, a CDRH2 sequence of SEQ ID NO: 52, and a CDRH3 sequence of SEQ ID NO: 53. In some embodiments, the scFv comprises a variable heavy chain region set forth in SEQ ID NO: 60 and a variable light chain region set forth in SEQ ID NO: 61. In some embodiments, the variable heavy and variable light chains are connected by a linker. In some embodiments, the linker is set forth in SEQ ID NO: 62. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO: 63 or a sequence that exhibits at least 85%, 86%, 87%, 88%, 89%, 90%, 91%, 92%, 93%, 94%, 95%, 96%, 97%, 98%, or 99% sequence identity to SEQ ID NO: 63. In some embodiments, the scFv comprises the sequence of amino acids set forth in SEQ ID NO: 64 or a sequence that exhibits at least 85%, 86%, 87%, 88%, 89%, 90%, 91%, 92%, 93%, 94%, 95%, 96%, 97%, 98%, or 99% sequence identity to SEQ ID NO: 64. In some embodiments, the scFv is that of BREYANZI® (lisocabtagene maraleucel). In some embodiments, the CAR is that of BREYANZI® (lisocabtagene maraleucel).

In some embodiments the scFv is derived from SJ25C1. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDRH1, H2 and H3 set forth in SEQ ID NO: 65-67, respectively, and CDRL1, L2 and L3 sequences set forth in SEQ ID NO: 68-70, respectively. In some embodiments, the SJ25C1 antibody comprises the heavy chain variable region (V_{H}) comprising the amino acid sequence of SEQ ID NO: 71 and the light chain variable region (V_{L}) comprising the amino acid sequence of SEQ ID NO: 72.

In some embodiments, the scFv comprises a variable light chain containing the CDRL1 sequence of SEQ ID NO: 73, a CDRL2 sequence of SEQ ID NO: 74, and a CDRL3 sequence of SEQ ID NO: 75 and/or a variable heavy chain containing a CDRH1 sequence of SEQ ID NO: 76, a CDRH2 sequence of SEQ ID NO: 77, and a CDRH3 sequence of SEQ ID NO: 78. In some embodiments, the scFv comprises a variable heavy chain region set forth in SEQ ID NO: 71 and a variable light chain region set forth in SEQ ID NO: 72. In some embodiments, the variable heavy and variable light chain are connected by a linker. In some embodiments, the linker is set forth in SEQ ID NO: 79. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO: 80 or a sequence that exhibits at least 85%, 86%, 87%, 88%, 89%, 90%, 91%, 92%, 93%, 94%, 95%, 96%, 97%, 98%, or 99% sequence identity to SEQ ID NO: 80.

In some embodiments, the antigen or antigen binding domain is BCMA. In some embodiments, the scFv contains a VH and a VL derived from an antibody or an antibody fragment specific to BCMA. In some embodiments, the antibody or antibody fragment that binds BCMA is or contains a VH and a VL from an antibody or antibody fragment set forth in International Patent Applications, Publication Number WO 2016/090327 and WO 2016/090320.

In some embodiments, the antibody or antibody fragment that binds BCMA can be any anti-BCMA antibody described or derived from any anti-BCMA antibody described. See, e.g., Carpenter et al., Clin Cancer Res., 2013, 19(8):2048-2060; U.S. Pat. Nos. 9,034,324 9,765,342; U.S. Patent publication No. US2016/0046724, US20170183418; and International published PCT App. No. WO 2016090320, WO2016090327, WO2016094304, WO2016014565, WO106014789, WO2010104949, WO2017/025038, or WO2017173256. Any of such anti-BCMA antibodies or antigen-binding fragments can be used in the anti-BCMA CAR. In some embodiments, the anti-BCMA CAR contains an antigen-binding domain that is an scFv containing a variable heavy (V_{H}) and/or a variable light (V_{L}) region. In some embodiments, the scFv containing a variable heavy (V_{H}) and/or a variable light (V_{L}) region is derived from an antibody described in WO 2016090320 or WO2016090327. In some embodiments, the antigen or antigen binding domain is GPRC5D. In some embodiments, the scFv contains a VH and a VL derived from an antibody or an antibody fragment specific to GPRC5D. In some embodiments, the antibody or antibody fragment that binds GPRC5D is or contains a VH and a VL from an antibody or antibody fragment set forth in International Patent Applications, Publication Number WO 2016/090329, WO 2016/090312, and WO 2020/092854.

In some embodiments, the antibody or antibody fragment that binds BCMA includes a V_{H} and a V_{L} region, wherein the V_{H} region includes a CDR-H1 set forth in SEQ ID NO: 113, a CDR-H2 set forth in SEQ ID NO: 114, and a CDR-H3 set forth in SEQ ID NO: 115, and the V_{L} region includes a CDR-L1 set forth in SEQ ID NO: 116, a CDR-L2 set forth in SEQ ID NO: 117, and a CDR-H3 set forth in SEQ ID NO: 118. In some embodiments, the antibody or antibody fragment that binds BCMA includes a V_{H} region that has the sequence of amino acids set forth in SEQ ID NO: 119 or a sequence of amino acids that exhibits at least 90%, at least 91%, at least 92%, at least 93%, at least 94%, at least 95%, at least 96%, at least 97%, at least 98%, or at least 99% to SEQ ID NO: 119, and a V_{L} region that has the sequence of amino acids set forth in SEQ ID NO: 120 or a sequence of amino acids that exhibits at least 90%, at least 91%, at least 92%, at least 93%, at least 94%, at least 95%, at least 96%, at least 97%, at least 98%, or at least 99% to SEQ ID NO: 120. In some embodiments, the antibody or antibody fragment that binds BCMA includes a V_{H} region that has the sequence of amino acids set forth in SEQ ID NO: 119 and a V_{L} region that has the sequence of amino acids set forth in SEQ ID NO: 120. In some embodiments, the antibody or antibody fragment that binds BCMA is an scFv that has the sequence of amino acids set forth in SEQ ID NO: 121 or a sequence of amino acids that exhibits at least 90%, at least 91%, at least 92%, at least 93%, at least 94%, at least 95%, at least 96%, at least 97%, at least 98%, or at least 99% to SEQ ID NO: 121. In some embodiments, the antibody or antibody fragment that binds BCMA is an scFv as set forth in SEQ ID NO: 121. In some embodiments, the scFv is that of ABECMA® (idecabtagene vicleucel). In some embodiments, the CAR has the sequence of amino acids set forth in SEQ ID NO: 122 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO: 122. In some embodiments, the CAR is that of ABECMA® (idecabtagene vicleucel).

In some embodiments, the antibody or antibody fragment that binds BCMA includes a V_{H} and a V_{L} region, wherein the V_{H} region includes a CDR-H1 set forth in SEQ ID NO: 123, a CDR-H2 set forth in SEQ ID NO: 124, and a CDR-H3 set forth in SEQ ID NO: 125, and the V_{L} region includes a CDR-L1 set forth in SEQ ID NO: 126, a CDR-L2 set forth in SEQ ID NO: 127, and a CDR-H3 set forth in SEQ ID NO: 128. In some embodiments, the antibody or antibody fragment that binds BCMA includes a V_{H} region that has the sequence of amino acids set forth in SEQ ID NO: 129 or a sequence of amino acids that exhibits at least 90%, at least 91%, at least 92%, at least 93%, at least 94%, at least 95%, at least 96%, at least 97%, at least 98%, or at least 99% to SEQ ID NO: 129, and a V_{L} region that has the sequence of amino acids set forth in SEQ ID NO: 130 or a sequence of amino acids that exhibits at least 90%, at least 91%, at least 92%, at least 93%, at least 94%, at least 95%, at least 96%, at least 97%, at least 98%, or at least 99% to SEQ ID NO: 130. In some embodiments, the antibody or antibody fragment that binds BCMA includes a V_{H} region that has the sequence of amino acids set forth in SEQ ID NO: 129 and a V_{L} region that has the sequence of amino acids set forth in SEQ ID NO: 130. In some embodiments, the antibody or antibody fragment that binds BCMA is an scFv that has the sequence of amino acids set forth in SEQ ID NO: 131 or a sequence of amino acids that exhibits at least 90%, at least 91%, at least 92%, at least 93%, at least 94%, at least 95%, at least 96%, at least 97%, at least 98%, or at least 99% to SEQ ID NO: 131. In some embodiments, the antibody or antibody fragment that binds BCMA is an scFv as set forth in SEQ ID NO: 131. In some embodiments, the scFv is that of orvacabtagene autoleucel. In some embodiments, the CAR has the sequence of amino acids set forth in SEQ ID NO: 132 or a sequence of amino acids that exhibits at least 90%, at least 91%, at least 92%, at least 93%, at least 94%, at least 95%, at least 96%, at least 97%, at least 98%, or at least 99% sequence identity to SEQ ID NO: 132. In some embodiments, the CAR is that of orvacabtagene autoleucel.

In some embodiments, the antigen is CD20. In some embodiments, the scFv contains a VH and a VL derived from an antibody or an antibody fragment specific to CD20. In some embodiments, the antibody or antibody fragment that binds CD20 is an antibody that is or is derived from Rituximab, such as is Rituximab scFv.

In some embodiments, the antigen is CD22. In some embodiments, the scFv contains a VH and a VL derived from an antibody or an antibody fragment specific to CD22. In some embodiments, the antibody or antibody fragment that binds CD22 is an antibody that is or is derived from m971, such as is m971 scFv.

In some embodiments, the antigen is ROR1. In some embodiments, the scFv contains a V_{H} and a V_{L} derived from an antibody or an antibody fragment specific to ROR1. In some embodiments, the antibody or antibody fragment that binds ROR1 is or contains a V_{H} and a V_{L} from an antibody or antibody fragment set forth in International Patent Applications, Publication Number WO 2014/031687, WO 2016/115559 and WO 2020/160050, the contents of each of which are incorporated by reference in their entirety.

In some embodiments, the antigen is FcRL5. In some embodiments, the scFv contains a V_{H} and a V_{L} derived from an antibody or an antibody fragment specific to FcRL5. In some embodiments, the antibody or antibody fragment that binds FcRL5 is or contains a V_{H} and a V_{L} from an antibody or antibody fragment set forth in International Patent Applications, Publication Number WO 2016/090337 and WO 2017/096120, the contents of each of which are incorporated by reference in their entirety.

In some embodiments, the antigen is mesothelin. In some embodiments, the scFv contains a V_{H} and a V_{L} derived from an antibody or an antibody fragment specific to mesothelin. In some embodiments, the antibody or antibody fragment that binds mesothelin is or contains a V_{H} and a V_{L} from an antibody or antibody fragment set forth in US2018/0230429, the contents of which are incorporated by reference in their entirety.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv.

In some embodiments, the antibody portion of the recombinant receptor, e.g., CAR, further includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635.

In some embodiments, the constant region or portion is of a human IgG, such as IgG4 or IgG1. In some embodiments, the spacer has the sequence ESKYGPPCPPCP (set forth in SEQ ID NO: 81), and is encoded by the sequence set forth in SEQ ID NO: 82. In some embodiments, the spacer has the sequence set forth in SEQ ID NO: 83. In some embodiments, the spacer has the sequence set forth in SEQ ID NO: 84. In some embodiments, the constant region or portion is of IgD. In some embodiments, the spacer has the sequence set forth in SEQ ID NO: 85. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NO: 81, 83, 84 or 85. In some embodiments, the spacer has the sequence set forth in SEQ ID NO: 86-94. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NO: 86-94.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM. For example, in some aspects, the antigen recognition domain (e.g. extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g. scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD3 transmembrane domains. In some embodiments, the receptor, e.g., CAR, further includes a portion of one or more additional molecules such as Fc receptor 7, CD8, CD4, CD25, or CD16. For example, in some aspects, the CAR or other chimeric receptor includes a chimeric molecule between CD3-zeta (CD3-ζ) or Fc receptor 7 and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

An exemplary polypeptide for a truncated EGFR (e.g. tEGFR) comprises the sequence of amino acids set forth in SEQ ID NO: 43 or 44 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 43 or 44. An exemplary T2A linker sequence comprises the sequence of amino acids set forth in SEQ ID NO: 47 or 48 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 47 or 48.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

For example, in some embodiments, the CAR contains an antibody, e.g., an antibody fragment, such as an scFv, specific to an antigen including any as described, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, such as an scFv, specific to an antigen including any as described, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P01747.1) or variant thereof, such as a transmembrane domain that comprises the sequence of amino acids set forth in SEQ ID NO: 95 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 95; in some embodiments, the transmembrane-domain containing portion of the recombinant receptor comprises the sequence of amino acids set forth in SEQ ID NO: 96 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 97 or 98 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 97 or 98. In some embodiments, the intracellular domain comprises an intracellular costimulatory signaling domain of 4-1BB (e.g. (Accession No. Q07011.1) or functional variant or portion thereof, such as the sequence of amino acids set forth in SEQ ID NO: 99 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 99.

In some embodiments, the intracellular signaling domain of the recombinant receptor, e.g. the CAR, comprises a human CD3 zeta stimulatory signaling domain or functional variant thereof, such as an 112 AA cytoplasmic domain of isoform 3 of human CD3((Accession No.: P20963.2) or a CD3 zeta signaling domain as described in U.S. Pat. No. 7,446,190 or U.S. Pat. No. 8,911,993. For example, in some embodiments, the intracellular signaling domain comprises the sequence of amino acids as set forth in SEQ ID NO: 100, 101 or 102 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 100, 101 or 102.

In some aspects, the spacer contains only a hinge region of an IgG, such as only a hinge of IgG4 or IgG1, such as the hinge only spacer set forth in SEQ ID NO: 81. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains, such as set forth in SEQ ID NO: 84. In some embodiments, the spacer is an Ig hinge, e.g., an IgG4 hinge, linked to a CH3 domain only, such as set forth in SEQ ID NO: 83. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in 43 or 44) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered to express the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a resistance marker or selection marker. In some embodiments, the resistance marker or selection marker is or comprises a polypeptide that confers resistance to exogenous agents or drugs. In some embodiments, the resistance marker or selection marker is an antibiotic resistance gene. In some embodiments, the resistance marker or selection marker is an antibiotic resistance gene confers antibiotic resistance to a mammalian cell. In some embodiments, the resistance marker or selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 47 or 48, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 47 or 48.

In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NO: 43 or 44, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 43 or 44. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used herein include 2A sequences from the foot-and-mouth disease virus (F2A, e.g., SEQ ID NO: 45), equine rhinitis A virus (E2A, e.g., SEQ ID NO: 46), Thosea asigna virus (T2A, e.g., SEQ ID NO: 47 or 48), and porcine teschovirus-1 (P2A, e.g., SEQ ID NO: 49 or 50) as described in U.S. Patent Publication No. 20070116690.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

ii. Chimeric Auto-Antibody Receptor (CAAR)

In some embodiments, the recombinant protein is a chimeric autoantibody receptor (CAAR). In some embodiments, the CAAR binds, e.g., specifically binds, or recognizes, an autoantibody. In some embodiments, a cell expressing the CAAR, such as a T cell engineered to express a CAAR, can be used to bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent Application Pub. No. US 2017/0051035.

In some embodiments, the CAAR comprises an autoantibody binding domain, a transmembrane domain, and one or more intracellular signaling region or domain (also interchangeably called a cytoplasmic signaling domain or region). In some embodiments, the intracellular signaling region comprises an intracellular signaling domain. In some embodiments, the intracellular signaling domain is or comprises a primary signaling domain, a signaling domain that is capable of stimulating and/or inducing a primary activation signal in a T cell, a signaling domain of a T cell receptor (TCR) component (e.g. an intracellular signaling domain or region of a CD3-zeta (CD3ζ) chain or a functional variant or signaling portion thereof), and/or a signaling domain comprising an immunoreceptor tyrosine-based activation motif (ITAM).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g. an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

iii. T Cell Receptors (TCRs)

In some embodiments, the recombinant protein is a T cell receptor (TCR). In some embodiments, the TCR recognizes an peptide epitope or T cell epitope of a target polypeptide, such as an antigen of a tumor, viral or autoimmune protein. In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the 3-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or CP, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling device or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or 7 and 6 chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ T cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007).

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric ap TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See e.g., Soo Hoo, W. F. et al. PNAS (USA) 89, 4759 (1992); Wülfing, C. and Plütckthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an a chain variable region sequence fused to the N terminus of an a chain extracellular constant domain sequence, and a second segment constituted by a p chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an a chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from 10 to 45 amino acids or from about 10 to about 45 amino acids, such as 10 to 30 amino acids or 26 to 41 amino acids residues, for example 29, 30, 31 or 32 amino acids. In some embodiments, the linker has the formula -PGGG-(SGGGG)5-P- wherein P is proline, G is glycine and S is serine (SEQ ID NO: 38). In some embodiments, the linker has the sequence GSADDAKKDAAKKDGKS (SEQ ID NO: 39).

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10⁻⁵ and 10-12 M and all individual values and ranges therein. In some embodiments, the target antigen is an MHIC-peptide complex or ligand.

In some embodiments, nucleic acid or nucleic acids encoding a TCR, such as α and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as XG10, XGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector.

b. Homology Arms

In some embodiments, the nucleic acid molecule contains one or more homology sequences (also called “homology arms”) on the 5′ and 3′ ends, linked to or surrounding the transgene. The homology arms allow the DNA repair mechanisms, e.g., homologous recombination machinery, to recognize the homology and use the nucleic acid molecule as a template for repair, and the nucleic acid sequence between the homology arms are copied into the DNA being repaired, effectively inserting or integrating the transgene into the target site of integration in the genome between the location of the homology.

In some aspects, upon integration of the transgene, the entire recombinant protein is encoded by the transgene, and the entire coding sequence or a portion of the coding sequences of the gene is deleted. In some embodiments, the transgene comprises a sequence of nucleotides that is in-frame with one or more exons of the open reading frame of the gene comprised in the one or more homology arms. In some aspects, the entire recombinant protein is encoded by the transgene, and only a portion of the gene is deleted, and the remaining portion of the gene is expressed.

In some embodiments, the homology arm sequences include sequences that are homologous to the genomic sequences surrounding the genetic disruption, e.g., a target site within the gene. In some embodiments, the nucleic acid molecule comprises the following components: [5′ homology arm]-[transgene]-[3′ homology arm]. In some embodiments, the 5′ homology arm sequences include contiguous sequences that are homologous to sequences located near the genetic disruption on the 5′ side. In some embodiments, the 3′ homology arm sequences include contiguous sequences that are homologous to sequences located near the genetic disruption on the 3′ side. In some aspects, the target site is determined by targeting of the one or more gene-editing agents capable of introducing a genetic disruption, e.g., Cas, e.g., Cas9 or Cas12, and gRNA targeting a specific site within the gene.

In some aspects, the transgene within the nucleic acid molecule can be used to guide the location of target sites and/or homology arms. In some aspects, the target site of genetic disruption can be used as a guide to design nucleic acid molecules and/or homology arms used for HDR. In some embodiments, the genetic disruption can be targeted near a desired site of targeted integration of the transgene. In some aspects, the homology arms are designed to target integration within an exon of the open reading frame of the gene, and the homology arm sequences are determined based on the desired location of integration surrounding the genetic disruption, including exon and intron sequences surrounding the genetic disruption. In some embodiments, the location of the target site, relative location of the one or more homology arms, and the transgene for insertion can be designed depending on the requirement for efficient targeting and the length of the nucleic acid molecule or vector that can be used. In some aspects, the homology arms are designed to target integration within an intron of the open reading frame of the gene. In some aspects, the homology arms are designed to target integration within an exon of the open reading frame of the gene.

In some aspects, the target integration site (site for targeted integration) within the T cell stimulation-associated locus is located within an open reading frame at the endogenous T cell stimulation-associated locus. In some embodiments, the target integration site is at or near any of the target sites described herein. In some aspects, the target location for integration is at or around the target site for genetic disruption, e.g., within less than 500, 450, 400, 350, 300, 250, 200, 150, 100 or 50 bp of the target site for genetic disruption.

In some embodiments, the 5′ homology arm sequences include contiguous sequences of approximately 10, 20, 30, 40, 50, 100, 200, 300, 400, 500, 600, 700, 800, 900, 1000, 1500, 2000, 3000, 4000, or 5000 base pairs 5′ of the target site for genetic disruption, starting near the target site at the gene. In some embodiments, the 3′ homology arm sequences include contiguous sequences of approximately 10, 20, 30, 40, 50, 100, 200, 300, 400, 500, 600, 700, 800, 900, 1000, 1500, 2000, 3000, 4000, or 5000 base pairs 3′ ofthe target site for genetic disruption, starting near the target site at the gene. Thus, upon integration via HDR, the transgene is targeted for integration at or near the target site for genetic disruption, e.g., a target site within an exon or intron of the gene.

In some aspects, the homology arms contain sequences that are homologous to a portion of an open reading frame sequence at the gene. In some aspects, the homology arm sequences contain sequences homologous to contiguous portion of an open reading frame sequence, including exons and introns, at the gene. In some aspects, the homology arm contains sequences that are identical to a contiguous portion of an open reading frame sequence, including exons and introns, at the gene.

In some embodiments, the nucleic acid molecule contains homology arms for targeting integration of the transgene at the gene. In some embodiments, the genetic disruption is introduced using any of the gene-editing agents for genetic disruption, e.g., targeted nucleases and/or gRNAs described herein. In some embodiments, the nucleic acid molecule comprises about 500 to 1000, e.g., 500 to 900 or 600 to 700, base pairs of homology on either side of the genetic disruption introduced by the targeted nucleases and/or gRNAs. In some embodiments, the nucleic acid molecule comprises about 500, 600, 700, 800, 900 or 1000 base pairs of 5′ homology arm sequences, which is homologous to 500, 600, 700, 800, 900 or 1000 base pairs of sequences 5′ of the genetic disruption at the gene, the transgene, and about 500, 600, 700, 800, 900 or 1000 base pairs of 3′ homology arm sequences, which is homologous to 500, 600, 700, 800, 900 or 1000 base pairs of sequences 3′ of the genetic disruption at the gene.

In some aspects, the boundary between the transgene and the one or more homology arm sequences, is designed such that upon HDR and targeted integration of the transgene, the sequences within the transgene that encode one or more polypeptides, e.g., chain(s), domain(s) or region(s) of a recombinant receptor, is integrated in-frame with one or more exons of the open reading frame sequence at the gene, and/or generates an in-frame fusion of the transgene that encode a polypeptide and one or more exons of the open reading frame sequence at the gene. In some embodiments, all or a portion of the gene product of the gene is encoded by the nucleic acid sequences of the endogenous open reading frame, and a polypeptide of the recombinant receptor or a portion thereof is encoded by the integrated transgene, optionally, separated by a multicistronic element, such as a 2A element.

In some embodiments, the one or more homology arm sequences include sequences that are homologous, substantially identical or identical to sequences that surround or flank the target site that are within an open reading frame sequence at the gene. In some aspects, the one or more homology arm sequences contain introns and exons of a partial sequence of an open reading frame at the gene. In some aspects, the boundary of the 5′ homology arm sequence and the transgene is such that, in a case of a transgene that does not contain a heterologous promoter, the coding portion of the transgene is fused in-frame with an upstream exon or a portion thereof, e.g., exon 1, 2, 3, 4 or 5, depending on the location of targeted integration, of the open reading frame of the gene.

In some aspects, the boundary of the 5′ homology arm sequence and the transgene is such that, the upstream exons or a portion thereof, e.g., exons 1, 2, 3, 4, or 5, of the open reading frame of the gene, is fused in-frame with the coding portions of the transgene. Thus, upon targeted integration, transcription and translation, the encoded recombinant receptor that is a contiguous polypeptide is produced, from a fusion DNA sequence of an open reading frame sequence of the gene and the transgene. In some aspects, the upstream exons or a portion thereof encode all or a portion of the gene product of the gene. In some aspects, upon targeted integration, a multicistronic element, e.g., a 2A element or an internal ribosome entry site (IRES) separates the open reading frame sequence of the gene and the transgene. In some aspects, when expressed and translated from the modified gene, the polypeptide is cleaved to generate all or a portion of the polypeptide encoded by the gene and a recombinant receptor.

In some embodiments, exemplary 5′ homology arm for targeting integration at the endogenous TRAC locus comprises the sequence set forth in SEQ ID NO: 245 or 248, or a sequence that exhibits at least 85%, 86%, 87%, 88%, 89%, 90%, 91%, 92%, 93%, 94%, 95%, 96%, 97%, 98%, 99% or more sequence identity to SEQ ID NO: 245 or 248 or a partial sequence thereof.

In some embodiments, exemplary 3′ homology arm for targeting integration at the endogenous TRAC locus comprises the sequence set forth in SEQ ID NO: 246 or 249, or a sequence that exhibits at least 85%, 86%, 87%, 88%, 89%, 90%, 91%, 92%, 93%, 94%, 95%, 96%, 97%, 98%, 99% or more sequence identity to SEQ ID NO: 246 or 249 or a partial sequence thereof.

In some aspects, the target site can determine the relative location and sequences of the homology arms. The homology arm can typically extend at least as far as the region in which end resection by the DNA repair mechanism can occur after the genetic disruption, e.g., DSB, is introduced, e.g., in order to allow the resected single stranded overhang to find a complementary region within the template polynucleotide. The overall length could be limited by parameters such as plasmid size, viral packaging limits or construct size limit.

In some embodiments, the homology arm comprises about 500 to 1000, e.g., 600 to 900 or 700 to 800, base pairs of homology on either side of the target site at the gene. In some embodiments, the homology arm comprises about at least or less than or about 200, 300, 400, 500, 600, 700, 800, 900 or 1000 base pairs of homology 5′ of the target site, 3′ of the target site, or both 5′ and 3′ of the target site at the gene.

In some embodiments, the homology arm comprises at or about 10, 20, 30, 40, 50, 100, 200, 300, 400, 500, 600, 700, 800, 900, 1000, 1500, 2000, 3000, 4000, or 5000 base pairs of homology 3′ of the target site at the gene. In some embodiments, the homology arm comprises at or about 100 to 500, 200 to 400 or 250 to 350, base pairs of homology 3′ of the transgene and/or target site at the gene. In some embodiments, the homology arm comprises less than about 100, 90, 80, 70, 60, 50, 40, 30, 20, 15, or 10 base pairs of homology 5′ of the target site at the gene.

In some embodiments, the homology arm comprises at or about 10, 20, 30, 40, 50, 100, 200, 300, 400, 500, 600, 700, 800, 900, 1000, 1500, 2000, 3000, 4000, or 5000 base pairs of homology 5′ of the target site at the gene. In some embodiments, the homology arm comprises at or about 100 to 500, 200 to 400 or 250 to 350, base pairs of homology 5′ of the transgene and/or target site at the gene. In some embodiments, the homology arm comprises less than about 100, 90, 80, 70, 60, 50, 40, 30, 20, 15, or 10 base pairs of homology 3′ of the target site at the gene.

In some embodiments, the 3′ end of the 5′ homology arm is the position next to the 5′ end of the transgene. In some embodiments, the 5′ homology arm can extend at least at or about 10, 20, 30, 40, 50, 100, 200, 300, 400, 500, 600, 700, 800, 900, 1000, 1500, 2000, 3000, 4000, or 5000 nucleotides 5′ from the 5′ end of the transgene.

In some embodiments, the 5′ end of the 3′ homology arm is the position next to the 3′ end of the transgene. In some embodiments, the 3′ homology arm can extend at least at or about 10, 20, 30, 40, 50, 100, 200, 300, 400, 500, 600, 700, 800, 900, 1000, 1500, 2000, 3000, 4000, or 5000 nucleotides 3′ from the 3′ end of the transgene.

In some embodiments, for targeted insertion, the homology arms, e.g., the 5′ and 3′ the homology arms, may each comprise about 1000 base pairs (bp) of sequence flanking the most distal target sites (e.g., 1000 bp of sequence on either side of the mutation).

Exemplary homology arm lengths include at least at or about 50, 100, 200, 250, 300, 400, 500, 600, 700, 750, 800, 900, 1000, 2000, 3000, 4000, or 5000 nucleotides. In some embodiments, the homology arm length is at or about 50-100, 100-250, 250-500, 500-750, 750-1000, 1000-2000, 2000-3000, 3000-4000, or 4000-5000 nucleotides. Exemplary homology arm lengths include less than at or about or is or is about 50, 100, 200, 250, 300, 400, 500, 600, 700, 750, 800, 900, 1000, 2000, 3000, 4000, or 5000 nucleotides. In some embodiments, the homology arm length is at or about 50-100, 100-250, 250-500, 500-750, 750-1000, 1000-2000, 2000-3000, 3000-4000, or 4000-5000 nucleotides. Exemplary homology arm lengths include from at or about 100 to at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or 750 to at or about 1000 nucleotides.

In some of any such embodiments, the transgene is integrated by a nucleic acid molecule introduced into the immune cells. In some of any embodiments, the nucleic acid molecule comprises the structure [5′ homology arm]-[transgene]-[3′ homology arm]. In certain embodiments, the 5′ homology arm and the 3′ homology arm comprises nucleic acid sequences homologous to nucleic acid sequences surrounding the at least at or about one target site. In some embodiments, the 5′ homology arm comprises nucleic acid sequences that are homologous to nucleic acid sequences 5′ of the target site. In some of any embodiments, the 3′ homology arm comprises nucleic acid sequences that are homologous to nucleic acid sequences 3′ of the target site. In certain embodiments, the 5′ homology arm and the 3′ homology arm independently are at least at or about or at least at or about 10, 20, 30, 40, 50, 100, 200, 300, 400, 500, 600, 700, 800, 900, 1000, 1500, or 2000 nucleotides, or less than at or about 10, 20, 30, 40, 50, 100, 200, 300, 400, 500, 600, 700, 800, 900, 1000, 1500, or 2000 nucleotides. In some embodiments, the 5′ homology arm and the 3′ homology arm independently are between at or about 50 and at or about 100, 100 and at or about 250, 250 and at or about 500, 500 and at or about 750, 750 and at or about 1000, 1000 and at or about 2000 nucleotides. In some of any such embodiments, the 5′ homology arm and the 3′ homology arm independently are between at or about 50 and at or about 100 nucleotides in length, at or about 100 and at or about 250 nucleotides in length, at or about 250 and at or about 500 nucleotides in length, at or about 500 and at or about 750 nucleotides in length, at or about 750 and at or about 1000 nucleotides in length, or at or about 1000 and at or about 2000 nucleotides in length.

In some of any embodiments, the 5′ homology arm and the 3′ homology arm independently are from at or about 100 to at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or from at or about 750 to at or about 1000 nucleotides. In some of any embodiments, the 5′ homology arm and the 3′ homology arm independently are from at or about 100 to at or about at or about 1000 nucleotides, from at or about 100 to at or about 750 nucleotides, from at or about 100 to at or about 600 nucleotides, from at or about 100 to at or about 400 nucleotides, from at or about 100 to at or about 300 nucleotides, from at or about 100 to at or about 200 nucleotides, from at or about 200 to at or about 1000 nucleotides, from at or about 200 to at or about 750 nucleotides, from at or about 200 to at or about 600 nucleotides, from at or about 200 to at or about 400 nucleotides, from at or about 200 to at or about 300 nucleotides, from at or about 300 to at or about 1000 nucleotides, from at or about 300 to at or about 750 nucleotides, from at or about 300 to at or about 600 nucleotides, from at or about 300 to at or about 400 nucleotides, from at or about 400 to at or about 1000 nucleotides, from at or about 400 to at or about 750 nucleotides, from at or about 400 to at or about 600 nucleotides, from at or about 600 to at or about 1000 nucleotides, from at or about 600 to at or about 750 nucleotides or from at or about 750 to at or about 1000 nucleotides in length. In some embodiments, the 5′ homology arm and the 3′ homology arm independently are at or about 200, 300, 400, 500, 600, 700 or 800 nucleotides in length, or any value between any of the foregoing. In some embodiments, the 5′ homology arm and the 3′ homology arm independently are greater than at or about 300 nucleotides in length, optionally wherein the 5′ homology arm and the 3′ homology arm independently are at or about 400, 500 or 600 nucleotides in length or any value between any of the foregoing. In some embodiments, the 5′ homology arm and the 3′ homology arm independently are greater than at or about 300 nucleotides in length.

In some embodiments, one or more of the homology arms contain a sequence of nucleotides are homologous to sequences that encode a gene product of the gene. In some embodiments, one or more homology arms are connected or linked in frame with the transgene.

In some embodiments, alternative HDR is employed. In some embodiments, alternative HDR proceeds more efficiently when the nucleic acid molecule has extended homology 5′ to the target site (i.e., in the 5′ direction of the target site strand). Accordingly, in some embodiments, the nucleic acid molecule has a longer homology arm and a shorter homology arm, wherein the longer homology arm can anneal 5′ of the target site. In some embodiments, the arm that can anneal 5′ to the target site is at least 25, 50, 75, 100, 125, 150, 175, or 200, 300, 400, 500, 600, 700, 800, 900, 1000, 1500, 2000, 3000, 4000, or 5000 nucleotides from the target site or the 5′ or 3′ end of the transgene. In some embodiments, the arm that can anneal 5′ to the target site is at least 10%, 20%, 30%, 40%, or 50% longer than the arm that can anneal 3′ to the target site. In some embodiments, the arm that can anneal 5′ to the target site is at least 2×, 3×, 4×, or 5× longer than the arm that can anneal 3′ to the target site. Depending on whether a ssDNA template can anneal to the intact strand or the targeted strand, the homology arm that anneals 5′ to the target site may be at the 5′ end of the ssDNA template or the 3′ end of the ssDNA template, respectively.

Similarly, in some embodiments, the nucleic acid molecule has a 5′ homology arm, a transgene, and a 3′ homology arm, such that the nucleic acid molecule contains extended homology to the 5′ of the target site. For example, the 5′ homology arm and the 3′ homology arm may be substantially the same length, but the transgene may extend farther 5′ of the target site than 3′ of the target site. In some embodiments, the homology arm extends at least 10%, 20%, 30%, 40%, 50%, 2×, 3×, 4×, or 5× further to the 5′ end of the target site than the 3′ end of the target site.

In some embodiments alternative HDR proceeds more efficiently when the nucleic acid molecule is centered on the target site. Accordingly, in some embodiments, the nucleic acid molecule has two homology arms that are essentially the same size. In some embodiments, the first homology arm (e.g., 5′ homology arm) of a nucleic acid molecule may have a length that is within 10%, 9%, 8%, 7%, 6%, 5%, 4%, 3%, 2%, or 1% of the second homology arm (e.g., 3′ homology arm) of the nucleic acid molecule.

Similarly, in some embodiments, the nucleic acid molecule has a 5′ homology arm, a transgene, and a 3′ homology arm, such that the nucleic acid molecule extends substantially the same distance on either side of the target site. For example, the homology arms may have different lengths, but the transgene may be selected to compensate for this. For example, the transgene may extend further 5′ from the target site than it does 3′ of the target site, but the homology arm 5′ of the target site is shorter than the homology arm 3′ of the target site, to compensate. The converse is also possible, e.g., that the transgene may extend further 3′ from the target site than it does 5′ of the target site, but the homology arm 3′ of the target site is shorter than the homology arm 5′ of the target site, to compensate.

In some embodiments, the length of the nucleic acid molecule, including the transgene and the one or more homology arms, is between or between about 1000 to about 20,000 base pairs, such as about 1000, 1500, 2000, 2500, 3000, 3500, 4000, 4500, 5000, 6000, 7000, 8000, 9000, 10000, 11000, 12000, 13000, 14000, 15000, 16000, 17000, 18000, 19000 or 20000 base pairs. In some embodiments, the length of the nucleic acid molecule is limited by the maximum length of polynucleotide that can be prepared, synthesized or assembled and/or introduced into the cell, and the type of polynucleotide. In some aspects, the limited capacity of the nucleic acid molecule can determine the length of the transgene and/or the one or more homology arms. In some aspects, the combined total length of the transgene and the one or more homology arms must be within the maximum length or capacity of the polynucleotide. For example, in some aspects, the transgene portion of the nucleic acid molecule is about 1000, 1500, 2000, 2500, 3000, 3500 or 4000 base pairs, and if the maximum length of the nucleic acid molecule is about 5000 base pairs, the remaining portion of the sequence can be divided among the one or more homology arms, e.g., such that the 3′ or 5′ homology arms can be approximately 500, 750, 1000, 1250, 1500, 1750 or 2000 base pairs.

### 5. Cultivation

In some embodiments, the provided methods involve cultivating the immune cells, e.g., T cells. In some embodiments, the cultivating is carried out after the introducing of the one or more gene-editing agents. In some embodiments, the cultivating is carried out after the introducing of the nucleic acid molecule. In some embodiments, the cultivating is initiated immediately following the introducing of the nucleic acid molecule.

In some embodiments, the cultivating is effected under conditions to result in integration of the transgene into the target site. It is within the level of a skilled artisan to assess or determine if the cultivating has resulted in integration and hence to empirically determine the conditions for the cultivating. In some embodiments, integration can be assessed by measuring the level of expression of the recombinant protein encoded by the transgene. A number of well-known methods for assessing expression level of recombinant molecules may be used, such as detection by affinity-based methods, e.g., immunoaffinity-based methods, e.g., in the context of cell surface proteins, such as by flow cytometry. In some examples, the expression is measured by detection of a transduction marker and/or reporter construct. In some embodiments, a sequence encoding a truncated surface protein is included within the nucleic acid molecule and used as a marker of expression.

In certain embodiments, the cultivating is for, for about, or for at least 18 hours, 24 hours, 30 hours, 36 hours, 40 hours, 48 hours, 54 hours, 60 hours, 72 hours, 84 hours, 96 hours, or more than 96 hours. In some embodiments, the cultivating is performed for an amount of time between 30 minutes and 2 hours, between 1 hour and 8 hours, between 6 hours and 12 hours, between 12 hours and 18 hours, between 16 hours and 24 hours, between 18 hours and 30 hours, between 24 hours and 48 hours, between 24 hours and 72 hours, between 42 hours and 54 hours, between 60 hours and 120 hours between 96 hours and 120 hours, between 90 hours and between 1 days and 7 days, between 3 days and 8 days, between 1 day and 3 days, between 4 days and 6 days, or between 4 days and 5 days. In some embodiments, the cultivating is carried out for no more than 14 days. In some embodiments, the cultivating is carried out for no more than 12 days. In some embodiments, the cultivating is carried out for no more than 10 days. In some embodiments, the cultivating is carried out for no more than 8 days. In some embodiments, the cultivating is carried out for no more than 6 days. In some embodiments, the cultivating is carried out for no more than 5 days. In some embodiments, the cultivating is carried out for between or between about 12 hours and 36 hours, inclusive. In some embodiments, the cultivating is carried out for between or between about 18 hours and 30 hours, inclusive. In some embodiments, the cultivating is carried out for between or between about 22 hours and 26 hours, inclusive. In particular embodiments, the cultivating is for or for about 24 hours.

In some embodiments, the cultivating is under conditions to maintain a target amount of carbon dioxide in the cell culture. In some aspects, the amount of carbon dioxide (CO₂) is between 10% and 0% (v/v) of said gas, such as between 8% and 2% (v/v) of said gas, for example an amount of or about 5% (v/v) CO₂.

In some embodiments, the cultivating is carried out in a cell medium. In some embodiments, the cell medium is any described in Section I-B-2.

In some embodiments, the cultivating is carried out under conditions to induce expansion of the immune cells, e.g., T cells. In particular embodiments, the cultivating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to promote growth, division, and/or expansion of the immune cells, e.g., T cells. In some embodiments, the cultivating is carried out for a time period until a desired or threshold density, concentration, or number of cells is achieved.

In some embodiments, the cultivating is carried out in a bioreactor. Examples of suitable bioreactors for the cultivating include GE Xuri W25, GE Xuri W5, Sartorius BioSTAT RM 20|50, Finesse SmartRocker Bioreactor Systems, and Pall XRS Bioreactor Systems. In some embodiments, the bioreactor is used to perfuse and/or mix the immune cells, e.g., T cells, during at least a portion of the cultivating.

In some embodiments, the cultivating is carried out under conditions in which there is minimal or no further expansion of the immune cells, e.g., T cells. In some embodiments, the immune cells, e.g., T cells, are not cultivated under conditions that increase the amount of immune cells, e.g., T cells, during the cultivating. In some embodiments, the immune cells, e.g., T cells, are cultivated under conditions that may result in expansion, but the cultivating conditions are not carried out for purposes of expanding the immune cells, e.g., T cells.

In some embodiments, the cultivating occurs in an incubator. In some embodiments, the immune cells, e.g., T cells, are transferred into a container for the cultivating. In some embodiments, the container is a vial. In particular embodiments, the container is a bag. In some embodiments, the immune cells, e.g., T cells, are transferred into the container under closed or sterile conditions. In some embodiments, the container, e.g., the vial or bag, is then placed into an incubator for all or a portion of the cultivating. In particular embodiments, the incubator is set at, at about, or at least 16° C., 24° C., or 35° C. In some embodiments, the incubator is set at 37° C., at about at 37° C., or at 37° C.±2° C., ±1° C., ±0.5° C., or ±0.1° C.

In certain embodiments, the cultivating is performed under static conditions, such as conditions that do not involve centrifugation, shaking, rotating, rocking, or perfusion of media. In some embodiments, the cultivating is performed under gentle mixing conditions, e.g., involving rocking.

### D. Harvest

In some embodiments, the provided methods involve harvesting the genetically engineered immune cells, e.g., T cells, expressing the recombinant protein. In some embodiments, the harvesting is carried out following the engineering. In some embodiments, the harvesting is performed carried out following the cultivating.

In some embodiments, the harvesting is carried out between or between about 18 hours and 60 hours, 18 hours and 54 hours, 18 hours and 48 hours, 18 hours and 42 hours, 18 hours and 36 hours, 18 hours and 30 hours, 18 hours and 24 hours, 24 hours and 60 hours, 24 hours and 54 hours, 24 hours and 48 hours, 24 hours and 42 hours, 24 hours and 36 hours, 24 hours and 30 hours, 30 hours and 60 hours, 30 hours and 54 hours, 30 hours and 48 hours, 30 hours and 42 hours, 30 hours and 36 hours, 36 hours and 60 hours, 36 hours and 54 hours, 36 hours and 48 hours, 36 hours and 42 hours, 42 hours and 60 hours, 42 hours and 54 hours, 42 hours and 48 hours, 48 hours and 60 hours, 48 hours and 54 hours, 54 hours and 60 hours, each inclusive, after the adding of the sample. In some embodiments, the harvesting is carried out between or between about 36 hours and 60 hours, inclusive, after the adding of the sample. In some embodiments, the harvesting is carried out between or between about 42 hours and 54 hours, inclusive, after the adding of the sample. In some embodiments, the harvesting is carried out between or between about 46 hours and 50 hours, inclusive, after the adding of the sample. In some embodiments, the harvesting is carried out at or about 48 hours after the adding of the sample.

In some embodiments, the harvesting is carried out between or between about 18 hours and 60 hours, 18 hours and 54 hours, 18 hours and 48 hours, 18 hours and 42 hours, 18 hours and 36 hours, 18 hours and 30 hours, 18 hours and 24 hours, 24 hours and 60 hours, 24 hours and 54 hours, 24 hours and 48 hours, 24 hours and 42 hours, 24 hours and 36 hours, 24 hours and 30 hours, 30 hours and 60 hours, 30 hours and 54 hours, 30 hours and 48 hours, 30 hours and 42 hours, 30 hours and 36 hours, 36 hours and 60 hours, 36 hours and 54 hours, 36 hours and 48 hours, 36 hours and 42 hours, 42 hours and 60 hours, 42 hours and 54 hours, 42 hours and 48 hours, 48 hours and 60 hours, 48 hours and 54 hours, 54 hours and 60 hours, each inclusive, after the adding of the stimulatory reagent. In some embodiments, the harvesting is carried out between or between about 36 hours and 60 hours, inclusive, after the adding of the stimulatory reagent. In some embodiments, the harvesting is carried out between or between about 42 hours and 54 hours, inclusive, after the adding of the stimulatory reagent. In some embodiments, the harvesting is carried out between or between about 46 hours and 50 hours, inclusive, after the adding of the stimulatory reagent. In some embodiments, the harvesting is carried out at or about 48 hours after the adding of the stimulatory reagent.

In some embodiments, the harvesting is carried out between or between about 12 hours and 36 hours, 12 hours and 30 hours, 12 hours and 24 hours, 12 hours and 18 hours, 18 hours and 36 hours, 18 hours and 30 hours, 18 hours and 24 hours, 24 hours and 36 hours, 24 hours and 30 hours, or 30 hours and 36 hours, each inclusive, after the engineering, e.g., the introducing of the one or more gene-editing agents or the nucleic acid molecule. In some embodiments, the harvesting is carried out between or between about 12 hours and 36 hours, inclusive, after the engineering, e.g., the introducing of the one or more gene-editing agents or the nucleic acid molecule. In some embodiments, the harvesting is carried out between or between about 18 hours and 30 hours, inclusive, after the engineering, e.g., the introducing of the one or more gene-editing agents or the nucleic acid molecule. In some embodiments, the harvesting is carried out between or between about 22 hours and 26 hours, each inclusive, after the engineering, e.g., the introducing of the one or more gene-editing agents or the nucleic acid molecule. In some embodiments, the harvesting is carried out at or about 24 hours after the engineering, e.g., the introducing of the one or more gene-editing agents or the nucleic acid molecule.

### E. Formulation

In some embodiments, the provided methods involve formulating the harvested genetically engineered immune cells, e.g., T cells. In some embodiments, the harvested genetically engineered immune cells, e.g., T cells, are formulated in a container, such as a bag or vial.

In some embodiments, the harvested genetically engineered immune cells, e.g., T cells, are formulated for administration to a subject. In some embodiments, the harvested genetically engineered immune cells, e.g., T cells, are formulated in a pharmaceutically acceptable buffer, which may, in some aspects, include a pharmaceutically acceptable carrier or excipient. In some embodiments, the harvested genetically engineered immune cells, e.g., T cells, are formulated in the presence of a pharmaceutically acceptable excipient. Exemplary formulations are described in Section II.

In some embodiments, the harvested genetically engineered immune cells, e.g., T cells, are formulated for cryopreservation. In some embodiments, the harvested genetically engineered immune cells, e.g., T cells, are formulated in the presence of a cryoprotectant. In some embodiments, the harvested genetically engineered immune cells, e.g., T cells, are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the harvested genetically engineered immune cells, e.g., T cells, are frozen, e.g., cryoprotected or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the harvested genetically engineered immune cells, e.g., T cells, are frozen, e.g., cryoprotected or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

### II. COMPOSITIONS AND USES

Also provided herein are immune cells, e.g., T cells, such as CAR-expressing or TCR-expressing immune cells, e.g., T cells, produced by any of the methods described herein, as well as compositions, including pharmaceutical compositions and formulations, containing such immune cells, e.g., T cells. Also provided herein are methods of using and uses of the compositions, such as in the treatment of diseases, conditions, or disorders, for instance in which the antigen targeted by the CAR or TCR is expressed, or in detection, diagnostic, or prognostic methods.

In some embodiments, the term “pharmaceutical formulation” refers to a preparation which is in such form as to permit the biological activity of an active ingredient contained therein to be effective, and which contains no additional components which are unacceptably toxic to a subject to which the formulation would be administered.

In some embodiments, a “pharmaceutically acceptable carrier” refers to an ingredient in a pharmaceutical formulation, other than an active ingredient, which is nontoxic to a subject. A pharmaceutically acceptable carrier can include a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include methylparaben, propylparaben, sodium benzoate, and benzalkonium chloride. In some aspects, a mixture of two or more preservatives is used. The preservative or mixtures thereof can be present in an amount of about 0.0001% to about 2% by weight of the total composition. Carriers are described by, e.g., Remington's Pharmaceutical Sciences 16th edition, Osol, A. Ed. (1980). Pharmaceutically acceptable carriers are generally nontoxic to recipients at the dosages and concentrations employed and include buffers such as phosphate, citrate, or other organic acids; antioxidants such as ascorbic acid or methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose, or sorbitol; salt-forming counter-ions such as sodium; metal complexes (e.g., Zn-protein complexes); and non-ionic surfactants such as polyethylene glycol (PEG).

Buffering agents in some aspects are included in the compositions. Suitable buffering agents include citric acid, sodium citrate, phosphoric acid, potassium phosphate, and various other acids and salts. In some aspects, a mixture of two or more buffering agents is used. The buffering agent or mixtures thereof can be present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, or intranasal administration, or, if desired for local treatment, intralesional administration. Parenteral infusions include intramuscular, intravenous, intraarterial, intraperitoneal, or subcutaneous administration. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous or allogenic. For example, immunoresponsive cells or progenitors can be obtained from one subject and administered to the same subject or a different, compatible subject. Peripheral blood derived immunoresponsive cells or their progeny (e.g., in vivo, ex vivo, or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell or an agent that treats or ameliorates symptoms of neurotoxicity), it will generally be formulated in a unit dosage injectable form (solution, suspension, or emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. In some embodiments, the term “parenteral” includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations can be easier to prepare than gels, other viscous compositions, and solid compositions. Additionally, liquid compositions can be more convenient to administer, especially by injection. Viscous compositions, on the other hand, can be formulated within the appropriate viscosity range to provide longer contact periods with specific tissues. Liquid or viscous compositions can contain carriers, which can be a solvent or dispersing medium containing, for example, water, saline, phosphate buffered saline, polyol (for example, glycerol, propylene glycol, or liquid polyethylene glycol), and suitable mixtures of any of the foregoing.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished by, e.g., filtration through sterile filtration membranes.

### III. DEFINITIONS

Unless defined otherwise, all terms of art, notations, and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that smaller ranges between each intervening value between the upper and lower limit of that range and any other stated or intervening value in that stated range is encompassed within the claimed subject matter. The upper and lower limits of these smaller ranges may independently be included in the smaller ranges and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for a cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for a cell known to be positive for the marker, and/or at a level substantially similar as compared to that for a cell known to be negative for the marker.

As used herein, a composition refers to any mixture of two or more products, substances, or compounds, including cells. It may be a solution, a suspension, liquid, powder, a paste, aqueous, non-aqueous, or any combination of the foregoing.

As used herein, a “subject” is a mammal, such as a human or other animal, and typically is human.

### IV. EXEMPLARY EMBODIMENTS

1. A method for producing genetically engineered T cells, comprising:
(a) adding a whole blood sample comprising a plurality of T cells to a stationary phase in an internal cavity of a chromatography column, the stationary phase comprising a selection agent that specifically binds to a selection marker expressed on the surface of the plurality of T cells, wherein specific binding of the selection agent to the selection marker effects the immobilization of the plurality of T cells on the stationary phase;
(b) adding a T cell stimulatory reagent to the plurality of T cells immobilized on the stationary phase, wherein the T cell stimulatory reagent comprises (i) a primary agent that specifically binds to a member of a TCR/CD3 complex and (ii) a secondary agent that specifically binds to a T cell costimulatory molecule;
(c) incubating the plurality of T cells immobilized on the stationary phase in the presence of the T cell stimulatory reagent under conditions to stimulate T cells of the plurality of T cells;
(d) collecting T cells of the plurality of T cells from the chromatography column that are no longer immobilized after the incubating; and
(e) introducing by non-viral gene delivery a nucleic acid molecule comprising a transgene encoding a recombinant protein under conditions for targeted integration of the transgene into a target site of a gene in one or more of the collected T cells;
wherein the method produces genetically engineered T cells expressing the recombinant protein.

2. The method of embodiment 1, wherein the method comprises further incubating the collected T cells prior to the introducing of the nucleic acid molecule.

3. The method of embodiment 1 or embodiment 2, wherein the nucleic acid molecule is a DNA molecule.

4. The method of any one of embodiments 1-3, wherein the targeted integration is by Programmable Addition via Site-specific Targeting Elements (PASTE).

5. The method of embodiment 4, wherein the PASTE comprises introducing one or more gene-editing agents for editing the gene in the one or more of the collected T cells.

6. The method of any one of embodiments 1-3, wherein the targeted integration is by homology directed repair (HDR).

7. The method of embodiment 6, wherein the HDR comprises introducing one or more gene-editing agents for inducing a genetic disruption in the gene in the one or more of the collected T cells.

8. The method of any one of embodiments 1-7, wherein the introducing of the nucleic acid molecule and/or the one or more gene-editing agents is by electroporation.

9. The method of any one of embodiments 1-8, wherein the conditions for targeted integration comprise cultivating the collected T cells under conditions to integrate the transgene into the target site.

10. A method for producing genetically engineered T cells, comprising:
(a) adding a whole blood sample comprising a plurality of T cells to a stationary phase in an internal cavity of a chromatography column, the stationary phase comprising a selection agent that specifically binds to a selection marker expressed on the surface of the plurality of T cells, wherein specific binding of the selection agent to the selection marker effects the immobilization of the plurality of T cells on the stationary phase;
(b) adding a T cell stimulatory reagent to the plurality of T cells immobilized on the stationary phase, wherein the T cell stimulatory reagent comprises (i) a primary agent that specifically binds to a member of a TCR/CD3 complex and (ii) a secondary agent that specifically binds to a T cell costimulatory molecule;
(c) incubating the plurality of T cells immobilized on the stationary phase in the presence of the T cell stimulatory reagent under conditions to stimulate T cells of the plurality of T cells;
(d) collecting T cells of the plurality of T cells from the chromatography column that are no longer immobilized after the incubating;
(e) further incubating the collected T cells;
(f) after the further incubating, introducing into T cells of the collected T cells (i) a nucleic acid molecule comprising a transgene encoding a recombinant protein, wherein the nucleic acid molecule is a DNA molecule, and the introducing of the nucleic acid molecule is by non-viral gene delivery, and (ii) one or more gene-editing agents for inducing a genetic disruption in a gene in the T cells of the collected T cells, wherein the introducing of the nucleic acid molecule and/or the one or more gene-editing agents is by electroporation; and
(g) cultivating the collected T cells under conditions to integrate by homology directed repair (HDR) the transgene into a target site of the gene in one or more of the collected T cells;
wherein the method produces genetically engineered T cells expressing the recombinant protein.

11. The method of any one of embodiments 1-10, wherein the nucleic acid molecule is a naked DNA molecule and/or a closed-ended DNA molecule.

12. The method of any one of embodiments 1-11, wherein the nucleic acid molecule is a closed-ended linear duplex (CELiD) DNA molecule, a minicircle DNA molecule, a minimalistic immunological-defined gene expression (MIDGE) DNA molecule, a ministring DNA molecule, a dumbbell-shaped linear duplex closed-ended DNA molecule, or a Doggybone™ DNA molecule.

13. The method of any one of embodiments 1-12, wherein the T cell stimulatory reagent is added in a cell medium.

14. The method of any one of embodiments 1-13, wherein the T cell stimulatory reagent is added in an amount between or between about 0.1 μg and 20 μg, 0.4 μg and 8 μg, 0.8 μg and 4 μg, or 1 μg and 2 μg, each inclusive and each per 10⁶ T cells of the plurality of T cells immobilized or expected to be immobilized on the stationary phase.

15. The method of any one of embodiments 1-14, wherein the binding capacity of the stationary phase is between or between about 0.5 billion and 5 billion T cells expressing the selection marker, 0.5 billion and 3 billion T cells expressing the selection marker, or 1 billion and 2 billion T cells expressing the selection marker, each inclusive.

16. The method of any one of embodiments 1-15, wherein the T cell stimulatory reagent is added in an amount between or between about 0.1 mg and 20 mg, 0.4 mg and 8 mg, 0.8 mg and 4 mg, or 1 mg and 3 mg, each inclusive.

17. The method of any one of embodiments 1-16, wherein the adding of the T cell stimulatory reagent is carried out within or within about 60 minutes, 30 minutes, or 15 minutes after the adding of the whole blood sample.

18. The method of any one of embodiments 1-17, wherein the incubating is carried out in a cell medium.

19. The method of any one of embodiments 1-18, wherein the incubating is carried out at a temperature between or between about 35° C. and about 39° C.

20. The method of any one of embodiments 1-19, wherein the incubating is carried out for between or between about 0.5 hour and 8 hours, 2 hours and 6 hours, or 3 hours and 5 hours, each inclusive.

21. The method of any one of embodiments 1-20, wherein the collecting comprises adding a wash buffer to the stationary phase to collect the T cells of the plurality of T cells.

22. The method of embodiment 21, wherein the wash buffer is a cell medium.

23. The method of embodiment 21 or embodiment 22, wherein the wash buffer does not comprise a competition agent.

24. The method of any one of embodiments 1-23, wherein the collecting is carried out between or between about 0.5 hours and 8 hours, 2 hours and 6 hours, or 3 hours and 5 hours, each inclusive, after the adding of the T cell stimulatory reagent.

25. The method of any one of embodiments 2-24, wherein the further incubating is carried out in the presence of the T cell stimulatory reagent.

26. The method of any one of embodiments 2-25, wherein the further incubating is carried out in a cell medium.

27. The method of any one of embodiments 2-26, wherein the further incubating is carried out at a temperature between or between about 35° C. and about 39° C.

28. The method of any one of embodiments 2-27, wherein the further incubating is carried out for between or between about 10 hours and 30 hours, 16 hours and 24 hours, or 18 hours and 22 hours, each inclusive.

29. The method of any one of embodiments 5 and 7-28, wherein the method comprises removing the T cell stimulatory reagent from the collected T cells prior to the introducing of the one or more gene-editing agents.

30. The method of any one of embodiments 1-29, wherein the method comprises removing the T cell stimulatory reagent from the collected T cells prior to the introducing of the nucleic acid molecule.

31. The method of embodiment 29 or embodiment 30, wherein the removing is carried out after the further incubating.

32. The method of any one of embodiments 29-31, wherein the removing comprises washing the collected T cells.

33. The method of any one of embodiments 5 and 7-32, wherein:
the T cell stimulatory reagent comprises an oligomer of streptavidin or a streptavidin mutein molecule;
the primary agent comprises a first streptavidin-binding partner that is bound to a streptavidin or streptavidin mutein molecule of the oligomer;
the secondary agent comprises a second streptavidin-binding partner that is bound to a streptavidin or streptavidin mutein molecule of the oligomer; and
the method comprises disrupting the binding between the first and second streptavidin-binding partners and the streptavidin or streptavidin mutein molecules prior to the introducing of the one or more gene-editing agents.

34. The method of any one of embodiments 1-33, wherein:
the T cell stimulatory reagent comprises an oligomer of streptavidin or a streptavidin mutein molecule;
the primary agent comprises a first streptavidin-binding partner that is bound to a streptavidin or streptavidin mutein molecule of the oligomer;
the secondary agent comprises a second streptavidin-binding partner that is bound to a streptavidin or streptavidin mutein molecule of the oligomer; and
the method comprises disrupting the binding between the first and second streptavidin-binding partners and the streptavidin or streptavidin mutein molecules prior to the introducing of the nucleic acid molecule.

35. The method of embodiment 33 or embodiment 34, wherein the disrupting is carried out after the further incubating.

36. The method of any one of embodiments 33-35, wherein the disrupting is by adding a competition agent to the collected T cells that reverses the binding between the first and second streptavidin-binding partners and the streptavidin or streptavidin mutein molecules.

37. The method of any one of embodiments 23-36, wherein the competition agent is biotin.

38. The method of any one of embodiments 5 and 7-37, wherein the introducing of the one or more gene-editing agents is carried out prior to or concurrently with the introducing of the nucleic acid molecule.

39. The method of any one of embodiments 5 and 7-38, wherein the introducing of the one or more gene-editing agents is carried out between or between about 12 hours and 36 hours, 18 hours and 30 hours, or 22 hours and 26 hours, each inclusive, after the adding of the T cell stimulatory reagent.

40. The method of any one of embodiments 1-39, wherein the nucleic acid molecule is introduced in a cell medium comprising the nucleic acid molecule.

41. The method of any one of embodiments 1-40, wherein the introducing of the nucleic acid molecule is carried out between or between about 12 hours and 36 hours, 18 hours and 30 hours, or 22 hours and 26 hours, each inclusive, after the adding of the T cell stimulatory reagent.

42. The method of any one of embodiments 9-41, wherein the cultivating is carried out in the presence of the nucleic acid molecule.

43. The method of any one of embodiments 9-42, wherein the cultivating is carried out in a cell medium.

44. The method of any one of embodiments 9-43, wherein the cultivating is carried out at a temperature between or between about 35° C. and about 39° C.

45. The method of any one of embodiments 9-44, wherein the cultivating is carried out for between or between about 12 hours and 36 hours, 18 hours and 30 hours, or 22 hours and 26 hours, each inclusive.

46. The method of any one of embodiments 13-45, wherein the cell medium is a basal medium.

47. The method of any one of embodiments 13-46, wherein the cell medium is a serum free medium.

48. The method of any one of embodiments 13-47, wherein the cell medium comprises no cytokines or comprises recombinant IL-2, IL-7, and IL-15.

49. The method of any one of embodiments 1-48, wherein the method comprises harvesting the genetically engineered T cells expressing the recombinant protein.

50. The method of embodiment 49, wherein the harvesting is carried out between or between about 36 hours and 60 hours, 42 hours and 54 hours, or 46 hours and 50 hours, each inclusive, after the adding of the whole blood sample.

51. The method of embodiment 49 or embodiment 50, wherein the harvesting is carried out between or between about 36 hours and 60 hours, 42 hours and 54 hours, or 46 hours and 50 hours, each inclusive, after the adding of the T cell stimulatory reagent.

52. The method of any one of embodiments 49-51, wherein the harvesting is carried out between or between about 12 hours and 36 hours, 18 hours and 30 hours, or 22 hours and 26 hours, each inclusive, after the introducing of the one or more gene-editing agents.

53. The method of any one of embodiments 49-52, wherein the harvesting is carried out between or between about 12 hours and 36 hours, 18 hours and 30 hours, or 22 hours and 26 hours, each inclusive, after the introducing of the nucleic acid molecule.

54. The method of any one of embodiments 49-53, wherein the method comprises formulating the harvested genetically engineered T cells for cryopreservation or administration to a subject.

55. The method of embodiment 54, wherein the harvested genetically engineered T cells are formulated in the presence of a cryoprotectant or a pharmaceutically acceptable excipient.

56. The method of any one of embodiments 1-55, wherein the plurality of T cells are primary T cells from a human subject.

57. The method of any one of embodiments 1-56, wherein the selection marker is selected from the group consisting of CD3, CD4, CD8, CD45RA, CD27, CD28, and CCR7.

58. The method of any one of embodiments 1-57, wherein the selection marker is CD3, CD4, or CD8.

59. The method of any one of embodiments 1-58, wherein the selection marker is CD3.

60. The method of any one of embodiments 1-59, wherein the selection agent comprises an antibody or antibody fragment that specifically binds to the selection marker.

61. The method of embodiment 60, wherein the antibody or antibody fragment of the selection agent is a monovalent antibody fragment.

62. The method of embodiment 60 or embodiment 61, wherein the antibody or antibody fragment of the selection agent is a Fab fragment.

63. The method of any one of embodiments 1-32 and 37-62, wherein:
the T cell stimulatory reagent comprises an oligomer of streptavidin or a streptavidin mutein molecule;
the primary agent comprises a first streptavidin-binding partner that is bound to a streptavidin or streptavidin mutein molecule of the oligomer; and
the secondary agent comprises a second streptavidin-binding partner that is bound to a streptavidin or streptavidin mutein molecule of the oligomer.

64. The method of any one of embodiments 33-63, wherein the T cell stimulatory reagent consists or consists essentially of the oligomer, primary agent, and secondary agent.

65. The method of any one of embodiments 33-64, wherein the oligomer comprises between or between about 500 and 5000 tetramers, 1000 and 4000 tetramers, or 2000 and 3000 tetramers, each inclusive, of the streptavidin or streptavidin mutein molecule.

66. The method of any one of embodiments 33-65, wherein the oligomer is of the streptavidin mutein molecule.

67. The method of any one of embodiments 33-66, wherein the streptavidin mutein molecule comprises the amino acid sequence IGAR (SEQ ID NO: 133) or VTAR (SEQ ID NO: 134) at sequence positions corresponding to positions 44 to 47 of the sequence of amino acids set forth in SEQ ID NO: 1.

68. The method of any one of embodiments 33-67, wherein the streptavidin mutein molecule begins N-terminally in the region of amino acid positions 10 to 16 of SEQ ID NO: 1 and terminates C-terminally in the region of amino acid positions 133 to 142 of SEQ ID NO: 1.

69. The method of any one of embodiments 33-68, wherein the streptavidin mutein molecule comprises the amino acid sequence set forth in any one of SEQ ID NO: 3-6, 27, 28, 104, 105, and 136.

70. The method of any one of embodiments 33-69, wherein the streptavidin mutein molecule comprises the amino acid sequence set forth in SEQ ID NO: 6.

71. The method of any one of embodiments 33-70, wherein:
the first streptavidin-binding partner is at the C-terminus of the primary agent; and/or the second streptavidin-binding partner is at the C-terminus of the secondary agent.

72. The method of any one of embodiments 33-71, wherein the first and/or second streptavidin-binding partner is a streptavidin-binding peptide.

73. The method of embodiment 72, wherein the streptavidin-binding peptide of the first and/or second streptavidin-binding partner comprises the amino acid sequence set forth in any one of SEQ ID NO: 7, 8, and 15-19.

74. The method of embodiment 72 or embodiment 73, wherein the streptavidin-binding peptide of the first and/or second streptavidin-binding partner comprises the amino acid sequence set forth in SEQ ID NO: 16.

75. The method of any one of embodiments 1-74, wherein the member of the TCR/CD3 complex is CD3.

76. The method of any one of embodiments 1-75, wherein the T cell costimulatory molecule is CD28, CD90 (Thy-1), CD95 (Apo-/Fas), CD137 (4-1BB), CD154 (CD40L), ICOS, LAT, CD27, OX40, or HVEM.

77. The method of any one of embodiments 1-76, wherein the T cell costimulatory molecule is CD28.

78. The method of any one of embodiments 1-77, wherein:
the primary agent comprises an antibody or antibody fragment that specifically binds to the member of the TCR/CD3 complex; and/or
the secondary agent comprises an antibody or antibody fragment that specifically binds to the T cell costimulatory agent.

79. The method of embodiment 78, wherein:
the antibody or antibody fragment of the primary agent comprises a heavy chain, and the first streptavidin-binding partner is fused to the C-terminus of the heavy chain of the primary agent; and/or
the antibody or antibody fragment of the secondary agent comprises a heavy chain, and the second streptavidin-binding partner is fused to the C-terminus of the heavy chain of the secondary agent.

80. The method of embodiment 78 or embodiment 79, wherein the antibody or antibody fragment of the primary and/or secondary agent is a monovalent antibody fragment.

81. The method of any one of embodiments 78-80, wherein the antibody or antibody fragment of the primary and/or secondary agent is a Fab fragment.

82. The method of any one of embodiments 1-81, wherein the primary agent comprises an anti-CD3 antibody or antibody fragment, and the secondary agent comprises an anti-CD28 antibody or antibody fragment.

83. The method of any one of embodiments 1-82, wherein the primary agent comprises an anti-CD3 Fab fragment, and the secondary agent comprises an anti-CD28 Fab fragment.

84. The method of any one of embodiments 1-83, wherein the gene is the T cell receptor alpha constant (TRAC) gene.

85. The method of any one of embodiments 1-84, wherein the target site is within the sequence set forth in SEQ ID NO: 250.

86. The method of any one of embodiments 1-3 and 6-85, wherein the nucleic acid molecule comprises a 5′ homology arm and a 3′ homology arm comprising sequences homologous to nucleic acid sequences surrounding the target site, the nucleic acid molecule comprising the structure [5′ homology arm]-[transgene]-[3′ homology arm].

87. The method of embodiment 86, wherein the 5′ homology arm and the 3′ homology arm comprise sequences homologous to sequences of the TRAC gene surrounding the target site.

88. The method of embodiment 86 or embodiment 87, wherein the 5′ homology arm comprises a sequence comprising at least or at least about 150, 200, 250, 300, 350, 400, 450, 500, 550, or 600 contiguous nucleotides of a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 248.

89. The method of any one of embodiments 86-88, wherein the 5′ homology arm comprises at least or at least about 150, 200, 250, 300, 350, 400, 450, 500, 550, or 600 contiguous nucleotides of the sequence set forth in SEQ ID NO: 248.

90. The method of any one of embodiments 86-89, wherein the 5′ homology arm comprises the sequence set forth in SEQ ID NO: 248.

91. The method of any one of embodiments 86-90, wherein the 3′ homology arm comprises a sequence comprising at least or at least about 150, 200, 250, 300, 350, 400, 450, 500, 550, or 600 contiguous nucleotides of a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 249.

92. The method of any one of embodiments 86-91, wherein the 3′ homology arm comprises at least or at least about 150, 200, 250, 300, 350, 400, 450, 500, 550, or 600 contiguous nucleotides of the sequence set forth in SEQ ID NO: 249.

93. The method of any one of embodiments 86-92, wherein the 3′ homology arm comprises the sequence set forth in SEQ ID NO: 249.

94. The method of any one of embodiments 1-93, wherein transcription of the integrated transgene is under the control of a promoter comprised by the nucleic acid molecule.

95. The method of embodiment 94, wherein the promoter is a human elongation factor 1 alpha (EF1α) promoter.

96. The method of embodiment 94 or embodiment 95, wherein the promoter comprises the sequence set forth in SEQ ID NO: 247.

97. The method of any one of embodiments 1-96, wherein the recombinant protein is a recombinant receptor.

98. The method of embodiment 97, wherein the recombinant receptor is a T cell receptor or a chimeric antigen receptor.

99. The method of any one of embodiments 5 and 7-98, wherein the one or more gene-editing agents comprise (i) a gene-editing nuclease or nuclease combination or (ii) a nucleic acid molecule comprising one or more sequences encoding the gene-editing nuclease or nuclease combination.

100. The method of any one of embodiments 5 and 7-99, wherein the one or more gene-editing agents comprise a gene-editing nuclease or nuclease combination.

101. The method of embodiment 99 or embodiment 100, wherein the gene-editing nuclease or nuclease combination specifically recognizes a nucleic acid sequence near or comprising the target site.

102. The method of any one of embodiments 99-101, wherein the gene-editing nuclease or nuclease combination specifically recognizes a nucleic acid sequence comprising the target site.

103. The method of embodiment 101 or embodiment 102, wherein the nucleic acid sequence comprising the target site comprises the sequence set forth in SEQ ID NO: 250.

104. The method of any one of embodiments 99-103, wherein the gene-editing nuclease or nuclease combination is a zinc finger nuclease, a transcription activator-like effector nuclease, or a CRISPR-Cas combination.

105. The method of any one of embodiments 99-104, wherein the gene-editing nuclease or nuclease combination is a CRISPR-Cas combination.

106. The method of embodiment 104 or embodiment 105, wherein the CRISPR-Cas combination comprises a CRISPR-Cas nickase, reverse transcriptase, and serine integrase.

107. The method of embodiment 105 or embodiment 106, wherein the CRISPR-Cas combination comprises a guide RNA comprising a targeting sequence that is complementary to the nucleic acid sequence comprising the target site.

108. The method of embodiment 105 or embodiment 107, wherein the CRISPR-Cas combination is a ribonucleoprotein complex comprising the guide RNA and a Cas protein.

109. The method of embodiment 108, wherein the Cas protein is a S. pyogenes Cas protein.

110. The method of any one of embodiments 104-109, wherein the CRISPR-Cas combination is a CRISPR-Cas9 combination or a CRISPR-Cas12 combination.

111. The method of any one of embodiments 107-110, wherein the targeting sequence comprises the sequence set forth in any one of SEQ ID NO: 144-175.

112. The method of any one of embodiments 107-111, wherein the targeting sequence comprises the sequence set forth in SEQ ID NO: 148.

113. A genetically engineered T cell produced by the method of any one of embodiments 1-112, wherein the genetically engineered T cell expresses the recombinant protein.

114. The genetically engineered T cell of embodiment 113, wherein the transgene is integrated into the target site of the gene in the genetically engineered T cell.

115. The genetically engineered T cell of embodiment 114, wherein the gene is the T cell receptor alpha constant (TRAC) gene.

116. The genetically engineered T cell of embodiment 114 or embodiment 115, wherein the target site is within the sequence set forth in SEQ ID NO: 250.

117. The genetically engineered T cell of any one of embodiments 113-116, wherein the recombinant protein is a recombinant receptor.

118. The genetically engineered T cell of embodiment 117, wherein the recombinant receptor is a T cell receptor or a chimeric antigen receptor.

119. A population of T cells comprising a plurality of the genetically engineered T cell of any one of embodiments 113-118.

120. The population of embodiment 119, wherein the plurality of genetically engineered T cells are at least 10%, 15%, or 20% of the population of T cells.

121. The population of embodiment 119 or embodiment 120, wherein the gene is disrupted in at least 85%, 90%, or 95% of the T cells of the population of T cells.

122. The population of embodiment 121, wherein the gene is the T cell receptor alpha constant (TRAC) gene.

123. A pharmaceutical composition comprising the population of T cells of any one of embodiments 119-122 and a pharmaceutically acceptable excipient.

124. A method of treatment, comprising administering to a subject having a disease or condition the pharmaceutical composition of embodiment 123.

125. The method of embodiment 124, wherein the recombinant protein is a recombinant receptor that targets an antigen expressed on a target cell associated with the disease or condition.

126. A method of cytolytic killing of a target cell, comprising contacting a target cell with the population of any one of embodiments 119-122.

127. A method of cytolytic killing of a target cell, comprising contacting a target cell with the pharmaceutical composition of embodiment 123.

128. The method of embodiment 126 or embodiment 127, wherein the contacting is performed ex vivo.

129. The method of embodiment 126 or embodiment 127, wherein the contacting is performed in vivo.

130. The method of embodiment 129, wherein the contacting is by administering the pharmaceutical composition to a subject having a disease or condition.

131. The method of embodiment 130, wherein the target cell is associated with the disease or condition, and the recombinant protein is a recombinant receptor that targets an antigen expressed on the target cell.

132. The method of embodiment 125 or embodiment 131, wherein the recombinant receptor is a T cell receptor or a chimeric antigen receptor.

133. The pharmaceutical composition of embodiment 123 for use in treating a disease or disorder in a subject.

134. The pharmaceutical composition of embodiment 133, wherein the recombinant protein is a recombinant receptor that targets an antigen expressed on a cell associated with the disease or condition.

135. The pharmaceutical composition of embodiment 134, wherein the recombinant receptor is a T cell receptor or a chimeric antigen receptor.

136. Use of the pharmaceutical composition of embodiment 123 for treating a disease or disorder in a subject.

137. Use of the pharmaceutical composition of embodiment 123 for the manufacture of a medicament for treating a disease or disorder in a subject.

138. The use of embodiment 136 or embodiment 137, wherein the recombinant protein is a recombinant receptor that targets an antigen expressed on a cell associated with the disease or condition.

139. The use of embodiment 138, wherein the recombinant receptor is a T cell receptor or a chimeric antigen receptor.

### V. EXAMPLES

The following examples are included for illustrative purposes only and are not intended to limit the scope of the invention.

### Example 1: Engineering of T Cells from a Whole Blood Sample Using CRISPR-Based Gene Editing with Naked DNA Constructs

A study is carried out in which engineered T cells are produced from a whole blood sample using an ex vivo manufacturing process involving on-column stimulation of T cells prior to engineering of the T cells using naked DNA constructs containing a transgene encoding a recombinant protein. Clustered regularly interspaced short palindromic repeats (CRISPR)-associated protein (Cas) is used for targeted integration of the transgene via homology directed repair (HDR). For comparison, a similar process is carried out in which T cells from an apheresis sample are engineered.

In phosphate buffered saline (PBS) buffer, 10 mg of multimerized streptavidin mutein (Strep-Tactin® m2, SEQ ID NO: 6) are coupled to 8 g (with respect to initial dry weight) of epoxy-activated polystytrene resin (CY17030; Cytosorbents) via solvent-exposed primary amine groups. Subsequently, 1.8 mg of a selection agent comprising an anti-CD3 Fab fragment and a streptavidin-binding peptide (Twin-Strep-tag®, SEQ ID NO: 16) carboxyl-terminally fused to the heavy chain of the Fab fragment are added to a 50% suspension (resin bed volume vs. total volume) of the Strep-Tactin®-coated resin. The Twin-Strep-tag® streptavidin-binding peptide contains a sequential arrangement of two streptavidin-binding modules. The peptide-tagged anti-CD3 Fab fragment is recombinantly produced (see International Patent App. Pub. Nos. WO 2013/011011 and WO 2013/124474). The anti-CD3 Fab fragment is derived from the CD3 binding monoclonal antibody produced by the hybridoma cell line OKT3 (ATCC® CRL-8001™; see also U.S. Pat. No. 4,361,549) and contains the heavy chain variable domain (SEQ ID NO: 31) and light chain variable domain (SEQ ID NO: 32) of the anti-CD3 antibody OKT3 described in Arakawa et al J. Biochem. 120, 657-662 (1996). The suspension is incubated under gentle shaking for one hour at room temperature in order to allow, via the Twin-Strep-tag®, the reversible binding of the selection agent to the resin via the immobilized Strep-Tactin®. The anti-CD3 Fab fragment functionalized resin is then added to a plastic full-scale column to give a bed volume of 18-20 mL. Prior to use, the column is equilibrated with PBS containing 0.5% bovine serum albumin.

For on-column T cell stimulation, an anti-CD3/anti-CD28 stimulatory reagent is prepared using an oligomeric streptavidin mutein reagent produced as described in WO 2018/197949 (see also Poltorak et al., Scientific Reports (2020)). The oligomeric streptavidin mutein reagent has an average hydrodynamic radius of 90-120 nm and contains an average of 2000-2800 tetramers of a streptavidin mutein (Strep-Tactin® m2, SEQ ID NO: 6). The oligomeric streptavidin mutein reagent is mixed at room temperature with the peptide-tagged anti-CD3 Fab fragment described above as well as with an anti-CD28 Fab fragment also individually fused at the carboxy-terminus of its heavy chain to a streptavidin-binding peptide sequence (Twin-Strep-tag®, SEQ ID NO: 16). The anti-CD28 Fab fragment is derived from antibody CD28.3 (deposited as a synthetic single chain Fv construct under GenBank Accession No. AF451974.1; see also Vanhove et al., BLOOD, 15 Jul. 2003, Vol. 102, No. 2, pages 564-570) and contains the heavy chain variable domain (SEQ ID NO: 33) and the light chain variable domain (SEQ ID NO: 34) of the anti-CD28 antibody CD28.3. To prepare the anti-CD3/anti-CD28 stimulatory reagent, 0.3 mg of oligomeric streptavidin mutein reagent, 0.5 μg of peptide-tagged anti-CD3 Fab fragments, and 0.5 μg of peptide-tagged anti-CD28 Fab fragment is used.

For T cell engineering, naked DNA constructs containing the recombinant protein-encoding transgene flanked by 5′ and 3′ homology arms are prepared. The homology arms include nucleic acid sequences homologous to sequences surrounding the target integration site in the human TCR α constant region (TRAC) gene. For Cas targeting of the TRAC locus, a guide RNA (gRNA) having a targeting domain sequence is used.

A whole blood sample from a human donor is loaded onto the anti-CD3 affinity chromatography column. Following loading and washing, cells while immobilized on-column are incubated in the presence of an activation medium that includes serum-free basal media containing recombinant IL-2, IL-15, and IL-7 and the anti-CD3/anti-CD28 stimulatory reagent. Following addition of the activation medium, the column is heated and incubated at 37° C. Following incubation, during which time cells spontaneously detach from the column, the cells are eluted using serum-free basal media containing recombinant IL-2, IL-15, and IL-7. Eluted cells are collected in a culture bag and incubated in static culture in an incubator at 37° C.

Following incubation, the cells are electroporated with ribonucleoprotein (RNP) complexes containing Streptococcus pyogenes (S. pyogenes) Cas and the TRAC-targeting gRNA. The naked DNA construct is present with the RNP complexes and the TRAC-target gRNA during electroporation. Following electroporation, the cells are incubated in serum-free basal media containing recombinant IL-2, IL-15, and IL-7. Incubation occurs in static culture in an incubator at 37° C. After incubation, the cells are harvested, washed, and cryopreserved. Recombinant protein expression is assessed. For comparison, an apheresis sample is loaded onto the anti-CD3 affinity chromatography column and subjected to the manufacturing process.

### Example 2: Assessment of Engineering of T Cells from a Whole Blood Sample Using CRISPR-Based Gene Editing with Naked DNA Constructs

A study was carried out in which engineered T cells were produced from a whole blood sample using an ex vivo manufacturing process involving on-column stimulation of T cells prior to engineering of the T cells using naked double-stranded DNA constructs containing a transgene encoding a recombinant protein, in this case a chimeric antigen receptor (CAR). Clustered regularly interspaced short palindromic repeats (CRISPR)-associated protein (Cas) was used for targeted integration of the transgene via homology directed repair (HDR).

In phosphate buffered saline (PBS) buffer, 10 mg of multimerized streptavidin mutein (Strep-Tactin® m2, SEQ ID NO: 6) was coupled to 8 g (with respect to initial dry weight) of epoxy-activated polystytrene resin (CY17030; Cytosorbents) via solvent-exposed primary amine groups. Subsequently, 1.8 mg of a selection agent comprising an anti-CD3 Fab fragment and a streptavidin-binding peptide (Twin-Strep-tag®, SEQ ID NO: 16) carboxyl-terminally fused to the heavy chain of the Fab fragment was added to a 50% (1:1) suspension (resin bed volume vs. total volume) of the Strep-Tactin®-coated resin. The Twin-Strep-tag® streptavidin-binding peptide contained a sequential arrangement of two streptavidin-binding modules. The peptide-tagged anti-CD3 Fab fragment was recombinantly produced (see International Patent App. Pub. Nos. WO 2013/011011 and WO 2013/124474). The anti-CD3 Fab fragment was derived from the CD3 binding monoclonal antibody produced by the hybridoma cell line OKT3 (ATCC® CRL-8001™; see also U.S. Pat. No. 4,361,549) and contained the heavy chain variable domain (SEQ ID NO: 31) and light chain variable domain (SEQ ID NO: 32) of the anti-CD3 antibody OKT3 described in Arakawa et al J. Biochem. 120, 657-662 (1996). The suspension was incubated under gentle shaking conditions for one hour at room temperature in order to allow, via the Twin-Strep-tag®, the reversible binding of the selection agent to the resin via the immobilized Strep-Tactin®. The anti-CD3 Fab fragment functionalized resin was then added to a plastic full-scale column to give a wet bed volume of about 20 mL. Prior to use, the column was equilibrated with PBS containing 0.5% bovine serum albumin.

For on-column T cell stimulation, an anti-CD3/anti-CD28 stimulatory reagent was prepared using an oligomeric streptavidin mutein reagent produced as described in WO 2018/197949 (see also Poltorak et al., Scientific Reports (2020)). The oligomeric streptavidin mutein reagent had an average hydrodynamic radius of 90-120 nm and contained an average of 2000-2800 tetramers of a streptavidin mutein (Strep-Tactin® m2, SEQ ID NO: 6). The oligomeric streptavidin mutein reagent was mixed at room temperature with the peptide-tagged anti-CD3 Fab fragment described above as well as with an anti-CD28 Fab fragment also individually fused at the carboxy-terminus of its heavy chain to a streptavidin-binding peptide sequence (Twin-Strep-tag®, SEQ ID NO: 16). The anti-CD28 Fab fragment was derived from antibody CD28.3 (deposited as a synthetic single chain Fv construct under GenBank Accession No. AF451974.1; see also Vanhove et al., BLOOD, 15 Jul. 2003, Vol. 102, No. 2, pages 564-570) and contained the heavy chain variable domain (SEQ ID NO: 33) and the light chain variable domain (SEQ ID NO: 34) of the anti-CD28 antibody CD28.3. To prepare the anti-CD3/anti-CD28 stimulatory reagent, 0.3 mg of oligomeric streptavidin mutein reagent, 0.5 μg of peptide-tagged anti-CD3 Fab fragments, and 0.5 μg of peptide-tagged anti-CD28 Fab fragment was used.

For T cell engineering, naked DNA constructs containing the recombinant protein-encoding transgene flanked by 5′ and 3′ homology arms were prepared. Specifically, a double-stranded linear HDR template flanked by Cas9 target sequence sites (CTSs) was used (see Shy et al., Nat Biotechnol, 2022 Aug. 25). Each CTS included a truncated gRNA target sequence and an NGG Protospacer-Adjacent-Motif (PAM), with 8 scrambled bases included upstream of the truncated gRNA target sequence. The 5′ CTS was tggctctctctctcagctggtacacgg (SEQ ID NO: 251), and the 3′ CTS was ccagcacctctctcagctggtacacgg (SEQ ID NO: 252).

### 5′ CTS (SEQ ID NO: 251)

tggctctctctctcagctggtacacgg

### 3′ CTS (SEQ ID NO: 252)

ccagcacctctctcagctggtacacgg

The homology arms included nucleic acid sequences homologous to sequences surrounding the target integration site in exon 1 of the human TCR α constant region (TRAC) gene (5′ homology arm sequence set forth in SEQ ID NO: 253; 3′ homology arm sequence set forth in SEQ ID NO: 254; target site sequence for TRAC set forth in SEQ ID NO: 250). Targeted knock-in of the recombinant protein-encoding transgene resulted in transgene expression under the control of the endogenous TRAC promoter.

5′ homology arm (SEQ ID NO: 253)
gggaaatgagatcatgtcctaaccctgatcctcttgtcccacagatatccagaaccctgaccctgccgtgggaagc gga
   3′ homology arm (SEQ ID NO: 254)
atccagaaccccgaccctgctgtttatcagctcagggattctaaatccagtgacaagtctgtctgcctattcaccgatt ttg

### TRAC Exon 1 Target Site (SEQ ID NO: 250)

attcaccgattttgattctc

A whole blood sample from a healthy human donor was loaded onto the anti-CD3 affinity chromatography column at a loading rate of 25 mL/min. Following loading and washing, cells while immobilized on-column were incubated in the presence of an activation medium that included recombinant IL-2, IL-15, and IL-7 and 1 mg of the anti-CD3/anti-CD28 stimulatory reagent. Following addition of the activation medium, the column was heated and incubated at 36-38° C. for 4.5 hours. Following incubation, during which time cells spontaneously detached from the column, the cells were eluted using complete medium containing recombinant IL-2, IL-15, and IL-7 (1:1000).

Immediately following elution, the cells were washed and electroporated with the naked DNA construct and ribonucleoprotein (RNP) complexes containing 1.9 μM TRAC-targeting gRNA and 0.28 μM Streptococcus pyogenes (S. pyogenes) Cas9 per million cells. The TRAC-targeting gRNA was 5′ agagtctctcagctggtaca 3′ (SEQ ID NO: 255). The naked DNA construct was present with the RNP complexes during electroporation, with 1 μg of naked DNA construct used per million cells. D-biotin for dissociating the anti-CD3/anti-CD28 stimulatory reagent was not added during the wash step, allowing the anti-CD3/anti-CD28 stimulatory reagent to remain bound to cells during and following electroporation.

### TRAC-Targeting Guide RNA (SEQ ID NO: 255)

agagtctctcagctggtaca

Following electroporation, the cells were incubated overnight at 37° C. in static culture in a complete medium containing recombinant IL-2, IL-15, and IL-7, 1 μM of the CDC7 inhibitor XL413, and 1 μM of the DNA-dependent protein kinase (DNA-PK) inhibitor M-3814. In one condition, additional anti-CD3/anti-CD28 stimulatory reagent was not added following electroporation (TRAC KO, CAR KI), whereas in another condition, additional anti-CD3/anti-CD28 stimulatory was added following electroporation (TRAC KO, CAR KI, Anti-CD3/Anti-CD28). After overnight incubation, the cells were harvested and washed. In this process, recombinant protein-expressing cells were produced in 24 hours following the addition of the whole blood sample to the chromatography column.

Recombinant protein expression was assessed by fluorescent activated cell sorting (FACS). For comparison, cells were left unedited (non-edited) or were subjected to TRAC knockout without transgene knock-in (TRAC KO).

FIG. 1A shows depletion, total nucleated cell count (TNC), and purity for positive fractions collected from multiple whole blood sample loadings, including for cells ultimately subjected to engineering (indicated by a square). Results shown were pre-gated on CD45+ viable, single cells. Together, these results show that the use of the anti-CD3 affinity chromatography column resulted in the selection of CD3-positive T cells from whole blood samples.

FIG. 1B shows CD3 and CAR expression in cells following engineering. As shown, both conditions involving transgene knock-in (TRAC KO, CAR KI; TRAC KO, CAR KI, Anti-CD3/anti-CD28) resulted in CAR-expressing cells, with the presence of anti-CD3/anti-CD28 stimulatory reagent following electroporation not affecting CAR expression. FIG. 1C shows viability, KO efficiency, and KI efficiency over time in cells following engineering. As shown, CAR expression was already at 37% on day seven following electroporation, ultimately further increasing to 48%. FIG. 1D shows cell growth of total cells, KO cells, and KI cells over time following engineering.

Together, these results show that the non-viral engineering of T cells selected from whole blood samples via affinity column chromatography can be successfully performed within 24 hours following the on-column stimulation and elution of the T cells. Without wanting to be limited to a specific mechanism of action, using whole blood, as opposed to apheresed blood, can result in T cells that are in good condition and that can withstand electroporation conditions.

In addition, these results demonstrate that efficient transgene knock-in can be achieved without a 24-hour incubation between the initiation of stimulation and gene editing. Prior reports have indicated that HDR is most efficient for cells in S phase (see Lin et al., 2014, eLife 3:e04766), and that during stimulation, T cells enter an extended G1 phase before entering their first S phase within 30 hours (see Lewis and Ly, 2021, Front. Cell Dev. Biol. 9). Here, the results surprisingly demonstrate that a 24-hour incubation between the initiation of stimulation and gene editing, for instance to allow stimulated T cells to enter S phase, is not needed for efficient transgene knock-in. Without wishing to be bound by theory, by not dissociating or removing the anti-CD3/anti-CD28 stimulatory reagent from T cells prior to electroporation, T cell stimulation can continue following electroporation and during overnight incubation, which may improve transgene knock-in efficiency without the need for extended incubation prior to electroporation.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| No. | Sequence | Description |
|---|---|---|
| 1 | DPSKDSKAQVSAAEAGITGTWYNQLGSTFIVTAGADGALT | Streptavidin |
| | GTYESAVGNAESRYVLTGRYDSAPATDGSGTALGWTVAW | (UniProt No. |
| | KNNYRNAHSATTWSGQYVGGAEARINTQWLLTSGTTEAN | P22629) |
| | AWKSTLVGHDTFTKVKPSAASIDAAKKAGVNNGNPLDAV | Species: |
| | QQ | Streptomyces |
| | | avidinii |
| 2 | MEAGITGTWYNQLGSTFIVTAGADGALTGTYESAVGNAES | Minimal |
| | RYVLTGRYDSAPATDGSGTALGWTVAWKNNYRNAHSAT | streptavidin with |
| | TWSGQYVGGAEARINTQWLLTSGTTEANAWKSTLVGHDT | initial methionine |
| | FTKVKPSAAS | |
| 3 | DPSKDSKAQVSAAEAGITGTWYNQLGSTFIVTAGADGALT | Mutein |
| | GTYVTARGNAESRYVLTGRYDSAPATDGSGTALGWTVA | Streptavidin Val⁴⁴- |
| | WKNNYRNAHSATTWSGQYVGGAEARINTQWLLTSGTTE | Thr⁴⁵-Ala⁴⁶-Arg⁴⁷ |
| | ANAWKSTLVGHDTFTKVKPSAASIDAAKKAGVNNGNPLD | |
| | AVQQ | |
| 4 | MEAGITGTWYNQLGSTFIVTAGADGALTGTYVTARGNAE | Minimal mutein |
| | SRYVLTGRYDSAPATDGSGTALGWTVAWKNNYRNAHSA | Streptavidin Val⁴⁴- |
| | TTWSGQYVGGAEARINTQWLLTSGTTEANAWKSTLVGHD | Thr⁴⁵-Ala⁴⁶-Arg⁴⁷ |
| | TFTKVKPSAAS | with initial |
| | | methionine |
| 5 | DPSKDSKAQVSAAEAGITGTWYNQLGSTFIVTAGADGALT | Mutein |
| | GTYIGARGNAESRYVLTGRYDSAPATDGSGTALGWTVAW | Streptavidin Ile⁴⁴- |
| | KNNYRNAHSATTWSGQYVGGAEARINTQWLLTSGTTEAN | Gly⁴⁵-Ala⁴⁶-Arg⁴⁷ |
| | AWKSTLVGHDTFTKVKPSAASIDAAKKAGVNNGNPLDAV | |
| | QQ | |
| 6 | MEAGITGTWYNQLGSTFIVTAGADGALTGTYIGARGNAES | Minimal mutein |
| | RYVLTGRYDSAPATDGSGTALGWTVAWKNNYRNAHSAT | Streptavidin Ile⁴⁴- |
| | TWSGQYVGGAEARINTQWLLTSGTTEANAWKSTLVGHDT | Gly⁴⁵-Ala⁴⁶-Arg⁴⁷ |
| | FTKVKPSAAS | with initial |
| | | methionine |
| 7 | WRHPQFGG | Streptavidin- |
| | | binding peptide, |
| | | Strep-tag ® |
| 8 | WSHPQFEK | Strep-tag ® II |
| 9 | His-Pro-Xaa | Streptavidin- |
| | | Binding peptide |
| | | Xaa is selected |
| | | from Gln, Asp, and |
| | | Met |
| 10 | His-Pro-Gln-Phe | Streptavidin- |
| | | binding peptide |
| 11 | Xaa₁-Xaa₂-His-Pro-Gln-Phe-Xaa₃-Xaa₄ | Streptavidin- |
| | | binding peptide |
| | | Xaa₁ is Trp, Lys or |
| | | Arg; |
| | | Xaa₂ is any amino |
| | | acid; |
| | | Xaa₃ is Gly or Glu |
| | | Xaa₄ is Gly, Lys or |
| | | Arg |
| 12 | Trp-Xaa₁-His-Pro-Gln-Phe-Xaa₂-Xaa₃ | Streptavidin- |
| | | binding peptide |
| | | Xaa₁ is any amino |
| | | acid; |
| | | Xaa₂ is Gly or Glu |
| | | Xaa₃ is Gly, Lys or |
| | | Arg |
| 13 | Trp-Ser-His-Pro-Gln-Phe-Glu-Lys-(Xaa)ₙ-Trp-Ser-His-Pro-Gln- | Sequential modules |
| | Phe-Glu-Lys | of streptavidin- |
| | | binding peptide |
| | | Xaa is any amino |
| | | acid; |
| | | n is either 8 or 12 |
| 14 | Trp-Ser-His-Pro-Gln-Phe-Glu-Lys-(GlyGlyGlySer)n-Trp-Ser- | Sequential modules |
| | His-Pro-Gln-Phe-Glu-Lys | of streptavidin- |
| | | binding peptide |
| | | n is 2 or 3 |
| 15 | SAWSHPQFEKGGGSGGGSGGGSWSHPQFEK | Twin-Strep-tag |
| 16 | SAWSHPQFEKGGGSGGGSGGSAWSHPQFEK | Twin-Strep-tag |
| 17 | WSHPQFEKGGGSGGGSGGGSWSHPQFEK | Twin-Strep-tag |
| 18 | WSHPQFEKGGGSGGGSWSHPQFEK | Twin-Strep-tag |
| 19 | WSHPQFEKGGGSGGGSGGSAWSHPQFEK | Twin-Strep-tag |
| 20 | Tyr-Pro-Tyr-Asp-Val-Pro-Asp-Tyr-Ala | HA-tag |
| 21 | Tyr-Thr-Asp-Ile-Glu-Met-Asn-Arg-Leu-Gly-Lys | VSV-G-tag |
| 22 | Gln-Pro-Glu-Leu-Ala-Pro-Glu-Asp-Pro-Glu-Asp | HSV-tag |
| 23 | Ala-Ser-Met-Thr-Gly-Gly-Gln-Gln-Met-Gly | T7 epitope |
| 24 | Gln-Pro-Glu-Leu-Ala-Pro-Glu-Asp-Pro-Glu-Asp | HSV epitope |
| 25 | Glu-Gln-Lys-Leu-Ile-Ser-Glu-Glu-Asp-Leu | Myc epitope |
| 26 | Gly-Lys-Pro-Ile-Pro-Asn-Pro-Leu-Leu-Gly-Leu-Asp-Ser-Thr | V5-tag |
| 27 | EAGITGTWYNQLGSTFIVTAGADGALTGTYVTARGNAESR | Minimal mutein |
| | YVLTGRYDSAPATDGSGTALGWTVAWKNNYRNAHSATT | Streptavidin Val⁴⁴- |
| | WSGQYVGGAEARINTQWLLTSGTTEENAGYSTLVGHDTF | Thr⁴⁵-Ala⁴⁶-Arg⁴⁷ |
| | TKVKPSAAS | and Glu¹¹⁷, Gly¹²², |
| | | Tyr¹²¹ (mutein m1- |
| | | 9) without initial |
| | | methionine |
| 28 | DPSKDSKAQVSAAEAGITGTWYNQLGSTFIVTAGADGALT | Mutein |
| | GTYVTARGNAESRYVLTGRYDSAPATDGSGTALGWTVA | Streptavidin Val⁴⁴- |
| | WKNNYRNAHSATTWSGQYVGGAEARINTQWLLTSGTTEE | Thr⁴⁵-Ala⁴⁶-Arg⁴⁷ |
| | NAGYSTLVGHDTFTKVKPSAAS | and Glu¹¹⁷, Gly¹²⁰ |
| | | Tyr¹²¹ (mutein m1-9) |
| 29 | AMQVQLKQSGPGLVQPSQSLSITCTVSGFSLTTFGVHWVR | Variable Heavy |
| | QSPGKGLEWLGVIWASGITDYNVPFMSRLSITKDNSKSQV | chain of Fab |
| | FFKLNSLQPDDTAIYYCAKNDPGTGFAYWGQGTLVTVSA | fragment m13B8.2 |
| | GSTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCGSAWSHPQFEKGGGSGGGS | |
| | GGSAWSHPQFEK | |
| 30 | AMDIQMTQSPASLSASVGETVTFTCRASEMIYSYLAWYQQ | Variable Light |
| | KQGKSPQLLVHDAKTLAEGVPSRFSGGGSGTQFSLKINTL | chain of Fab |
| | QPEDFGTYYCQAHYGNPPTFGGGTKLEIKRGIAAPSVFIFPP | Fragment m13B8.2 |
| | SDEQLKSGTASVVCLLNNFYPREAKVQWKVDNALQSGNS | |
| | QESVTEQDSKDSTYSLSSTLTLSKADYEKHKVYACEVTHQ | |
| | GLSSPVTKSFNRGECGS | |
| 31 | Gln ValGlnLeuGlnGlnSerGlyAlaGluLeuAlaArgProGlyAlaSerVal | Variable Heavy |
| | LysMetSerCysLysAlaSerGlyTyrThrPheThrArgTyrThrMetHisTrp | chain of anti-CD3 |
| | ValLysGlnArgProGlyGlnGlyLeuGluTrpIleGlyTyrIleAsnProSer | antibody OKT3 |
| | ArgGlyTyrThrAsnTyrAsnGlnLysPheLysAspLysAlaThrLeuThrThr | |
| | AspLysSerSerSerThrAlaTyrMetGlnLeuSerSerLeuThrSerGluAsp | |
| | SerAlaValTyrTyrCysAlaArgTyrTyrAspAspHisTyrCysLeuAspTyr | |
| | TrpGlyGlnGlyThrThrLeuThrValSerSer | |
| 32 | GlnIle ValLeuThrGlnSerProAlaIleMetSerAlaSerProGlyGluLysVal | Variable Light |
| | ThrMetThrCysSerAlaSerSerSerValSerTyrMetAsnTrpTyrGlnGln | chain of anti-CD3 |
| | LysSerGlyThrSerProLysArgTrpIleTyrAspThrSerLysLeuAlaSer | antibody OKT3 |
| | GlyValProAlaHisPheArgGlySerGlySerGlyThrSerTyrSerLeuThrIle | |
| | SerGlyMetGluAlaGluAspAlaAlaThrTyrTyrCysGlnGlnTrpSerSer | |
| | AsnProPheThrPheGlySerGlyThrLysLeuGluIleAsn | |
| 33 | LeuGlnGlnSerGlyAlaGluLeuValLysProGlyAlaSerValArgLeuSer | Variable Heavy |
| | CysLysAlaSerGlyTyrThrPheThrGluTyrIleIleHisTrpIleLysLeuArg | chain of anti-CD28 |
| | SerGlyGlnGlyLeuGluTrpIleGlyTrpPheTyrProGlySerAsnAspIle | antibody CD28.3 |
| | GlnTyrAsnAlaLysPheLysGlyLysAlaThrLeuThrAlaAspLysSerSer | |
| | SerThrValTyrMetGluLeuThrGlyLeuThrSerGluAspSerAlaValTyr | |
| | PheCysAlaArgArgAspAspPheSerGlyTyrAspAlaLeuProTyrTrpGly | |
| | GlnGlyThrMetValThrVal | |
| 34 | AspIleGlnMetThrGlnSerProAlaSerLeuSerValSerValGlyGluThr | Variable Light |
| | ValThrIleThrCysArgThrAsnGluAsnIleTyrSerAsnLeuAlaTrpTyrGln | chain of anti-CD28 |
| | GlnLysGlnGlyLysSerProGInLeuLeuIleTyrAlaAlaThrHisLeuVal | antibody CD28.3 |
| | GluGlyValProSerArgPheSerGlySerGlySerGlyThrGlnTyrSerLeu | |
| | LysIleThrSerLeuGlnSerGluAspPheGlyAsnTyrTyrCysGlnHisPheTrp | |
| | GlyThrProCysThrPheGlyGlyGlyThrLysLeuGluIleLysArg | |
| 35 | His-Asn-His-Arg-His-Lys-His-Gly-Gly-Gly-Cys | MAT tag |
| 36 | GlnValGlnLeuValGlnSerGlyAlaGluValLysLysProGlyAla1ThrVal | Variable Heavy |
| | LysIleSerCysLysValSerGlyPheAsnIleLysAspThrTyrIleHisTrp | chain of huOKT8 |
| | ValGlnGlnAlaProGlyLysGlyLeuGluTrpMetGlyArgIleAspProAla | |
| | AsnAspAsnThrLeuTyrAlaSerLysPheGlnGlyArgValThrIleThrAla | |
| | AspThrSerThrAspThrAlaTyr65MetGluLeuSerSerLeuArgSerGluAsp | |
| | ThrAlaValTyrTyrCysAlaArgGlyTyrGlyTyrTyrValPheAspHisTrp | |
| | GlyGlnGlyThrLeuValThrValSerSer | |
| 37 | Asp ValGlnIleThrGlnSerProSerSerLeuSerAlaSerValGlyAspArg | Variable Light |
| | ValThrIleThrCysArgThrSerArgSerIleSerGlnTyrLeuAlaTrpTyrGln | chain of huOKT8 |
| | GlnLysProGlyLysValProLysLeuLeuIleTyrSerGlySerThrLeuGln | |
| | SerGlyValProSerArgPheSerGlySerGlySerGlyThrAspPheThrLeuThr | |
| | IleSerSerLeuGlnProGluAspValAlaThrTyrTyrCysGlnGlnHisAsn | |
| | GluAsnProLeuThrPheGlyGlyGlyThrLysValGluIleLys | |
| 38 | PGGG-(SGGGG)₅-P | Linker |
| | | P is proline, G is |
| | | glycine and S is |
| | | serine |
| 39 | GSADDAKKDAAKKDGKS | Linker |
| 40 | MLLLVTSLLLCELPHPAFLLIP | GMCSFR alpha |
| | | chain |
| 41 | atgcttctcctggtgacaagccttctgctctgtgagttaccacacccagcattcctcctgatccc | GMCSFR alpha |
| | a | chain |
| 42 | MALPVTALLLPLALLLHA | CD8 alpha signal |
| | | peptide |
| 43 | MLLLVTSLLLCELPHPAFLLIPRKVCNGIGIGEFKDSLSINAT | truncated |
| | NIKHFKNCTSISGDLHILPVAFRGDSFTHTPPLDPQELDILKT | epidermal growth |
| | VKEITGFLLIQAWPENRTDLHAFENLEIIRGRTKQHGQFSLA | factor receptor |
| | VVSLNITSLGLRSLKEISDGDVIISGNKNLCYANTINWKKLF | (tEGFR) |
| | GTSGQKTKIISNRGENSCKATGQVCHALCSPEGCWGPEPR | |
| | DCVSCRNVSRGRECVDKCNLLEGEPREFVENSECIQCHPEC | |
| | LPQAMNITCTGRGPDNCIQCAHYIDGPHCVKTCPAGVMGE | |
| | NNTLVWKYADAGHVCHLCHPNCTYGCTGPGLEGCPTNGP | |
| | KIPSIATGMVGALLLLLVVALGIGLFM | |
| 44 | RKVCNGIGIGEFKDSLSINATNIKHFKNCTSISGDLHILPVAF | truncated |
| | RGDSFTHTPPLDPQELDILKTVKEITGFLLIQAWPENRTDLH | epidermal growth |
| | AFENLEIIRGRTKQHGQFSLAVVSLNITSLGLRSLKEISDGD | factor receptor |
| | VIISGNKNLCYANTINWKKLFGTSGQKTKIISNRGENSCKA | (tEGFR) |
| | TGQVCHALCSPEGCWGPEPRDCVSCRNVSRGRECVDKCN | |
| | LLEGEPREFVENSECIQCHPECLPQAMNITCTGRGPDNCIQ | |
| | CAHYIDGPHCVKTCPAGVMGENNTLVWKYADAGHVCHL | |
| | CHPNCTYGCTGPGLEGCPTNGPKIPSIATGMVGALLLLLVV | |
| | ALGIGLFM | |
| 45 | VKQTLNFDLLKLAGDVESNPGP | F2A |
| 46 | QCTNYALLKLAGDVESNPGP | E2A |
| 47 | LEGGGEGRGSLLTCGDVEENPGPR | T2A |
| 48 | EGRGSLLTCGDVEENPGP | T2A |
| 49 | GSGATNFSLLKQAGDVEENPGP | P2A |
| 50 | ATNFSLLKQAGDVEENPGP | P2A |
| 51 | DYGVS | CDR H1 |
| 52 | VIWGSETTYYNSALKS | CDR H2 |
| 53 | YAMDYWG | CDR H3 |
| 54 | HYYYGGSYAMDY | CDR H3 |
| 55 | RASQDISKYLN | CDR L1 |
| 56 | SRLHSGV | CDR L2 |
| 57 | HTSRLHS | CDR L2 |
| 58 | GNTLPYTFG | CDR L3 |
| 59 | QQGNTLPYT | CDR L3 |
| 60 | EVKLQESGPGLVAPSQSLSVTCTVSGVSLPDYGVSWIRQPP | VH |
| | RKGLEWLGVIWGSETTYYNSALKSRLTIIKDNSKSQVFLK | |
| | MNSLQTDDTAIYYCAKHYYYGGSYAMDYWGQGTSVTVSS | |
| 61 | DIQMTQTTSSLSASLGDRVTISCRASQDISKYLNWYQQKPD | VL |
| | GTVKLLIYHTSRLHSGVPSRFSGSGSGTDYSLTISNLEQEDI | |
| | ATYFCQQGNTLPYTFGGGTKLEIT | |
| 62 | GSTSGSGKPGSGEGSTKG | Linker |
| 63 | gacatccagatgacccagaccacctccagcctgagcgccagcctgggcgaccgggtgacc | scFv |
| | atcagctgccgggccagccaggacatcagcaagtacctgaactggtatcagcagaagccc | |
| | gacggcaccgtcaagctgctgatctaccacaccagccggctgcacagcggcgtgcccagc | |
| | cggtttagcggcagcggctccggcaccgactacagcctgaccatctccaacctggaacagg | |
| | aagatatcgccacctacttttgccagcagggcaacacactgccctacacctttggcggcgga | |
| | acaaagctggaaatcaccggcagcacctccggcagcggcaagcctggcagcggcgagg | |
| | gcagcaccaagggcgaggtgaagctgcaggaaagcggccctggcctggtggcccccagc | |
| | cagagcctgagcgtgacctgcaccgtgagcggcgtgagcctgcccgactacggcgtgagc | |
| | tggatccggcagccccccaggaagggcctggaatggctgggcgtgatctggggcagcga | |
| | gaccacctactacaacagcgccctgaagagccggctgaccatcatcaaggacaacagcaa | |
| | gagccaggtgttcctgaagatgaacagcctgcagaccgacgacaccgccatctactactgc | |
| | gccaagcactactactacggcggcagctacgccatggactactggggccagggcaccagc | |
| | gtgaccgtgagcagc | |
| 64 | DIQMTQTTSSLSASLGDRVTISCRASQDISKYLNWYQQKPD | scFv |
| | GTVKLLIYHTSRLHSGVPSRFSGSGSGTDYSLTISNLEQEDI | |
| | ATYFCQQGNTLPYTFGGGTKLEITGSTSGSGKPGSGEGSTK | |
| | GEVKLQESGPGLVAPSQSLSVTCTVSGVSLPDYGVSWIRQP | |
| | PRKGLEWLGVIWGSETTYYNSALKSRLTIIKDNSKSQVFLK | |
| | MNSLQTDDTAIYYCAKHYYYGGSYAMDYWGQGTSVTVSS | |
| 65 | SYWMN | CDR H1 |
| 66 | QIYPGDGDTNYNGKFKG | CDR H2 |
| 67 | KTISSVVDFYFDY | CDR H3 |
| 68 | QQYNRYPYT | CDR L3 |
| 69 | SYWMN | CDR H1 |
| 70 | QIYPGDGDTNYNGKFKG | CDR H2 |
| 71 | EVKLQQSGAELVRPGSSVKISCKASGYAFSSYWMNWVKQ | VH |
| | RPGQGLEWIGQIYPGDGDTNYNGKFKGQATLTADKSSSTA | |
| | YMQLSGLTSEDSAVYFCARKTISSVVDFYFDYWGQGTTVT | |
| | VSS | |
| 72 | DIELTQSPKFMSTSVGDRVSVTCKASQNVGTNVAWYQQK | VL |
| | PGQSPKPLIYSATYRNSGVPDRFTGSGSGTDFTLTITNVQSK | |
| | DLADYFCQQYNRYPYTSGGGTKLEIKR | |
| 73 | KASQNVGTNVA | CDR L1 |
| 74 | SATYRNS | CDR L2 |
| 75 | QQYNRYPYT | CDR L3 |
| 76 | SYWMN | CDR H1 |
| 77 | QIYPGDGDTNYNGKFKG | CDR H2 |
| 78 | KTISSVVDFYFDY | CDR H3 |
| 79 | GGGGSGGGGSGGGGS | Linker |
| 80 | EVKLQQSGAELVRPGSSVKISCKASGYAFSSYWMNWVKQ | scFv |
| | RPGQGLEWIGQIYPGDGDTNYNGKFKGQATLTADKSSSTA | |
| | YMQLSGLTSEDSAVYFCARKTISSVVDFYFDYWGQGTTVT | |
| | VSSGGGGSGGGGSGGGGSDIELTQSPKFMSTSVGDRVSVT | |
| | CKASQNVGTNVAWYQQKPGQSPKPLIYSATYRNSGVPDR | |
| | FTGSGSGTDFTLTITNVQSKDLADYFCQQYNRYPYTSGGG | |
| | TKLEIKR | |
| 81 | ESKYGPPCPPCP | spacer (IgG4hinge) |
| | | Species: Homo |
| | | sapiens |
| 82 | gaatctaagtacggaccgccctgccccccttgccct | spacer (IgG4hinge) |
| | | Species: Homo |
| | | sapiens |
| 83 | ESKYGPPCPPCPGQPREPQVYTLPPSQEEMTKNQVSLTCLV | Hinge-CH3 spacer |
| | KGFYPSDIAVEWESNGQPENNYKTTPPVLDSDGSFFLYSRL | Species: Homo |
| | TVDKSRWQEGNVFSCSVMHEALHNHYTQKSLSLSLGK | sapiens |
| 84 | ESKYGPPCPPCPAPEFLGGPSVFLFPPKPKDTLMISRTPEVT | Hinge-CH2—CH3 |
| | CVVVDVSQEDPEVQFNWYVDGVEVHNAKTKPREEQFNST | spacer |
| | YRVVSVLTVLHQDWLNGKEYKCKVSNKGLPSSIEKTISKA | |
| | KGQPREPQVYTLPPSQEEMTKNQVSLTCLVKGFYPSDIAV | Species: Homo |
| | EWESNGQPENNYKTTPPVLDSDGSFFLYSRLTVDKSRWQE | sapiens |
| | GNVFSCSVMHEALHNHYTQKSLSLSLGK | |
| 85 | RWPESPKAQASSVPTAQPQAEGSLAKATTAPATTRNTGRG | IgD-hinge-Fc |
| | GEEKKKEKEKEEQEERETKTPECPSHTQPLGVYLLTPAVQ | Species: Homo |
| | DLWLRDKATFTCFVVGSDLKDAHLTWEVAGKVPTGGVEE | sapiens |
| | GLLERHSNGSQSQHSRLTLPRSLWNAGTSVTCTLNHPSLPP | |
| | QRLMALREPAAQAPVKLSLNLLASSDPPEAASWLLCEVSG | |
| | FSPPNILLMWLEDQREVNTSGFAPARPPPQPGSTTFWAWS | |
| | VLRVPAPPSPQPATYTCVVSHEDSRTLLNASRSLEVSYVTDH | |
| 86 | GluValValValLysTyrGlyProProCysProProCysPro | Exemplary IgG |
| | | Hinge |
| 87 | X₁PPX₂P | Exemplary IgG |
| | X₁ is glycine, cysteine or arginine | Hinge |
| | X₂ is cysteine or threonine | |
| 88 | GluProLysSerCysAspLysThrHisThrCysProProCysPro | Exemplary IgG |
| | | Hinge |
| 89 | GluArgLysCysCysValGluCysProProCysPro | Exemplary IgG |
| | | Hinge |
| 90 | ELKTPLGDTHTCPRCPEPKSCDTPPPCPRCPEPKSCDTPPPC | Exemplary IgG |
| | PRCPEPKSCDTPPPCPRCP | Hinge |
| 91 | GluSerLysTyrGlyProProCysProSerCysPro | Exemplary IgG |
| | | Hinge |
| 92 | GluSerLysTyrGlyProProCysProProCysPro | Exemplary IgG |
| | | Hinge |
| 93 | TyrGlyProProCysProProCysPro | Exemplary IgG |
| | | Hinge |
| 94 | LysTyrGlyProProCysProProCysPro | Exemplary IgG |
| | | Hinge |
| 95 | FWVLVVVGGVLACYSLLVTVAFIIFWV | CD28 (amino acids |
| | | 153-179 of |
| | | Accession No. |
| | | P10747) |
| | | Species: Homo |
| | | sapien |
| 96 | IEVMYPPPYLDNEKSNGTIIHVKGKHLCPSPLFPGPSKPFW | CD28 (amino acids |
| | VLVVVGGVLACYSLLVTVAFIIFWV | 114-179 of |
| | | Accession No. |
| | | P10747) |
| | | Species: Homo |
| | | sapiens |
| 97 | RSKRSRLLHSDYMNMTPRRPGPTRKHYQPYAPPRDFAAY | CD28 (amino acids |
| | RS | 180-220 of |
| | | P10747) |
| | | Species: Homo |
| | | sapiens |
| 98 | RSKRSRGGHSDYMNMTPRRPGPTRKHYQPYAPPRDFAAY | CD28 (LL to GG) |
| | RS | Species: Homo |
| | | sapiens |
| 99 | KRGRKKLLYIFKQPFMRPVQTTQEEDGCSCRFPEEEEGGCE | 4-1BB (amino |
| | L | acids 214-255 of |
| | | Q07011.1) |
| | | Species: Homo |
| | | sapiens |
| 100 | RVKFSRSADAPAYQQGQNQLYNELNLGRREEYDVLDKRR | CD3 zeta |
| | GRDPEMGGKPRRKNPQEGLYNELQKDKMAEAYSEIGMK | Species: Homo |
| | GERRRGKGHDGLYQGLSTATKDTYDALHMQALPPR | sapiens |
| 101 | RVKFSRSAEPPAYQQGQNQLYNELNLGRREEYDVLDKRR | CD3 zeta |
| | GRDPEMGGKPRRKNPQEGLYNELQKDKMAEAYSEIGMK | Species: Homo |
| | GERRRGKGHDGLYQGLSTATKDTYDALHMQALPPR | sapiens |
| 102 | RVKFSRSADAPAYKQGQNQLYNELNLGRREEYDVLDKRR | CD3 zeta |
| | GRDPEMGGKPRRKNPQEGLYNELQKDKMAEAYSEIGMK | Species: Homo |
| | GERRRGKGHDGLYQGLSTATKDTYDALHMQALPPR | sapiens |
| 103 | EAGITGTWYNQLGSTFIVTAGADGALTGTYESAVGNAESR | Minimal |
| | YVLTGRYDSAPATDGSGTALGWTVAWKNNYRNAHSATT | streptavidin |
| | WSGQYVGGAEARINTQWLLTSGTTEANAWKSTLVGHDTF | without initial |
| | TKVKPSAAS | methionine |
| 104 | EAGITGTWYNQLGSTFIVTAGADGALTGTYVTARGNAESR | Minimal mutein |
| | YVLTGRYDSAPATDGSGTALGWTVAWKNNYRNAHSATT | Streptavidin Val⁴⁴- |
| | WSGQYVGGAEARINTQWLLTSGTTEANAWKSTLVGHDTF | Thr⁴⁵-Ala⁴⁶-Arg⁴⁷ |
| | TKVKPSAAS | without initial |
| | | methionine |
| 105 | EAGITGTWYNQLGSTFIVTAGADGALTGTYIGARGNAESR | Minimal mutein |
| | YVLTGRYDSAPATDGSGTALGWTVAWKNNYRNAHSATT | Streptavidin Ile44- |
| | WSGQYVGGAEARINTQWLLTSGTTEANAWKSTLVGHDTF | Gly45-Ala-46- |
| | TKVKPSAAS | Arg47 without |
| | | initial methionine |
| 106 | ASTKGPSVFPLAPCSRSTSESTAALGCLVKDYFPEPVTVSW | Human IgG2 Fc |
| | NSGALTSGVHTFPAVLQSSGLYSLSSVVTVPSSNFGTQTYT | (Uniprot P01859) |
| | CNVDHKPSNTKVDKTVERKCCVECPPCPAPPVAGPSVFLF | Species: Homo |
| | PPKPKDTLMISRTPEVTCVVVDVSHEDPEVQFNWYVDGVE | sapiens |
| | VHNAKTKPREEQFNSTFRVVSVLTVVHQDWLNGKEYKCK | |
| | VSNKGLPAPIEKTISKTKGQPREPQVYTLPPSREEMTKNQV | |
| | SLTCLVKGFYPSDISVEWESNGQPENNYKTTPPMLDSDGSF | |
| | FLYSKLTVDKSRWQQGNVFSCSVMHEALHNHYTQKSLSL | |
| | SPGK | |
| 107 | ASTKGPSVFPLAPCSRSTSESTAALGCLVKDYFPEPVTVSW | Human IgG4 Fc |
| | NSGALTSGVHTFPAVLQSSGLYSLSSVVTVPSSSLGTKTYT | (Uniprot P01861) |
| | CNVDHKPSNTKVDKRVESKYGPPCPSCPAPEFLGGPSVFLF | Species: Homo |
| | PPKPKDTLMISRTPEVTCVVVDVSQEDPEVQFNWYVDGVE | sapiens |
| | VHNAKTKPREEQFNSTYRVVSVLTVLHQDWLNGKEYKCK | |
| | VSNKGLPSSIEKTISKAKGQPREPQVYTLPPSQEEMTKNQV | |
| | SLTCLVKGFYPSDIAVEWESNGQPENNYKTTPPVLDSDGSF | |
| | FLYSRLTVDKSRWQEGNVFSCSVMHEALHNHYTQKSLSLS | |
| | LGK | |
| 108 | GVQVETISPGDGRTFPKRGQTCVVHYTGMLEDGKKMDSS | FKBP |
| | RDRNKPFKFMLGKQEVIRGWEEGVAQMSVGQRAKLTISP | |
| | DYAYGATGHPGIIPPHATLVFDVELLKLE | |
| 109 | GVQVETISPGDGRTFPKRGQTCVVHYTGMLEDGKKVDSS | FKBP12v36 |
| | RDRNKPFKFMLGKQEVIRGWEEGVAQMSVGQRAKLTISP | |
| | DYAYGATGHPGIIPPHATLVFDVELLKLE | |
| 110 | MGSNKSKPKDASQRRR | Modified acylation |
| | | motif |
| 111 | Met-Gly-Cys-Xaa-Cys | Dual acylation |
| | | motif |
| 112 | Cys-Ala-Ala-Xaa | Acylation region |
| 113 | DYSIN | CDR-H1 |
| 114 | WINTETREPAYAYDFRG | CDR-H2 |
| 115 | DYSYAMDY | CDR-H3 |
| 116 | RASESVTILGSHLIH | CDR-L1 |
| 117 | LASNVQT | CDR-L2 |
| 118 | LQSRTIPRT | CDR-L3 |
| 119 | QIQLVQSGPELKKPGETVKISCKASGYTFTDYSINWVKRAP | Variable heavy |
| | GKGLKWMGWINTETREPAYAYDFRGRFAFSLETSASTAY | (VH) Anti-BCMA |
| | LQINNLKYEDTATYFCALDYSYAMDYWGQGTSVTVSS | |
| 120 | DIVLTQSPPSLAMSLGKRATISCRASESVTILGSHLIHWYQQ | Variable light (VL) |
| | KPGQPPTLLIQLASNVQTGVPARFSGSGSRTDFTLTIDPVEE | Anti-BCMA |
| | DDVAVYYCLQSRTIPRTFGGGTKLEIK | |
| 121 | DIVLTQSPPSLAMSLGKRATISCRASESVTILGSHLIHWYQQ | scFv |
| | KPGQPPTLLIQLASNVQTGVPARFSGSGSRTDFTLTIDPVEE | |
| | DDVAVYYCLQSRTIPRTFGGGTKLEIKGSTSGSGKPGSGEG | |
| | STKGQIQLVQSGPELKKPGETVKISCKASGYTFTDYSINWV | |
| | KRAPGKGLKWMGWINTETREPAYAYDFRGRFAFSLETSA | |
| | STAYLQINNLKYEDTATYFCALDYSYAMDYWGQGTSVTV | |
| | SS | |
| 122 | DIVLTQSPPSLAMSLGKRATISCRASESVTILGSHLIHWYQQ | anti-BCMA CAR |
| | KPGQPPTLLIQLASNVQTGVPARFSGSGSRTDFTLTIDPVEE | |
| | DDVAVYYCLQSRTIPRTFGGGTKLEIKGSTSGSGKPGSGEG | |
| | STKGQIQLVQSGPELKKPGETVKISCKASGYTFTDYSINWV | |
| | KRAPGKGLKWMGWINTETREPAYAYDFRGRFAFSLETSA | |
| | STAYLQINNLKYEDTATYFCALDYSYAMDYWGQGTSVTV | |
| | SSAAATTTPAPRPPTPAPTIASQPLSLRPEACRPAAGGAVHT | |
| | RGLDFACDIYIWAPLAGTCGVLLLSLVITLYCKRGRKKLLY | |
| | IFKQPFMRPVQTTQEEDGCSCRFPEEEEGGCELRVKFSRSA | |
| | DAPAYQQGQNQLYNELNLGRREEYDVLDKRRGRDPEMG | |
| | GKPRRKNPQEGLYNELQKDKMAEAYSEIGMKGERRRGKG | |
| | HDGLYQGLSTATKDTYDALHMQALPPR | |
| 123 | DYYVY | CDR-H1 |
| 124 | WINPNSGGTNYAQKFQG | CDR-H2 |
| 125 | SQRDGYMDY | CDR-H3 |
| 126 | TGTSSDVG | CDR-L1 |
| 127 | EDSKRPS | CDR-L2 |
| 128 | SSNTRSSTLV | CDR-L3 |
| 129 | EVQLVQSGAEMKKPGASLKLSCKASGYTFIDYYVYWMRQ | Variable heavy |
| | APGQGLESMGWINPNSGGTNYAQKFQGRVTMTRDTSISTA | (VH) Anti-BCMA |
| | YMELSRLRSDDTAMYYCARSQRDGYMDYWGQGTLVTVSS | |
| 130 | QSALTQPASVSASPGQSIAISCTGTSSDVGWYQQHPGKAPK | Variable light (VL) |
| | LMIYEDSKRPSGVSNRFSGSKSGNTASLTISGLQAEDEADY | Anti-BCMA |
| | YCSSNTRSSTLVFGGGTKLTVLG | |
| 131 | QSALTQPASVSASPGQSIAISCTGTSSDVGWYQQHPGKAPK | scFv |
| | LMIYEDSKRPSGVSNRFSGSKSGNTASLTISGLQAEDEADY | |
| | YCSSNTRSSTLVFGGGTKLTVLGSRGGGGSGGGGSGGGGS | |
| | LEMAEVQLVQSGAEMKKPGASLKLSCKASGYTFIDYYVY | |
| | WMRQAPGQGLESMGWINPNSGGTNYAQKFQGRVTMTRD | |
| | TSISTAYMELSRLRSDDTAMYYCARSQRDGYMDYWGQGT | |
| | LVTVSS | |
| 132 | QSALTQPASVSASPGQSIAISCTGTSSDVGWYQQHPGKAPK | anti-BCMA CAR |
| | LMIYEDSKRPSGVSNRFSGSKSGNTASLTISGLQAEDEADY | |
| | YCSSNTRSSTLVFGGGTKLTVLGSRGGGGSGGGGSGGGGS | |
| | LEMAEVQLVQSGAEMKKPGASLKLSCKASGYTFIDYYVY | |
| | WMRQAPGQGLESMGWINPNSGGTNYAQKFQGRVTMTRD | |
| | TSISTAYMELSRLRSDDTAMYYCARSQRDGYMDYWGQGT | |
| | LVTVSSESKYGPPCPPCPAPPVAGPSVFLFPPKPKDTLMISR | |
| | TPEVTCVVVDVSQEDPEVQFNWYVDGVEVHNAKTKPREE | |
| | QFQSTYRVVSVLTVLHQDWLNGKEYKCKVSNKGLPSSIEK | |
| | TISKAKGQPREPQVYTLPPSQEEMTKNQVSLTCLVKGFYPS | |
| | DIAVEWESNGQPENNYKTTPPVLDSDGSFFLYSRLTVDKS | |
| | RWQEGNVFSCSVMHEALHNHYTQKSLSLSLGKMFWVLV | |
| | VVGGVLACYSLLVTVAFIIFWVKRGRKKLLYIFKQPFMRP | |
| | VQTTQEEDGCSCRFPEEEEGGCELRVKFSRSADAPAYQQG | |
| | QNQLYNELNLGRREEYDVLDKRRGRDPEMGGKPRRKNPQ | |
| | EGLYNELQKDKMAEAYSEIGMKGERRRGKGHDGLYQGLS | |
| | TATKDTYDALHMQALPPR | |
| 133 | IGAR | Sequence of mutein |
| | | streptavidin at |
| | | sequence positions |
| | | corresponding to |
| | | positions 44 to 47 |
| | | of streptavidin |
| 134 | VTAR | Sequence of mutein |
| | | streptavidin at |
| | | sequence positions |
| | | corresponding to |
| | | positions 44 to 47 |
| | | of streptavidin |
| 135 | AEAGITGTWYNQLGSTFIVTAGADGALTGTYESAVGNAES | Minimal |
| | RYVLTGRYDSAPATDGSGTALGWTVAWKNNYRNAHSAT | streptavidin |
| | TWSGQYVGGAEARINTQWLLTSGTTEANAWKSTLVGHDT | |
| | FTKVKPSAAS | |
| 136 | MEAGITGTWYNQLGSTFIVTAGADGALTGTYVTARGNAE | Mutein |
| | SRYVLTGRYDSAPATDGSGTALGWTVAWKNNYRNAHSA | Streptavidin Val⁴⁴- |
| | TTWSGQYVGGAEARINTQWLLTSGTTEENAGYSTLVGHD | Thr⁴⁵-Ala⁴⁶-Arg⁴⁷ |
| | TFTKVKPSAAS | and Glu¹¹⁷, Gly¹²⁰, |
| | | Tyr¹²¹ (mutein m1- |
| | | 9) with initial |
| | | methionine |
| 137 | PNIQNPDPAVYQLRDSKSSDKSVCLFTDFDSQTNVSQSKDS | Human TCR alpha |
| | DVYITDKTVLDMRSMDFKSNSAVAWSNKSDFACANAFNN | constant (Uniprot |
| | SIIPEDTFFPSPESSCDVKLVEKSFETDTNLNFQNLSVIGFRIL | P01848) |
| | LLKVAGFNLLMTLRLWSS | |
| 138 | NIQNPDPAVYQLRDSKSSDKSVCLFTDFDSQTNVSQSKDS | Human TCR alpha |
| | DVYITDKTVLDMRSMDFKSNSAVAWSNKSDFACANAFNN | constant (Genbank |
| | SIIPEDTFFPSPESSCDVKLVEKSFETDTNLNFQNLSVIGFRIL | Accession No. |
| | LLKVAGFNLLMTLRLWSS | CAA26636.1) |
| 139 | ttttgaaacccttcaaaggcagagacttgtccagcctaacctgcctgctgctcctagctcctga | Human TCR alpha |
| | ggctcagggcccttggcttctgtccgctctgctcagggccctccagcgtggccactgctcag | constant |
| | ccatgctcctgctgctcgtcccagtgctcgaggtgatttttaccctgggaggaaccagagccc | (GenBank: |
| | agtcggtgacccagcttggcagccacgtctctgtctctgaaggagccctggttctgctgaggt | X02592.1) |
| | gcaactactcatcgtctgttccaccatatctcttctggtatgtgcaataccccaaccaaggactc | |
| | cagcttctcctgaagtacacatcagcggccaccctggttaaaggcatcaacggttttgaggct | |
| | gaatttaagaagagtgaaacctccttccacctgacgaaaccctcagcccatatgagcgacgc | |
| | ggctgagtacttctgtgctgtgagtgatctcgaaccgaacagcagtgcttccaagataatctttg | |
| | gatcagggaccagactcagcatccggccaaatatccagaaccctgaccctgccgtgtacca | |
| | gctgagagactctaaatccagtgacaagtctgtctgcctattcaccgattttgattctcaaacaa | |
| | atgtgtcacaaagtaaggattctgatgtgtatatcacagacaaaactgtgctagacatgaggtc | |
| | tatggacttcaagagcaacagtgctgtggcctggagcaacaaatctgactttgcatgtgcaaa | |
| | cgccttcaacaacagcattattccagaagacaccttcttccccagcccagaaagttcctgtgat | |
| | gtcaagctggtcgagaaaagctttgaaacagatacgaacctaaactttcaaaacctgtcagtg | |
| | attgggttccgaatcctcctcctgaaagtggccgggtttaatctgctcatgacgctgcggctgt | |
| | ggtccagctgagatctgcaagattgtaagacagcctgtgctccctcgctccttcctctgcattg | |
| | cccctcttctccctctccaaacagagggaactctcctacccccaaggaggtgaaagctgctac | |
| | cacctctgtgcccccccggtaatgccaccaactggatcctacccgaatttatgattaagattgct | |
| | gaagagctgccaaacactgctgccaccccctctgttcccttattgctgcttgtcactgcctgac | |
| | attcacggcagaggcaaggctgctgcagcctcccctggctgtgcacattccctcctgctcccc | |
| | agagactgcctccgccatcccacagatgatggatcttcagtgggttctcttgggctctaggtcc | |
| | tggagaatgttgtgaggggtttatttttttttaatagtgttcataaagaaatacatagtattcttcttct | |
| | caagacgtggggggaaattatctcattatcgaggccctgctatgctgtgtgtctgggcgtgttg | |
| | tatgtcctgctgccgatgccttcattaaaatgatttggaa | |
| 140 | EDLNKVFPPEVAVFEPSEAEISHTQKATLVCLATGFFPDHV | Human TCR beta |
| | ELSWWVNGKEVHSGVSTDPQPLKEQPALNDSRYCLSSRLR | constant 1 (Uniprot |
| | VSATFWQNPRNHFRCQVQFYGLSENDEWTQDRAKPVTQI | P01850) |
| | VSAEAWGRADCGFTSVSYQQGVLSATILYEILLGKATLYA | |
| | VLVSALVLMAMVKRKDF | |
| 141 | DLKNVFPPEVAVFEPSEAEISHTQKATLVCLATGFYPDHVE | Human TCR beta |
| | LSWWVNGKEVHSGVSTDPQPLKEQPALNDSRYCLSSRLR | constant 2 (Uniprot |
| | VSATFWQNPRNHFRCQVQFYGLSENDEWTQDRAKPVTQI | A0A5B9) |
| | VSAEAWGRADCGFTSESYQQGVLSATILYEILLGKATLYA | |
| | VLVSALVLMAMVKRKDSRG | |
| 142 | EDLKNVFPPEVAVFEPSEAEISHTQKATLVCLATGFYPDHV | Human TCR beta |
| | ELSWWVNGKEVHSGVSTDPQPLKEQPALNDSRYCLSSRLR | constant (Uniprot |
| | VSATFWQNPRNHFRCQVQFYGLSENDEWTQDRAKPVTQI | Accession No. |
| | VSAEAWGRADCGFTSESYQQGVLSATILYEILLGKATLYA | A0A0G2JNG9) |
| | VLVSALVLMAMVKRKDSRG | |
| 143 | ctggtctagaatattccacatctgctctcactctgccatggactcctggaccttctgctgtgtgtc | Human TCR beta |
| | cctttgcatcctggtagcgaagcatacagatgctggagttatccagtcaccccgccatgaggt | constant |
| | gacagagatgggacaagaagtgactctgagatgtaaaccaatttcaggccacaactccctttt | (GenBank: |
| | ctggtacagacagaccatgatgcggggactggagttgctcatttactttaacaacaacgttccg | X00437.1) |
| | atagatgattcagggatgcccgaggatcgattctcagctaagatgcctaatgcatcattctcca | |
| | ctctgaagatccagccctcagaacccagggactcagctgtgtacttctgtgccagcagtttctc | |
| | gacctgttcggctaactatggctacaccttcggttcggggaccaggttaaccgttgtagagga | |
| | cctgaacaaggtgttcccacccgaggtcgctgtgtttgagccatcagaagcagagatctccc | |
| | acacccaaaaggccacactggtgtgcctggccacaggcttcttccccgaccacgtggagct | |
| | gagctggtgggtgaatgggaaggaggtgcacagtggggtcagcacagacccgcagcccc | |
| | tcaaggagcagcccgccctcaatgactccagatactgcctgagcagccgcctgagggtctc | |
| | ggccaccttctggcagaacccccgcaaccacttccgctgtcaagtccagttctacgggctctc | |
| | ggagaatgacgagtggacccaggatagggccaaacccgtcacccagatcgtcagcgccg | |
| | aggcctggggtagagcagactgtggctttacctcggtgtcctaccagcaaggggtcctgtct | |
| | gccaccatcctctatgagatcctgctagggaaggccaccctgtatgctgtgctggtcagcgcc | |
| | cttgtgttgatggccatggtcaagagaaaggatttctgaaggcagccctggaagtggagttag | |
| | gagcttctaacccgtcatggttcaatacacattcttcttttgccagcgcttctgaagagctgctct | |
| | cacctctctgcatcccaatagatatccccctatgtgcatgcacacctgcacactcacggctgaa | |
| | atctccctaacccagggggac | |
| 144 | agagucucucagcugguaca | TRAC gRNA |
| 145 | ucucucagcugguacacggc | TRAC-10 |
| 146 | uggauuuagagucucucagc | TRAC-110 |
| 147 | acacggcagggucaggguuc | TRAC-116 |
| 148 | gagaaucaaaaucggugaau | TRAC-16 |
| 149 | gcugguacacggcaggguca | TRAC-4 |
| 150 | cucagcugguacacggc | TRAC-49 |
| 151 | ugguacacggcaggguc | TRAC-2 |
| 152 | gcuagacaugaggucua | TRAC-30 |
| 153 | gucagauuuguugcucc | TRAC-43 |
| 154 | ucagcugguacacggca | TRAC-23 |
| 155 | gcagacagacuugucac | TRAC-34 |
| 156 | gguacacggcaggguca | TRAC-25 |
| 157 | cuucaagagcaacagugcug | TRAC-128 |
| 158 | agagcaacagugcuguggcc | TRAC-105 |
| 159 | aaagucagauuuguugcucc | TRAC-106 |
| 160 | acaaaacugugcuagacaug | TRAC-123 |
| 161 | aaacugugcuagacaug | TRAC-64 |
| 162 | ugugcuagacaugaggucua | TRAC-97 |
| 163 | ggcuggggaagaaggugucuuc | TRAC-148 |
| 164 | gcuggggaagaaggugucuuc | TRAC-147 |
| 165 | ggggaagaaggugucuuc | TRAC-234 |
| 166 | guuuugucugugauauacacau | TRAC-167 |
| 167 | ggcagacagacuugucacuggauu | TRAC-177 |
| 168 | gcagacagacuugucacuggauu | TRAC-176 |
| 169 | gacagacuugucacuggauu | TRAC-257 |
| 170 | gugaauaggcagacagacuuguca | TRAC-233 |
| 171 | gaauaggcagacagacuuguca | TRAC-231 |
| 172 | gagucucucagcugguacacgg | TRAC-163 |
| 173 | gucucucagcugguacacgg | TRAC-241 |
| 174 | gguacacggcagggucaggguu | TRAC-179 |
| 175 | guacacggcagggucaggguu | TRAC-178 |
| 176 | cacccagaucgucagcgccg | TRBC-40 |
| 177 | caaacacagcgaccucgggu | TRBC-52 |
| 178 | ugacgaguggacccaggaua | TRBC-25 |
| 179 | ggcucucggagaaugacgag | TRBC-35 |
| 180 | ggccucggcgcugacgaucu | TRBC-50 |
| 181 | gaaaaacguguucccacccg | TRBC-39 |
| 182 | augacgaguggacccaggau | TRBC-49 |
| 183 | aguccaguucuacgggcucu | TRBC-51 |
| 184 | cgcugucaaguccaguucua | TRBC-26 |
| 185 | aucgucagcgccgaggccug | TRBC-47 |
| 186 | ucaaacacagcgaccucggg | TRBC-45 |
| 187 | cguagaacuggacuugacag | TRBC-34 |
| 188 | aggccucggcgcugacgauc | TRBC-227 |
| 189 | ugacagcggaagugguugcg | TRBC-41 |
| 190 | uugacagcggaagugguugc | TRBC-30 |
| 191 | ucuccgagagcccguagaac | TRBC-206 |
| 192 | cgggugggaacacguuuuuc | TRBC-32 |
| 193 | gacagguuuggcccuauccu | TRBC-276 |
| 194 | gaucgucagcgccgaggccu | TRBC-274 |
| 195 | ggcucaaacacagcgaccuc | TRBC-230 |
| 196 | ugagggucucggccaccuuc | TRBC-235 |
| 197 | aggcuucuaccccgaccacg | TRBC-38 |
| 198 | ccgaccacguggagcugagc | TRBC-223 |
| 199 | ugacagguuuggcccuaucc | TRBC-221 |
| 200 | cuugacagcggaagugguug | TRBC-48 |
| 201 | agaucgucagcgccgaggcc | TRBC-216 |
| 202 | gcgcugacgaucugggugac | TRBC-210 |
| 203 | ugaggggggcugcuccuug | TRBC-268 |
| 204 | guugcggggguucugccaga | TRBC-193 |
| 205 | agcucagcuccacguggucg | TRBC-246 |
| 206 | gcggcugcucaggcaguauc | TRBC-228 |
| 207 | gcggggguucugccagaagg | TRBC-43 |
| 208 | uggcucaaacacagcgaccu | TRBC-272 |
| 209 | acuggacuugacagcggaag | TRBC-33 |
| 210 | gacagcggaagugguugcgg | TRBC-44 |
| 211 | gcugucaaguccaguucuac | TRBC-211 |
| 212 | guaucuggagucauugaggg | TRBC-253 |
| 213 | cucggcgcugacgaucu | TRBC-18 |
| 214 | ccucggcgcugacgauc | TRBC-6 |
| 215 | ccgagagcccguagaac | TRBC-85 |
| 216 | ccagaucgucagcgccg | TRBC-129 |
| 217 | gaaugacgaguggaccc | TRBC-93 |
| 218 | gggugacagguuuggcccuauc | TRBC-415 |
| 219 | ggugacagguuuggcccuauc | TRBC-414 |
| 220 | gugacagguuuggcccuauc | TRBC-310 |
| 221 | gacagguuuggcccuauc | TRBC-308 |
| 222 | gauacugccugagcagccgccu | TRBC-401 |
| 223 | gaccacguggagcugagcuggugg | TRBC-468 |
| 224 | guggagcugagcuggugg | TRBC-462 |
| 225 | ggggggcugcuccuugaggggcu | TRBC-424 |
| 226 | gggggcugcuccuugaggggcu | TRBC-423 |
| 227 | gcgggcugcuccuugaggggcu | TRBC-422 |
| 228 | gggcugcuccuugaggggcu | TRBC-420 |
| 229 | ggcugcuccuugaggggcu | TRBC-419 |
| 230 | gcugcuccuugaggggcu | TRBC-418 |
| 231 | ggugaaugggaaggaggugcacag | TRBC-445 |
| 232 | gugaaugggaaggaggugcacag | TRBC-444 |
| 233 | gaaugggaaggaggugcacag | TRBC-442 |
| 234 | agcgctctcgtacagagttggcattataatacgactcactataggggagaatcaaaatcggtg | TRAC gRNA |
| | aatgttttagagctagaaatagcaagttaaaataaggctagtccgttatcaacttgaaaaagtgg | |
| | caccgagtcggtgcttttttt | |
| 235 | gagaaucaaaaucggugaauguuuuagagcuagaaauagcaaguuaaaauaaggcu | TRAC gRNA |
| | aguccguuaucaacuugaaaaaguggcaccgagucggugcuuuu | |
| 236 | KKPYSIGLDIGTNSVGWAVVTDDYKVPAKKMKVLGNTDK | Streptococcus |
| | SHIEKNLLGALLFDSGNTAEDRRLKRTARRRYTRRRNRILY | mutans Cas9 |
| | LQEIFSEEMGKVDDSFFHRLEDSFLVTEDKRGERHPIFGNL | |
| | EEEVKYHENFPTIYHLRQYLADNPEKVDLRLVYLALAHIIK | |
| | FRGHFLIEGKFDTRNNDVQRLFQEFLAVYDNTFENSSLQEQ | |
| | NVQVEEILTDKISKSAKKDRVLKLFPNEKSNGRFAEFLKLI | |
| | VGNQADFKKHFELEEKAPLQFSKDTYEEELEVLLAQIGDN | |
| | YAELFLSAKKLYDSILLSGILTVTDVGTKAPLSASMIQRYN | |
| | EHQMDLAQLKQFIRQKLSDKYNEVFSDVSKDGYAGYIDG | |
| | KTNQEAFYKYLKGLLNKIEGSGYFLDKIEREDFLRKQRTFD | |
| | NGSIPHQIHLQEMRAIIRRQAEFYPFLADNQDRIEKLLTFRIP | |
| | YYVGPLARGKSDFAWLSRKSADKITPWNFDEIVDKESSAE | |
| | AFINRMTNYDLYLPNQKVLPKHSLLYEKFTVYNELTKVKY | |
| | KTEQGKTAFFDANMKQEIFDGVFKVYRKVTKDKLMDFLE | |
| | KEFDEFRIVDLTGLDKENKVFNASYGTYHDLCKILDKDFL | |
| | DNSKNEKILEDIVLTLTLFEDREMIRKRLENYSDLLTKEQV | |
| | KKLERRHYTGWGRLSAELIHGIRNKESRKTILDYLIDDGNS | |
| | NRNFMQLINDDALSFKEEIAKAQVIGETDNLNQVVSDIAGS | |
| | PAIKKGILQSLKIVDELVKIMGHQPENIVVEMARENQFTNQ | |
| | GRRNSQQRLKGLTDSIKEFGSQILKEHPVENSQLQNDRLFL | |
| | YYLQNGRDMYTGEELDIDYLSQYDIDHIIPQAFIKDNSIDN | |
| | RVLTSSKENRGKSDDVPSKDVVRKMKSYWSKLLSAKLITQ | |
| | RKFDNLTKAERGGLTDDDKAGFIKRQLVETRQITKHVARI | |
| | LDERFNTETDENNKKIRQVKIVTLKSNLVSNFRKEFELYKV | |
| | REINDYHHAHDAYLNAVIGKALLGVYPQLEPEFVYGDYPH | |
| | FHGHKENKATAKKFFYSNIMNFFKKDDVRTDKNGEIIWKK | |
| | DEHISNIKKVLSYPQVNIVKKVEEQTGGFSKESILPKGNSD | |
| | KLIPRKTKKFYWDTKKYGGFDSPIVAYSILVIADIEKGKSK | |
| | KLKTVKALVGVTIMEKMTFERDPVAFLERKGYRNVQEENI | |
| | IKLPKYSLFKLENGRKRLLASARELQKGNEIVLPNHLGTLL | |
| | YHAKNIHKVDEPKHLDYVDKHKDEFKELLDVVSNFSKKY | |
| | TLAEGNLEKIKELYAQNNGEDLKELASSFINLLTFTAIGAPA | |
| | TFKFFDKNIDRKRYTSTTEILNATLIHQSITGLYETRIDLNKL | |
| | GGD | |
| 237 | DKKYSIGLDIGTNSVGWAVITDEYKVPSKKFKVLGNTDRH | Streptococcus |
| | SIKKNLIGALLFDSGETAEATRLKRTARRRYTRRKNRICYL | pyogenes Cas9 |
| | QEIFSNEMAKVDDSFFHRLEESFLVEEDKKHERHPIFGNIV | |
| | DEVAYHEKYPTIYHLRKKLVDSTDKADLRLIYLALAHMIK | |
| | FRGHFLIEGDLNPDNSDVDKLFIQLVQTYNQLFEENPINAS | |
| | GVDAKAILSARLSKSRRLENLIAQLPGEKKNGLFGNLIALS | |
| | LGLTPNFKSNFDLAEDAKLQLSKDTYDDDLDNLLAQIGDQ | |
| | YADLFLAAKNLSDAILLSDILRVNTEITKAPLSASMIKRYDE | |
| | HHQDLTLLKALVRQQLPEKYKEIFFDQSKNGYAGYIDGGA | |
| | SQEEFYKFIKPILEKMDGTEELLVKLNREDLLRKQRTFDNG | |
| | SIPHQIHLGELHAILRRQEDFYPFLKDNREKIEKILTFRIPYY | |
| | VGPLARGNSRFAWMTRKSEETITPWNFEEVVDKGASAQSF | |
| | IERMTNFDKNLPNEKVLPKHSLLYEYFTVYNELTKVKYVT | |
| | EGMRKPAFLSGEQKKAIVDLLFKTNRKVTVKQLKEDYFK | |
| | KIECFDSVEISGVEDRFNASLGTYHDLLKIIKDKDFLDNEEN | |
| | EDILEDIVLTLTLFEDREMIEERLKTYAHLFDDKVMKQLKR | |
| | RRYTGWGRLSRKLINGIRDKQSGKTILDFLKSDGFANRNF | |
| | MQLIHDDSLTFKEDIQKAQVSGQGDSLHEHIANLAGSPAIK | |
| | KGILQTVKVVDELVKVMGRHKPENIVIEMARENQTTQKG | |
| | QKNSRERMKRIEEGIKELGSQILKEHPVENTQLQNEKLYLY | |
| | YLQNGRDMYVDQELDINRLSDYDVDHIVPQSFLKDDSIDN | |
| | KVLTRSDKNRGKSDNVPSEEVVKKMKNYWRQLLNAKLIT | |
| | QRKFDNLTKAERGGLSELDKAGFIKRQLVETRQITKHVAQI | |
| | LDSRMNTKYDENDKLIREVKVITLKSKLVSDFRKDFQFYK | |
| | VREINNYHHAHDAYLNAVVGTALIKKYPKLESEFVYGDY | |
| | KVYDVRKMIAKSEQEIGKATAKYFFYSNIMNFFKTEITLAN | |
| | GEIRKRPLIETNGETGEIVWDKGRDFATVRKVLSMPQVNIV | |
| | KKTEVQTGGFSKESILPKRNSDKLIARKKDWDPKKYGGFD | |
| | SPTVAYSVLVVAKVEKGKSKKLKSVKELLGITIMERSSFEK | |
| | NPIDFLEAKGYKEVKKDLIIKLPKYSLFELENGRKRMLASA | |
| | GELQKGNELALPSKYVNFLYLASHYEKLKGSPEDNEQKQL | |
| | FVEQHKHYLDEIIEQISEFSKRVILADANLDKVLSAYNKHR | |
| | DKPIREQAENIIHLFTLTNLGAPAAFKYFDTTIDRKRYTSTK | |
| | EVLDATLIHQSITGLYETRIDLSQLGGD | |
| 238 | TKPYSIGLDIGTNSVGWAVTTDNYKVPSKKMKVLGNTSK | Streptococcus |
| | KYIKKNLLGVLLFDSGITAEGRRLKRTARRRYTRRRNRILY | thermophilus Cas9 |
| | LQEIFSTEMATLDDAFFQRLDDSFLVPDDKRDSKYPIFGNL | |
| | VEEKAYHDEFPTIYHLRKYLADSTKKADLRLVYLALAHMI | |
| | KYRGHFLIEGEFNSKNNDIQKNFQDFLDTYNAIFESDLSLE | |
| | NSKQLEEIVKDKISKLEKKDRILKLFPGEKNSGIFSEFLKLIV | |
| | GNQADFRKCFNLDEKASLHFSKESYDEDLETLLGYIGDDY | |
| | SDVFLKAKKLYDAILLSGFLTVTDNETEAPLSSAMIKRYNE | |
| | HKEDLALLKEYIRNISLKTYNEVFKDDTKNGYAGYIDGKT | |
| | NQEDFYVYLKKLLAEFEGADYFLEKIDREDFLRKQRTFDN | |
| | GSIPYQIHLQEMRAILDKQAKFYPFLAKNKERIEKILTFRIP | |
| | YYVGPLARGNSDFAWSIRKRNEKITPWNFEDVIDKESSAE | |
| | AFINRMTSFDLYLPEEKVLPKHSLLYETFNVYNELTKVRFI | |
| | AESMRDYQFLDSKQKKDIVRLYFKDKRKVTDKDIIEYLHA | |
| | IYGYDGIELKGIEKQFNSSLSTYHDLLNIINDKEFLDDSSNE | |
| | AIIEEIIHTLTIFEDREMIKQRLSKFENIFDKSVLKKLSRRHYT | |
| | GWGKLSAKLINGIRDEKSGNTILDYLIDDGISNRNFMQLIH | |
| | DDALSFKKKIQKAQIIGDEDKGNIKEVVKSLPGSPAIKKGIL | |
| | QSIKIVDELVKVMGGRKPESIVVEMARENQYTNQGKSNSQ | |
| | QRLKRLEKSLKELGSKILKENIPAKLSKIDNNALQNDRLYL | |
| | YYLQNGKDMYTGDDLDIDRLSNYDIDHIIPQAFLKDNSIDN | |
| | KVLVSSASNRGKSDDVPSLEVVKKRKTFWYQLLKSKLISQ | |
| | RKFDNLTKAERGGLSPEDKAGFIQRQLVETRQITKHVARLL | |
| | DEKFNNKKDENNRAVRTVKIITLKSTLVSQFRKDFELYKV | |
| | REINDFHHAHDAYLNAVVASALLKKYPKLEPEFVYGDYP | |
| | KYNSFRERKSATEKVYFYSNIMNIFKKSISLADGRVIERPLI | |
| | EVNEETGESVWNKESDLATVRRVLSYPQVNVVKKVEEQN | |
| | HGLDRGKPKGLFNANLSSKPKPNSNENLVGAKEYLDPKK | |
| | YGGYAGISNSFTVLVKGTIEKGAKKKITNVLEFQGISILDRI | |
| | NYRKDKLNFLLEKGYKDIELIIELPKYSLFELSDGSRRMLA | |
| | SILSTNNKRGEIHKGNQIFLSQKFVKLLYHAKRISNTINENH | |
| | RKYVENHKKEFEELFYYILEFNENYVGAKKNGKLLNSAFQ | |
| | SWQNHSIDELCSSFIGPTGSERKGLFELTSRGSAADFEFLGV | |
| | KIPRYRDYTPSSLLKDATLIHQSVTGLYETRIDLAKLGEG | |
| | KKPYTIGLDIGTNSVGWAVLTDQYDLVKRKMKIAGDSEK | |
| 239 | KQIKKNFWGVRLFDEGQTAADRRMARTARRRIERRRNRIS | Listeria innocua |
| | YLQGIFAEEMSKTDANFFCRLSDSFYVDNEKRNSRHPFFAT | Cas9 |
| | IEEEVEYHKNYPTIYHLREELVNSSEKADLRLVYLALAHIIK | |
| | YRGNFLIEGALDTQNTSVDGIYKQFIQTYNQVFASGIEDGS | |
| | LKKLEDNKDVAKILVEKVTRKEKLERILKLYPGEKSAGMF | |
| | AQFISLIVGSKGNFQKPFDLIEKSDIECAKDSYEEDLESLLA | |
| | LIGDEYAELFVAAKNAYSAVVLSSIITVAETETNAKLSASM | |
| | IERFDTHEEDLGELKAFIKLHLPKHYEEIFSNTEKHGYAGYI | |
| | DGKTKQADFYKYMKMTLENIEGADYFIAKIEKENFLRKQR | |
| | TFDNGAIPHQLHLEELEAILHQQAKYYPFLKENYDKIKSLV | |
| | TFRIPYFVGPLANGQSEFAWLTRKADGEIRPWNIEEKVDFG | |
| | KSAVDFIEKMTNKDTYLPKENVLPKHSLCYQKYLVYNELT | |
| | KVRYINDQGKTSYFSGQEKEQIFNDLFKQKRKVKKKDLEL | |
| | FLRNMSHVESPTIEGLEDSFNSSYSTYHDLLKVGIKQEILDN | |
| | PVNTEMLENIVKILTVFEDKRMIKEQLQQFSDVLDGVVLK | |
| | KLERRHYTGWGRLSAKLLMGIRDKQSHLTILDYLMNDDG | |
| | LNRNLMQLINDSNLSFKSIIEKEQVTTADKDIQSIVADLAGS | |
| | PAIKKGILQSLKIVDELVSVMGYPPQTIVVEMARENQTTGK | |
| | GKNNSRPRYKSLEKAIKEFGSQILKEHPTDNQELRNNRLYL | |
| | YYLQNGKDMYTGQDLDIHNLSNYDIDHIVPQSFITDNSIDN | |
| | LVLTSSAGNREKGDDVPPLEIVRKRKVFWEKLYQGNLMS | |
| | KRKFDYLTKAERGGLTEADKARFIHRQLVETRQITKNVAN | |
| | ILHQRFNYEKDDHGNTMKQVRIVTLKSALVSQFRKQFQLY | |
| 240 | KVRDVNDYHHAHDAYLNGVVANTLLKVYPQLEPEFVYG | Neisseria |
| | DYHQFDWFKANKATAKKQFYTNIMLFFAQKDRIIDENGEI | meningitidis Cas9 |
| | LWDKKYLDTVKKVMSYRQMNIVKKTEIQKGEFSKATIKP | |
| | KGNSSKLIPRKTNWDPMKYGGLDSPNMAYAVVIEYAKGK | |
| | NKLVFEKKIIRVTIMERKAFEKDEKAFLEEQGYRQPKVLAK | |
| | LPKYTLYECEEGRRRMLASANEAQKGNQQVLPNHLVTLL | |
| | HHAANCEVSDGKSLDYIESNREMFAELLAHVSEFAKRYTL | |
| | AEANLNKINQLFEQNKEGDIKAIAQSFVDLMAFNAMGAPA | |
| | SFKFFETTIERKRYNNLKELLNSTIIYQSITGLYESRKRLDD | |
| | MAAFKPNSINYILGLDIGIASVGWAMVEIDEEENPIRLIDLG | |
| | VRVFERAEVPKTGDSLAMARRLARSVRRLTRRRAHRLLRT | |
| | RRLLKREGVLQAANFDENGLIKSLPNTPWQLRAAALDRKL | |
| | TPLEWSAVLLHLIKHRGYLSQRKNEGETADKELGALLKGV | |
| | AGNAHALQTGDFRTPAELALNKFEKESGHIRNQRSDYSHT | |
| | FSRKDLQAELILLFEKQKEFGNPHVSGGLKEGIETLLMTQR | |
| | PALSGDAVQKMLGHCTFEPAEPKAAKNTYTAERFIWLTKL | |
| | NNLRILEQGSERPLTDTERATLMDEPYRKSKLTYAQARKL | |
| | LGLEDTAFFKGLRYGKDNAEASTLMEMKAYHAISRALEK | |
| | EGLKDKKSPLNLSPELQDEIGTAFSLFKTDEDITGRLKDRIQ | |
| | PEILEALLKHISFDKFVQISLKALRRIVPLMEQGKRYDEACA | |
| | EIYGDHYGKKNTEEKIYLPPIPADEIRNPVVLRALSQARKVI | |
| | NGVVRRYGSPARIHIETAREVGKSFKDRKEIEKRQEENRKD | |
| | REKAAAKFREYFPNFVGEPKSKDILKLRLYEQQHGKCLYS | |
| | GKEINLGRLNEKGYVEIDHALPFSRTWDDSFNNKVLVLGS | |
| | ENQNKGNQTPYEYFNGKDNSREWQEFKARVETSRFPRSK | |
| | KQRILLQKFDEDGFKERNLNDTRYVNRFLCQFVADRMRLT | |
| | GKGKKRVFASNGQITNLLRGFWGLRKVRAENDRHHALDA | |
| | VVVACSTVAMQQKITRFVRYKEMNAFDGKTIDKETGEVL | |
| | HQKTHFPQPWEFFAQEVMIRVFGKPDGKPEFEEADTLEKL | |
| | RTLLAEKLSSRPEAVHEYVTPLFVSRAPNRKMSGQGHMET | |
| | VKSAKRLDEGVSVLRVPLTQLKLKDLEKMVNREREPKLY | |
| | EALKARLEAHKDDPAKAFAEPFYKYDKAGNRTQQVKAV | |
| | RVEQVQKTGVWVRNHNGIADNATMVRVDVFEKGDKYYL | |
| | VPIYSWQVAKGILPDRAVVQGKDEEDWQLIDDSFNFKFSL | |
| | HPNDLVEVITKKARMFGYFASCHRGTGNINIRIHDLDHKIG | |
| | KNGILEGIGVKTALSFQKYQIDELGKEIRPCRLKKRPPVR | |
| 241 | MDKKYSIGLDIGTNSVGWAVITDEYKVPSKKFKVLGNTDR | Streptococcus |
| | HSIKKNLIGALLFDSGETAEATRLKRTARRRYTRRKNRICY | pyogenes Cas9 |
| | LQEIFSNEMAKVDDSFFHRLEESFLVEEDKKHERHPIFGNIV | |
| | DEVAYHEKYPTIYHLRKKLVDSTDKADLRLIYLALAHMIK | |
| | FRGHFLIEGDLNPDNSDVDKLFIQLVQTYNQLFEENPINAS | |
| | GVDAKAILSARLSKSRRLENLIAQLPGEKKNGLFGNLIALS | |
| | LGLTPNFKSNFDLAEDAKLQLSKDTYDDDLDNLLAQIGDQ | |
| | YADLFLAAKNLSDAILLSDILRVNTEITKAPLSASMIKRYDE | |
| | HHQDLTLLKALVRQQLPEKYKEIFFDQSKNGYAGYIDGGA | |
| | SQEEFYKFIKPILEKMDGTEELLVKLNREDLLRKQRTFDNG | |
| | SIPHQIHLGELHAILRRQEDFYPFLKDNREKIEKILTFRIPYY | |
| | VGPLARGNSRFAWMTRKSEETITPWNFEEVVDKGASAQSF | |
| | IERMTNFDKNLPNEKVLPKHSLLYEYFTVYNELTKVKYVT | |
| | EGMRKPAFLSGEQKKAIVDLLFKTNRKVTVKQLKEDYFK | |
| | KIECFDSVEISGVEDRFNASLGTYHDLLKIIKDKDFLDNEEN | |
| | EDILEDIVLTLTLFEDREMIEERLKTYAHLFDDKVMKQLKR | |
| | RRYTGWGRLSRKLINGIRDKQSGKTILDFLKSDGFANRNF | |
| | MQLIHDDSLTFKEDIQKAQVSGQGDSLHEHIANLAGSPAIK | |
| | KGILQTVKVVDELVKVMGRHKPENIVIEMARENQTTQKG | |
| | QKNSRERMKRIEEGIKELGSQILKEHPVENTQLQNEKLYLY | |
| | YLQNGRDMYVDQELDINRLSDYDVDHIVPQSFLKDDSIDN | |
| | KVLTRSDKNRGKSDNVPSEEVVKKMKNYWRQLLNAKLIT | |
| | QRKFDNLTKAERGGLSELDKAGFIKRQLVETRQITKHVAQI | |
| | LDSRMNTKYDENDKLIREVKVITLKSKLVSDFRKDFQFYK | |
| | VREINNYHHAHDAYLNAVVGTALIKKYPKLESEFVYGDY | |
| | KVYDVRKMIAKSEQEIGKATAKYFFYSNIMNFFKTEITLAN | |
| | GEIRKRPLIETNGETGEIVWDKGRDFATVRKVLSMPQVNIV | |
| | KKTEVQTGGFSKESILPKRNSDKLIARKKDWDPKKYGGFD | |
| | SPTVAYSVLVVAKVEKGKSKKLKSVKELLGITIMERSSFEK | |
| | NPIDFLEAKGYKEVKKDLIIKLPKYSLFELENGRKRMLASA | |
| | GELQKGNELALPSKYVNFLYLASHYEKLKGSPEDNEQKQL | |
| | FVEQHKHYLDEIIEQISEFSKRVILADANLDKVLSAYNKHR | |
| | DKPIREQAENIIHLFTLTNLGAPAAFKYFDTTIDRKRYTSTK | |
| | EVLDATLIHQSITGLYETRIDLSQLGGD | |
| 242 | MDKKYSIGLDIGTNSVGWAVITDEYKVPSKKFKVLGNTDR | S. pyogenes Cas9 |
| | HSIKKNLIGALLFDSGETAEATRLKRTARRRYTRRKNRICY | |
| | LQEIFSNEMAKVDDSFFHRLEESFLVEEDKKHERHPIFGNIV | |
| | DEVAYHEKYPTIYHLRKKLVDSTDKADLRLIYLALAHMIK | |
| | FRGHFLIEGDLNPDNSDVDKLFIQLVQTYNQLFEENPINAS | |
| | GVDAKAILSARLSKSRRLENLIAQLPGEKKNGLFGNLIALS | |
| | LGLTPNFKSNFDLAEDAKLQLSKDTYDDDLDNLLAQIGDQ | |
| | YADLFLAAKNLSDAILLSDILRVNTEITKAPLSASMIKRYDE | |
| | HHQDLTLLKALVRQQLPEKYKEIFFDQSKNGYAGYIDGGA | |
| | SQEEFYKFIKPILEKMDGTEELLVKLNREDLLRKQRTFDNG | |
| | SIPHQIHLGELHAILRRQEDFYPFLKDNREKIEKILTFRIPYY | |
| | VGPLARGNSRFAWMTRKSEETITPWNFEEVVDKGASAQSF | |
| | IERMTNFDKNLPNEKVLPKHSLLYEYFTVYNELTKVKYVT | |
| | EGMRKPAFLSGEQKKAIVDLLFKTNRKVTVKQLKEDYFK | |
| | KIECFDSVEISGVEDRFNASLGTYHDLLKIIKDKDFLDNEEN | |
| | EDILEDIVLTLTLFEDREMIEERLKTYAHLFDDKVMKQLKR | |
| | RRYTGWGRLSRKLINGIRDKQSGKTILDFLKSDGFANRNF | |
| | MQLIHDDSLTFKEDIQKAQVSGQGDSLHEHIANLAGSPAIK | |
| | KGILQTVKVVDELVKVMGRHKPENIVIEMARENQTTQKG | |
| | QKNSRERMKRIEEGIKELGSQILKEHPVENTQLQNEKLYLY | |
| | YLQNGRDMYVDQELDINRLSDYDVDHIVPQSFLKDDSIDN | |
| | KVLTRSDKNRGKSDNVPSEEVVKKMKNYWRQLLNAKLIT | |
| | QRKFDNLTKAERGGLSELDKAGFIKRQLVETRQITKHVAQI | |
| | LDSRMNTKYDENDKLIREVKVITLKSKLVSDFRKDFQFYK | |
| | VREINNYHHAHDAYLNAVVGTALIKKYPKLESEFVYGDY | |
| | KVYDVRKMIAKSEQEIGKATAKYFFYSNIMNFFKTEITLAN | |
| | GEIRKRPLIETNGETGEIVWDKGRDFATVRKVLSMPQVNIV | |
| | KKTEVQTGGFSKESILPKRNSDKLIARKKDWDPKKYGGFD | |
| | SPTVAYSVLVVAKVEKGKSKKLKSVKELLGITIMERSSFEK | |
| | NPIDFLEAKGYKEVKKDLIIKLPKYSLFELENGRKRMLASA | |
| | GELQKGNELALPSKYVNFLYLASHYEKLKGSPEDNEQKQL | |
| | FVEQHKHYLDEIIEQISEFSKRVILADANLDKVLSAYNKHR | |
| | DKPIREQAENIIHLFTLTNLGAPAAFKYFDTTIDRKRYTSTK | |
| | EVLDATLIHQSITGLYETRIDLSQLGGD | |
| 243 | MAAFKPNPINYILGLDIGIASVGWAMVEIDEDENPICLIDLG | N. meningitidis |
| | VRVFERAEVPKTGDSLAMARRLARSVRRLTRRRAHRLLR | Cas9 |
| | ARRLLKREGVLQAADFDENGLIKSLPNTPWQLRAAALDRK | |
| | LTPLEWSAVLLHLIKHRGYLSQRKNEGETADKELGALLKG | |
| | VADNAHALQTGDFRTPAELALNKFEKESGHIRNQRGDYSH | |
| | TFSRKDLQAELILLFEKQKEFGNPHVSGGLKEGIETLLMTQ | |
| | RPALSGDAVQKMLGHCTFEPAEPKAAKNTYTAERFIWLTK | |
| | LNNLRILEQGSERPLTDTERATLMDEPYRKSKLTYAQARK | |
| | LLGLEDTAFFKGLRYGKDNAEASTLMEMKAYHAISRALE | |
| | KEGLKDKKSPLNLSPELQDEIGTAFSLFKTDEDITGRLKDRI | |
| | QPEILEALLKHISFDKFVQISLKALRRIVPLMEQGKRYDEAC | |
| | AEIYGDHYGKKNTEEKIYLPPIPADEIRNPVVLRALSQARK | |
| | VINGVVRRYGSPARIHIETAREVGKSFKDRKEIEKRQEENR | |
| | KDREKAAAKFREYFPNFVGEPKSKDILKLRLYEQQHGKCL | |
| | YSGKEINLGRLNEKGYVEIDHALPFSRTWDDSFNNKVLVL | |
| | GSENQNKGNQTPYEYFNGKDNSREWQEFKARVETSRFPRS | |
| | KKQRILLQKFDEDGFKERNLNDTRYVNRFLCQFVADRMR | |
| | LTGKGKKRVFASNGQITNLLRGFWGLRKVRAENDRHHAL | |
| | DAVVVACSTVAMQQKITRFVRYKEMNAFDGKTIDKETGE | |
| | VLHQKTHFPQPWEFFAQEVMIRVFGKPDGKPEFEEADTPE | |
| | KLRTLLAEKLSSRPEAVHEYVTPLFVSRAPNRKMSGQGHM | |
| | ETVKSAKRLDEGVSVLRVPLTQLKLKDLEKMVNREREPKL | |
| | YEALKARLEAHKDDPAKAFAEPFYKYDKAGNRTQQVKA | |
| | VRVEQVQKTGVWVRNHNGIADNATMVRVDVFEKGDKYY | |
| | LVPIYSWQVAKGILPDRAVVQGKDEEDWQLIDDSFNFKFS | |
| | LHPNDLVEVITKKARMFGYFASCHRGTGNINIRIHDLDHKI | |
| | GKNGILEGIGVKTALSFQKYQIDELGKEIRPCRLKKRPPVR | |
| 244 | MKRNYILGLDIGITSVGYGIIDYETRDVIDAGVRLFKEANV | S. aureus Cas9 |
| | ENNEGRRSKRGARRLKRRRRHRIQRVKKLLFDYNLLTDHS | |
| | ELSGINPYEARVKGLSQKLSEEEFSAALLHLAKRRGVHNV | |
| | NEVEEDTGNELSTKEQISRNSKALEEKYVAELQLERLKKD | |
| | GEVRGSINRFKTSDYVKEAKQLLKVQKAYHQLDQSFIDTY | |
| | IDLLETRRTYYEGPGEGSPFGWKDIKEWYEMLMGHCTYFP | |
| | EELRSVKYAYNADLYNALNDLNNLVITRDENEKLEYYEKF | |
| | QIIENVFKQKKKPTLKQIAKEILVNEEDIKGYRVTSTGKPEF | |
| | TNLKVYHDIKDITARKEIIENAELLDQIAKILTIYQSSEDIQE | |
| | ELTNLNSELTQEEIEQISNLKGYTGTHNLSLKAINLILDELW | |
| | HTNDNQIAIFNRLKLVPKKVDLSQQKEIPTTLVDDFILSPVV | |
| | KRSFIQSIKVINAIIKKYGLPNDIIIELAREKNSKDAQKMINE | |
| | MQKRNRQTNERIEEIIRTTGKENAKYLIEKIKLHDMQEGKC | |
| | LYSLEAIPLEDLLNNPFNYEVDHIIPRSVSFDNSFNNKVLVK | |
| | QEENSKKGNRTPFQYLSSSDSKISYETFKKHILNLAKGKGR | |
| | ISKTKKEYLLEERDINRFSVQKDFINRNLVDTRYATRGLMN | |
| | LLRSYFRVNNLDVKVKSINGGFTSFLRRKWKFKKERNKGY | |
| | KHHAEDALIIANADFIFKEWKKLDKAKKVMENQMFEEKQ | |
| | AESMPEIETEQEYKEIFITPHQIKHIKDFKDYKYSHR VDKKP | |
| | NRELINDTLYSTRKDDKGNTLIVNNLNGLYDKDNDKLKKL | |
| | INKSPEKLLMYHHDPQTYQKLKLIMEQYGDEKNPLYKYYE | |
| | ETGNYLTKYSKKDNGPVIKKIKYYGNKLNAHLDITDDYPN | |
| | SRNKVVKLSLKPYRFDVYLDNGVYKFVTVKNLDVIKKEN | |
| | YYEVNSKCYEEAKKLKKISNQAEFIASFYNNDLIKINGELY | |
| | RVIGVNNDLLNRIEVNMIDITYREYLENMNDKRPPRIIKTIA | |
| | SKTQSIKKYSTDILGNLYEVKSKKHPQIIKKG | |
| 245 | gggaaatgagatcatgtcctaaccctgatcctcttgtcccacagatatccagaaccctgaccct | TRAC 5′ homology |
| | gccgtg | arm |
| 246 | taccagctgagagactctaaatccagtgacaagtctgtctgcctattcaccgattttg | TRAC 3′ homology |
| | | arm |
| 247 | ggatctgcga tcgctccggt gcccgtcagt gggcagagcg cacatcgccc | Human elongation |
| | acagtccccg agaagttggg gggaggggtc ggcaattgaa ccggtgccta | factor 1 alpha |
| | gagaaggtgg cgcggggtaa actgggaaag tgatgtcgtg tactggctcc gcctttttcc | (EF1a) promoter |
| | cgagggtggg ggagaaccgt atataagtgc agtagtcgcc gtgaacgttc tttttcgcaa | |
| | cgggtttgcc gccagaacac agctgaagct tcgaggggct cgcatctctc | |
| | cttcacgcgc ccgccgccct acctgaggcc gccatccacg ccggttgagt | |
| | cgcgttctgc cgcctcccgc ctgtggtgcc tcctgaactg cgtccgccgt ctaggtaagt | |
| | ttaaagctca ggtcgagacc gggcctttgt ccggcgctcc cttggagcct acctagactc | |
| | agccggctct ccacgctttg cctgaccctg cttgctcaac tctacgtctt tgtttcgttt | |
| | tctgttctgc gccgttacag atccaagctg tgaccggcgc ctac | |
| 248 | ctctatcaat gagagagcaa tctcctggta atgtgataga tttcccaact taatgccaac | TRAC 5′ |
| | ataccataaa cctcccattc tgctaatgcc cagcctaagt tggggagacc actccagatt | homology arm |
| | ccaagatgta cagtttgctt tgctgggcct ttttcccatg cctgccttta ctctgccaga | |
| | gttatattgc tggggttttg aagaagatcc tattaaataa aagaataagc agtattatta | |
| | agtagccctg catttcaggt ttccttgagt ggcaggccag gcctggccgt gaacgttcac | |
| | tgaaatcatg gcctcttggc caagattgat agcttgtgcc tgtccctgag tcccagtcca | |
| | tcacgagcag ctggtttcta agatgctatt tcccgtataa agcatgagac cgtgacttgc | |
| | cagccccaca gagccccgcc cttgtccatc actggcatct ggactccagc | |
| | ctgggttggg gcaaagaggg aaatgagatc atgtcctaac cctgatcctc ttgtcccaca | |
| | gatatccaga accctgaccc tgccgtgtac cagctgagag actctaaatc | |
| | cagtgacaag tctgtctgcc tattcaccga t | |
| 249 | tttgattctc aaacaaatgt gtcacaaagt aaggattctg atgtgtatat cacagacaaa | TRAC 3′ |
| | actgtgctag acatgaggtc tatggacttc aagagcaaca gtgctgtggc | homology arm |
| | ctggagcaac aaatctgact ttgcatgtgc aaacgccttc aacaacagca ttattccaga | |
| | agacaccttc ttccccagcc caggtaaggg cagctttggt gccttcgcag gctgtttcct | |
| | tgcttcagga atggccaggt tctgcccaga gctctggtca atgatgtcta aaactcctct | |
| | gattggtggt ctcggcctta tccattgcca ccaaaaccct ctttttacta agaaacagtg | |
| | agccttgttc tggcagtcca gagaatgaca cgggaaaaaa gcagatgaag | |
| | agaaggtggc aggagagggc acgtggccca gcctcagtct ctccaactga | |
| | gttcctgcct gcctgccttt gctcagactg tttgcccctt actgctcttc taggcctcat | |
| | tctaagcccc ttctccaagt tgcctctcct tatttctccc tgtctgccaa aaaatctttc | |
| | ccagctcact aagtcagtct cacgcagtca ctcattaacc caccaatcac tgattgtg | |
| 250 | attcaccgat tttgattctc | TRAC exon 1 target |
| | | site |
| 251 | tggctctctctctcagctggtacacgg | 5′ CTS |
| 252 | ccagcacctctctcagctggtacacgg | 3′ CTS |
| 253 | gggaaatgagatcatgtcctaaccctgatcctcttgtcccacagatatccagaaccctgaccct | TRAC 5′ homology |
| | gccgtgggaagcgga | arm |
| 254 | atccagaaccccgaccctgctgtttatcagctcagggattctaaatccagtgacaagtctgtct | TRAC 3′ homology |
| | gcctattcaccgattttg | arm |
| 255 | agagtctctcagctggtaca | TRAC gRNA |

1. A method for producing genetically engineered T cells, comprising:
(a) adding a whole blood sample comprising a plurality of T cells to a stationary phase in an internal cavity of a chromatography column, the stationary phase comprising a selection agent that specifically binds to a selection marker expressed on the surface of the plurality of T cells, wherein specific binding of the selection agent to the selection marker effects the immobilization of the plurality of T cells on the stationary phase;
(b) adding a T cell stimulatory reagent to the plurality of T cells immobilized on the stationary phase, wherein the T cell stimulatory reagent comprises (i) a primary agent that specifically binds to a member of a TCR/CD3 complex and (ii) a secondary agent that specifically binds to a T cell costimulatory molecule;
(c) incubating the plurality of T cells immobilized on the stationary phase in the presence of the T cell stimulatory reagent under conditions to stimulate T cells of the plurality of T cells;
(d) collecting T cells of the plurality of T cells from the chromatography column that are no longer immobilized after the incubating; and
(e) introducing by non-viral gene delivery a nucleic acid molecule comprising a transgene encoding a recombinant protein under conditions for targeted integration of the transgene into a target site of a gene in one or more of the collected T cells;
wherein the method produces genetically engineered T cells expressing the recombinant protein.

2. The method of claim 1, wherein the method comprises further incubating the collected T cells prior to the introducing of the nucleic acid molecule.

3. The method of claim 1 or claim 2, wherein the nucleic acid molecule is a DNA molecule, optionally a single-stranded DNA molecule or a double-stranded DNA molecule.

4. The method of any one of claims 1-3, wherein the targeted integration is by Programmable Addition via Site-specific Targeting Elements (PASTE).

5. The method of claim 4, wherein the PASTE comprises introducing one or more gene-editing agents for editing the gene in the one or more of the collected T cells.

6. The method of any one of claims 1-3, wherein the targeted integration is by homology directed repair (HDR).

7. The method of claim 6, wherein the HDR comprises introducing one or more gene-editing agents for inducing a genetic disruption in the gene in the one or more of the collected T cells.

8. The method of any one of claims 1-7, wherein the introducing of the nucleic acid molecule and/or the one or more gene-editing agents is by electroporation.

9. The method of any one of claims 1-8, wherein the conditions for targeted integration comprise cultivating the collected T cells under conditions to integrate the transgene into the target site.

10. A method for producing genetically engineered T cells, comprising:
(a) adding a whole blood sample comprising a plurality of T cells to a stationary phase in an internal cavity of a chromatography column, the stationary phase comprising a selection agent that specifically binds to a selection marker expressed on the surface of the plurality of T cells, wherein specific binding of the selection agent to the selection marker effects the immobilization of the plurality of T cells on the stationary phase;
(b) adding a T cell stimulatory reagent to the plurality of T cells immobilized on the stationary phase, wherein the T cell stimulatory reagent comprises (i) a primary agent that specifically binds to a member of a TCR/CD3 complex and (ii) a secondary agent that specifically binds to a T cell costimulatory molecule;
(c) incubating the plurality of T cells immobilized on the stationary phase in the presence of the T cell stimulatory reagent under conditions to stimulate T cells of the plurality of T cells;
(d) collecting T cells of the plurality of T cells from the chromatography column that are no longer immobilized after the incubating;
(e) further incubating the collected T cells;
(f) after the further incubating, introducing into T cells of the collected T cells (i) a nucleic acid molecule comprising a transgene encoding a recombinant protein, wherein the nucleic acid molecule is a DNA molecule, optionally a single-stranded DNA molecule or a double-stranded DNA molecule, and the introducing of the nucleic acid molecule is by non-viral gene delivery, and (ii) one or more gene-editing agents for inducing a genetic disruption in a gene in the T cells of the collected T cells, wherein the introducing of the nucleic acid molecule and/or the one or more gene-editing agents is by electroporation; and
(g) cultivating the collected T cells under conditions to integrate by homology directed repair (HDR) the transgene into a target site of the gene in one or more of the collected T cells;
wherein the method produces genetically engineered T cells expressing the recombinant protein.

11. The method of any one of claims 1-10, wherein the nucleic acid molecule is a double-stranded DNA molecule, a naked DNA molecule, and/or a closed-ended DNA molecule.

12. The method of any one of claims 1-11, wherein the nucleic acid molecule is a closed-ended linear duplex (CELiD) DNA molecule, a minicircle DNA molecule, a minimalistic immunological-defined gene expression (MIDGE) DNA molecule, a ministring DNA molecule, a dumbbell-shaped linear duplex closed-ended DNA molecule, or a Doggybone™ DNA molecule.

13. The method of any one of claims 1-12, wherein the T cell stimulatory reagent is added in a cell medium.

14. The method of any one of claims 1-13, wherein the T cell stimulatory reagent is added in an amount between or between about 0.1 μg and 20 μg, 0.4 μg and 8 μg, 0.8 μg and 4 μg, or 1 μg and 2 μg, each inclusive and each per 10⁶ T cells of the plurality of T cells immobilized or expected to be immobilized on the stationary phase.

15. The method of any one of claims 1-14, wherein the binding capacity of the stationary phase is between or between about 0.5 billion and 5 billion T cells expressing the selection marker, 0.5 billion and 3 billion T cells expressing the selection marker, or 1 billion and 2 billion T cells expressing the selection marker, each inclusive.

16. The method of any one of claims 1-15, wherein the T cell stimulatory reagent is added in an amount between or between about 0.1 mg and 20 mg, 0.4 mg and 8 mg, 0.8 mg and 4 mg, or 1 mg and 3 mg, each inclusive.

17. The method of any one of claims 1-16, wherein the adding of the T cell stimulatory reagent is carried out within or within about 60 minutes, 30 minutes, or 15 minutes after the adding of the whole blood sample.

18. The method of any one of claims 1-17, wherein the incubating is carried out in a cell medium.

19. The method of any one of claims 1-18, wherein the incubating is carried out at a temperature between or between about 35° C. and about 39° C.

20. The method of any one of claims 1-19, wherein the incubating is carried out for between or between about 0.5 hour and 8 hours, 2 hours and 6 hours, or 3 hours and 5 hours, each inclusive.

21. The method of any one of claims 1-20, wherein the collecting comprises adding a wash buffer to the stationary phase to collect the T cells of the plurality of T cells.

22. The method of claim 21, wherein the wash buffer is a cell medium.

23. The method of claim 21 or claim 22, wherein the wash buffer does not comprise a competition agent.

24. The method of any one of claims 1-23, wherein the collecting is carried out between or between about 0.5 hours and 8 hours, 2 hours and 6 hours, or 3 hours and 5 hours, each inclusive, after the adding of the T cell stimulatory reagent.

25. The method of any one of claims 2-24, wherein the further incubating is carried out in the presence of the T cell stimulatory reagent.

26. The method of any one of claims 2-25, wherein the further incubating is carried out in a cell medium.

27. The method of any one of claims 2-26, wherein the further incubating is carried out at a temperature between or between about 35° C. and about 39° C.

28. The method of any one of claims 2-27, wherein the further incubating is carried out for between or between about 10 hours and 30 hours, 16 hours and 24 hours, or 18 hours and 22 hours, each inclusive.

29. The method of any one of claims 5 and 7-28, wherein the method comprises removing the T cell stimulatory reagent from the collected T cells prior to the introducing of the one or more gene-editing agents.

30. The method of any one of claims 1-29, wherein the method comprises removing the T cell stimulatory reagent from the collected T cells prior to the introducing of the nucleic acid molecule.

31. The method of claim 29 or claim 30, wherein the removing is carried out after the further incubating.

32. The method of any one of claims 29-31, wherein the removing comprises washing the collected T cells.

33. The method of any one of claims 5 and 7-32, wherein:
the T cell stimulatory reagent comprises an oligomer of streptavidin or a streptavidin mutein molecule;
the primary agent comprises a first streptavidin-binding partner that is bound to a streptavidin or streptavidin mutein molecule of the oligomer;
the secondary agent comprises a second streptavidin-binding partner that is bound to a streptavidin or streptavidin mutein molecule of the oligomer; and
the method comprises disrupting the binding between the first and second streptavidin-binding partners and the streptavidin or streptavidin mutein molecules prior to the introducing of the one or more gene-editing agents.

34. The method of any one of claims 1-33, wherein:
the T cell stimulatory reagent comprises an oligomer of streptavidin or a streptavidin mutein molecule;
the primary agent comprises a first streptavidin-binding partner that is bound to a streptavidin or streptavidin mutein molecule of the oligomer;
the secondary agent comprises a second streptavidin-binding partner that is bound to a streptavidin or streptavidin mutein molecule of the oligomer; and
the method comprises disrupting the binding between the first and second streptavidin-binding partners and the streptavidin or streptavidin mutein molecules prior to the introducing of the nucleic acid molecule.

35. The method of claim 33 or claim 34, wherein the disrupting is carried out after the further incubating.

36. The method of any one of claims 33-35, wherein the disrupting is by adding a competition agent to the collected T cells that reverses the binding between the first and second streptavidin-binding partners and the streptavidin or streptavidin mutein molecules.

37. The method of any one of claims 23-36, wherein the competition agent is biotin.

38. The method of any one of claims 5 and 7-37, wherein the introducing of the one or more gene-editing agents is carried out prior to or concurrently with the introducing of the nucleic acid molecule.

39. The method of any one of claims 5 and 7-38, wherein the introducing of the one or more gene-editing agents is carried out between or between about 12 hours and 36 hours, 18 hours and 30 hours, or 22 hours and 26 hours, each inclusive, after the adding of the T cell stimulatory reagent.

40. The method of any one of claims 1-39, wherein the nucleic acid molecule is introduced in a cell medium comprising the nucleic acid molecule.

41. The method of any one of claims 1-40, wherein the introducing of the nucleic acid molecule is carried out between or between about 12 hours and 36 hours, 18 hours and 30 hours, or 22 hours and 26 hours, each inclusive, after the adding of the T cell stimulatory reagent.

42. The method of any one of claims 9-41, wherein the cultivating is carried out in the presence of the nucleic acid molecule.

43. The method of any one of claims 9-42, wherein the cultivating is carried out in a cell medium.

44. The method of any one of claims 9-43, wherein the cultivating is carried out at a temperature between or between about 35° C. and about 39° C.

45. The method of any one of claims 9-44, wherein the cultivating is carried out for between or between about 12 hours and 36 hours, 18 hours and 30 hours, or 22 hours and 26 hours, each inclusive.

46. The method of any one of claims 13-45, wherein the cell medium is a basal medium.

47. The method of any one of claims 13-46, wherein the cell medium is a serum free medium.

48. The method of any one of claims 13-47, wherein the cell medium comprises no cytokines or comprises recombinant IL-2, IL-7, and IL-15.

49. The method of any one of claims 1-48, wherein the method comprises harvesting the genetically engineered T cells expressing the recombinant protein.

50. The method of claim 49, wherein the harvesting is carried out between or between about 36 hours and 60 hours, 42 hours and 54 hours, or 46 hours and 50 hours, each inclusive, after the adding of the whole blood sample.

51. The method of claim 49 or claim 50, wherein the harvesting is carried out between or between about 36 hours and 60 hours, 42 hours and 54 hours, or 46 hours and 50 hours, each inclusive, after the adding of the T cell stimulatory reagent.

52. The method of any one of claims 49-51, wherein the harvesting is carried out between or between about 12 hours and 36 hours, 18 hours and 30 hours, or 22 hours and 26 hours, each inclusive, after the introducing of the one or more gene-editing agents.

53. The method of any one of claims 49-52, wherein the harvesting is carried out between or between about 12 hours and 36 hours, 18 hours and 30 hours, or 22 hours and 26 hours, each inclusive, after the introducing of the nucleic acid molecule.

54. The method of any one of claims 49-53, wherein the method comprises formulating the harvested genetically engineered T cells for cryopreservation or administration to a subject.

55. The method of claim 54, wherein the harvested genetically engineered T cells are formulated in the presence of a cryoprotectant or a pharmaceutically acceptable excipient.

56. The method of any one of claims 1-55, wherein the plurality of T cells are primary T cells from a human subject.

57. The method of any one of claims 1-56, wherein the selection marker is selected from the group consisting of CD3, CD4, CD8, CD45RA, CD27, CD28, and CCR7.

58. The method of any one of claims 1-57, wherein the selection marker is CD3, CD4, or CD8.

59. The method of any one of claims 1-58, wherein the selection marker is CD3.

60. The method of any one of claims 1-59, wherein the selection agent comprises an antibody or antibody fragment that specifically binds to the selection marker.

61. The method of claim 60, wherein the antibody or antibody fragment of the selection agent is a monovalent antibody fragment.

62. The method of claim 60 or claim 61, wherein the antibody or antibody fragment of the selection agent is a Fab fragment.

63. The method of any one of claims 1-32 and 37-62, wherein:
the T cell stimulatory reagent comprises an oligomer of streptavidin or a streptavidin mutein molecule;
the primary agent comprises a first streptavidin-binding partner that is bound to a streptavidin or streptavidin mutein molecule of the oligomer; and
the secondary agent comprises a second streptavidin-binding partner that is bound to a streptavidin or streptavidin mutein molecule of the oligomer.

64. The method of any one of claims 33-63, wherein the T cell stimulatory reagent consists or consists essentially of the oligomer, primary agent, and secondary agent.

65. The method of any one of claims 33-64, wherein the oligomer comprises between or between about 500 and 5000 tetramers, 1000 and 4000 tetramers, or 2000 and 3000 tetramers, each inclusive, of the streptavidin or streptavidin mutein molecule.

66. The method of any one of claims 33-65, wherein the oligomer is of the streptavidin mutein molecule.

67. The method of any one of claims 33-66, wherein the streptavidin mutein molecule comprises the amino acid sequence IGAR (SEQ ID NO: 133) or VTAR (SEQ ID NO: 134) at sequence positions corresponding to positions 44 to 47 of the sequence of amino acids set forth in SEQ ID NO: 1.

68. The method of any one of claims 33-67, wherein the streptavidin mutein molecule begins N-terminally in the region of amino acid positions 10 to 16 of SEQ ID NO: 1 and terminates C-terminally in the region of amino acid positions 133 to 142 of SEQ ID NO: 1.

69. The method of any one of claims 33-68, wherein the streptavidin mutein molecule comprises the amino acid sequence set forth in any one of SEQ ID NO: 3-6, 27, 28, 104, 105, and 136.

70. The method of any one of claims 33-69, wherein the streptavidin mutein molecule comprises the amino acid sequence set forth in SEQ ID NO: 6.

71. The method of any one of claims 33-70, wherein:
the first streptavidin-binding partner is at the C-terminus of the primary agent; and/or
the second streptavidin-binding partner is at the C-terminus of the secondary agent.

72. The method of any one of claims 33-71, wherein the first and/or second streptavidin-binding partner is a streptavidin-binding peptide.

73. The method of claim 72, wherein the streptavidin-binding peptide of the first and/or second streptavidin-binding partner comprises the amino acid sequence set forth in any one of SEQ ID NO: 7, 8, and 15-19.

74. The method of claim 72 or claim 73, wherein the streptavidin-binding peptide of the first and/or second streptavidin-binding partner comprises the amino acid sequence set forth in SEQ ID NO: 16.

75. The method of any one of claims 1-74, wherein the member of the TCR/CD3 complex is CD3.

76. The method of any one of claims 1-75, wherein the T cell costimulatory molecule is CD28, CD90 (Thy-1), CD95 (Apo-/Fas), CD137 (4-1BB), CD154 (CD40L), ICOS, LAT, CD27, OX40, or HVEM.

77. The method of any one of claims 1-76, wherein the T cell costimulatory molecule is CD28.

78. The method of any one of claims 1-77, wherein:
the primary agent comprises an antibody or antibody fragment that specifically binds to the member of the TCR/CD3 complex; and/or
the secondary agent comprises an antibody or antibody fragment that specifically binds to the T cell costimulatory agent.

79. The method of claim 78, wherein:
the antibody or antibody fragment of the primary agent comprises a heavy chain, and the first streptavidin-binding partner is fused to the C-terminus of the heavy chain of the primary agent; and/or
the antibody or antibody fragment of the secondary agent comprises a heavy chain, and the second streptavidin-binding partner is fused to the C-terminus of the heavy chain of the secondary agent.

80. The method of claim 78 or claim 79, wherein the antibody or antibody fragment of the primary and/or secondary agent is a monovalent antibody fragment.

81. The method of any one of claims 78-80, wherein the antibody or antibody fragment of the primary and/or secondary agent is a Fab fragment.

82. The method of any one of claims 1-81, wherein the primary agent comprises an anti-CD3 antibody or antibody fragment, and the secondary agent comprises an anti-CD28 antibody or antibody fragment.

83. The method of any one of claims 1-82, wherein the primary agent comprises an anti-CD3 Fab fragment, and the secondary agent comprises an anti-CD28 Fab fragment.

84. The method of any one of claims 1-83, wherein the gene is the T cell receptor alpha constant (TRAC) gene.

85. The method of any one of claims 1-84, wherein the target site is within the sequence set forth in SEQ ID NO: 250.

86. The method of any one of claims 1-3 and 6-85, wherein the nucleic acid molecule comprises a 5′ homology arm and a 3′ homology arm comprising sequences homologous to nucleic acid sequences surrounding the target site, the nucleic acid molecule comprising the structure [5′ homology arm]-[transgene]-[3′ homology arm].

87. The method of claim 86, wherein the 5′ homology arm and the 3′ homology arm comprise sequences homologous to sequences of the TRAC gene surrounding the target site.

88. The method of claim 86 or claim 87, wherein the 5′ homology arm comprises a sequence comprising at least or at least about 150, 200, 250, 300, 350, 400, 450, 500, 550, or 600 contiguous nucleotides of a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 248.

89. The method of any one of claims 86-88, wherein the 5′ homology arm comprises at least or at least about 150, 200, 250, 300, 350, 400, 450, 500, 550, or 600 contiguous nucleotides of the sequence set forth in SEQ ID NO: 248.

90. The method of any one of claims 86-89, wherein the 5′ homology arm comprises the sequence set forth in SEQ ID NO: 248.

91. The method of any one of claims 86-90, wherein the 3′ homology arm comprises a sequence comprising at least or at least about 150, 200, 250, 300, 350, 400, 450, 500, 550, or 600 contiguous nucleotides of a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 249.

92. The method of any one of claims 86-91, wherein the 3′ homology arm comprises at least or at least about 150, 200, 250, 300, 350, 400, 450, 500, 550, or 600 contiguous nucleotides of the sequence set forth in SEQ ID NO: 249.

93. The method of any one of claims 86-92, wherein the 3′ homology arm comprises the sequence set forth in SEQ ID NO: 249.

94. The method of any one of claims 1-93, wherein transcription of the integrated transgene is under the control of a promoter comprised by the nucleic acid molecule.

95. The method of claim 94, wherein the promoter is a human elongation factor 1 alpha (EF1α) promoter.

96. The method of claim 94 or claim 95, wherein the promoter comprises the sequence set forth in SEQ ID NO: 247.

97. The method of any one of claims 1-96, wherein the recombinant protein is a recombinant receptor.

98. The method of claim 97, wherein the recombinant receptor is a T cell receptor or a chimeric antigen receptor.

99. The method of any one of claims 5 and 7-98, wherein the one or more gene-editing agents comprise (i) a gene-editing nuclease or nuclease combination or (ii) a nucleic acid molecule comprising one or more sequences encoding the gene-editing nuclease or nuclease combination.

100. The method of any one of claims 5 and 7-99, wherein the one or more gene-editing agents comprise a gene-editing nuclease or nuclease combination.

101. The method of claim 99 or claim 100, wherein the gene-editing nuclease or nuclease combination specifically recognizes a nucleic acid sequence near or comprising the target site.

102. The method of any one of claims 99-101, wherein the gene-editing nuclease or nuclease combination specifically recognizes a nucleic acid sequence comprising the target site.

103. The method of claim 101 or claim 102, wherein the nucleic acid sequence comprising the target site comprises the sequence set forth in SEQ ID NO: 250.

104. The method of any one of claims 99-103, wherein the gene-editing nuclease or nuclease combination is a zinc finger nuclease, a transcription activator-like effector nuclease, or a CRISPR-Cas combination.

105. The method of any one of claims 99-104, wherein the gene-editing nuclease or nuclease combination is a CRISPR-Cas combination.

106. The method of claim 104 or claim 105, wherein the CRISPR-Cas combination comprises a CRISPR-Cas nickase, reverse transcriptase, and serine integrase.

107. The method of claim 105 or claim 106, wherein the CRISPR-Cas combination comprises a guide RNA comprising a targeting sequence that is complementary to the nucleic acid sequence comprising the target site.

108. The method of claim 105 or claim 107, wherein the CRISPR-Cas combination is a ribonucleoprotein complex comprising the guide RNA and a Cas protein.

109. The method of claim 108, wherein the Cas protein is a S. pyogenes Cas protein.

110. The method of any one of claims 104-109, wherein the CRISPR-Cas combination is a CRISPR-Cas9 combination or a CRISPR-Cas12 combination.

111. The method of any one of claims 107-110, wherein the targeting sequence comprises the sequence set forth in any one of SEQ ID NO: 144-175.

112. The method of any one of claims 107-111, wherein the targeting sequence comprises the sequence set forth in SEQ ID NO: 148.

113. A genetically engineered T cell produced by the method of any one of claims 1-112, wherein the genetically engineered T cell expresses the recombinant protein.

114. The genetically engineered T cell of claim 113, wherein the transgene is integrated into the target site of the gene in the genetically engineered T cell.

115. The genetically engineered T cell of claim 114, wherein the gene is the T cell receptor alpha constant (TRAC) gene.

116. The genetically engineered T cell of claim 114 or claim 115, wherein the target site is within the sequence set forth in SEQ ID NO: 250.

117. The genetically engineered T cell of any one of claims 113-116, wherein the recombinant protein is a recombinant receptor.

118. The genetically engineered T cell of claim 117, wherein the recombinant receptor is a T cell receptor or a chimeric antigen receptor.

119. A population of T cells comprising a plurality of the genetically engineered T cell of any one of claims 113-118.

120. The population of claim 119, wherein the plurality of genetically engineered T cells are at least 10%, 15%, or 20% of the population of T cells.

121. The population of claim 119 or claim 120, wherein the gene is disrupted in at least 85%, 90%, or 95% of the T cells of the population of T cells.

122. The population of claim 121, wherein the gene is the T cell receptor alpha constant (TRAC) gene.

123. A pharmaceutical composition comprising the population of T cells of any one of claims 119-122 and a pharmaceutically acceptable excipient.

124. A method of treatment, comprising administering to a subject having a disease or condition the pharmaceutical composition of claim 123.

125. The method of claim 124, wherein the recombinant protein is a recombinant receptor that targets an antigen expressed on a target cell associated with the disease or condition.

126. A method of cytolytic killing of a target cell, comprising contacting a target cell with the population of any one of claims 119-122.

127. A method of cytolytic killing of a target cell, comprising contacting a target cell with the pharmaceutical composition of claim 123.

128. The method of claim 126 or claim 127, wherein the contacting is performed ex vivo.

129. The method of claim 126 or claim 127, wherein the contacting is performed in vivo.

130. The method of claim 129, wherein the contacting is by administering the pharmaceutical composition to a subject having a disease or condition.

131. The method of claim 130, wherein the target cell is associated with the disease or condition, and the recombinant protein is a recombinant receptor that targets an antigen expressed on the target cell.

132. The method of claim 125 or claim 131, wherein the recombinant receptor is a T cell receptor or a chimeric antigen receptor.

133. The pharmaceutical composition of claim 123 for use in treating a disease or disorder in a subject.

134. The pharmaceutical composition of claim 133, wherein the recombinant protein is a recombinant receptor that targets an antigen expressed on a cell associated with the disease or condition.

135. The pharmaceutical composition of claim 134, wherein the recombinant receptor is a T cell receptor or a chimeric antigen receptor.

136. Use of the pharmaceutical composition of claim 123 for treating a disease or disorder in a subject.

137. Use of the pharmaceutical composition of claim 123 for the manufacture of a medicament for treating a disease or disorder in a subject.

138. The use of claim 136 or claim 137, wherein the recombinant protein is a recombinant receptor that targets an antigen expressed on a cell associated with the disease or condition.

139. The use of claim 138, wherein the recombinant receptor is a T cell receptor or a chimeric antigen receptor.


SEQUENCE LISTING (255 sequences)

SEQ ID NO: 1 - protein, 159 aa, Streptomyces avidinii
  REGION 1..159: note = Streptavidin (UniProt No. P22629)
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 2 - protein, 127 aa, synthetic construct
  REGION 1..127: note = Minimal streptavidin with initial methionine
MEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 3 - protein, 159 aa, synthetic construct
  REGION 1..159: note = Mutein Streptavidin Val44-Thr45-Ala46-Arg47
DPSKDSKAQVSAAEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 4 - protein, 127 aa, synthetic construct
  REGION 1..127: note = Minimal mutein Streptavidin Val44-Thr45-Ala46-Arg47 with initial methionine
MEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 5 - protein, 159 aa, synthetic construct
  REGION 1..159: note = Mutein Streptavidin Ile44-Gly45-Ala46-Arg47
DPSKDSKAQVSAAEAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 6 - protein, 127 aa, synthetic construct
  REGION 1..127: note = Minimal mutein Streptavidin Ile44-Gly45-Ala46-Arg47 with initial methionine
MEAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 7 - protein, 8 aa, synthetic construct
  REGION 1..8: note = Streptavidin-binding peptide, Strep-tag(R)
WRHPQFGG

SEQ ID NO: 8 - protein, 8 aa, synthetic construct
  REGION 1..8: note = Strep-tag(R) II
WSHPQFEK

SEQ ID NO: 9 - skipped

SEQ ID NO: 10 - protein, 4 aa, synthetic construct
  REGION 1..4: note = Streptavidin-binding peptide
HPQF

SEQ ID NO: 11 - protein, 8 aa, synthetic construct
  REGION 1..8: note = Streptavidin-binding peptide
  VARIANT 1: note = Xaa = Trp, Lys or Arg
  VARIANT 2: note = Xaa = Any Amino Acid
  VARIANT 7: note = Xaa = Gly or GLu
  VARIANT 8: note = Xaa = Gly, Lys or Arg
XXHPQFXX

SEQ ID NO: 12 - protein, 8 aa, synthetic construct
  REGION 1..8: note = Streptavidin-binding peptide
  VARIANT 2: note = Xaa = Any Amino Acid
  VARIANT 7: note = Xaa = any amino acid
  VARIANT 8: note = Xaa = any amino acid
WXHPQFXX

SEQ ID NO: 13 - protein, 28 aa, synthetic construct
  REGION 1..17: note = Sequential modules of streptavidin-binding peptide
  VARIANT 9: note = Xaa = Any Amino Acid
  VARIANT 17..20: note = may be present or absent
WSHPQFEKXXXXXXXXXXXXWSHPQFEK

SEQ ID NO: 14 - protein, 28 aa, synthetic construct
  REGION 1..20: note = Sequential modules of streptavidin-binding peptide
  VARIANT 13..20: note = may be present or absent
WSHPQFEKGGGSGGGSGGGSWSHPQFEK

SEQ ID NO: 15 - protein, 30 aa, synthetic construct
  REGION 1..30: note = Twin-Strep-tag
SAWSHPQFEKGGGSGGGSGGGSWSHPQFEK

SEQ ID NO: 16 - protein, 30 aa, synthetic construct
  REGION 1..30: note = Twin-Strep-tag
SAWSHPQFEKGGGSGGGSGGSAWSHPQFEK

SEQ ID NO: 17 - protein, 28 aa, synthetic construct
  REGION 1..28: note = Twin-Strep-tag
WSHPQFEKGGGSGGGSGGGSWSHPQFEK

SEQ ID NO: 18 - protein, 24 aa, synthetic construct
  REGION 1..24: note = Twin-Strep-tag
WSHPQFEKGGGSGGGSWSHPQFEK

SEQ ID NO: 19 - protein, 28 aa, synthetic construct
  REGION 1..28: note = Twin-Strep-tag
WSHPQFEKGGGSGGGSGGSAWSHPQFEK

SEQ ID NO: 20 - protein, 9 aa, synthetic construct
  REGION 1..9: note = HA-tag
YPYDVPDYA

SEQ ID NO: 21 - protein, 11 aa, synthetic construct
  REGION 1..11: note = VSV-G-tag
YTDIEMNRLGK

SEQ ID NO: 22 - protein, 11 aa, synthetic construct
  REGION 1..11: note = HSV-tag
QPELAPEDPED

SEQ ID NO: 23 - protein, 10 aa, synthetic construct
  REGION 1..10: note = T7 epitope
ASMTGGQQMG

SEQ ID NO: 24 - protein, 11 aa, synthetic construct
  REGION 1..11: note = HSV epitope
QPELAPEDPED

SEQ ID NO: 25 - protein, 10 aa, synthetic construct
  REGION 1..10: note = Myc epitope
EQKLISEEDL

SEQ ID NO: 26 - protein, 14 aa, synthetic construct
  REGION 1..14: note = V5-tag
GKPIPNPLLGLDST

SEQ ID NO: 27 - protein, 126 aa, synthetic construct
  REGION 1..126: note = Minimal mutein Streptavidin Val44-Thr45-Ala46-Arg47 and Glu117, Gly120, Tyr121 (mutein m1-9) without initial methionine
EAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEENAGYSTLVGHDTFTKVKPSAAS

SEQ ID NO: 28 - protein, 139 aa, synthetic construct
  REGION 1..139: note = Mutein Streptavidin Val44-Thr45-Ala46-Arg47 and Glu117, Gly120, Tyr121 (mutein m1-9)
DPSKDSKAQVSAAEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEENAGYSTLVGHDTFTKVKPSAAS

SEQ ID NO: 29 - protein, 253 aa, synthetic construct
  REGION 1..253: note = Variable Heavy chain of Fab fragment m13B8.2
AMQVQLKQSGPGLVQPSQSLSITCTVSGFSLTTFGVHWVRQSPGKGLEWLGVIWASGITDYNVPFMSRLSITKDNSKSQVFFKLNSLQPDDTAIYYCAKNDPGTGFAYWGQGTLVTVSAGSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGSAWSHPQFEKGGGSGGGSGGSAWSHPQFEK

SEQ ID NO: 30 - protein, 218 aa, synthetic construct
  REGION 1..218: note = Variable Light chain of Fab Fragment m13B8.2
AMDIQMTQSPASLSASVGETVTFTCRASEMIYSYLAWYQQKQGKSPQLLVHDAKTLAEGVPSRFSGGGSGTQFSLKINTLQPEDFGTYYCQAHYGNPPTFGGGTKLEIKRGIAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGS

SEQ ID NO: 31 - protein, 119 aa, synthetic construct [sequence omitted]

SEQ ID NO: 32 - protein, 106 aa, synthetic construct [sequence omitted]

SEQ ID NO: 33 - protein, 116 aa, synthetic construct
  REGION 1..116: note = Variable Heavy chain of anti-CD28 antibody CD28.3
LQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTV

SEQ ID NO: 34 - protein, 108 aa, synthetic construct
  REGION 1..108: note = Variable Light chain of anti-CD28 antibody CD28.3
DIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKR

SEQ ID NO: 35 - protein, 11 aa, synthetic construct
  REGION 1..11: note = MAT tag
HNHRHKHGGGC

SEQ ID NO: 36 - protein, 118 aa, synthetic construct
  REGION 1..118: note = Variable Heavy chain of huOKT8
QVQLVQSGAEVKKPGATVKISCKVSGFNIKDTYIHWVQQAPGKGLEWMGRIDPANDNTLYASKFQGRVTITADTSTDTAYMELSSLRSEDTAVYYCARGYGYYVFDHWGQGTLVTVSS

SEQ ID NO: 37 - protein, 107 aa, synthetic construct
  REGION 1..107: note = Variable Light chain of huOKT8
DVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIK

SEQ ID NO: 38 - protein, 30 aa, synthetic construct
  REGION 1..30: note = Linker
PGGGSGGGGSGGGGSGGGGSGGGGSGGGGP

SEQ ID NO: 39 - protein, 17 aa, synthetic construct
  REGION 1..17: note = Linker
GSADDAKKDAAKKDGKS

SEQ ID NO: 40 - protein, 22 aa, synthetic construct
  REGION 1..22: note = GMCSFR alpha chain
MLLLVTSLLLCELPHPAFLLIP

SEQ ID NO: 41 - DNA, 66 bp, synthetic construct
  misc_feature 1..66: note = GMCSFR alpha chain
  source 1..66: mol_type = other DNA
atgcttctcctggtgacaagccttctgctctgtgagttaccacacccagcattcctcctgatccca

SEQ ID NO: 42 - protein, 18 aa, synthetic construct
  REGION 1..18: note = CD8 alpha signal peptide
MALPVTALLLPLALLLHA

SEQ ID NO: 43 - protein, 357 aa, synthetic construct
  REGION 1..357: note = truncated epidermal growth factor receptor (tEGFR)
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 44 - protein, 335 aa, synthetic construct
  REGION 1..335: note = truncated epidermal growth factor receptor (tEGFR)
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 45 - protein, 22 aa, synthetic construct
  REGION 1..22: note = F2A
VKQTLNFDLLKLAGDVESNPGP

SEQ ID NO: 46 - protein, 20 aa, synthetic construct
  REGION 1..20: note = E2A
QCTNYALLKLAGDVESNPGP

SEQ ID NO: 47 - protein, 24 aa, synthetic construct
  REGION 1..24: note = T2A
LEGGGEGRGSLLTCGDVEENPGPR

SEQ ID NO: 48 - protein, 18 aa, synthetic construct
  REGION 1..18: note = T2A
EGRGSLLTCGDVEENPGP

SEQ ID NO: 49 - protein, 22 aa, synthetic construct
  REGION 1..22: note = P2A
GSGATNFSLLKQAGDVEENPGP

SEQ ID NO: 50 - protein, 19 aa, synthetic construct
  REGION 1..19: note = P2A
ATNFSLLKQAGDVEENPGP

SEQ ID NO: 51 - protein, 5 aa, synthetic construct
  REGION 1..5: note = CDR H1
DYGVS

SEQ ID NO: 52 - protein, 16 aa, synthetic construct
  REGION 1..16: note = CDR H2
VIWGSETTYYNSALKS

SEQ ID NO: 53 - protein, 7 aa, synthetic construct
  REGION 1..7: note = CDR H3
YAMDYWG

SEQ ID NO: 54 - protein, 12 aa, synthetic construct
  REGION 1..12: note = CDR H3
HYYYGGSYAMDY

SEQ ID NO: 55 - protein, 11 aa, synthetic construct
  REGION 1..11: note = CDR L1
RASQDISKYLN

SEQ ID NO: 56 - protein, 7 aa, synthetic construct
  REGION 1..7: note = CDR L2
SRLHSGV

SEQ ID NO: 57 - protein, 7 aa, synthetic construct
  REGION 1..7: note = CDR L2
HTSRLHS

SEQ ID NO: 58 - protein, 9 aa, synthetic construct
  REGION 1..9: note = CDR L3
GNTLPYTFG

SEQ ID NO: 59 - protein, 9 aa, synthetic construct
  REGION 1..9: note = CDR L3
QQGNTLPYT

SEQ ID NO: 60 - protein, 120 aa, synthetic construct
  REGION 1..120: note = VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 61 - protein, 107 aa, synthetic construct
  REGION 1..107: note = VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 62 - protein, 18 aa, synthetic construct
  REGION 1..18: note = Linker
GSTSGSGKPGSGEGSTKG

SEQ ID NO: 63 - DNA, 735 bp, synthetic construct
  misc_feature 1..735: note = scFv
  source 1..735: mol_type = other DNA
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 64 - protein, 245 aa, synthetic construct
  REGION 1..245: note = scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 65 - protein, 5 aa, synthetic construct
  REGION 1..5: note = CDR H1
SYWMN

SEQ ID NO: 66 - protein, 17 aa, synthetic construct
  REGION 1..17: note = CDR H2
QIYPGDGDTNYNGKFKG

SEQ ID NO: 67 - protein, 13 aa, synthetic construct
  REGION 1..13: note = CDR H3
KTISSVVDFYFDY

SEQ ID NO: 68 - protein, 9 aa, synthetic construct
  REGION 1..9: note = CDR L3
QQYNRYPYT

SEQ ID NO: 69 - protein, 5 aa, synthetic construct
  REGION 1..5: note = CDR H1
SYWMN

SEQ ID NO: 70 - protein, 17 aa, synthetic construct
  REGION 1..17: note = CDR H2
QIYPGDGDTNYNGKFKG

SEQ ID NO: 71 - protein, 122 aa, synthetic construct
  REGION 1..122: note = VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 72 - protein, 108 aa, synthetic construct
  REGION 1..108: note = VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 73 - protein, 11 aa, synthetic construct
  REGION 1..11: note = CDR L1
KASQNVGTNVA

SEQ ID NO: 74 - protein, 7 aa, synthetic construct
  REGION 1..7: note = CDR L2
SATYRNS

SEQ ID NO: 75 - protein, 9 aa, synthetic construct
  REGION 1..9: note = CDR L3
QQYNRYPYT

SEQ ID NO: 76 - protein, 5 aa, synthetic construct
  REGION 1..5: note = CDR H1
SYWMN

SEQ ID NO: 77 - protein, 17 aa, synthetic construct
  REGION 1..17: note = CDR H2
QIYPGDGDTNYNGKFKG

SEQ ID NO: 78 - protein, 13 aa, synthetic construct
  REGION 1..13: note = CDR H3
KTISSVVDFYFDY

SEQ ID NO: 79 - protein, 15 aa, synthetic construct
  REGION 1..15: note = Linker
GGGGSGGGGSGGGGS

SEQ ID NO: 80 - protein, 245 aa, synthetic construct
  REGION 1..245: note = scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 81 - protein, 12 aa, Homo sapiens
  REGION 1..12: note = spacer (IgG4hinge)
ESKYGPPCPPCP

SEQ ID NO: 82 - DNA, 36 bp, Homo sapiens
  misc_feature 1..36: note = spacer (IgG4hinge)
  source 1..36: mol_type = unassigned DNA
gaatctaagtacggaccgccctgccccccttgccct

SEQ ID NO: 83 - protein, 119 aa, Homo sapiens
  REGION 1..119: note = Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 229 aa, Homo sapiens
  REGION 1..229: note = Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 282 aa, Homo sapiens
  REGION 1..282: note = IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 86 - protein, 14 aa, synthetic construct
  REGION 1..14: note = Exemplary IgG Hinge
EVVVKYGPPCPPCP

SEQ ID NO: 87 - skipped

SEQ ID NO: 88 - protein, 15 aa, synthetic construct
  REGION 1..15: note = Exemplary IgG Hinge
EPKSCDKTHTCPPCP

SEQ ID NO: 89 - protein, 12 aa, synthetic construct
  REGION 1..12: note = Exemplary IgG Hinge
ERKCCVECPPCP

SEQ ID NO: 90 - protein, 61 aa, synthetic construct
  REGION 1..61: note = Exemplary IgG Hinge
ELKTPLGDTHTCPRCPEPKSCDTPPPCPRCPEPKSCDTPPPCPRCPEPKSCDTPPPCPRCP

SEQ ID NO: 91 - protein, 12 aa, synthetic construct
  REGION 1..12: note = Exemplary IgG Hinge
ESKYGPPCPSCP

SEQ ID NO: 92 - protein, 12 aa, synthetic construct
  REGION 1..12: note = Exemplary IgG Hinge
ESKYGPPCPPCP

SEQ ID NO: 93 - protein, 9 aa, synthetic construct
  REGION 1..9: note = Exemplary IgG Hinge
YGPPCPPCP

SEQ ID NO: 94 - protein, 10 aa, synthetic construct
  REGION 1..10: note = Exemplary IgG Hinge
KYGPPCPPCP

SEQ ID NO: 95 - protein, 27 aa, Homo sapiens
  REGION 1..27: note = CD28 (amino acids 153-179 of Accession No. P10747)
FWVLVVVGGVLACYSLLVTVAFIIFWV

SEQ ID NO: 96 - protein, 66 aa, Homo sapiens
  REGION 1..66: note = CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 97 - protein, 41 aa, Homo sapiens
  REGION 1..41: note = CD28 (amino acids 180-220 of P10747)
RSKRSRLLHSDYMNMTPRRPGPTRKHYQPYAPPRDFAAYRS

SEQ ID NO: 98 - protein, 41 aa, Homo sapiens
  REGION 1..41: note = CD28 (LL to GG)
RSKRSRGGHSDYMNMTPRRPGPTRKHYQPYAPPRDFAAYRS

SEQ ID NO: 99 - protein, 42 aa, Homo sapiens
  REGION 1..42: note = 4-1BB (amino acids 214-255 of Q07011.1)
KRGRKKLLYIFKQPFMRPVQTTQEEDGCSCRFPEEEEGGCEL

SEQ ID NO: 100 - protein, 112 aa, Homo sapiens
  REGION 1..112: note = CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 112 aa, Homo sapiens
  REGION 1..112: note = CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 112 aa, Homo sapiens
  REGION 1..112: note = CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 126 aa, synthetic construct
  REGION 1..126: note = Minimal streptavidin without initial methionine
EAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 104 - protein, 126 aa, synthetic construct
  REGION 1..126: note = Minimal mutein Streptavidin Val44-Thr45-Ala46-Arg47 without initial methionine
EAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 105 - protein, 126 aa, synthetic construct
  REGION 1..126: note = Minimal mutein Streptavidin Ile44-Gly45-Ala-46-Arg47 without initial methionine
EAGITGTWYNQLGSTFIVTAGADGALTGTYIGARGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 106 - protein, 326 aa, Homo sapiens
  REGION 1..326: note = Human IgG2 Fc (Uniprot P01859)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 107 - protein, 327 aa, Homo sapiens
  REGION 1..327: note = Human IgG4 Fc (Uniprot P01861)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 108 - protein, 107 aa, synthetic construct
  REGION 1..107: note = FKBP
GVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLE

SEQ ID NO: 109 - protein, 107 aa, synthetic construct
  REGION 1..107: note = FKBP12v36
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 110 - protein, 16 aa, synthetic construct
  REGION 1..16: note = Modified acylation motif
MGSNKSKPKDASQRRR

SEQ ID NO: 111 - protein, 5 aa, synthetic construct
  REGION 1..5: note = Dual acylation motif
  VARIANT 4: note = Xaa = Any Amino Acid
MGCXC

SEQ ID NO: 112 - skipped

SEQ ID NO: 113 - protein, 5 aa, synthetic construct
  REGION 1..5: note = CDR-H1
DYSIN

SEQ ID NO: 114 - protein, 17 aa, synthetic construct
  REGION 1..17: note = CDR-H2
WINTETREPAYAYDFRG

SEQ ID NO: 115 - protein, 8 aa, synthetic construct
  REGION 1..8: note = CDR-H3
DYSYAMDY

SEQ ID NO: 116 - protein, 15 aa, synthetic construct
  REGION 1..15: note = CDR-L1
RASESVTILGSHLIH

SEQ ID NO: 117 - protein, 7 aa, synthetic construct
  REGION 1..7: note = CDR-L2
LASNVQT

SEQ ID NO: 118 - protein, 9 aa, synthetic construct
  REGION 1..9: note = CDR-L3
LQSRTIPRT

SEQ ID NO: 119 - protein, 117 aa, synthetic construct
  REGION 1..117: note = Variable heavy (VH) Anti-BCMA
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 120 - protein, 111 aa, synthetic construct
  REGION 1..111: note = Variable light (VL) Anti-BCMA
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 121 - protein, 246 aa, synthetic construct
  REGION 1..246: note = scFv
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 122 - protein, 472 aa, synthetic construct
  REGION 1..472: note = anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 5 aa, synthetic construct
  REGION 1..5: note = CDR-H1
DYYVY

SEQ ID NO: 124 - protein, 17 aa, synthetic construct
  REGION 1..17: note = CDR-H2
WINPNSGGTNYAQKFQG

SEQ ID NO: 125 - protein, 9 aa, synthetic construct
  REGION 1..9: note = CDR-H3
SQRDGYMDY

SEQ ID NO: 126 - protein, 8 aa, synthetic construct
  REGION 1..8: note = CDR-L1
TGTSSDVG

SEQ ID NO: 127 - protein, 7 aa, synthetic construct
  REGION 1..7: note = CDR-L2
EDSKRPS

SEQ ID NO: 128 - protein, 10 aa, synthetic construct
  REGION 1..10: note = CDR-L3
SSNTRSSTLV

SEQ ID NO: 129 - protein, 118 aa, synthetic construct
  REGION 1..118: note = Variable heavy (VH) Anti-BCMA
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 130 - protein, 105 aa, synthetic construct
  REGION 1..105: note = Variable light (VL) Anti-BCMA
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 131 - protein, 244 aa, synthetic construct
  REGION 1..244: note = scFv
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 132 - protein, 654 aa, synthetic construct
  REGION 1..654: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 4 aa, synthetic construct
  REGION 1..4: note = Sequence of mutein streptavidin at sequence positions corresponding to positions 44 to 47 of streptavidin
IGAR

SEQ ID NO: 134 - protein, 4 aa, synthetic construct
  REGION 1..4: note = Sequence of mutein streptavidin at sequence positions corresponding to positions 44 to 47 of streptavidin
VTAR

SEQ ID NO: 135 - protein, 127 aa, synthetic construct
  REGION 1..127: note = Minimal streptavidin
AEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 136 - protein, 127 aa, synthetic construct
  REGION 1..127: note = Mutein Streptavidin Val44-Thr45-Ala46-Arg47 and Glu117, Gly120, Tyr121 (mutein m1-9) with initial methionine
MEAGITGTWYNQLGSTFIVTAGADGALTGTYVTARGNAESRYVLTGRYDSAPATDGSGTALGWTVAWKNNYRNAHSATTWSGQYVGGAEARINTQWLLTSGTTEENAGYSTLVGHDTFTKVKPSAAS

SEQ ID NO: 137 - protein, 142 aa, synthetic construct
  REGION 1..142: note = Human TCR alpha constant (Uniprot P01848)
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 138 - protein, 141 aa, synthetic construct
  REGION 1..141: note = Human TCR alpha constant (Genbank Accession No. CAA26636.1)
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 139 - DNA, 1508 bp, synthetic construct
  misc_feature 1..1508: note = Human TCR alpha constant (GenBank: X02592.1)
  source 1..1508: mol_type = other DNA
ttttgaaacccttcaaaggcagagacttgtccagcctaacctgcctgctgctcctagctcctgaggctcagggcccttggcttctgtccgctctgctcagggccctccagcgtggccactgctcagccatgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtgatctcgaaccgaacagcagtgcttccaagataatctttggatcagggaccagactcagcatccggccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagatctgcaagattgtaagacagcctgtgctccctcgctccttcctctgcattgcccctcttctccctctccaaacagagggaactctcctacccccaaggaggtgaaagctgctaccacctctgtgcccccccggtaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctggagaatgttgtgaggggtttatttttttttaatagtgttcataaagaaatacatagtattcttcttctcaagacgtggggggaaattatctcattatcgaggccctgctatgctgtgtgtctgggcgtgttgtatgtcctgctgccgatgccttcattaaaatgatttggaa

SEQ ID NO: 140 - protein, 177 aa, synthetic construct
  REGION 1..177: note = Human TCR beta constant 1 (Uniprot P01850)
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 141 - protein, 178 aa, synthetic construct
  REGION 1..178: note = Human TCR beta constant 2 (Uniprot A0A5B9)
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 142 - protein, 179 aa, synthetic construct
  REGION 1..179: note = Human TCR beta constant (Uniprot Accession No. A0A0G2JNG9)
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 143 - DNA, 1151 bp, synthetic construct
  misc_feature 1..1151: note = Human TCR beta constant (GenBank: X00437.1)
  source 1..1151: mol_type = other DNA
ctggtctagaatattccacatctgctctcactctgccatggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttctcgacctgttcggctaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaaggcagccctggaagtggagttaggagcttctaacccgtcatggttcaatacacattcttcttttgccagcgcttctgaagagctgctctcacctctctgcatcccaatagatatccccctatgtgcatgcacacctgcacactcacggctgaaatctccctaacccagggggac

SEQ ID NO: 144 - RNA, 20 nt, synthetic construct
  misc_feature 1..20: note = TRAC gRNA
  source 1..20: mol_type = other RNA
agagtctctcagctggtaca

SEQ ID NO: 145 - RNA, 20 nt, synthetic construct
  misc_feature 1..20: note = TRAC-10
  source 1..20: mol_type = other RNA
tctctcagctggtacacggc

SEQ ID NO: 146 - RNA, 20 nt, synthetic construct
  misc_feature 1..20: note = TRAC-110
  source 1..20: mol_type = other RNA
tggatttagagtctctcagc

SEQ ID NO: 147 - RNA, 20 nt, synthetic construct
  misc_feature 1..20: note = TRAC-116
  source 1..20: mol_type = other RNA
acacggcagggtcagggttc

SEQ ID NO: 148 - RNA, 20 nt, synthetic construct
  misc_feature 1..20: note = TRAC-16
  source 1..20: mol_type = other RNA
gagaatcaaaatcggtgaat

SEQ ID NO: 149 - RNA, 20 nt, synthetic construct
  misc_feature 1..20: note = TRAC-4
  source 1..20: mol_type = other RNA
gctggtacacggcagggtca

SEQ ID NO: 150 - RNA, 17 nt, synthetic construct
  misc_feature 1..17: note = TRAC-49
  source 1..17: mol_type = other RNA
ctcagctggtacacggc

SEQ ID NO: 151 - RNA, 17 nt, synthetic construct
  misc_feature 1..17: note = TRAC-2
  source 1..17: mol_type = other RNA
tggtacacggcagggtc

SEQ ID NO: 152 - RNA, 17 nt, synthetic construct
  misc_feature 1..17: note = TRAC-30
  source 1..17: mol_type = other RNA
gctagacatgaggtcta

SEQ ID NO: 153 - RNA, 17 nt, synthetic construct
  misc_feature 1..17: note = TRAC-43
  source 1..17: mol_type = other RNA
gtcagatttgttgctcc

SEQ ID NO: 154 - RNA, 17 nt, synthetic construct
  misc_feature 1..17: note = TRAC-23
  source 1..17: mol_type = other RNA
tcagctggtacacggca

SEQ ID NO: 155 - RNA, 17 nt, synthetic construct
  misc_feature 1..17: note = TRAC-34
  source 1..17: mol_type = other RNA
gcagacagacttgtcac

SEQ ID NO: 156 - RNA, 17 nt, synthetic construct
  misc_feature 1..17: note = TRAC-25
  source 1..17: mol_type = other RNA
ggtacacggcagggtca

SEQ ID NO: 157 - RNA, 20 nt, synthetic construct
  misc_feature 1..20: note = TRAC-128
  source 1..20: mol_type = other RNA
cttcaagagcaacagtgctg

SEQ ID NO: 158 - RNA, 20 nt, synthetic construct
  misc_feature 1..20: note = TRAC-105
  source 1..20: mol_type = other RNA
agagcaacagtgctgtggcc

SEQ ID NO: 159 - RNA, 20 nt, synthetic construct
  misc_feature 1..20: note = TRAC-106
  source 1..20: mol_type = other RNA
aaagtcagatttgttgctcc

SEQ ID NO: 160 - RNA, 20 nt, synthetic construct
  misc_feature 1..20: note = TRAC-123
  source 1..20: mol_type = other RNA
acaaaactgtgctagacatg

SEQ ID NO: 161 - RNA, 17 nt, synthetic construct
  misc_feature 1..17: note = TRAC-64
  source 1..17: mol_type = other RNA
aaactgtgctagacatg

SEQ ID NO: 162 - RNA, 20 nt, synthetic construct
  misc_feature 1..20: note = TRAC-97
  source 1..20: mol_type = other RNA
tgtgctagacatgaggtcta

SEQ ID NO: 163 - RNA, 22 nt, synthetic construct
  misc_feature 1..22: note = TRAC-148
  source 1..22: mol_type = other RNA
ggctggggaagaaggtgtcttc

SEQ ID NO: 164 - RNA, 21 nt, synthetic construct
  misc_feature 1..21: note = TRAC-147
  source 1..21: mol_type = other RNA
gctggggaagaaggtgtcttc

SEQ ID NO: 165 - RNA, 18 nt, synthetic construct
  misc_feature 1..18: note = TRAC-234
  source 1..18: mol_type = other RNA
ggggaagaaggtgtcttc

SEQ ID NO: 166 - RNA, 22 nt, synthetic construct
  misc_feature 1..22: note = TRAC-167
  source 1..22: mol_type = other RNA
gttttgtctgtgatatacacat

SEQ ID NO: 167 - RNA, 24 nt, synthetic construct
  misc_feature 1..24: note = TRAC-177
  source 1..24: mol_type = other RNA
ggcagacagacttgtcactggatt

SEQ ID NO: 168 - RNA, 23 nt, synthetic construct
  misc_feature 1..23: note = TRAC-176
  source 1..23: mol_type = other RNA
gcagacagacttgtcactggatt

SEQ ID NO: 169 - RNA, 20 nt, synthetic construct
  misc_feature 1..20: note = TRAC-257
  source 1..20: mol_type = other RNA
gacagacttgtcactggatt

SEQ ID NO: 170 - RNA, 24 nt, synthetic construct
  misc_feature 1..24: note = TRAC-233
  source 1..24: mol_type = other RNA
gtgaataggcagacagacttgtca

SEQ ID NO: 171 - RNA, 22 nt, synthetic construct
  misc_feature 1..22: note = TRAC-231
  source 1..22: mol_type = other RNA
gaataggcagacagacttgtca

SEQ ID NO: 172 - RNA, 22 nt, synthetic construct
  misc_feature 1..22: note = TRAC-163
  source 1..22: mol_type = other RNA
gagtctctcagctggtacacgg

SEQ ID NO: 173 - RNA, 20 nt, synthetic construct
  misc_feature 1..20: note = TRAC-241
  source 1..20: mol_type = other RNA
gtctctcagctggtacacgg

SEQ ID NO: 174 - RNA, 22 nt, synthetic construct
  misc_feature 1..22: note = TRAC-179
  source 1..22: mol_type = other RNA
ggtacacggcagggtcagggtt

SEQ ID NO: 175 - RNA, 21 nt, synthetic construct
  misc_feature 1..21: note = TRAC-178
  source 1..21: mol_type = other RNA
gtacacggcagggtcagggtt

SEQ ID NO: 176 - RNA, 20 nt, synthetic construct
  misc_feature 1..20: note = TRBC-40
  source 1..20: mol_type = other RNA
cacccagatcgtcagcgccg

SEQ ID NO: 177 - RNA, 20 nt, synthetic construct
  misc_feature 1..20: note = TRBC-52
  source 1..20: mol_type = other RNA
caaacacagcgacctcgggt

SEQ ID NO: 178 - RNA, 20 nt, synthetic construct
  misc_feature 1..20: note = TRBC-25
  source 1..20: mol_type = other RNA
tgacgagtggacccaggata

SEQ ID NO: 179 - RNA, 20 nt, synthetic construct
  misc_feature 1..20: note = TRBC-35
  source 1..20: mol_type = other RNA
ggctctcggagaatgacgag

SEQ ID NO: 180 - RNA, 20 nt, synthetic construct
  misc_feature 1..20: note = TRBC-50
  source 1..20: mol_type = other RNA
ggcctcggcgctgacgatct

SEQ ID NO: 181 - RNA, 20 nt, synthetic construct
  misc_feature 1..20: note = TRBC-39
  source 1..20: mol_type = other RNA
gaaaaacgtgttcccacccg

SEQ ID NO: 182 - RNA, 20 nt, synthetic construct
  misc_feature 1..20: note = TRBC-49
  source 1..20: mol_type = other RNA
atgacgagtggacccaggat

SEQ ID NO: 183 - RNA, 20 nt, synthetic construct
  misc_feature 1..20: note = TRBC-51
  source 1..20: mol_type = other RNA
agtccagttctacgggctct

SEQ ID NO: 184 - RNA, 20 nt, synthetic construct
  misc_feature 1..20: note = TRBC-26
  source 1..20: mol_type = other RNA
cgctgtcaagtccagttcta

SEQ ID NO: 185 - RNA, 20 nt, synthetic construct
  misc_feature 1..20: note = TRBC-47
  source 1..20: mol_type = other RNA
atcgtcagcgccgaggcctg

SEQ ID NO: 186 - RNA, 20 nt, synthetic construct
  misc_feature 1..20: note = TRBC-45
  source 1..20: mol_type = other RNA
tcaaacacagcgacctcggg

SEQ ID NO: 187 - RNA, 20 nt, synthetic construct
  misc_feature 1..20: note = TRBC-34
  source 1..20: mol_type = other RNA
cgtagaactggacttgacag

SEQ ID NO: 188 - RNA, 20 nt, synthetic construct
  misc_feature 1..20: note = TRBC-227
  source 1..20: mol_type = other RNA
aggcctcggcgctgacgatc

SEQ ID NO: 189 - RNA, 20 nt, synthetic construct
  misc_feature 1..20: note = TRBC-41
  source 1..20: mol_type = other RNA
tgacagcggaagtggttgcg

SEQ ID NO: 190 - RNA, 20 nt, synthetic construct
  misc_feature 1..20: note = TRBC-30
  source 1..20: mol_type = other RNA
ttgacagcggaagtggttgc

SEQ ID NO: 191 - RNA, 20 nt, synthetic construct
  misc_feature 1..20: note = TRBC-206
  source 1..20: mol_type = other RNA
tctccgagagcccgtagaac

SEQ ID NO: 192 - RNA, 20 nt, synthetic construct
  misc_feature 1..20: note = TRBC-32
  source 1..20: mol_type = other RNA
cgggtgggaacacgtttttc

SEQ ID NO: 193 - RNA, 20 nt, synthetic construct
  misc_feature 1..20: note = TRBC-276
  source 1..20: mol_type = other RNA
gacaggtttggccctatcct

SEQ ID NO: 194 - RNA, 20 nt, synthetic construct
  misc_feature 1..20: note = TRBC-274
  source 1..20: mol_type = other RNA
gatcgtcagcgccgaggcct

SEQ ID NO: 195 - RNA, 20 nt, synthetic construct
  misc_feature 1..20: note = TRBC-230
  source 1..20: mol_type = other RNA
ggctcaaacacagcgacctc

SEQ ID NO: 196 - RNA, 20 nt, synthetic construct
  misc_feature 1..20: note = TRBC-235
  source 1..20: mol_type = other RNA
tgagggtctcggccaccttc

SEQ ID NO: 197 - RNA, 20 nt, synthetic construct
  misc_feature 1..20: note = TRBC-38
  source 1..20: mol_type = other RNA
aggcttctaccccgaccacg

SEQ ID NO: 198 - RNA, 20 nt, synthetic construct
  misc_feature 1..20: note = TRBC-223
  source 1..20: mol_type = other RNA
ccgaccacgtggagctgagc

SEQ ID NO: 199 - RNA, 20 nt, synthetic construct
  misc_feature 1..20: note = TRBC-221
  source 1..20: mol_type = other RNA
tgacaggtttggccctatcc

SEQ ID NO: 200 - RNA, 20 nt, synthetic construct
  misc_feature 1..20: note = TRBC-48
  source 1..20: mol_type = other RNA
cttgacagcggaagtggttg

SEQ ID NO: 201 - RNA, 20 nt, synthetic construct
  misc_feature 1..20: note = TRBC-216
  source 1..20: mol_type = other RNA
agatcgtcagcgccgaggcc

SEQ ID NO: 202 - RNA, 20 nt, synthetic construct
  misc_feature 1..20: note = TRBC-210
  source 1..20: mol_type = other RNA
gcgctgacgatctgggtgac

SEQ ID NO: 203 - RNA, 20 nt, synthetic construct
  misc_feature 1..20: note = TRBC-268
  source 1..20: mol_type = other RNA
tgagggcgggctgctccttg

SEQ ID NO: 204 - RNA, 20 nt, synthetic construct
  misc_feature 1..20: note = TRBC-193
  source 1..20: mol_type = other RNA
gttgcgggggttctgccaga

SEQ ID NO: 205 - RNA, 20 nt, synthetic construct
  misc_feature 1..20: note = TRBC-246
  source 1..20: mol_type = other RNA
agctcagctccacgtggtcg

SEQ ID NO: 206 - RNA, 20 nt, synthetic construct
  misc_feature 1..20: note = TRBC-228
  source 1..20: mol_type = other RNA
gcggctgctcaggcagtatc

SEQ ID NO: 207 - RNA, 20 nt, synthetic construct
  misc_feature 1..20: note = TRBC-43
  source 1..20: mol_type = other RNA
gcgggggttctgccagaagg

SEQ ID NO: 208 - RNA, 20 nt, synthetic construct
  misc_feature 1..20: note = TRBC-272
  source 1..20: mol_type = other RNA
tggctcaaacacagcgacct

SEQ ID NO: 209 - RNA, 20 nt, synthetic construct
  misc_feature 1..20: note = TRBC-33
  source 1..20: mol_type = other RNA
actggacttgacagcggaag

SEQ ID NO: 210 - RNA, 20 nt, synthetic construct
  misc_feature 1..20: note = TRBC-44
  source 1..20: mol_type = other RNA
gacagcggaagtggttgcgg

SEQ ID NO: 211 - RNA, 20 nt, synthetic construct
  misc_feature 1..20: note = TRBC-211
  source 1..20: mol_type = other RNA
gctgtcaagtccagttctac

SEQ ID NO: 212 - RNA, 20 nt, synthetic construct
  misc_feature 1..20: note = TRBC-253
  source 1..20: mol_type = other RNA
gtatctggagtcattgaggg

SEQ ID NO: 213 - RNA, 17 nt, synthetic construct
  misc_feature 1..17: note = TRBC-18
  source 1..17: mol_type = other RNA
ctcggcgctgacgatct

SEQ ID NO: 214 - RNA, 17 nt, synthetic construct
  misc_feature 1..17: note = TRBC-6
  source 1..17: mol_type = other RNA
cctcggcgctgacgatc

SEQ ID NO: 215 - RNA, 17 nt, synthetic construct
  misc_feature 1..17: note = TRBC-85
  source 1..17: mol_type = other RNA
ccgagagcccgtagaac

SEQ ID NO: 216 - RNA, 17 nt, synthetic construct
  misc_feature 1..17: note = TRBC-129
  source 1..17: mol_type = other RNA
ccagatcgtcagcgccg

SEQ ID NO: 217 - RNA, 17 nt, synthetic construct
  misc_feature 1..17: note = TRBC-93
  source 1..17: mol_type = other RNA
gaatgacgagtggaccc

SEQ ID NO: 218 - RNA, 22 nt, synthetic construct
  misc_feature 1..22: note = TRBC-415
  source 1..22: mol_type = other RNA
gggtgacaggtttggccctatc

SEQ ID NO: 219 - RNA, 21 nt, synthetic construct
  misc_feature 1..21: note = TRBC-414
  source 1..21: mol_type = other RNA
ggtgacaggtttggccctatc

SEQ ID NO: 220 - RNA, 20 nt, synthetic construct
  misc_feature 1..20: note = TRBC-310
  source 1..20: mol_type = other RNA
gtgacaggtttggccctatc

SEQ ID NO: 221 - RNA, 18 nt, synthetic construct
  misc_feature 1..18: note = TRBC-308
  source 1..18: mol_type = other RNA
gacaggtttggccctatc

SEQ ID NO: 222 - RNA, 22 nt, synthetic construct
  misc_feature 1..22: note = TRBC-401
  source 1..22: mol_type = other RNA
gatactgcctgagcagccgcct

SEQ ID NO: 223 - RNA, 24 nt, synthetic construct
  misc_feature 1..24: note = TRBC-468
  source 1..24: mol_type = other RNA
gaccacgtggagctgagctggtgg

SEQ ID NO: 224 - RNA, 18 nt, synthetic construct
  misc_feature 1..18: note = TRBC-462
  source 1..18: mol_type = other RNA
gtggagctgagctggtgg

SEQ ID NO: 225 - RNA, 24 nt, synthetic construct
  misc_feature 1..24: note = TRBC-424
  source 1..24: mol_type = other RNA
gggcgggctgctccttgaggggct

SEQ ID NO: 226 - RNA, 23 nt, synthetic construct
  misc_feature 1..23: note = TRBC-423
  source 1..23: mol_type = other RNA
ggcgggctgctccttgaggggct

SEQ ID NO: 227 - RNA, 22 nt, synthetic construct
  misc_feature 1..22: note = TRBC-422
  source 1..22: mol_type = other RNA
gcgggctgctccttgaggggct

SEQ ID NO: 228 - RNA, 20 nt, synthetic construct
  misc_feature 1..20: note = TRBC-420
  source 1..20: mol_type = other RNA
gggctgctccttgaggggct

SEQ ID NO: 229 - RNA, 19 nt, synthetic construct
  misc_feature 1..19: note = TRBC-419
  source 1..19: mol_type = other RNA
ggctgctccttgaggggct

SEQ ID NO: 230 - RNA, 18 nt, synthetic construct
  misc_feature 1..18: note = TRBC-418
  source 1..18: mol_type = other RNA
gctgctccttgaggggct

SEQ ID NO: 231 - RNA, 24 nt, synthetic construct
  misc_feature 1..24: note = TRBC-445
  source 1..24: mol_type = other RNA
ggtgaatgggaaggaggtgcacag

SEQ ID NO: 232 - RNA, 23 nt, synthetic construct
  misc_feature 1..23: note = TRBC-444
  source 1..23: mol_type = other RNA
gtgaatgggaaggaggtgcacag

SEQ ID NO: 233 - RNA, 21 nt, synthetic construct
  misc_feature 1..21: note = TRBC-442
  source 1..21: mol_type = other RNA
gaatgggaaggaggtgcacag

SEQ ID NO: 234 - DNA, 149 bp, synthetic construct
  misc_feature 1..149: note = TRAC gRNA
  source 1..149: mol_type = other DNA
agcgctctcgtacagagttggcattataatacgactcactataggggagaatcaaaatcggtgaatgttttagagctagaaatagcaagttaaaataaggctagtccgttatcaacttgaaaaagtggcaccgagtcggtgcttttttt

SEQ ID NO: 235 - RNA, 100 nt, synthetic construct
  misc_feature 1..100: note = TRAC gRNA
  source 1..100: mol_type = other RNA
gagaatcaaaatcggtgaatgttttagagctagaaatagcaagttaaaataaggctagtccgttatcaacttgaaaaagtggcaccgagtcggtgctttt

SEQ ID NO: 236 - protein, 1344 aa, Streptococcus mutans
  REGION 1..1344: note = Streptococcus mutans Cas9
KKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 237 - protein, 1367 aa, Streptococcus pyogenes
  REGION 1..1367: note = Streptococcus pyogenes Cas9
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 238 - protein, 1387 aa, Streptococcus thermophilus
  REGION 1..1387: note = Streptococcus thermophilus Cas9
TKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 239 - protein, 1333 aa, Listeria innocua
  REGION 1..1333: note = Listeria innocua Cas9
KKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 240 - protein, 1082 aa, Neisseria meningitidis
  REGION 1..1082: note = Neisseria meningitidis Cas9
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 241 - protein, 1368 aa, Streptococcus pyogenes [sequence omitted]

SEQ ID NO: 242 - protein, 1368 aa, Streptococcus pyogenes [sequence omitted]

SEQ ID NO: 243 - protein, 1082 aa, Neisseria meningitidis
  REGION 1..1082: note = N. meningitidis Cas9
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 244 - protein, 1053 aa, Staphylococcus aureus
  REGION 1..1053: note = S. aureus Cas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 245 - DNA, 70 bp, synthetic construct
  misc_feature 1..70: note = TRAC 5' homology arm
  source 1..70: mol_type = other DNA
gggaaatgagatcatgtcctaaccctgatcctcttgtcccacagatatccagaaccctgaccctgccgtg

SEQ ID NO: 246 - DNA, 58 bp, synthetic construct
  misc_feature 1..58: note = TRAC 3' homology arm
  source 1..58: mol_type = other DNA
taccagctgagagactctaaatccagtgacaagtctgtctgcctattcaccgattttg

SEQ ID NO: 247 - DNA, 544 bp, Homo sapiens
  misc_feature 1..544: note = Human elongation factor 1 alpha (EF1-alpha) promoter
  source 1..544: mol_type = unassigned DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 248 - DNA, 611 bp, synthetic construct
  misc_feature 1..611: note = TRAC 5 homology arm
  source 1..611: mol_type = other DNA
ctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 249 - DNA, 628 bp, synthetic construct
  misc_feature 1..628: note = TRAC 3 homology arm
  source 1..628: mol_type = other DNA
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtg

SEQ ID NO: 250 - DNA, 20 bp, synthetic construct
  misc_feature 1..20: note = TRAC exon 1 target site
  source 1..20: mol_type = other DNA
attcaccgattttgattctc

SEQ ID NO: 251 - DNA, 27 bp, synthetic construct
  misc_feature 1..27: note = 5' CTS
  source 1..27: mol_type = other DNA
tggctctctctctcagctggtacacgg

SEQ ID NO: 252 - DNA, 27 bp, synthetic construct
  misc_feature 1..27: note = 3' CTS
  source 1..27: mol_type = other DNA
ccagcacctctctcagctggtacacgg

SEQ ID NO: 253 - DNA, 79 bp, synthetic construct
  misc_feature 1..79: note = TRAC 5' homology arm
  source 1..79: mol_type = other DNA
gggaaatgagatcatgtcctaaccctgatcctcttgtcccacagatatccagaaccctgaccctgccgtgggaagcgga

SEQ ID NO: 254 - DNA, 82 bp, synthetic construct
  misc_feature 1..82: note = TRAC 3' homology arm
  source 1..82: mol_type = other DNA
atccagaaccccgaccctgctgtttatcagctcagggattctaaatccagtgacaagtctgtctgcctattcaccgattttg

SEQ ID NO: 255 - DNA, 20 bp, synthetic construct
  misc_feature 1..20: note = TRAC gRNA
  source 1..20: mol_type = other DNA
agagtctctcagctggtaca